(19) United States
(12) Patent Application Publication
(10) Pub. No.: US 2006/0116368 A1
(43) Pub. Date: Jun. 1, 2006

(54) 4-Piperidinecarboxamide modulators of vanilloid VR1 receptor

(76) Inventors: Raul R. Calvo, Royersford, PA (US); Wing S. Cheung, Plainsboro, NJ (US); Mark R. Player, Phoenixville, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/288,624

(22) Filed: Nov. 29, 2005

Related U.S. Application Data
(60) Provisional application No. 60/631,436, filed on Nov. 29, 2004.
(60) Provisional application No. 60/712,496, filed on Aug. 30, 2005.

(51) Int. Cl. C07D 279/16; C07D 265/36; A61K 31/5415; A61K 31/538

(52) U.S. Cl. 514/224.2; 544/105; 544/52; 514/230.5

(57) ABSTRACT

This invention is directed to vanilloid receptor VR1 ligands. More particularly, this invention relates to hetero isonipecotic amides that are potent modulators of VR1 which are useful for the treatment and prevention of disease conditions in mammals.

### CROSS-REFERENCE TO RELATED APPLICATIONS

This application claims priority to U.S. Provisional Application Nos. 60/631,436, filed Nov. 29, 2004, 60/732,035, filed Nov. 1, 2005 and 60/712,496 filed Aug. 30, 2005, all of which are hereby incorporated by reference in its entirety.

### STATEMENT REGARDING FEDERALLY SPONSORED RESEARCH OR DEVELOPMENT

The research and development of the invention described below was not federally sponsored.

### BACKGROUND OF THE INVENTION

This invention is directed to novel vanilloid receptor VR1 ligands. More particularly, this invention relates to novel 4-piperidincarboxamides that are potent modulators of VR1.

Noxious chemical, thermal and mechanical stimuli excite peripheral nerve endings of small diameter sensory neurons (nociceptors) in sensory ganglia (e.g., dorsal root, nodose and trigeminal ganglia) and initiate signals that are perceived as pain. These neurons are crucial for the detection of harmful or potentially harmful stimuli (heat) and tissue damage (local tissue acidosis and/or stretch) that arise from changes in the extracellular space during inflammatory or ischaemic conditions (Wall, P. D., and Meizack, R., Textbook of Pain, 1994, New York: Churchill Livingstone). Nociceptors transduce noxious stimuli into membrane depolarization that triggers action potential, conducts the action potential from the sensory sites to the synapses in the CNS, and conversion of action potentials invokes a perception of pain, discomfort, and appropriate mechanical/physical protective reflexes. At the molecular level, nociception is carried out by ion channels or receptors. Plant derived vanilloid compounds (capsaicin and its ultrapotent analog, resiniferatoxin, etc.) are known to selectively depolarize nociceptors and elicit sensations of burning pain—the sensation that is typically obtained by hot chili peppers. Therefore, capsaicin mimics the action of physiological/endogenous stimuli that activates the “nociceptive pathway”. Recent advances in pain biology have identified receptors for vanilloids, protons (i.e., acidic solutions), and for heat. Because nociceptors are involved with unwanted pain and inflammatory conditions in human beings and animals, modulation of their nociceptive pathway is important in palliative and other therapies.

Walpole and colleagues at Sandoz reported on the first competitive antagonist of the sensory neuron excitants capsaicin and resineriferatoxin (Walpole, C. S. J. et. al., J. Med. Chem. 1994, 37, 1942). Subsequently, capsazepine has been shown to be a vanilloid receptor antagonist.

### SUMMARY OF THE INVENTION

The present invention is directed to compounds of formula (II) 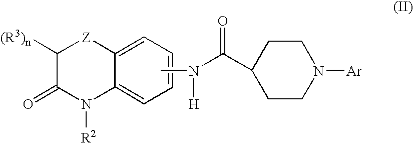
wherein
Ar is an aryl selected from the group consisting of benzo[b]thiophenyl, naphthyl, biphenyl, isoquinolinyl, thiophenyl, pyridazinyl, and benzothiazolyl;
Z is O or S;
R² is hydrogen or C₁₋₆alkyl optionally substituted with —OR⁴;
n is 1 or 2;
R³ is independently hydrogen, C₁₋₆alkyl, —COOR⁴, or —CH₂COOR⁴;
R⁴ is hydrogen or C₁₋₃alkyl; and
enantiomers, diastereomers, tautomers, solvates, and pharmaceutically acceptable salts thereof.

In another embodiment, the present invention is directed to compounds of formula (II) wherein Z is O and n is 1.

In another embodiment, the present invention is directed to compounds of formula (II) wherein Z is O and R² is hydrogen.

In another embodiment, the present invention is directed to compounds of formula (III): 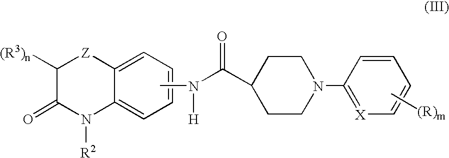
wherein
X is CH or N;
m is an integer from 0 to 4;
R is independently selected from the group consisting of halogen; R¹; fluorinated C₁₋₁₀alkyl; phenyl; amino; cyano; CF₃O—; a 3 membered cyclic heteroalkyl containing 1 heteroatom that is N, O or S wherein said 3 membered cyclic heteroalkyl is optionally substituted with a substituent that is halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a 4 to 5 membered cyclic heteroalkyl containing 1-3 heteroatoms that independently are N, O or S wherein said 4 to 5 membered cyclic heteroalkyl is optionally substituted with 1 to 2 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a 6 to 7 membered cyclic heteroalkyl containing 1-3 heteroatoms that independently are N, O or S wherein said 6 to 7 membered cyclic heteroalkyl is optionally substituted with 1 to 3 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a heteroaryl wherein said heteroaryl is cinnoline, furan, imidazole, indazole, indole, indoline, indolizine, isobenzofuran, isoindole, isoindoline, isoquinoline, isothiazole, isoxazole, naphthyridine, oxadiazole, oxazole, pthalazine, pteridine, pyran, pyrazine, pyrazole, pyridazine, pyridine, pyrimidine, pyrrole, pyrrolizine, quinoline, quinolizine, quinazoline, quinoxaline, tetrazole, thiadiazole, triazine, or triazole wherein said heteroaryl is optionally substituted with 1 to 3 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; hydroxyl; R¹O—; R¹S—; R¹SO₂—; R¹S(O)—; R¹SO₂NH—; -LCOY; and C₆₋₁₀aryl;
R¹ is C₁₋₁₀alkyl;
L is —NH—, a direct bond, —O—, or —CH₂—;
Y is H, R¹, HO, R¹O—, R¹S—, —NH₂, R¹NH—, or (R¹)₂N—;
Z is O or S;
R² is hydrogen or C₁₋₆alkyl optionally substituted with —OR⁴;
n is 1 or 2;
R³ is independently hydrogen, C₁₋₆alkyl, —COOR⁴, or —CH₂COOR⁴;
R⁴ is hydrogen or C₁₋₃alkyl; and
enantiomers, diastereomers, tautomers, solvates, and pharmaceutically acceptable salts thereof.
   In another embodiment, the present invention is directed to compounds of formula (IIa): 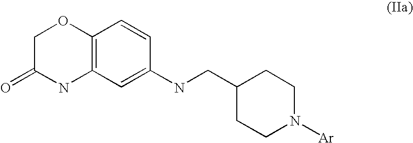
wherein Ar is an aryl selected from the group consisting of benzo[b]thiophenyl, naphthyl, biphenyl, isoquinolinyl, thiophenyl, pyridazinyl, and benzothiazolyl; and
   enantiomers, diastereomers, tautomers, solvates, and pharmaceutically acceptable salts thereof.
   The present invention is also drawn to compounds of formula (IIIa): 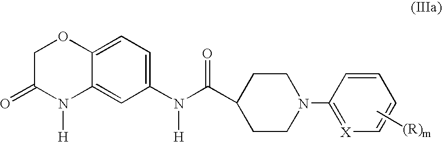
   wherein
X is CH or N;
m is an integer from 0 to 4;
R is independently selected from the group consisting of halogen; R¹; fluorinated C₁₋₁₀alkyl; phenyl; amino; cyano; CF₃O—; a 3 membered cyclic heteroalkyl containing 1 heteroatom that is N, O or S wherein said 3 membered cyclic heteroalkyl is optionally substituted with a substituent that is halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a 4 to 5 membered cyclic heteroalkyl containing 1-3 heteroatoms that independently are N, O or S wherein said 4 to 5 membered cyclic heteroalkyl is optionally substituted with 1 to 2 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a 6 to 7 membered cyclic heteroalkyl containing 1-3 heteroatoms that independently are N, O or S wherein said 6 to 7 membered cyclic heteroalkyl is optionally substituted with 1 to 3 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a heteroaryl wherein said heteroaryl is cinnoline, furan, imidazole, indazole, indole, indoline, indolizine, isobenzofuran, isoindole, isoindoline, isoquinoline, isothiazole, isoxazole, naphthyridine, oxadiazole, oxazole, pthalazine, pteridine, pyran, pyrazine, pyrazole, pyridazine, pyridine, pyrimidine, pyrrole, pyrrolizine, quinoline, quinolizine, quinazoline, quinoxaline, tetrazole, thiadiazole, triazine, or triazole wherein said heteroaryl is optionally substituted with 1 to 3 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; hydroxyl; R¹O—; R¹S—; R¹SO₂—; R¹S(O)—; R¹SO₂NH—; -LCOY; and C₆₋₁₀aryl;
wherein R¹ is C₁₋₁₀alkyl; L is —NH—, a direct bond, —O—, or —CH₂—; and Y is H, R¹, HO, R¹O—, R¹S—, —NH₂, R¹NH—, or (R¹)₂N—; and
   enantiomers, diastereomers, tautomers, solvates, and pharmaceutically acceptable salts thereof.

### DETAILED DESCRIPTION OF THE INVENTION

As used herein, the following underlined terms are intended to have the following meanings:

“C_{a-b}” (where a and b are integers) refers to a radical containing from a to b carbon atoms inclusive. For example, C₁₋₃ denotes a radical containing 1, 2 or 3 carbon atoms.

“Fluorinated alkyl” refers to a saturated branched or straight chain hydrocarbon radical derived by removal of 1 hydrogen atom from the parent alkane; the parent alkane contains from 1 to 6 carbon atoms with 1 or more hydrogen atoms substituted with fluorine atoms up to and including substitution of all hydrogen atoms with fluorine. Preferred fluorinated alkyls include trifluoromethyl substituted alkyls and perfluorinated alkyls; more preferred fluorinated alkyls include trifluoromethyl, perfluoroethyl, 2,2,2-trifluoroethyl, perfluoropropyl, 3,3,3-trifluoroprop-1-yl, 3,3,3-trifluoroprop-2-yl, 1,1,1,3,3,3-hexafluoroprop-2-yl; a particularly preferred fluorinated alkyl is trifluoromethyl.

“Fluorinated alkanyloxy” refers to a radical derived from a fluorinated alkyl radical attached to an oxygen atom with the oxygen atom having one open valence for attachment to a parent structure.

“Alkyl:” refers to a saturated or unsaturated, branched, straight-chain or cyclic monovalent hydrocarbon radical derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl, cycloprop-1-en-1-yl; cycloprop-2-en-1-yl, prop-1-yn-1-yl, prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like. Where specific levels of saturation are intended, the nomenclature “alkanyl”, “alkenyl” and/or “alkynyl” is used, as defined below. In preferred embodiments, the alkyl groups are (C₁₋₈) alkyl, with (C₁₋₃) being particularly preferred.]

“Alkanyl:” refers to a saturated branched, straight-chain or cyclic monovalent hydrocarbon radical derived by the removal of one hydrogen atom from a single carbon atom of a parent alkane. Typical alkanyl groups include, but are not limited to, methanyl; ethanyl; propanyls such as propan-1-yl, propan-2-yl, cyclopropan-1-yl, etc.; butyanyls such as butan-1-yl, butan-2-yl, 2-methyl-propan-1-yl, 2-methyl-propan-2-yl, cyclobutan-1-yl, etc.; and the like. In preferred embodiments, the alkanyl groups are (C₁₋₈) alkanyl, with (C₁₋₃) being particularly preferred.

“Alkenyl:” refers to an unsaturated branched, straight-chain or cyclic monovalent hydrocarbon radical having at least one carbon-carbon double bond derived by the removal of one hydrogen atom from a single carbon atom of a parent alkene. The radical may be in either the cis or trans conformation about the double bond(s). Typical alkenyl groups include, but are not limited to, ethenyl; propenyls such as prop-1-en-1-yl, prop-1-en-2-yl, prop-2-en-1-yl, prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. In preferred embodiments, the alkenyl group is (C₂₋₈) alkenyl, with (C₂₋₃) being particularly preferred.

“Alkynyl:” refers to an unsaturated branched, straight-chain or cyclic monovalent hydrocarbon radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like. In preferred embodiments, the alkynyl group is (C₂₋₈) alkynyl, with (C₂₋₃) being particularly preferred.

“Alkyldiyl:” refers to a saturated or unsaturated, branched, straight-chain or cyclic divalent hydrocarbon radical derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers can form bonds with the same or different atoms. Typical alkyldiyls include, but are not limited to methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyidiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methylprop-2-en-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. Where specific levels of saturation are intended, the nomenclature alkandiyl, alkendiyl and/or alkyndiyl is used. In preferred embodiments, the alkyldiyl group is (C₁₋₈) alkyldiyl, with (C₁₋₈) being particularly preferred. Also preferred are saturated acyclic alkandiyl radicals in which the radical centers are at the terminal carbons, e.g., methandiyl; ethan-1,2-diyl; propan-1,3-diyl; butan-1,4-diyl; and the like (also referred to as alkylenos, as defined infra).

“Vic Alkyldiyl:” refers to a saturated or unsaturated, branched, straight-chain or cyclic hydrocarbon radical having two adjacent monovalent radical centers derived by the removal of one hydrogen atom from each of two adjacent carbon atoms of a parent alkane, alkene or alkyne. The two monovalent radical centers can form bonds with the same or different atom(s). Typical vic alkyldiyls include, but are not limited to vic ethyldiyls such as ethan-1,2-diyl, ethen-1,2-diyl; vic propyldiyls such as propan-1,2-diyl, cyclopropan-1,2-diyl, prop-1-en-1,2-diyl, prop-2-en-1,2-diyl, cycloprop-1-en-1,2-diyl, etc.; vic butyldiyls such as butan-1,2-diyl, 2-methyl-propan-1,2-diyl, cyclobutan-1,2-diyl, but-1-en-1,2-diyl, cyclobut-1-en-1,2-diyl, buta-1,3-dien-1,2-diyl, cyclobuta-1,3-dien-1,2-diyl, but-3-yn-1,2-diyl, etc.; and the like. Where specific levels of saturation are intended, the nomenclature vic alkandiyl, vic alkendiyl and/or vic alkyndiyl is used. In preferred embodiments, the vic alkyldiyl group is (C₂₋₈) vic alkyldiyl, with (C₂₋₃) being particularly preferred.

“Gem Alkyldiyl:” refers to a saturated or unsaturated, branched, straight-chain or cyclic hydrocarbon radical having one divalent radical center derived by the removal of two hydrogen atoms from a single carbon atom of a parent alkane, alkene or alkyne. The divalent radical center forms bonds with two different atoms. Typical gem alkyldiyls include, but are not limited to gem methanyldiyl; gem ethyldiyls such as ethan-1,1-diyl, ethen-1,1-diyl; gem propyldiyls such as propan-1,1-diyl, propan-2,2-diyl, cyclopropan-1,1-diyl, prop-1-en-1,1-diyl, cycloprop-2-en-1,1-diyl, prop-2-yn-1,1-diyl, etc.; butyldiyls such as butan-1,1-diyl, butan-2,2-diyl, 2-methyl-propan-1,2-diyl, cyclobutan-1,1-diyl, but-1-en-1,1-diyl, 2-methyl-prop-1-en-1,1-diyl, 2-methyl-prop-2-en-1,1-diyl, cyclobut-2-en-1,1-diyl, buta-1,3-dien-1,1-diyl, etc.; and the like. Where specific levels of saturation are intended, the nomenclature gem alkandiyl, gem alkendiyl and/or gem alkyndiyl is used. In preferred embodiments, the gem alkyldiyl group is (C₁₋₆) gem alkyldiyl, with (C₁₋₃) being particularly preferred.

“Alkyleno:” refers to a saturated or unsaturated, straight-chain or branched acyclic bivalent hydrocarbon bridge radical derived by the removal of one hydrogen atom from each of the two terminal carbon atoms of an acyclic parent alkane, alkene or alkyne. Typical alkyleno groups include, but are not limited to, methano; ethylenos such as ethano, etheno, ethyno; propylenos such as propano, propeno, prop-1,2-dieno, propyno, etc.; butylenos such as butano, 2-methyl-propano, but-1-eno, but-2-eno, 2-methyl-prop-1-eno, 2-methanylidene-propano, but-1,3-dieno, but-1-yno, but-2-yno, but-1,3-diyno, etc.; and the like. Where specific levels of saturation are intended, the nomenclature alkano, alkeno and/or alkyno is used. In preferred embodiments, the alkyleno group is (C₁₋₈) alkyleno, with (C₁₋₃) being particularly preferred. Also preferred are straight-chain saturated alkano radicals, e.g., methano, ethano, propano, butano, and the like.

“Alkylidene:” refers to a saturated or unsaturated, branched, straight-chain or cyclic divalent hydrocarbon radical derived by removal of two hydrogen atoms from the same carbon atom of a parent alkane, alkene or alkyne. The divalent radical center forms a double bond with a single atom. Typical alkylidene radicals include, but are not limited to, methanylidene, ethylidenes such as ethanylidene, ethenylidene; propylidenes such as propan-1-ylidene, propan-2-ylidene, cyclopropan-1-ylidene, prop-1-en-1-ylidene, prop-2-en-1-ylidene, cycloprop-2-en-1-ylidene, etc.; butylidenes such as butan-1-ylidene, butan-2-ylidene, 2-methyl-propan-1-ylidene, cyclobutan-1-ylidene, but-1-en-1-ylidene, but-2-en-1-ylidene, but-3-en-1-ylidene, buta-1,3-dien-1-ylidene; cyclobut-2-en-1-ylidene, etc.; and the like. Where specific levels of saturation are intended, the nomenclature alkanylidene, alkenylidene and/or alkynylidene is used. In preferred embodiments, the alkylidene group is (C₁₋₈) alkylidene, with (C₁₋₃) being particularly preferred. Also preferred are acyclic saturated alkanylidene radicals in which the divalent radical is at a terminal carbon, e.g., methanylidene, ethan-1-ylidene, propan-1-ylidene, butan-1-ylidene, 2-methyl-propan-1-ylidene, and the like.

“Alkylidyne:” refers to a saturated or unsaturated, branched or straight-chain trivalent hydrocarbon radical derived by removal of three hydrogen atoms from the same carbon atom of a parent alkane, alkene or alkyne. The trivalent radical center forms a triple bond with a single atom. Typical alkylidyne radicals include, but are not limited to, methanylidyne; ethanylidyne; propylidynes such as propan-1-ylidyne, prop-2-en-1-ylidyne, prop-2-yn-1-ylidyne; butylidynes such as butan-1-ylidyne, 2-methyl-propan-1-ylidyne, but-2-en-1-ylidyne, but-3-en-1-ylidyne, buta-2,3-dien-1-ylidyne, but-2-yn-1-ylidyne, but-3-yn-1-ylidyne, etc.; and the like. Where specific levels of saturation are intended, the nomenclature alkanylidyne, alkenylidyne and/or alkynylidyne is used. In preferred embodiments, the alkylidyne group is (C₁₋₈) alkylidyne, with (C₁₋₃) being particularly preferred. Also preferred are saturated alkanylidyne radicals, e.g., methanylidyne, ethanylidyne, propan-1-ylidyne, butan-1-ylidyne, 2-methyl-propan-1-ylidyne, and the like.

“Heteroalkyl, Heteroalkanyl, Heteroalkenyl, Heteroalkynyl, Heteroalkylidene, Heteroalkylidyne, Heteroalkyldiyl, Vic Heteralkyldiyl, Gem Heteroalkyldiyl, Heteroalkyleno and Heteroalkyldiylidene:” refer to alkyl, alkanyl, alkenyl, alkynyl, alkylidene, alkylidyne, alkyldiyl, vic alkyldiyl, gem alkyldiyl, alkyleno and alkyldiylidene radicals, respectively, in which one or more carbon atoms (and any necessary associated hydrogen atoms) are independently replaced with the same or different heteroatoms (including any necessary hydrogen or other atoms). Typical heteroatoms to replace the carbon atom(s) include, but are not limited to, N, P, O, S, Si, etc. Preferred heteroatoms are O, N and S. Thus, heteroalkyl, heteroalkanyl, heteroalkenyl, heteroalkynyl, heteroalkylidene, heteroalkylidyne, heteroalkyldiyl, vic heteroalkyldiyl, gem heteroalkyldiyl, heteroalkyleno and heteroalkyldiylidene radicals can contain one or more of the same or different heteroatomic groups, including, by way of example and not limitation, epoxy (—O—), epidioxy (—O—O—), thioether (—S—), epidithio (—SS—), epoxythio (—O—S—), epoxyimino (—O—NR′—), imino (—NR′—), biimmino (—NR′—NR′—), azino (═N—N═), azo (—N═N—), azoxy (—N—O—N—), azimino (—NR′—N═N—), phosphano (—PH—), λ⁴-sulfano (—SH₂—), sulfonyl (—S(O)₂—), and the like, where each R′ is independently hydrogen or (C₁-C₆) alkyl.

“Parent Aromatic Ring System:” refers to an unsaturated cyclic or polycyclic ring system having a conjugated Π electron system. Specifically included within the definition of “parent aromatic ring system” are fused ring systems in which one or more rings are aromatic and one or more rings are saturated or unsaturated, such as, for example, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like

“Aryl:” refers to a monovalent aromatic hydrocarbon radical derived by the removal of one hydrogen atom from a single carbon atom of a parent aromatic ring system. Typical aryl groups include, but are not limited to, radicals derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In preferred embodiments, the aryl group is (C₅₋₂₀) aryl, with (C₅₋₁₀) being particularly preferred. Particularly preferred aryl groups are phenyl and naphthyl groups.

“Arylalkyl:” refers to an acyclic alkyl group in which one of the hydrogen atoms bonded to a carbon atom, typically a terminal carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl and/or arylalkynyl is used. [In preferred embodiments, the arylalkyl group is (C₆₋₂₆) arylalkyl, e.g., the alkanyl, alkenyl or alkynyl moiety of the arylalkyl group is (C₁₋₆) and the aryl moiety is (C₅₋₂₀). In particularly preferred embodiments the arylalkyl group is (C₆₋₁₃), e.g., the alkanyl, alkenyl or alkynyl moiety of the arylalkyl group is (C₁₋₃) and the aryl moiety is (C₅₋₁₀). Even more preferred arylalkyl groups are phenylalkanyls.

“Alkanyloxy:” refers to a saturated branched, straight-chain or cyclic monovalent hydrocarbon alcohol radical derived by the removal of the hydrogen atom from the hydroxide oxygen of the alcohol. Typical alkanyloxy groups include, but are not limited to, methanyl; ethanyloxy; propanyloxy groups such as propan-1-yloxy (CH₃CH₂CH₂O—), propan-2-yloxy ((CH₃)₂CHO—), cyclopropan-1-yloxy, etc.; butyanyloxy groups such as butan-1-yloxy, butan-2-yloxy, 2-methyl-propan-1-yloxy, 2-methyl-propan-2-yloxy, cyclobutan-1-yloxy, etc.; and the like. In preferred embodiments, the alkanyloxy groups are (C₁₋₈) alkanyloxy groups, with (C₁₋₃) being particularly preferred.

“Parent Heteroaromatic Ring System:” refers to a parent aromatic ring system in which one or more carbon atoms are each independently replaced with a heteroatom. Typical heteratoms to replace the carbon atoms include, but are not limited to, N, P, O, S, Si etc. Specifically included within the definition of “parent heteroaromatic ring systems” are fused ring systems in which one or more rings are aromatic and one or more rings are saturated or unsaturated, such as, for example, arsindole, chromane, chromene, indole, indoline, xanthene, etc. Typical parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like.

“Heteroaryl:” refers to a monovalent heteroaromatic radical derived by the removal of one hydrogen atom from a single atom of a parent heteroaromatic ring system. Typical heteroaryl groups include, but are not limited to, radicals derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In preferred embodiments, the heteroaryl group is a 5-20 membered heteroaryl, with 5-10 membered heteroaryl being particularly preferred. Specific preferred heteroaryls for the present invention are quinoline, isoquinoline, pyridine, pyrimidine, furan, thiophene and imidazole.

“Substituted:” refers to a radical in which one or more hydrogen atoms are each independently replaced with the same or different substituent(s). Typical substituents include, but are not limited to, —X, —R, —O⁻, ═O, —OR, —O—OR, —SR, —S⁻, ═S, —NRR, ═NR, —CX₃, —CN, —OCN, —SCN, —NCO, —NCS, —NO, —NO₂, ═N₂, —N₃, —NHOH, —S(O)₂O⁻, —S(O)₂OH, —S(O)₂R, —P(O)(O⁻)₂, —P(O)(OH)₂, —C(O)R, —C(O)X, —C(S)R, —C(S)X, —C(O)OR, —C(O)O⁻, —C(S)OR, —C(O)SR, —C(S)SR, —C(O)NRR, —C(S)NRR and —C(NR)NRR, where each X is independently a halogen (preferably —F, —Cl or —Br) and each R is independently —H, alkyl, alkanyl, alkenyl, alkynyl, alkylidene, alkylidyne, aryl, arylalkyl, arylheteroalkyl, heteroaryl, heteroarylalkyl or heteroaryl-heteroalkyl, as defined herein. Preferred substituents include hydroxy, halogen, C₁₋₈alkyl, C₁₋₈alkanyloxy, fluorinated alkanyloxy, fluorinated alkyl, C₁₋₈alkylthio, C₃₋₈cycloalkyl, C₃₋₈cycloalkanyloxy, nitro, amino, C₁₋₈alkylamino, C₁₋₈dialkylamino, C₃₋₈cycloalkylamino, cyano, carboxy, C₁₋₇alkanyloxycarbonyl, C₁₋₇alkylcarbonyloxy, formyl, carbamoyl, phenyl, aroyl, carbamoyl, amidino, (C₁₋₈alkylamino)carbonyl, (arylamino)carbonyl and aryl(C₁₋₈alkyl)carbonyl.

“Aroyl” refers to arylacyl substituents.

“Acyl” refers to alkylcarbonyl substituents.

With reference to substituents, the term “independently” means that when more than one of such substituent is possible, such substituents may be the same or different from each other.

Throughout this disclosure, the terminal portion of the designated side chain is described first, followed by the adjacent functionality toward the point of attachment. Thus, for example, a “phenylC₁₋₆alkanylaminocarbonylC₁₋₆alkyl” substituent refers to a group of the formula

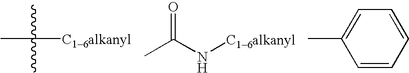

The invention is directed to compounds of formula (I) 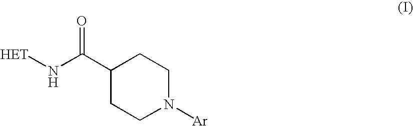
wherein
HET is a heteroaryl selected from the group consisting of 1,3-dihydro-benzoimidazol-2-one-5-yl, 3,4-dihydro-2h-benzo[1,4]oxazine, 1,2,3,4-tetrahydro-[1,8]naphthyridine, 1h-pyrrolo[2,3-b]pyridine, benzo[b]thiophene-4-yl, benzo[b]thiophene-5-yl, benzo[b]thiophene-6-yl, benzo[b]thiophene-7-yl, benzo[b]furan-4-yl, benzo[b]furan-5-yl, benzo[b]furan-6-yl, benzo[b]furan-7-yl, 4h-quinazoline-1-yl, 4h-quinazoline-2-yl, 4h-quinazoline-3-yl, quinazoline-2-yl, quinoxaline-2-yl, pteridine, carbazole, acridine, phenazine, pyrrole, thiazole-2-yl, thiazole-4-yl, thiazole-5-yl, imidazole-2-yl, imidazole-4-yl, imidazole-5-yl, 2-imidazoline-2-yl, pyrazole-3-yl, pyrazole-4-yl, pyrazole-5-yl, 2-pyrazoline-3-yl, isoxazole-3-yl, isoxazole-4-yl, isoxazole-5-yl, isothiazole-3-yl, isothiazole-4-yl, isothiazole-5-yl, benzisoxazole, phthalazine-4-yl, isoindole-4-yl, isoindole-5-yl, isoindole-6-yl, isoindole-7-yl, ih-indazole-4-yl, ih-indazole-5-yl, ih-indazole-6-yl, ih-indazole-7-yl, purine-2-yl, purine-6-yl, purine-8-yl, benzimidazole-4-yl, benzimidazole-5-yl, benzimidazole-6-yl, benzimidazole-7-yl, cinnoline-4-yl, cinnoline-7-yl, cinnoline-8-yl, quinoxaline-5-yl, quinoxaline-6-yl, 2,7-naphthyridine-2-yl, pyrazine-2-yl, pyrazine-3-yl, quinazoline-7-yl, 1,7-naphthyridine-2-yl, 1,7-naphthyridine-5-yl, 1,7-naphthyridine-6-yl, 1,7-naphthyridine-8-yl, 2,6-naphthyridine-3-yl, 2,6-naphthyridine-4-yl, 2,6-naphthyridine-5-yl, 2,6-naphthyridine-7-yl, 2,6-naphthyridine-8-yl, pyridazine-3-yl, pyridazine-4-yl, pyridazine-5-yl, quinoline-2-yl, quinoline-3-yl, quinoline-4-yl, indoline-4-yl, indoline-5-yl, indoline-6-yl, indoline-7-yl, isoquinoline-1-yl, isoquinoline-3-yl, isoquinoline-4-yl, cinnoline-3-yl, cinnoline-5-yl, cinnoline-6-yl, phthalazine-5-yl, phthalazine-6-yl, quinazoline-4-yl, quinazoline-5-yl, quinazoline-6-yl, quinazoline-8-yl, 1,6-naphthyridine-2-yl, 1,6-naphthyridine-3-yl, 1,6-naphthyridine-4-yl, 1,6-naphthyridine-5-yl, 1,6-naphthyridine-7-yl, 1,6-naphthyridine-8-yl, 1,8-naphthyridine-2-yl, 1,8-naphthyridine-3-yl, 1,8-naphthyridine-4-yl, 1,7-naphthyridine-3-yl, 1,7-naphthyridine-4-yl, 2,6-naphthyridine-1-yl, 2,7-naphthyridine-3-yl, 2,7-naphthyridine-4-yl, pyridine-2-yl, pyridine-3-yl, pyridine-4-yl, benzisothiazolyl, and benzothiazolyl;
Ar is an aryl selected from the group consisting of benzo[b]thiophenyl, naphthyl, biphenyl, isoquinolinyl, thiophenyl, pyridazinyl, and benzothiazolyl; and
   enantiomers, diastereomers, tautomers, solvates, and pharmaceutically acceptable salts thereof.

In another embodiment, the present invention is directed to compounds of formula (I) wherein
HET is a heteroaryl selected from the group consisting of 1,3-dihydro-benzoimidazol-2-one-5-yl, 3,4-dihydro-2h-benzo[1,4]oxazine, 1,2,3,4-tetrahydro-[1,8]naphthyridine, 1h-pyrrolo[2,3-b]pyridine, benzo[b]thiophene-4-yl, benzo[b]thiophene-5-yl, benzo[b]thiophene-6-yl, benzo[b]thiophene-7-yl, 4h-quinazoline-1-yl, 4h-quinazoline-2-yl, 4h-quinazoline-3-yl, quinazoline-2-yl, quinoxaline-2-yl, pteridine, carbazole, acridine, phenazine, pyrrole, imidazole-2-yl, imidazole-4-yl, imidazole-5-yl, 2-imidazoline-2-yl, pyrazole-3-yl, pyrazole-4-yl, pyrazole-5-yl, 2-pyrazoline-3-yl, isoxazole-3-yl, isoxazole-4-yl, isoxazole-5-yl, isothiazole-3-yl, isothiazole-4-yl, isothiazole-5-yl, benzisoxazole, phthalazine-4-yl, isoindole-4-yl, isoindole-5-yl, isoindole-6-yl, isoindole-7-yl, ih-indazole-4-yl, ih-indazole-5-yl, ih-indazole-6-yl, ih-indazole-7-yl, purine-2-yl, purine-6-yl, purine-8-yl, cinnoline-4-yl, cinnoline-7-yl, cinnoline-8-yl, quinoxaline-5-yl, quinoxaline-6-yl, 2,7-naphthyridine-2-yl, pyrazine-2-yl, pyrazine-3-yl, quinazoline-7-yl, 1,7-naphthyridine-2-yl, 1,7-naphthyridine-5-yl, 1,7-naphthyridine-6-yl, 1,7-naphthyridine-8-yl, 2,6-naphthyridine-3-yl, 2,6-naphthyridine-4-yl, 2,6-naphthyridine-5-yl, 2,6-naphthyridine-7-yl, 2,6-naphthyridine-8-yl, pyridazine-3-yl, pyridazine-4-yl, pyridazine-5-yl, indoline-4-yl, indoline-5-yl, indoline-6-yl, indoline-7-yl, cinnoline-3-yl, cinnoline-5-yl, cinnoline-6-yl, phthalazine-5-yl, phthalazine-6-yl, quinazoline-4-yl, quinazoline-5-yl, quinazoline-6-yl, quinazoline-8-yl, 1,6-naphthyridine-2-yl, 1,6-naphthyridine-3-yl, 1,6-naphthyridine-4-yl, 1,6-naphthyridine-5-yl, 1,6-naphthyridine-7-yl, 1,6-naphthyridine-8-yl, 1,8-naphthyridine-2-yl, 1,8-naphthyridine-3-yl, 1,8-naphthyridine-4-yl, 1,7-naphthyridine-3-yl, 1,7-naphthyridine-4-yl, 2,6-naphthyridine-1-yl, 2,7-naphthyridine-3-yl, 2,7-naphthyridine-4-yl, and benzisothiazole;
Ar is an aryl selected from the group consisting of benzo[b]thiophenyl, naphthalenyl, biphenyl, quinolinyl, isoquinolinyl, thiophenyl, pyrimidinyl, pyridazinyl, phenyl, benzothiazole, and pyridinyl; and
   enantiomers, diastereomers, tautomers, solvates, and pharmaceutically acceptable salts thereof.

In another embodiment, the present invention is directed to compounds of formula (I) wherein
HET is a heteroaryl selected from the group consisting of 1,3-dihydro-benzoimidazol-2-one-5-yl, 3,4-dihydro-2h-benzo[1,4]oxazine, 1,2,3,4-tetrahydro-[1,8]naphthyridine, 1h-pyrrolo[2,3-b]pyridine, benzo[b]thiophene-4-yl, benzo[b]thiophene-5-yl, benzo[b]thiophene-6-yl, benzo[b]thiophene-7-yl, 4h-quinazoline-1-yl, 4h-quinazoline-2-yl, 4h-quinazoline-3-yl, quinazoline-2-yl, quinoxaline-2-yl, pteridine, carbazole, acridine, phenazine, pyrrole, thiazole-2-yl, thiazole-4-yl, thiazole-5-yl, imidazole-2-yl, imidazole-4-yl, imidazole-5-yl, 2-imidazoline-2-yl, pyrazole-3-yl, pyrazole-4-yl, pyrazole-5-yl, 2-pyrazoline-3-yl, isoxazole-3-yl, isoxazole-4-yl, isoxazole-5-yl, isothiazole-3-yl, isothiazole-4-yl, isothiazole-5-yl, benzisoxazole, phthalazine-4-yl, isoindole-4-yl, isoindole-5-yl, isoindole-6-yl, isoindole-7-yl, ih-indazole-4-yl, ih-indazole-5-yl, ih-indazole-6-yl, ih-indazole-7-yl, purine-2-yl, purine-6-yl, purine-8-yl, cinnoline-4-yl, cinnoline-7-yl, cinnoline-8-yl, quinoxaline-5-yl, quinoxaline-6-yl, 2,7-naphthyridine-2-yl, pyrazine-2-yl, pyrazine-3-yl, quinazoline-7-yl, 1,7-naphthyridine-2-yl, 1,7-naphthyridine-5-yl, 1,7-naphthyridine-6-yl, 1,7-naphthyridine-8-yl, 2,6-naphthyridine-3-yl, 2,6-naphthyridine-4-yl, 2,6-naphthyridine-5-yl, 2,6-naphthyridine-7-yl, 2,6-naphthyridine-8-yl, pyridazine-3-yl, pyridazine-4-yl, pyridazine-5-yl, indoline-4-yl, indoline-5-yl, indoline-6-yl, indoline-7-yl, cinnoline-3-yl, cinnoline-5-yl, cinnoline-6-yl, phthalazine-5-yl, phthalazine-6-yl, quinazoline-4-yl, quinazoline-5-yl, quinazoline-6-yl, quinazoline-8-yl, 1,6-naphthyridine-2-yl, 1,6-naphthyridine-3-yl, 1,6-naphthyridine-4-yl, 1,6-naphthyridine-5-yl, 1,6-naphthyridine-7-yl, 1,6-naphthyridine-8-yl, 1,8-naphthyridine-2-yl, 1,8-naphthyridine-3-yl, 1,8-naphthyridine-4-yl, 1,7-naphthyridine-3-yl, 1,7-naphthyridine-4-yl, 2,6-naphthyridine-1-yl, 2,7-naphthyridine-3-yl, 2,7-naphthyridine-4-yl, and benzisothiazole;
Ar is an aryl selected from the group consisting of thiophenyl, pyrimidinyl, pyridazinyl, phenyl, benzothiazole, and pyridinyl; and
enantiomers, diastereomers, tautomers, solvates, and pharmaceutically acceptable salts thereof.

In another embodiment, the present invention is directed to compounds of formula (I) wherein
HET is a heteroaryl selected from the group consisting of 1,3-dihydro-benzoimidazol-2-one-5-yl, 3,4-dihydro-2h-benzo[1,4]oxazine, 1,2,3,4-tetrahydro-[1,8]naphthyridine, 1h-pyrrolo[2,3-b]pyridine, benzo[b]thiophene-4-yl, benzo[b]thiophene-5-yl, benzo[b]thiophene-6-yl, benzo[b]thiophene-7-yl, 4h-quinazoline-1-yl, 4h-quinazoline-2-yl, 4h-quinazoline-3-yl, quinazoline-2-yl, quinoxaline-2-yl, pteridine, carbazole, acridine, phenazine, pyrrole, thiazole-2-yl, thiazole-4-yl, thiazole-5-yl, imidazole-2-yl, imidazole-4-yl, imidazole-5-yl, 2-imidazoline-2-yl, pyrazole-3-yl, pyrazole-4-yl, pyrazole-5-yl, 2-pyrazoline-3-yl, isoxazole-3-yl, isoxazole-4-yl, isoxazole-5-yl, isothiazole-3-yl, isothiazole-4-yl, isothiazole-5-yl, benzisoxazole, phthalazine-4-yl, isoindole-4-yl, isoindole-5-yl, isoindole-6-yl, isoindole-7-yl, ih-indazole-4-yl, ih-indazole-5-yl, ih-indazole-6-yl, ih-indazole-7-yl, purine-2-yl, purine-6-yl, purine-8-yl, benzimidazole-4-yl, benzimidazole-5-yl, benzimidazole-6-yl, benzimidazole-7-yl, cinnoline-4-yl, cinnoline-7-yl, cinnoline-8-yl, quinoxaline-5-yl, quinoxaline-6-yl, 2,7-naphthyridine-2-yl, pyrazine-2-yl, pyrazine-3-yl, quinazoline-7-yl, 1,7-naphthyridine-2-yl, 1,7-naphthyridine-5-yl, 1,7-naphthyridine-6-yl, 1,7-naphthyridine-8-yl, 2,6-naphthyridine-3-yl, 2,6-naphthyridine-4-yl, 2,6-naphthyridine-5-yl, 2,6-naphthyridine-7-yl, 2,6-naphthyridine-8-yl, pyridazine-3-yl, pyridazine-4-yl, pyridazine-5-yl, indoline-4-yl, indoline-5-yl, indoline-6-yl, indoline-7-yl, cinnoline-3-yl, cinnoline-5-yl, cinnoline-6-yl, phthalazine-5-yl, phthalazine-6-yl, quinazoline-4-yl, quinazoline-5-yl, quinazoline-6-yl, quinazoline-8-yl, 1,6-naphthyridine-2-yl, 1,6-naphthyridine-3-yl, 1,6-naphthyridine-4-yl, 1,6-naphthyridine-5-yl, 1,6-naphthyridine-7-yl, 1,6-naphthyridine-8-yl, 1,8-naphthyridine-2-yl, 1,8-naphthyridine-3-yl, 1,8-naphthyridine-4-yl, 1,7-naphthyridine-3-yl, 1,7-naphthyridine-4-yl, 2,6-naphthyridine-1-yl, 2,7-naphthyridine-3-yl, 2,7-naphthyridine-4-yl, benzisothiazolyl, and benzothiazolyl;
Ar is an aryl selected from the group consisting of phenyl, benzothiazole, and pyridinyl; and
   enantiomers, diastereomers, tautomers, solvates, and pharmaceutically acceptable salts thereof.

In another embodiment, the present invention is directed to compounds of formula (I) wherein
HET is a phthalazine-4-yl, isoindole-4-yl, isoindole-5-yl, isoindole-6-yl, isoindole-7-yl, ih-indazole-4-yl, ih-indazole-5-yl, ih-indazole-6-yl, ih-indazole-7-yl, purine-2-yl, purine-6-yl, purine-8-yl, benzimidazole-4-yl, benzimidazole-5-yl, benzimidazole-6-yl, benzimidazole-7-yl, cinnoline-4-yl, cinnoline-7-yl, cinnoline-8-yl, quinoxaline-5-yl, quinoxaline-6-yl, 2,7-naphthyridine-2-yl, pyrazine-2-yl, pyrazine-3-yl, quinazoline-7-yl, 1,7-naphthyridine-2-yl, 1,7-naphthyridine-5-yl, 1,7-naphthyridine-6-yl, 1,7-naphthyridine-8-yl, 2,6-naphthyridine-3-yl, 2,6-naphthyridine-4-yl, 2,6-naphthyridine-5-yl, 2,6-naphthyridine-7-yl, 2,6-naphthyridine-8-yl, pyridazine-3-yl, pyridazine-4-yl, pyridazine-5-yl, quinoline-2-yl, quinoline-3-yl, quinoline-4-yl, indoline-4-yl, indoline-5-yl, indoline-6-yl, indoline-7-yl, isoquinoline-1-yl, isoquinoline-3-yl, isoquinoline-4-yl, cinnoline-3-yl, cinnoline-5-yl, cinnoline-6-yl, phthalazine-5-yl, phthalazine-6-yl, quinazoline-4-yl, quinazoline-5-yl, quinazoline-6-yl, quinazoline-8-yl, 1,6-naphthyridine-2-yl, 1,6-naphthyridine-3-yl, 1,6-naphthyridine-4-yl, 1,6-naphthyridine-5-yl, 1,6-naphthyridine-7-yl, 1,6-naphthyridine-8-yl, 1,8-naphthyridine-2-yl, 1,8-naphthyridine-3-yl, 1,8-naphthyridine-4-yl, 1,7-naphthyridine-3-yl, 1,7-naphthyridine-4-yl, 2,6-naphthyridine-1-yl, 2,7-naphthyridine-3-yl, 2,7-naphthyridine-4-yl, pyridine-2-yl, pyridine-3-yl, pyridine-4-yl, benzisothiazolyl, and benzothiazolyl;
Ar is an aryl selected from the group consisting of benzo[b]thiophenyl, naphthyl, biphenyl, isoquinolinyl, thiophenyl, pyridazinyl, and benzothiazolyl; and
   enantiomers, diastereomers, tautomers, solvates, and pharmaceutically acceptable salts thereof.

In another embodiment, the present invention is directed to compounds of formula (I) wherein
HET is a heteroaryl selected from the group consisting of quinoline-2-yl, quinoline-3-yl, quinoline-4-yl, indoline-4-yl, indoline-5-yl, indoline-6-yl, indoline-7-yl, isoquinoline-1-yl, isoquinoline-3-yl, isoquinoline-4-yl, cinnoline-3-yl, cinnoline-5-yl, cinnoline-6-yl, phthalazine-5-yl, phthalazine-6-yl, quinazoline-4-yl, quinazoline-5-yl, quinazoline-6-yl, quinazoline-8-yl, 1,6-naphthyridine-2-yl, 1,6-naphthyridine-3-yl, 1,6-naphthyridine-4-yl, 1,6-naphthyridine-5-yl, 1,6-naphthyridine-7-yl, 1,6-naphthyridine-8-yl, 1,8-naphthyridine-2-yl, 1,8-naphthyridine-3-yl, 1,8-naphthyridine-4-yl, 1,7-naphthyridine-3-yl, 1,7-naphthyridine-4-yl, 2,6-naphthyridine-1-yl, 2,7-naphthyridine-3-yl, 2,7-naphthyridine-4-yl, pyridine-2-yl, pyridine-3-yl, pyridine-4-yl, benzisothiazolyl, and benzothiazolyl;
Ar is an aryl selected from the group consisting of benzo[b]thiophenyl, naphthyl, biphenyl, isoquinolinyl, thiophenyl, pyridazinyl, and benzothiazolyl; and
   enantiomers, diastereomers, tautomers, solvates, and pharmaceutically acceptable salts thereof.

In another embodiment, the present invention is directed to compounds of formula (I) wherein
HET is a heteroaryl selected from the group consisting of 1,3-dihydro-benzoimidazol-2-one-5-yl, 3,4-dihydro-2h-benzo[1,4]oxazine, 1,2,3,4-tetrahydro-[1,8]naphthyridine, 1h-pyrrolo[2,3-b]pyridine, benzo[b]thiophene-4-yl, benzo[b]thiophene-5-yl, benzo[b]thiophene-6-yl, benzo[b]thiophene-7-yl, benzo[b]furan-4-yl, benzo[b]furan-5-yl, benzo[b]furan-6-yl, benzo[b]furan-7-yl, 4h-quinazoline-1-yl, 4h-quinazoline-2-yl, 4h-quinazoline-3-yl, quinazoline-2-yl, quinoxaline-2-yl, pteridine, carbazole, acridine, phenazine, pyrrole, thiazole-2-yl, thiazole-4-yl, thiazole-5-yl, imidazole-2-yl, imidazole-4-yl, imidazole-5-yl, 2-imidazoline-2-yl, pyrazole-3-yl, pyrazole-4-yl, pyrazole-5-yl, 2-pyrazoline-3-yl, isoxazole-3-yl, isoxazole-4-yl, isoxazole-5-yl, isothiazole-3-yl, isothiazole-4-yl, isothiazole-5-yl, benzisoxazole, phthalazine-4-yl, isoindole-4-yl, isoindole-5-yl, isoindole-6-yl, isoindole-7-yl, ih-indazole-4-yl, ih-indazole-5-yl, ih-indazole-6-yl, ih-indazole-7-yl, purine-2-yl, purine-6-yl, purine-8-yl, benzimidazole-4-yl, benzimidazole-5-yl, benzimidazole-6-yl, benzimidazole-7-yl, cinnoline-4-yl, cinnoline-7-yl, cinnoline-8-yl, quinoxaline-5-yl, quinoxaline-6-yl, 2,7-naphthyridine-2-yl, pyrazine-2-yl, pyrazine-3-yl, quinazoline-7-yl, 1,7-naphthyridine-2-yl, 1,7-naphthyridine-5-yl, 1,7-naphthyridine-6-yl, 1,7-naphthyridine-8-yl, 2,6-naphthyridine-3-yl, 2,6-naphthyridine-4-yl, 2,6-naphthyridine-5-yl, 2,6-naphthyridine-7-yl, 2,6-naphthyridine-8-yl, pyridazine-3-yl, pyridazine-4-yl, pyridazine-5-yl, quinoline-2-yl, quinoline-3-yl, quinoline-4-yl, indoline-4-yl, indoline-5-yl, indoline-6-yl, indoline-7-yl, isoquinoline-1-yl, isoquinoline-3-yl, isoquinoline-4-yl, cinnoline-3-yl, cinnoline-5-yl, cinnoline-6-yl, phthalazine-5-yl, phthalazine-6-yl, quinazoline-4-yl, quinazoline-5-yl, quinazoline-6-yl, quinazoline-8-yl, 1,6-naphthyridine-2-yl, 1,6-naphthyridine-3-yl, 1,6-naphthyridine-4-yl, 1,6-naphthyridine-5-yl, 1,6-naphthyridine-7-yl, 1,6-naphthyridine-8-yl, 1,8-naphthyridine-2-yl, 1,8-naphthyridine-3-yl, 1,8-naphthyridine-4-yl, 1,7-naphthyridine-3-yl, 1,7-naphthyridine-4-yl, 2,6-naphthyridine-1-yl, 2,7-naphthyridine-3-yl, 2,7-naphthyridine-4-yl, pyridine-2-yl, pyridine-3-yl, pyridine-4-yl, benzisothiazolyl, and benzothiazolyl;
Ar is an aryl selected from the group consisting of benzo[b]thiophenyl, naphthalenyl, biphenyl, quinolinyl, isoquinolinyl, thiophenyl, pyrimidinyl, pyridazinyl, phenyl, benzothiazolyl, and pyridinyl; provided that when HET is benzimidazolyl, Ar is not pyrimidinyl or phenyl; and provided that when HET is pyrazolyl, Ar is not phenyl; and provided that when het is benzofuranyl, Ar is not pyridinyl; and provided that when het is benzothiazolyl, Ar is not pyrimidinyl; and provided that when HET is isoquinolinyl, Ar is not pyridinyl; and provided that when HET is pyridinyl, Ar is not pyrimidinyl, pyridinyl, or phenyl; and provided that when HET is quinolinyl, Ar is not pyrimidinyl or pyridinyl; and provided that when HET is thiazolyl, Ar is not quinolinyl; and
   enantiomers, diastereomers, tautomers, solvates, and pharmaceutically acceptable salts thereof.

In another embodiment, the present invention is directed to compounds of formula (I) wherein
HET is a heteroaryl selected from the group consisting of 1,3-dihydro-benzoimidazol-2-one-5-yl, 3,4-dihydro-2h-benzo[1,4]oxazine, 1,2,3,4-tetrahydro-[1,8]naphthyridine, 1h-pyrrolo[2,3-b]pyridine, benzo[b]thiophene-4-yl, benzo[b]thiophene-5-yl, benzo[b]thiophene-6-yl, benzo[b]thiophene-7-yl, benzo[b]furan-4-yl, benzo[b]furan-5-yl, benzo[b]furan-6-yl, benzo[b]furan-7-yl, 4h-quinazoline-1-yl, 4h-quinazoline-2-yl, 4h-quinazoline-3-yl, quinazoline-2-yl, quinoxaline-2-yl, pteridine, carbazole, acridine, phenazine, pyrrole, thiazole-2-yl, thiazole-4-yl, thiazole-5-yl, imidazole-2-yl, imidazole-4-yl, imidazole-5-yl, 2-imidazoline-2-yl, pyrazole-3-yl, pyrazole-4-yl, pyrazole-5-yl, 2-pyrazoline-3-yl, isoxazole-3-yl, isoxazole-4-yl, isoxazole-5-yl, isothiazole-3-yl, isothiazole-4-yl, isothiazole-5-yl, benzisoxazole, phthalazine-4-yl, isoindole-4-yl, isoindole-5-yl, isoindole-6-yl, isoindole-7-yl, ih-indazole-4-yl, ih-indazole-5-yl, ih-indazole-6-yl, ih-indazole-7-yl, purine-2-yl, purine-6-yl, purine-8-yl, benzimidazole-4-yl, benzimidazole-5-yl, benzimidazole-6-yl, benzimidazole-7-yl, cinnoline-4-yl, cinnoline-7-yl, cinnoline-8-yl, quinoxaline-5-yl, quinoxaline-6-yl, 2,7-naphthyridine-2-yl, pyrazine-2-yl, pyrazine-3-yl, quinazoline-7-yl, 1,7-naphthyridine-2-yl, 1,7-naphthyridine-5-yl, 1,7-naphthyridine-6-yl, 1,7-naphthyridine-8-yl, 2,6-naphthyridine-3-yl, 2,6-naphthyridine-4-yl, 2,6-naphthyridine-5-yl, 2,6-naphthyridine-7-yl, 2,6-naphthyridine-8-yl, pyridazine-3-yl, pyridazine-4-yl, pyridazine-5-yl, quinoline-2-yl, quinoline-3-yl, quinoline-4-yl, indoline-4-yl, indoline-5-yl, indoline-6-yl, indoline-7-yl, isoquinoline-1-yl, isoquinoline-3-yl, isoquinoline-4-yl, cinnoline-3-yl, cinnoline-5-yl, cinnoline-6-yl, phthalazine-5-yl, phthalazine-6-yl, quinazoline-4-yl, quinazoline-5-yl, quinazoline-6-yl, quinazoline-8-yl, 1,6-naphthyridine-2-yl, 1,6-naphthyridine-3-yl, 1,6-naphthyridine-4-yl, 1,6-naphthyridine-5-yl, 1,6-naphthyridine-7-yl, 1,6-naphthyridine-8-yl, 1,8-naphthyridine-2-yl, 1,8-naphthyridine-3-yl, 1,8-naphthyridine-4-yl, 1,7-naphthyridine-3-yl, 1,7-naphthyridine-4-yl, 2,6-naphthyridine-1-yl, 2,7-naphthyridine-3-yl, 2,7-naphthyridine-4-yl, pyridine-2-yl, pyridine-3-yl, pyridine-4-yl, benzisothiazolyl, and benzothiazolyl;
Ar is an aryl selected from the group consisting of thiophenyl, pyrimidinyl, pyridazinyl, phenyl, benzothiazolyl, and pyridinyl; provided that when HET is benzimidazolyl, Ar is not pyrimidinyl or phenyl; and provided that when HET is pyrazolyl, Ar is not phenyl; and provided that when HET is benzofuranyl, Ar is not pyridinyl; and provided that when HET is benzothiazolyl, Ar is not pyrimidinyl; and provided that when HET is isoquinolinyl, Ar is not pyridinyl; and provided that when HET is pyridinyl, Ar is not pyrimidinyl, pyridinyl or phenyl; and provided that when HET is pyridinyl, Ar is not phenyl, pyridinyl; and provided that when HET is quinolinyl, Ar is not pyrimidinyl; and
   enantiomers, diastereomers, tautomers, solvates, and pharmaceutically acceptable salts thereof.

In another embodiment, the present invention is directed to compounds of formula (I) wherein
HET is a heteroaryl selected from the group consisting of 1,3-dihydro-benzoimidazol-2-one-5-yl, 3,4-dihydro-2h-benzo[1,4]oxazine, 1,2,3,4-tetrahydro-[1,8]naphthyridine, 1h-pyrrolo[2,3-b]pyridine, benzo[b]thiophene-4-yl, benzo[b]thiophene-5-yl, benzo[b]thiophene-6-yl, benzo[b]thiophene-7-yl, benzo[b]furan-4-yl, benzo[b]furan-5-yl, benzo[b]furan-6-yl, benzo[b]furan-7-yl, 4h-quinazoline-1-yl, 4h-quinazoline-2-yl, 4h-quinazoline-3-yl, quinazoline-2-yl, quinoxaline-2-yl, pteridine, carbazole, acridine, phenazine, pyrrole, thiazole-2-yl, thiazole-4-yl, thiazole-5-yl, imidazole-2-yl, imidazole-4-yl, imidazole-5-yl, 2-imidazoline-2-yl, pyrazole-3-yl, pyrazole-4-yl, pyrazole-5-yl, 2-pyrazoline-3-yl, isoxazole-3-yl, isoxazole-4-yl, isoxazole-5-yl, isothiazole-3-yl, isothiazole-4-yl, isothiazole-5-yl, benzisoxazole, phthalazine-4-yl, isoindole-4-yl, isoindole-5-yl, isoindole-6-yl, isoindole-7-yl, ih-indazole-4-yl, ih-indazole-5-yl, ih-indazole-6-yl, ih-indazole-7-yl, purine-2-yl, purine-6-yl, purine-8-yl, benzimidazole-4-yl, benzimidazole-5-yl, benzimidazole-6-yl, benzimidazole-7-yl, cinnoline-4-yl, cinnoline-7-yl, cinnoline-8-yl, quinoxaline-5-yl, quinoxaline-6-yl, 2,7-naphthyridine-2-yl, pyrazine-2-yl, pyrazine-3-yl, quinazoline-7-yl, 1,7-naphthyridine-2-yl, 1,7-naphthyridine-5-yl, 1,7-naphthyridine-6-yl, 1,7-naphthyridine-8-yl, 2,6-naphthyridine-3-yl, 2,6-naphthyridine-4-yl, 2,6-naphthyridine-5-yl, 2,6-naphthyridine-7-yl, 2,6-naphthyridine-8-yl, pyridazine-3-yl, pyridazine-4-yl, pyridazine-5-yl, quinoline-2-yl, quinoline-3-yl, quinoline-4-yl, indoline-4-yl, indoline-5-yl, indoline-6-yl, indoline-7-yl, isoquinoline-1-yl, isoquinoline-3-yl, isoquinoline-4-yl, cinnoline-3-yl, cinnoline-5-yl, cinnoline-6-yl, phthalazine-5-yl, phthalazine-6-yl, quinazoline-4-yl, quinazoline-5-yl, quinazoline-6-yl, quinazoline-8-yl, 1,6-naphthyridine-2-yl, 1,6-naphthyridine-3-yl, 1,6-naphthyridine-4-yl, 1,6-naphthyridine-5-yl, 1,6-naphthyridine-7-yl, 1,6-naphthyridine-8-yl, 1,8-naphthyridine-2-yl, 1,8-naphthyridine-3-yl, 1,8-naphthyridine-4-yl, 1,7-naphthyridine-3-yl, 1,7-naphthyridine-4-yl, 2,6-naphthyridine-1-yl, 2,7-naphthyridine-3-yl, 2,7-naphthyridine-4-yl, pyridine-2-yl, pyridine-3-yl, pyridine-4-yl, benzisothiazolyl, and benzothiazolyl;
Ar is an aryl selected from the group consisting of phenyl, benzothiazole, and pyridine; provided that when HET is pyrazolyl, Ar is not phenyl; and provided that when HET is benzimidazolyl, Ar is not phenyl; and provided that when HET is benzofuranyl, Ar is not pyridinyl; and provided that when HET is isoquinolinyl, Ar is not pyridinyl; and provided that when HET is pyridinyl, Ar is not pyridinyl or phenyl; and
   enantiomers, diastereomers, tautomers, solvates, and pharmaceutically acceptable salts thereof.

In another embodiment, the present invention is directed to compounds of formula (I) wherein
HET is a heteroaryl selected from the group consisting of phthalazine-4-yl, isoindole-4-yl, isoindole-5-yl, isoindole-6-yl, isoindole-7-yl, ih-indazole-4-yl, ih-indazole-5-yl, ih-indazole-6-yl, ih-indazole-7-yl, purine-2-yl, purine-6-yl, purine-8-yl, benzimidazole-4-yl, benzimidazole-5-yl, benzimidazole-6-yl, benzimidazole-7-yl, cinnoline-4-yl, cinnoline-7-yl, cinnoline-8-yl, quinoxaline-5-yl, quinoxaline-6-yl, 2,7-naphthyridine-2-yl, pyrazine-2-yl, pyrazine-3-yl, quinazoline-7-yl, 1,7-naphthyridine-2-yl, 1,7-naphthyridine-5-yl, 1,7-naphthyridine-6-yl, 1,7-naphthyridine-8-yl, 2,6-naphthyridine-3-yl, 2,6-naphthyridine-4-yl, 2,6-naphthyridine-5-yl, 2,6-naphthyridine-7-yl, 2,6-naphthyridine-8-yl, pyridazine-3-yl, pyridazine-4-yl, pyridazine-5-yl, quinoline-2-yl, quinoline-3-yl, quinoline-4-yl, indoline-4-yl, indoline-5-yl, indoline-6-yl, indoline-7-yl, isoquinoline-1-yl, isoquinoline-3-yl, isoquinoline-4-yl, cinnoline-3-yl, cinnoline-5-yl, cinnoline-6-yl, phthalazine-5-yl, phthalazine-6-yl, quinazoline-4-yl, quinazoline-5-yl, quinazoline-6-yl, quinazoline-8-yl, 1,6-naphthyridine-2-yl, 1,6-naphthyridine-3-yl, 1,6-naphthyridine-4-yl, 1,6-naphthyridine-5-yl, 1,6-naphthyridine-7-yl, 1,6-naphthyridine-8-yl, 1,8-naphthyridine-2-yl, 1,8-naphthyridine-3-yl, 1,8-naphthyridine-4-yl, 1,7-naphthyridine-3-yl, 1,7-naphthyridine-4-yl, 2,6-naphthyridine-1-yl, 2,7-naphthyridine-3-yl, 2,7-naphthyridine-4-yl, pyridine-2-yl, pyridine-3-yl, pyridine-4-yl, benzisothiazolyl, and benzothiazolyl;
Ar is an aryl selected from the group consisting of benzo[b]thiophenyl, naphthalenyl, biphenyl, quinolinyl, isoquinolinyl, thiophenyl, pyrimidinyl, pyridazinyl, phenyl, benzothiazolyl, and pyridinyl; provided that when HET is benzimidazolyl, Ar is not pyrimidinyl or phenyl; and provided that when HET is benzothiazolyl, Ar is not pyrimidinyl; and provided that when HET is isoquinolinyl, Ar is not pyridinyl; and provided that when HET is pyridinyl, Ar is not pyrimidinyl, pyridinyl or phenyl; and provided that when HET is quinolinyl, Ar is not pyrimidinyl; and
   enantiomers, diastereomers, tautomers, solvates, and pharmaceutically acceptable salts thereof.

In another embodiment, the present invention is directed to compounds of formula (I) wherein
HET is a heteroaryl selected from the group consisting of phthalazine-4-yl, isoindole-4-yl, isoindole-5-yl, isoindole-6-yl, isoindole-7-yl, ih-indazole-4-yl, ih-indazole-5-yl, ih-indazole-6-yl, ih-indazole-7-yl, purine-2-yl, purine-6-yl, purine-8-yl, benzimidazole-4-yl, benzimidazole-5-yl, benzimidazole-6-yl, benzimidazole-7-yl, cinnoline-4-yl, cinnoline-7-yl, cinnoline-8-yl, quinoxaline-5-yl, quinoxaline-6-yl, 2,7-naphthyridine-2-yl, pyrazine-2-yl, pyrazine-3-yl, quinazoline-7-yl, 1,7-naphthyridine-2-yl, 1,7-naphthyridine-5-yl, 1,7-naphthyridine-6-yl, 1,7-naphthyridine-8-yl, 2,6-naphthyridine-3-yl, 2,6-naphthyridine-4-yl, 2,6-naphthyridine-5-yl, 2,6-naphthyridine-7-yl, 2,6-naphthyridine-8-yl, pyridazine-3-yl, pyridazine-4-yl, pyridazine-5-yl, quinoline-2-yl, quinoline-3-yl, quinoline-4-yl, indoline-4-yl, indoline-5-yl, indoline-6-yl, indoline-7-yl, isoquinoline-1-yl, isoquinoline-3-yl, isoquinoline-4-yl, cinnoline-3-yl, cinnoline-5-yl, cinnoline-6-yl, phthalazine-5-yl, phthalazine-6-yl, quinazoline-4-yl, quinazoline-5-yl, quinazoline-6-yl, quinazoline-8-yl, 1,6-naphthyridine-2-yl, 1,6-naphthyridine-3-yl, 1,6-naphthyridine-4-yl, 1,6-naphthyridine-5-yl, 1,6-naphthyridine-7-yl, 1,6-naphthyridine-8-yl, 1,8-naphthyridine-2-yl, 1,8-naphthyridine-3-yl, 1,8-naphthyridine-4-yl, 1,7-naphthyridine-3-yl, 1,7-naphthyridine-4-yl, 2,6-naphthyridine-1-yl, 2,7-naphthyridine-3-yl, 2,7-naphthyridine-4-yl, pyridine-2-yl, pyridine-3-yl, pyridine-4-yl, benzisothiazolyl, and benzothiazolyl;
Ar is an aryl selected from the group consisting of thiophenyl, pyrimidinyl, pyridazinyl, phenyl, benzothiazolyl, and pyridinyl; provided that when HET is benzimidazolyl, Ar is not pyrimidinyl or phenyl; and provided that when HET is benzothiazolyl, Ar is not pyrimidinyl; and provided that when HET is isoquinolin-5-yl, Ar is not pyridinyl; and provided that when HET is pyridinyl, Ar is not pyrimidinyl, pyridinyl, or phenyl; and provided that when HET is quinolinyl, Ar is not pyrimidinyl; and
   enantiomers, diastereomers, tautomers, solvates, and pharmaceutically acceptable salts thereof.

In another embodiment, the present invention is directed to compounds of formula (I) wherein
HET is a heteroaryl selected from the group consisting of phthalazine-4-yl, isoindole-4-yl, isoindole-5-yl, isoindole-6-yl, isoindole-7-yl, ih-indazole-4-yl, ih-indazole-5-yl, ih-indazole-6-yl, ih-indazole-7-yl, purine-2-yl, purine-6-yl, purine-8-yl, benzimidazole-4-yl, benzimidazole-5-yl, benzimidazole-6-yl, benzimidazole-7-yl, cinnoline-4-yl, cinnoline-7-yl, cinnoline-8-yl, quinoxaline-5-yl, quinoxaline-6-yl, 2,7-naphthyridine-2-yl, pyrazine-2-yl, pyrazine-3-yl, quinazoline-7-yl, 1,7-naphthyridine-2-yl, 1,7-naphthyridine-5-yl, 1,7-naphthyridine-6-yl, 1,7-naphthyridine-8-yl, 2,6-naphthyridine-3-yl, 2,6-naphthyridine-4-yl, 2,6-naphthyridine-5-yl, 2,6-naphthyridine-7-yl, 2,6-naphthyridine-8-yl, pyridazine-3-yl, pyridazine-4-yl, pyridazine-5-yl, quinoline-2-yl, quinoline-3-yl, quinoline-4-yl, indoline-4-yl, indoline-5-yl, indoline-6-yl, indoline-7-yl, isoquinoline-1-yl, isoquinoline-3-yl, isoquinoline-4-yl, cinnoline-3-yl, cinnoline-5-yl, cinnoline-6-yl, phthalazine-5-yl, phthalazine-6-yl, quinazoline-4-yl, quinazoline-5-yl, quinazoline-6-yl, quinazoline-8-yl, 1,6-naphthyridine-2-yl, 1,6-naphthyridine-3-yl, 1,6-naphthyridine-4-yl, 1,6-naphthyridine-5-yl, 1,6-naphthyridine-7-yl, 1,6-naphthyridine-8-yl, 1,8-naphthyridine-2-yl, 1,8-naphthyridine-3-yl, 1,8-naphthyridine-4-yl, 1,7-naphthyridine-3-yl, 1,7-naphthyridine-4-yl, 2,6-naphthyridine-1-yl, 2,7-naphthyridine-3-yl, 2,7-naphthyridine-4-yl, pyridine-2-yl, pyridine-3-yl, pyridine-4-yl, benzisothiazolyl, and benzothiazolyl;
Ar is an aryl selected from the group consisting of phenyl, benzothiazole, and pyridine; provided that when HET is benzimidazolyl, Ar is not phenyl;

and provided that when HET is isoquinolinyl, Ar is not pyridinyl; and

provided that when HET is pyridinyl, Ar is not pyridinyl or phenyl; and enantiomers, diastereomers, tautomers, solvates, and pharmaceutically acceptable salts thereof.

In another embodiment, the present invention is directed to compounds of formula (I) wherein
HET is a heteroaryl selected from the group consisting of quinoline-2-yl, quinoline-3-yl, quinoline-4-yl, indoline-4-yl, indoline-5-yl, indoline-6-yl, indoline-7-yl, isoquinoline-1-yl, isoquinoline-3-yl, isoquinoline-4-yl, cinnoline-3-yl, cinnoline-5-yl, cinnoline-6-yl, phthalazine-5-yl, phthalazine-6-yl, quinazoline-4-yl, quinazoline-5-yl, quinazoline-6-yl, quinazoline-8-yl, 1,6-naphthyridine-2-yl, 1,6-naphthyridine-3-yl, 1,6-naphthyridine-4-yl, 1,6-naphthyridine-5-yl, 1,6-naphthyridine-7-yl, 1,6-naphthyridine-8-yl, 1,8-naphthyridine-2-yl, 1,8-naphthyridine-3-yl, 1,8-naphthyridine-4-yl, 1,7-naphthyridine-3-yl, 1,7-naphthyridine-4-yl, 2,6-naphthyridine-1-yl, 2,7-naphthyridine-3-yl, 2,7-naphthyridine-4-yl, pyridine-2-yl, pyridine-3-yl, pyridine-4-yl, benzisothiazolyl, and benzothiazolyl;
Ar is an aryl selected from the group consisting of benzo[b]thiophenyl, naphthalenyl, biphenyl, quinolinyl, isoquinolinyl, thiophenyl, pyrimidinyl, pyridazinyl, phenyl, benzothiazolyl, and pyridinyl; provided that when HET is benzothiazolyl, Ar is not pyrimidiny; and provided that when HET is isoquinolinyl, Ar is not pyridinyl; and provided that when HET is pyridinyl, Ar is not pyrimidinyl, pyridinyl, or phenyl; and provided that when HET is quinolinyl, Ar is not pyrimidinyl; and provided that when HET is thiazolyl, Ar is not quinolinyl; and
   enantiomers, diastereomers, tautomers, solvates, and pharmaceutically acceptable salts thereof.

In another embodiment, the present invention is directed to compounds of formula (I) wherein
HET is a heteroaryl selected from the group consisting of quinoline-2-yl, quinoline-3-yl, quinoline-4-yl, indoline-4-yl, indoline-5-yl, indoline-6-yl, indoline-7-yl, isoquinoline-1-yl, isoquinoline-3-yl, isoquinoline-4-yl, cinnoline-3-yl, cinnoline-5-yl, cinnoline-6-yl, phthalazine-5-yl, phthalazine-6-yl, quinazoline-4-yl, quinazoline-5-yl, quinazoline-6-yl, quinazoline-8-yl, 1,6-naphthyridine-2-yl, 1,6-naphthyridine-3-yl, 1,6-naphthyridine-4-yl, 1,6-naphthyridine-5-yl, 1,6-naphthyridine-7-yl, 1,6-naphthyridine-8-yl, 1,8-naphthyridine-2-yl, 1,8-naphthyridine-3-yl, 1,8-naphthyridine-4-yl, 1,7-naphthyridine-3-yl, 1,7-naphthyridine-4-yl, 2,6-naphthyridine-1-yl, 2,7-naphthyridine-3-yl, 2,7-naphthyridine-4-yl, pyridine-2-yl, pyridine-3-yl, pyridine-4-yl, benzisothiazolyl, and benzothiazolyl;
Ar is an aryl selected from the group consisting of thiophenyl, pyrimidinyl, pyridazinyl, phenyl, benzothiazolyl, and pyridinyl; provided that when HET is isoquinolinyl, Ar is not pyridinyl; and provided that when HET is pyridinyl, Ar is not pyridinyl, phenyl, 2-quinazolinyl, 5-quinoxalinyl or pyrimidinyl; and provided that when HET is quinolinyl, Ar is not pyrimidinyl; and
   enantiomers, diastereomers, tautomers, solvates, and pharmaceutically acceptable salts thereof.

In another embodiment, the present invention is directed to compounds of formula (I) wherein
HET is a heteroaryl selected from the group consisting of quinoline-2-yl, quinoline-3-yl, quinoline-4-yl, indoline-4-yl, indoline-5-yl, indoline-6-yl, indoline-7-yl, isoquinoline-1-yl, isoquinoline-3-yl, isoquinoline-4-yl, cinnoline-3-yl, cinnoline-5-yl, cinnoline-6-yl, phthalazine-5-yl, phthalazine-6-yl, quinazoline-4-yl, quinazoline-5-yl, quinazoline-6-yl, quinazoline-8-yl, 1,6-naphthyridine-2-yl, 1,6-naphthyridine-3-yl, 1,6-naphthyridine-4-yl, 1,6-naphthyridine-5-yl, 1,6-naphthyridine-7-yl, 1,6-naphthyridine-8-yl, 1,8-naphthyridine-2-yl, 1,8-naphthyridine-3-yl, 1,8-naphthyridine-4-yl, 1,7-naphthyridine-3-yl, 1,7-naphthyridine-4-yl, 2,6-naphthyridine-1-yl, 2,7-naphthyridine-3-yl, 2,7-naphthyridine-4-yl, pyridine-2-yl, pyridine-3-yl, pyridine-4-yl, benzisothiazolyl, and benzothiazolyl;
Ar is an aryl selected from the group consisting of phenyl, benzothiazolyl, and pyridinyl; provided that when HET is isoquinolinyl, Ar is not pyridinyl; and provided that when HET is pyridinyl, Ar is not pyridinyl or phenyl; and
   enantiomers, diastereomers, tautomers, solvates, and pharmaceutically acceptable salts thereof.

In another embodiment, the present invention is directed to compounds of formula (II) 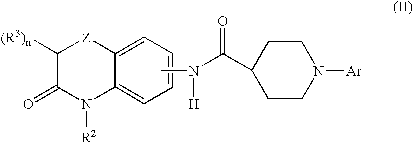
wherein
Ar is an aryl selected from the group consisting of benzo[b]thiophenyl, naphthyl, biphenyl, isoquinolinyl, thiophenyl, pyridazinyl, and benzothiazolyl;
Z is O or S;
R² is hydrogen or C₁₋₆alkyl optionally substituted with —OR⁴;
n is 1 or 2;
R³ is independently hydrogen, C₁₋₆alkyl, —COOR⁴, or —CH₂COOR⁴;
R⁴ is hydrogen or C₁₋₃alkyl; and
enantiomers, diastereomers, tautomers, solvates, and pharmaceutically acceptable salts thereof.

In another embodiment, the present invention is directed to compounds of formula (II) wherein Z is O and n is 1.

In another embodiment, the present invention is directed to compounds of formula (II) wherein Z is O and R² is hydrogen.

In another embodiment, the present invention is directed to compounds of formula (III): 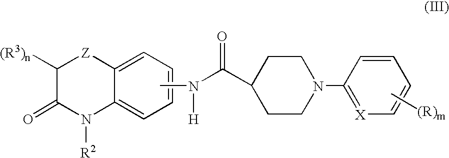
wherein
X is CH or N;
m is an integer from 0 to 4;
R is independently selected from the group consisting of halogen; R¹; fluorinated C₁₋₁₀alkyl; phenyl; amino; cyano; CF₃O—; a 3 membered cyclic heteroalkyl containing 1 heteroatom that is N, O or S wherein said 3 membered cyclic heteroalkyl is optionally substituted with a substituent that is halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a 4 to 5 membered cyclic heteroalkyl containing 1-3 heteroatoms that independently are N, O or S wherein said 4 to 5 membered cyclic heteroalkyl is optionally substituted with 1 to 2 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a 6 to 7 membered cyclic heteroalkyl containing 1-3 heteroatoms that independently are N, O or S wherein said 6 to 7 membered cyclic heteroalkyl is optionally substituted with 1 to 3 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a heteroaryl wherein said heteroaryl is cinnoline, furan, imidazole, indazole, indole, indoline, indolizine, isobenzofuran, isoindole, isoindoline, isoquinoline, isothiazole, isoxazole, naphthyridine, oxadiazole, oxazole, pthalazine, pteridine, pyran, pyrazine, pyrazole, pyridazine, pyridine, pyrimidine, pyrrole, pyrrolizine, quinoline, quinolizine, quinazoline, quinoxaline, tetrazole, thiadiazole, triazine, or triazole wherein said heteroaryl is optionally substituted with 1 to 3 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; hydroxyl; R¹⁰—; R¹S—; R¹SO₂—; R¹S(O)—; R¹SO₂NH—; -LCOY; and C₆₋₁₀aryl;
R¹ is C₁₋₁₀alkyl;
L is —NH—, a direct bond, —O—, or —CH₂—;
Y is H, R¹, HO, R¹O—, R¹S—, —NH₂, R¹NH—, or (R¹)₂N—;
Z is O or S;
R² is hydrogen or C₁₋₆alkyl optionally substituted with —OR⁴;
n is 1 or 2;
R³ is independently hydrogen, C₁₋₆alkyl, —COOR⁴, or —CH₂COOR⁴;
R⁴ is hydrogen or C₁₋₃alkyl; and
enantiomers, diastereomers, tautomers, solvates, and pharmaceutically acceptable salts thereof.

In another embodiment, the present invention is directed to compounds of formula (III) wherein Z is O and n is 1.

In another embodiment, the present invention is directed to compounds of formula (III) wherein Z is O and R² is hydrogen.

In another embodiment, the present invention is directed to compounds of formula (IIa): 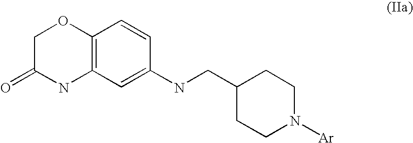
wherein Ar is an aryl selected from the group consisting of benzo[b]thiophenyl, naphthyl, biphenyl, isoquinolinyl, thiophenyl, pyridazinyl, and benzothiazolyl; and
   enantiomers, diastereomers, tautomers, solvates, and pharmaceutically acceptable salts thereof.

In another embodiment, the present invention is directed to compounds of formula (IIIa): 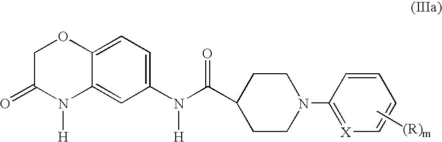
wherein
X is CH or N;
m is an integer from 0 to 4;
R is independently selected from the group consisting of halogen; R¹; fluorinated C₁₋₁₀alkyl; phenyl; amino; cyano; CF₃O—; a 3 membered cyclic heteroalkyl containing 1 heteroatom that is N, O or S wherein said 3 membered cyclic heteroalkyl is optionally substituted with a substituent that is halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a 4 to 5 membered cyclic heteroalkyl containing 1-3 heteroatoms that independently are N, O or S wherein said 4 to 5 membered cyclic heteroalkyl is optionally substituted with 1 to 2 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a 6 to 7 membered cyclic heteroalkyl containing 1-3 heteroatoms that independently are N, O or S wherein said 6 to 7 membered cyclic heteroalkyl is optionally substituted with 1 to 3 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a heteroaryl wherein said heteroaryl is cinnoline, furan, imidazole, indazole, indole, indoline, indolizine, isobenzofuran, isoindole, isoindoline, isoquinoline, isothiazole, isoxazole, naphthyridine, oxadiazole, oxazole, pthalazine, pteridine, pyran, pyrazine, pyrazole, pyridazine, pyridine, pyrimidine, pyrrole, pyrrolizine, quinoline, quinolizine, quinazoline, quinoxaline, tetrazole, thiadiazole, triazine, or triazole wherein said heteroaryl is optionally substituted with 1 to 3 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; hydroxyl; R¹O—; R¹S—; R¹SO₂—; R¹S(O)—; R¹SO₂NH—; -LCOY; and C₆₋₁₀aryl;
wherein R¹ is C₁₋₁₀alkyl; L is —NH—, a direct bond, —O—, or —CH₂—; and Y is H, R¹, HO, R¹O—, R¹S—, —NH₂, R¹NH—, or (R¹)₂N—; and
   enantiomers, diastereomers, tautomers, solvates, and pharmaceutically acceptable salts thereof.

In another embodiment, the present invention is directed to compounds of formula (IIIa) wherein X is CH.

In another embodiment, the present invention is directed to compounds of formula (IIIa) wherein m is 1 or 2.

In another embodiment, the present invention is directed to compounds of formula (IIIa) wherein R is independently C₁₋₄alkanyl, CF₃, or halo.

In another embodiment, the present invention is directed to compounds of formula (IIIa) wherein X is CH, m is 1 or 2 and R is independently C₁₋₄alkanyl, CF₃, or halo.

The compounds of Formulas (I), (II) and (IIa) in the embodiments above are optionally substituted as follows:
HET is optionally substituted at 1-3 substitutable atoms with a substituent that is independently selected from the group consisting of hydroxyl; R¹; R¹O—; R¹S—; CF₃O—; R¹S(O)—; R¹SO₂—; -LCOY; C₆₋₁₀aryl; a 3 to 7 membered cyclic heteroalkanyl containing from 1 to 3 heteroatoms wherein said heteroatoms independently are N, O or S; and a 5 to 10 membered heteroaryl selected from the group consisting of benzofuran, benzimidazole, benzisoxazole, benzthiazole, benzothiophene, benzoxazole, cinnoline, furan, imidazole, imidazoline, indazole, indole, indoline, indolizine, isobenzofuran, isoindole, isoindoline, isoquinoline, isothiazole, isoxazole, naphthyridine, oxadiazole, oxazole, pthalazine, pteridine, purine, pyran, pyrazine, pyrazole, pyridazine, pyridine, pyrimidine, pyrrole, pyrrolizine, quinoline, quinolizine, quinazoline, quinoxaline, tetrazole, thiadiazole, thiazole, thiophene, triazine, and triazole;
Ar is optionally substituted with one to four substituents independently selected from the group consisting of halogen; R¹; fluorinated C₁₋₁₀alkyl; phenyl; amino; cyano; CF₃O—; a 3 membered cyclic heteroalkyl containing 1 heteroatom that is N, O or S wherein said 3 membered cyclic heteroalkyl is optionally substituted with a substituent that is halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a 4 to 5 membered cyclic heteroalkyl containing 1-3 heteroatoms that independently are N, O or S wherein said 4 to 5 membered cyclic heteroalkyl is optionally substituted with 1 to 2 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a 6 to 7 membered cyclic heteroalkyl containing 1-3 heteroatoms that independently are N, O or S wherein said 6 to 7 membered cyclic heteroalkyl is optionally substituted with 1 to 3 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a heteroaryl wherein said heteroaryl is cinnoline, furan, imidazole, indazole, indole, indoline, indolizine, isobenzofuran, isoindole, isoindoline, isoquinoline, isothiazole, isoxazole, naphthyridine, oxadiazole, oxazole, pthalazine, pteridine, pyran, pyrazine, pyrazole, pyridazine, pyridine, pyrimidine, pyrrole, pyrrolizine, quinoline, quinolizine, quinazoline, quinoxaline, tetrazole, thiadiazole, triazine, or triazole wherein said heteroaryl is optionally substituted with 1 to 3 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; hydroxyl; R¹O—; R¹S—; R¹SO₂—; R¹S(O)—; R¹SO₂NH—; -LCOY; and C₆₋₁₀aryl;
   wherein R¹ is C₁₋₁₀alkyl; L is —NH—, a direct bond, —O—, or —CH₂—; and Y is H, R¹, HO, R¹O—, R¹S—, —NH₂, R¹NH—, or (R¹)₂N—.

In another embodiment, the present invention is directed to a compound selected from the group consisting of
1-(3-Trifluoromethyl-phenyl)-piperidine-4-carboxylic acid quinolin-3-ylamide;
1-Biphenyl-3-yl-piperidine-4-carboxylic acid quinolin-3-ylamide;
1-(4-Trifluoromethyl-phenyl)-piperidine-4-carboxylic acid quinolin-3-ylamide;
1-(2,3-Dimethyl-phenyl)-piperidine-4-carboxylic acid quinolin-3-ylamide;
1-(3,4-Dimethyl-phenyl)-piperidine-4-carboxylic acid quinolin-3-ylamide;
1-(3-Trifluoromethyl-phenyl)-piperidine-4-carboxylic acid (9-ethyl-9H-purin-6-yl)-amide;
1-(3-Trifluoromethyl-phenyl)-piperidine-4-carboxylic acid (1H-indol-4-yl)-amide;
1-(3-Trifluoromethyl-phenyl)-piperidine-4-carboxylic acid (1-acetyl-2,3-dihydro-1H-indol-7-yl)-amide;
1-(3-Trifluoromethyl-phenyl)-piperidine-4-carboxylic acid thieno[2,3-d]pyrimidin-4-ylamide;
1-(3-Trifluoromethyl-phenyl)-piperidine-4-carboxylic acid (1H-indazol-7-yl)-amide;
1-(3-Trifluoromethyl-phenyl)-piperidine-4-carboxylic acid (3-methyl-benzo[d]isothiazol-5-yl)-amide;
1-(3-Trifluoromethyl-phenyl)-piperidine-4-carboxylic acid benzothiazol-6-ylamide;
3-{[1-(3-Trifluoromethyl-phenyl)-piperidine-4-carbonyl]-amino}-1H-indole-2-carboxylic acid ethyl ester;
1-(3-Trifluoromethyl-phenyl)-piperidine-4-carboxylic acid benzo[1,3]dioxol-5-ylamide;
1-(3-Trifluoromethyl-phenyl)-piperidine-4-carboxylic acid (1-acetyl-2,3-dihydro-1H-indol-6-yl)-amide;
1-(3-Trifluoromethyl-phenyl)-piperidine-4-carboxylic acid (1-acetyl-2,3-dihydro-1H-indol-7-yl)-amide;
1-(3-Trifluoromethyl-phenyl)-piperidine-4-carboxylic acid (2-oxo-2,3-dihydro-1H-indol-4-yl)-amide;
1-(3-Trifluoromethyl-phenyl)-piperidine-4-carboxylic acid (2,3-dihydro-benzo[1,4]dioxin-6-yl)-amide;
1-(3-Trifluoromethyl-phenyl)-piperidine-4-carboxylic acid (2-methyl-benzothiazol-5-yl)-amide;
1-(3-Trifluoromethyl-phenyl)-piperidine-4-carboxylic acid (5-nitro-benzo[d]isothiazol-3-yl)-amide;
1-(3-Trifluoromethyl-phenyl)-piperidine-4-carboxylic acid (1H-indol-4-yl)-amide;
1-(3-Trifluoromethyl-phenyl)-piperidine-4-carboxylic acid (1H-indol-5-yl)-amide;
1-(3-Trifluoromethyl-phenyl)-piperidine-4-carboxylic acid (1H-indol-6-yl)-amide;
1-(3-Trifluoromethyl-phenyl)-piperidine-4-carboxylic acid (1-methyl-1H-indazol-5-yl)-amide;
1-(3-Trifluoromethyl-phenyl)-piperidine-4-carboxylic acid (2-mercapto-benzothiazol-6-yl)-amide;
1-(3-Trifluoromethyl-phenyl)-piperidine-4-carboxylic acid (8-hydroxy-quinolin-5-yl)-amide;
1-(3-Trifluoromethyl-phenyl)-piperidine-4-carboxylic acid (3,4-dihydro-2H-benzo[b][1,4]dioxepin-7-yl)-amide;
1-(3-Trifluoromethyl-phenyl)-piperidine-4-carboxylic acid (1-acetyl-2,3-dihydro-1H-indol-5-yl)-amide;
1-(3-Trifluoromethyl-phenyl)-piperidine-4-carboxylic acid (1H-indol-7-yl)-amide;
1-(3-Trifluoromethyl-phenyl)-piperidine-4-carboxylic acid (1,4-dioxo-1,2,3,4-tetrahydro-phthalazin-6-yl)-amide;
1-(3-Trifluoromethyl-phenyl)-piperidine-4-carboxylic acid (6-phenoxy-pyridin-3-yl)-amide;
1-(3-Trifluoromethyl-phenyl)-piperidine-4-carboxylic acid (6-acetyl-benzo[1,3]dioxol-5-yl)-amide;
1-(3-Trifluoromethyl-phenyl)-piperidine-4-carboxylic acid [4-(4,6-dimethoxy-pyrimidin-2-yl)-phenyl]-amide;
1-(3-Trifluoromethyl-phenyl)-piperidine-4-carboxylic acid (3-oxo-3,4-dihydro-2H-benzo[1,4]oxazin-6-yl)-amide;
1-(3-Trifluoromethyl-phenyl)-piperidine-4-carboxylic acid (4-dimethylamino-5-phenyl-pyrimidin-2-yl)-amide; and
3,5-Dimethyl-1-(5-{[1-(3-trifluoromethyl-phenyl)-piperidine-4-carbonyl]-amino}-pyridin-2-yl)-1H-pyrazole-4-carboxylic acid ethyl ester.

In another embodiment, the present invention is directed to a compound selected from the group consisting of
1-Phenyl-piperidine-4-carboxylic acid quinolin-3-ylamide;
1-Benzothiazol-5-yl-piperidine-4-carboxylic acid quinolin-3-ylamide;
1-Benzothiazol-5-yl-piperidine-4-carboxylic acid (2,3-dihydro-1H-indol-4-yl)-amide;
1-Phenyl-piperidine-4-carboxylic acid (2,3-dihydro-1H-indol-4-yl)-amide;
3,4,5,6-Tetrahydro-2H-[1,3′]bipyridinyl-4-carboxylic acid isoquinolin-1-ylamide;
1-Phenyl-piperidine-4-carboxylic acid isoquinolin-1-ylamide;
1-Phenyl-piperidine-4-carboxylic acid cinnolin-3-ylamide;
1-Benzothiazol-5-yl-piperidine-4-carboxylic acid cinnolin-3-ylamide;
1-Benzothiazol-5-yl-piperidine-4-carboxylic acid phthalazin-5-ylamide;
3,4,5,6-Tetrahydro-2H-[1,4′]bipyridinyl-4-carboxylic acid phthalazin-5-ylamide;
1-Phenyl-piperidine-4-carboxylic acid quinazolin-4-ylamide;
1-Benzothiazol-5-yl-piperidine-4-carboxylic acid quinazolin-4-ylamide;
1-Benzothiazol-5-yl-piperidine-4-carboxylic acid pyridin-3-ylamide;
1-Phenyl-piperidine-4-carboxylic acid benzo[d]isothiazol-3-ylamide;
3,4,5,6-Tetrahydro-2H-[1,2′]bipyridinyl-4-carboxylic acid benzo[d]isothiazol-3-ylamide;
1-Phenyl-piperidine-4-carboxylic acid benzothiazol-2-ylamide;
3,4,5,6-Tetrahydro-2H-[1,3′]bipyridinyl-4-carboxylic acid benzothiazol-2-ylamide;
1-Pyridazin-3-yl-piperidine-4-carboxylic acid phthalazin-1-ylamide;
1-Thiophen-2-yl-piperidine-4-carboxylic acid phthalazin-1-ylamide;
1-Thiophen-2-yl-piperidine-4-carboxylic acid (7H-purin-6-yl)-amide;
1-Pyrimidin-2-yl-piperidine-4-carboxylic acid (7H-purin-6-yl)-amide;
1-Pyrimidin-2-yl-piperidine-4-carboxylic acid cinnolin-4-ylamide;
1-Thiophen-2-yl-piperidine-4-carboxylic acid cinnolin-4-ylamide;
1-Thiophen-2-yl-piperidine-4-carboxylic acid quinoxalin-6-ylamide;
1-Pyridazin-3-yl-piperidine-4-carboxylic acid quinoxalin-6-ylamide;
1-Pyrimidin-2-yl-piperidine-4-carboxylic acid quinoxalin-6-ylamide;
1-Pyrimidin-2-yl-piperidine-4-carboxylic acid pyrazin-2-ylamide;
1-Thiophen-2-yl-piperidine-4-carboxylic acid pyrazin-2-ylamide;
1-Thiophen-2-yl-piperidine-4-carboxylic acid quinazolin-7-ylamide;
1-Pyrimidin-2-yl-piperidine-4-carboxylic acid quinazolin-7-ylamide;
1-Pyrimidin-2-yl-piperidine-4-carboxylic acid pyridazin-3-ylamide;
1-Thiophen-2-yl-piperidine-4-carboxylic acid pyridazin-3-ylamide;
1-Benzo[b]thiophen-3-yl-piperidine-4-carboxylic acid (2-oxo-2,3-dihydro-1H-benzoimidazol-5-yl)-amide;
1-Naphthalen-2-yl-piperidine-4-carboxylic acid (2-oxo-2,3-dihydro-1H-benzoimidazol-5-yl)-amide;
1-Naphthalen-2-yl-piperidine-4-carboxylic acid (3-oxo-3,4-dihydro-2H-benzo[1,4]oxazin-6-yl)-amide;
1-Biphenyl-3-yl-piperidine-4-carboxylic acid (3-oxo-3,4-dihydro-2H-benzo[1,4]oxazin-6-yl)-amide;
1-Biphenyl-3-yl-piperidine-4-carboxylic acid quinazolin-2-ylamide;
1-Quinolin-3-yl-piperidine-4-carboxylic acid quinazolin-2-ylamide;
1-Quinolin-3-yl-piperidine-4-carboxylic acid quinoxalin-2-ylamide;
1-Isoquinolin-5-yl-piperidine-4-carboxylic acid quinoxalin-2-ylamide;
1-Isoquinolin-5-yl-piperidine-4-carboxylic acid (9H-carbazol-3-yl)-amide;
1-Benzo[b]thiophen-3-yl-piperidine-4-carboxylic acid (9H-carbazol-3-yl)-amide;
1-Benzo[b]thiophen-3-yl-piperidine-4-carboxylic acid phenazin-2-ylamide;
1-Naphthalen-2-yl-piperidine-4-carboxylic acid phenazin-2-ylamide;
1-Naphthalen-2-yl-piperidine-4-carboxylic acid thiazol-2-ylamide;
1-Biphenyl-3-yl-piperidine-4-carboxylic acid thiazol-2-ylamide;
1-Biphenyl-3-yl-piperidine-4-carboxylic acid (1H-imidazol-2-yl)-amide;
1-Quinolin-3-yl-piperidine-4-carboxylic acid (1H-imidazol-2-yl)-amide;
1-Quinolin-3-yl-piperidine-4-carboxylic acid (2H-pyrazol-3-yl)-amide;
1-Isoquinolin-5-yl-piperidine-4-carboxylic acid (2H-pyrazol-3-yl)-amide;
1-Isoquinolin-5-yl-piperidine-4-carboxylic acid isoxazol-3-ylamide;
1-Benzo[b]thiophen-3-yl-piperidine-4-carboxylic acid isoxazol-3-ylamide;
1-Benzo[b]thiophen-3-yl-piperidine-4-carboxylic acid (3-methyl-isothiazol-5-yl)-amide;
1-Naphthalen-2-yl-piperidine-4-carboxylic acid (3-methyl-isothiazol-5-yl)-amide;
1-Isoquinolin-5-yl-piperidine-4-carboxylic acid [1,6]naphthyridin-4-ylamide;
1-Benzo[b]thiophen-3-yl-piperidine-4-carboxylic acid [1,6]naphthyridin-4-ylamide;
1-Benzo[b]thiophen-3-yl-piperidine-4-carboxylic acid [1,7]naphthyridin-8-ylamide;
1-Naphthalen-2-yl-piperidine-4-carboxylic acid [1,7]naphthyridin-8-ylamide;
1-Naphthalen-2-yl-piperidine-4-carboxylic acid acridin-9-ylamide;
1-Biphenyl-3-yl-piperidine-4-carboxylic acid acridin-9-ylamide;
1-Biphenyl-3-yl-piperidine-4-carboxylic acid (1-methyl-4,5-dihydro-1H-imidazol-2-yl)-amide;
1-Quinolin-3-yl-piperidine-4-carboxylic acid (1-methyl-4,5-dihydro-1H-imidazol-2-yl)-amide;
1-Quinolin-3-yl-piperidine-4-carboxylic acid (4,5-dihydro-1H-pyrazol-3-yl)-amide;
1-Isoquinolin-5-yl-piperidine-4-carboxylic acid (4,5-dihydro-1H-pyrazol-3-yl)-amide;
1-Isoquinolin-5-yl-piperidine-4-carboxylic acid [1,8]naphthyridin-2-ylamide;
1-Benzo[b]thiophen-3-yl-piperidine-4-carboxylic acid [1,8]naphthyridin-2-ylamide;
1-Benzo[b]thiophen-3-yl-piperidine-4-carboxylic acid [2,6]naphthyridin-1-ylamide;
1-Naphthalen-2-yl-piperidine-4-carboxylic acid [2,6]naphthyridin-1-ylamide;
1-Naphthalen-2-yl-piperidine-4-carboxylic acid benzo[d]isoxazol-5-ylamide;
1-Biphenyl-3-yl-piperidine-4-carboxylic acid benzo[d]isoxazol-5-ylamide;
1-Biphenyl-3-yl-piperidine-4-carboxylic acid (1H-pyrrol-3-yl)-amide;
1-Quinolin-3-yl-piperidine-4-carboxylic acid (1H-pyrrol-3-yl)-amide;
1-Quinolin-3-yl-piperidine-4-carboxylic acid (1H-pyrrolo[2,3-b]pyridin-3-yl)-amide;
1-Isoquinolin-5-yl-piperidine-4-carboxylic acid (1H-pyrrolo[2,3-b]pyridin-3-yl)-amide;
1-Isoquinolin-5-yl-piperidine-4-carboxylic acid pteridin-4-ylamide;
1-Benzo[b]thiophen-3-yl-piperidine-4-carboxylic acid pteridin-4-ylamide;
1-Benzo[b]thiophen-3-yl-piperidine-4-carboxylic acid (1,4-dihydro-quinazolin-6-yl)-amide;
1-Naphthalen-2-yl-piperidine-4-carboxylic acid (1,4-dihydro-quinazolin-6-yl)-amide;
1-Naphthalen-2-yl-piperidine-4-carboxylic acid [1,7]naphthyridin-4-ylamide;
1-Biphenyl-3-yl-piperidine-4-carboxylic acid [1,7]naphthyridin-4-ylamide;
1-Biphenyl-3-yl-piperidine-4-carboxylic acid [2,7]naphthyridin-4-ylamide;
1-Quinolin-3-yl-piperidine-4-carboxylic acid [2,7]naphthyridin-4-ylamide;
1-Quinolin-3-yl-piperidine-4-carboxylic acid [2,6]naphthyridin-1-ylamide; and
1-Isoquinolin-5-yl-piperidine-4-carboxylic acid [2,6]naphthyridin-1-ylamide.

In another embodiment, the present invention is directed to a compound selected from the group consisting of
1-(2,3-Dimethyl-phenyl)-piperidine-4-carboxylic acid (3-oxo-3,4-dihydro-2H-benzo[1,4]oxazin-6-yl)-amide;
1-(2,5-Bis-trifluoromethyl-phenyl)-piperidine-4-carboxylic acid (3-oxo-3,4-dihydro-2H-benzo[1,4]oxazin-6-yl)-amide;
1-(2-Bromo-3-fluoro-phenyl)-piperidine-4-carboxylic acid (3-oxo-3,4-dihydro-2H-benzo[1,4]oxazin-6-yl)-amide;
1-(2-Bromo-phenyl)-piperidine-4-carboxylic acid (3-oxo-3,4-dihydro-2H-benzo[1,4]oxazin-6-yl)-amide;
1-(2-Chloro-phenyl)-piperidine-4-carboxylic acid (3-oxo-3,4-dihydro-2H-benzo[1,4]oxazin-6-yl)-amide;
1-(2-Cyano-3-trifluoromethyl-phenyl)-piperidine-4-carboxylic acid (3-oxo-3,4-dihydro-2H-benzo[1,4]oxazin-6-yl)-amide;
1-(2-Cyano-5-trifluoromethyl-phenyl)-piperidine-4-carboxylic acid (3-oxo-3,4-dihydro-2H-benzo[1,4]oxazin-6-yl)-amide;
1-(2-Ethyl-phenyl)-piperidine-4-carboxylic acid (3-oxo-3,4-dihydro-2H-benzo[1,4]oxazin-6-yl)-amide;
1-(2-Fluoro-phenyl)-piperidine-4-carboxylic acid (3-oxo-3,4-dihydro-2H-benzo[1,4]oxazin-6-yl)-amide;
1-(2-Isopropyl-phenyl)-piperidine-4-carboxylic acid (3-oxo-3,4-dihydro-2H-benzo[1,4]oxazin-6-yl)-amide;
1-(2-Trifluoromethyl-phenyl)-piperidine-4-carboxylic acid (3-oxo-3,4-dihydro-2H-benzo[1,4]oxazin-6-yl)-amide;
1-(3,4,5-Trifluoro-phenyl)-piperidine-4-carboxylic acid (3-oxo-3,4-dihydro-2H-benzo[1,4]oxazin-6-yl)-amide;
1-(3,4-Difluoro-phenyl)-piperidine-4-carboxylic acid (3-oxo-3,4-dihydro-2H-benzo[1,4]oxazin-6-yl)-amide;
1-(3,4-Dimethyl-phenyl)-piperidine-4-carboxylic acid (3-oxo-3,4-dihydro-2H-benzo[1,4]oxazin-6-yl)-amide;
1-(3,5-Difluoro-phenyl)-piperidine-4-carboxylic acid (3-oxo-3,4-dihydro-2H-benzo[1,4]oxazin-6-yl)-amide;
1-(3-Bromo-phenyl)-piperidine-4-carboxylic acid (3-oxo-3,4-dihydro-2H-benzo[1,4]oxazin-6-yl)-amide;
1-(3-Chloro-2-methyl-phenyl)-piperidine-4-carboxylic acid (3-oxo-3,4-dihydro-2H-benzo[1,4]oxazin-6-yl)-amide;
1-(3-Chloro-phenyl)-piperidine-4-carboxylic acid (3-oxo-3,4-dihydro-2H-benzo[1,4]oxazin-6-yl)-amide;
1-(3-Fluoro-phenyl)-piperidine-4-carboxylic acid (3-oxo-3,4-dihydro-2H-benzo[1,4]oxazin-6-yl)-amide;
1-(3-Methoxy-phenyl)-piperidine-4-carboxylic acid (3-oxo-3,4-dihydro-2H-benzo[1,4]oxazin-6-yl)-amide;
1-(3-Trifluoromethyl-phenyl)-piperidine-4-carboxylic acid (2-oxo-2,3-dihydro-1H-pyrido[2,3-b][1,4]oxazin-7-yl)-amide;
1-(3-Trifluoromethyl-phenyl)-piperidine-4-carboxylic acid [4-(2-hydroxy-ethyl)-3-oxo-3,4-dihydro-2H-benzo[1,4]oxazin-6-yl]-amide;
1-(4-Bromo-2,3,5,6-tetrafluoro-phenyl)-piperidine-4-carboxylic acid (3-oxo-3,4-dihydro-2H-benzo[1,4]oxazin-6-yl)-amide;
1-(4-tert-Butyl-phenyl)-piperidine-4-carboxylic acid (3-oxo-3,4-dihydro-2H-benzo[1,4]oxazin-6-yl)-amide;
1-Benzothiazol-2-yl-piperidine-4-carboxylic acid (3-oxo-3,4-dihydro-2H-benzo[1,4]oxazin-6-yl)-amide;
1-m-Tolyl-piperidine-4-carboxylic acid (3-oxo-3,4-dihydro-2H-benzo[1,4]oxazin-6-yl)-amide;
1-o-Tolyl-piperidine-4-carboxylic acid (3-oxo-3,4-dihydro-2H-benzo[1,4]oxazin-6-yl)-amide;
1-Phenyl-piperidine-4-carboxylic acid (3-oxo-3,4-dihydro-2H-benzo[1,4]oxazin-6-yl)-amide;
3′-Methyl-3,4,5,6-tetrahydro-2H-[1,2′]bipyridinyl-4-carboxylic acid (3-oxo-3,4-dihydro-2H-benzo[1,4]oxazin-6-yl)-amide; and
3′-Nitro-3,4,5,6-tetrahydro-2H-[1,2′]bipyridinyl-4-carboxylic acid (3-oxo-3,4-dihydro-2H-benzo[1,4]oxazin-6-yl)-amide.

In another embodiment, the present invention is directed to a compound selected from the group consisting of
1-(2,3-Dimethyl-phenyl)-piperidine-4-carboxylic acid (3-oxo-3,4-dihydro-2H-benzo[1,4]oxazin-6-yl)-amide;
1-(2,5-Bis-trifluoromethyl-phenyl)-piperidine-4-carboxylic acid (3-oxo-3,4-dihydro-2H-benzo[1,4]oxazin-6-yl)-amide;
1-(2-Bromo-3-fluoro-phenyl)-piperidine-4-carboxylic acid (3-oxo-3,4-dihydro-2H-[1,4]oxazin-6-yl)-amide;
1-(2-Bromo-phenyl)-piperidine-4-carboxylic acid (3-oxo-3,4-dihydro-2H-benzo[1,4]oxazin-6-yl)-amide;
1-(2-Chloro-phenyl)-piperidine-4-carboxylic acid (3-oxo-3,4-dihydro-2H-benzo[1,4]oxazin-6-yl)-amide;
1-(2-Cyano-3-trifluoromethyl-phenyl)-piperidine-4-carboxylic acid (3-oxo-3,4-dihydro-2H-benzo[1,4]oxazin-6-yl)-amide;
1-(2-Cyano-5-trifluoromethyl-phenyl)-piperidine-4-carboxylic acid (3-oxo-3,4-dihydro-2H-benzo[1,4]oxazin-6-yl)-amide;
1-(2-Ethyl-phenyl)-piperidine-4-carboxylic acid (3-oxo-3,4-dihydro-2H-benzo[1,4]oxazin-6-yl)-amide;
1-(2-Fluoro-phenyl)-piperidine-4-carboxylic acid (3-oxo-3,4-dihydro-2H-benzo[1,4]oxazin-6-yl)-amide;
1-(2-Isopropyl-phenyl)-piperidine-4-carboxylic acid (3-oxo-3,4-dihydro-2H-benzo[1,4]oxazin-6-yl)-amide;
1-(2-Trifluoromethyl-phenyl)-piperidine-4-carboxylic acid (3-oxo-3,4-dihydro-2H-benzo[1,4]oxazin-6-yl)-amide;
1-(3,4,5-Trifluoro-phenyl)-piperidine-4-carboxylic acid (3-oxo-3,4-dihydro-2H-benzo[1,4]oxazin-6-yl)-amide;
1-(3,4-Difluoro-phenyl)-piperidine-4-carboxylic acid (3-oxo-3,4-dihydro-2H-benzo[1,4]oxazin-6-yl)-amide;
1-(3,4-Dimethyl-phenyl)-piperidine-4-carboxylic acid (3-oxo-3,4-dihydro-2H-benzo[1,4]oxazin-6-yl)-amide;
1-(3,5-Difluoro-phenyl)-piperidine-4-carboxylic acid (3-oxo-3,4-dihydro-2H-benzo[1,4]oxazin-6-yl)-amide;
1-(3-Bromo-phenyl)-piperidine-4-carboxylic acid (3-oxo-3,4-dihydro-2H-benzo[1,4]oxazin-6-yl)-amide;
1-(3-Chloro-2-methyl-phenyl)-piperidine-4-carboxylic acid (3-oxo-3,4-dihydro-2H-benzo[1,4]oxazin-6-yl)-amide;
1-(3-Chloro-phenyl)-piperidine-4-carboxylic acid (3-oxo-3,4-dihydro-2H-benzo[1,4]oxazin-6-yl)-amide;
1-(3-Fluoro-phenyl)-piperidine-4-carboxylic acid (3-oxo-3,4-dihydro-2H-benzo[1,4]oxazin-6-yl)-amide;
1-(3-Methoxy-phenyl)-piperidine-4-carboxylic acid (3-oxo-3,4-dihydro-2H-benzo[1,4]oxazin-6-yl)-amide;
1-(3-Trifluoromethyl-phenyl)-piperidine-4-carboxylic acid (2-oxo-2,3-dihydro-1H-pyrido[2,3-b][1,4]oxazin-7-yl)-amide;
1-(3-Trifluoromethyl-phenyl)-piperidine-4-carboxylic acid [4-(2-hydroxy-ethyl)-3-oxo-3,4-dihydro-2H-benzo[1,4]oxazin-6-yl]-amide;
1-(4-Bromo-2,3,5,6-tetrafluoro-phenyl)-piperidine-4-carboxylic acid (3-oxo-3,4-dihydro-2H-benzo[1,4]oxazin-6-yl)-amide;
1-(4-tert-Butyl-phenyl)-piperidine-4-carboxylic acid (3-oxo-3,4-dihydro-2H-benzo[1,4]oxazin-6-yl)-amide;
1-Benzothiazol-2-yl-piperidine-4-carboxylic acid (3-oxo-3,4-dihydro-2H-benzo[1,4]oxazin-6-yl)-amide;
1-m-Tolyl-piperidine-4-carboxylic acid (3-oxo-3,4-dihydro-2H-benzo[1,4]oxazin-6-yl)-amide;
1-o-Tolyl-piperidine-4-carboxylic acid (3-oxo-3,4-dihydro-2H-benzo[1,4]oxazin-6-yl)-amide;
1-Phenyl-piperidine-4-carboxylic acid (3-oxo-3,4-dihydro-2H-benzo[1,4]oxazin-6-yl)-amide;
3′-Methyl-3,4,5,6-tetrahydro-2H-[1,2′]bipyridinyl-4-carboxylic acid (3-oxo-3,4-dihydro-2H-benzo[1,4]oxazin-6-yl)-amide; and
3′-Nitro-3,4,5,6-tetrahydro-2H-[1,2′]bipyridinyl-4-carboxylic acid (3-oxo-3,4-dihydro-2H-benzo[1,4]oxazin-6-yl)-amide.

In another embodiment, the present invention is directed to a compound selected from the group consisting of
1-(3,4,5-Trifluoro-phenyl)-piperidine-4-carboxylic acid (3-oxo-3,4-dihydro-2H-benzo[1,4]oxazin-5-yl)-amide;
1-(3,4,5-Trifluoro-phenyl)-piperidine-4-carboxylic acid (3-oxo-3,4-dihydro-2H-benzo[1,4]oxazin-7-yl)-amide;
1-(3,4,5-Trifluoro-phenyl)-piperidine-4-carboxylic acid (3-oxo-3,4-dihydro-2H-benzo[1,4]oxazin-8-yl)-amide;
1-(3,4,5-Trifluoro-phenyl)-piperidine-4-carboxylic acid (4-methyl-3-oxo-3,4-dihydro-2H-benzo[1,4]oxazin-6-yl)-amide;
1-(3,4,5-Trifluoro-phenyl)-piperidine-4-carboxylic acid (3-oxo-3,4-dihydro-2H-benzo[1,4]thiazin-6-yl)-amide;
1-(3,4,5-Trifluoro-phenyl)-piperidine-4-carboxylic acid [2-(2-hydroxy-ethyl)-3-oxo-3,4-dihydro-2H-benzo[1,4]oxazin-6-yl]-amide;
2-Methyl-3-oxo-6-{[1-(3,4,5-trifluoro-phenyl)-piperidine-4-carbonyl]-amino}-3,4-dihydro-2H-benzo[1,4]oxazine-2-carboxylic acid ethyl ester;
2-Methyl-3-oxo-6-{[1-(3,4,5-trifluoro-phenyl)-piperidine-4-carbonyl]-amino}-3,4-dihydro-2H-benzo[1,4]oxazine-2-carboxylic acid;
3-Oxo-6-{[1-(3,4,5-trifluoro-phenyl)-piperidine-4-carbonyl]-amino}-3,4-dihydro-2H-benzo[1,4]oxazine-2-carboxylic acid ethyl ester;
3-Oxo-6-{[1-(3,4,5-trifluoro-phenyl)-piperidine-4-carbonyl]-amino}-3,4-dihydro-2H-benzo[1,4]oxazine-2-carboxylic acid;
(3-Oxo-6-{[1-(3,4,5-trifluoro-phenyl)-piperidine-4-carbonyl]-amino}-3,4-dihydro-2H-benzo[1,4]oxazin-2-yl)-acetic acid methyl ester; and
(3-Oxo-6-{[1-(3,4,5-trifluoro-phenyl)-piperidine-4-carbonyl]-amino}-3,4-dihydro-2H-benzo[1,4]oxazin-2-yl)-acetic acid.

The compounds of the present invention may also be present in the form of pharmaceutically acceptable salts. For use in medicine, the salts of the compounds of this invention refer to non-toxic “pharmaceutically acceptable salts” (Ref. International J. Pharm., 1986, 33, 201-217; J. Pharm. Sci., 1997 (January), 66, 1, 1). Other salts well known to those in the art may, however, be useful in the preparation of compounds according to this invention or of their pharmaceutically acceptable salts. Representative organic or inorganic acids include, but are not limited to, hydrochloric, hydrobromic, hydriodic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. Representative organic or inorganic bases include, but are not limited to, basic or cationic salts such as benzathine, chloroprocaine, choline, diethanolamine, ethylenediamine, meglumine, procaine, aluminum, calcium, lithium, magnesium, potassium, sodium and zinc.

The present invention includes within its scope prodrugs of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-d-tartaric acid and/or (+)-di-p-toluoyl-1-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

Even though the compounds of the present invention (including their pharmaceutically, acceptable salts and pharmaceutically acceptable solvates) can be administered alone, they will generally be administered in admixture with a pharmaceutical carrier, excipient or diluent selected with regard to the intended route of administration and standard pharmaceutical or veterinary practice. Thus, the present invention is directed to pharmaceutical and veterinary compositions comprising compounds of Formula (I) and one or more pharmaceutically acceptable carriers, excipients or diluents.

By way of example, in the pharmaceutical and veterinary compositions of the present invention, the compounds of the present invention may be admixed with any suitable binder(s), lubricant(s), suspending agent(s), coating agent(s), and/or solubilising agent(s).

Tablets or capsules of the compounds may be administered singly or two or more at a time, as appropriate. It is also possible to administer the compounds in sustained release formulations.

Alternatively, the compounds of Formula (I) can be administered by inhalation or in the form of a suppository or pessary, or they may be applied topically in the form of a lotion, solution, cream, ointment or dusting powder. An alternative means of transdermal administration is by use of a skin patch. For example, they can be incorporated into a cream consisting of an aqueous emulsion of polyethylene glycols or liquid paraffin. They can also be incorporated, at a concentration of between 1 and 10% by weight, into an ointment consisting of a white wax or white soft paraffin base together with such stabilizers and preservatives as may be required.

For some applications, preferably the compositions are administered orally in the form of tablets containing excipients such as starch or lactose, or in capsules or ovules either alone or in admixture with excipients, or in the form of elixirs, solutions or suspensions containing flavoring or coloring agents.

The compositions (as well as the compounds alone) can also be injected parenterally, for example intracavernosally, intravenously, intramuscularly or subcutaneously. In this case, the compositions will comprise a suitable carrier or diluent.

For parenteral administration, the compositions are best used in the form of a sterile aqueous solution which may contain other substances, for example enough salts or monosaccharides to make the solution isotonic with blood.

For buccal or sublingual administration the compositions may be administered in the form of tablets or lozenges which can be formulated in a conventional manner.

By way of further example, pharmaceutical and veterinary compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate the major site of absorption. For parenteral administration, the carrier will usually consist of sterile water and other ingredients may be added to increase solubility or preservation. Injectable suspensions or solutions may also be prepared utilizing aqueous carriers along with appropriate additives.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those skilled in that art. To be administered in the form of a transdermal delivery system, the dosage administration will, of course, be continuous rather than intermittent throughout the dosage regimen.

A therapeutically effective amount for use of the instant compounds or a pharmaceutical composition thereof comprises a dose range of from about 0.001 mg to about 1,000 mg, in particular from about 0.1 mg to about 500 mg or, more particularly from about 1 mg to about 250 mg of active ingredient per day for an average (70 kg) human.

For oral administration, a pharmaceutical composition is preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the subject to be treated.

It is also apparent to one skilled in the art that the therapeutically effective dose for active compounds of the invention or a pharmaceutical composition thereof will vary according to the desired effect. Therefore, optimal dosages to be administered may be readily determined and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level. The above dosages are thus exemplary of the average case. There can, of course, be individual instances where higher or lower dosage ranges are merited, and such are within the scope of this invention.

Compounds of this invention may be administered in any of the foregoing compositions and dosage regimens or by means of those compositions and dosage regimens established in the art whenever use of the compounds of the invention as vanilloid receptor modulators is required for a subject in need thereof.

The invention also provides a pharmaceutical or veterinary pack or kit comprising one or more containers filled with one or more of the ingredients of the pharmaceutical and veterinary compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. As modulators of the vanilloid VR1 ion channel, the compounds of Formula (I) are useful in methods for treating or preventing a disease or condition in a mammal which disease or condition is affected by the modulation of one or more vanilloid receptors.

As modulators of the vanilloid VR1 ion channel, the compounds of Formula (I) are useful in methods for treating or preventing a disease or condition in a mammal which disease or condition is affected by the modulation of one or more vanilloid receptors. Such methods comprises administering to a mammal in need of such treatment or prevention a therapeutically effective amount of a compound, salt or solvate of Formula (I). In particular, the compounds of Formula (I) are useful for in methods for preventing or treating a chronic- or acute-pain causing diseases or conditions and pulmonary dysfunction, and more particularly, in treating diseases or conditions that cause inflammatory pain, burning pain, itch or urinary incontinence, and chronic obstructive pulmonary disease.

By way of example only, the compounds of Formula (I) are useful for treating diseases and conditions selected from the group consisting of osteoarthritis, rheumatoid arthritis, fibromyalgia, migraine, headache, toothache, burn, sunburn, snake bite (in particular, venomous snake bite), spider bite, insect sting, neurogenic bladder, benign prostatic hypertrophy, interstitial cystitis, urinary tract infection, cough, asthma, chronic obstructive pulmonary disease, rhinitis, contact dermatitis/hypersensitivity, itch, eczema, anxiety, panic disorders, pharyngitis, mucositis, enteritis, cellulites, peripheral neuropathy, bilateral peripheral neuropathy, diabetic neuropathy, postherpetic neuralgia, trigeminal neuralgia, causalgia, sciatic neuritis, mandibular joint neuralgia, peripheral neuritis, polyneuritis, stump pain, phantom limb pain, bony fractures, post-operative ileus, irritable bowel syndrome, inflammatory bowel diseases such as Crohn's Disease and ulcerative colitis, cholecystitis, pancreatitis, postmastectomy pain syndrome, oral neuropathic pain, Charcot's pain, reflex sympathetic dystrophy, Guillain-Barre syndrome, meralgia paresthetica, burning-mouth syndrome, optic neuritis, postfebrile neuritis, migrating neuritis, segmental neuritis, Gombault's neuritis, neuronitis, cervicobrachial neuralgia, cranial neuralgia, geniculate neuralgia, glossopharyngial neuralgia, migrainous neuralgia, idiopathic neuralgia, intercostals neuralgia, mammary neuralgia, Morton's neuralgia, nasociliary neuralgia, occipital neuralgia, red neuralgia, Sluder's neuralgia, splenopalatine neuralgia, supraorbital neuralgia, vidian neuralgia, sinus headache, tension headache, labor, childbirth, intestinal gas, menstruation, hot flash, cancer, and trauma.

While the present invention comprises compositions comprising one or more of the compounds of Formula (I), the present invention also comprises compositions comprising intermediates used in the manufacture of compounds of Formulae (I).

### General Synthetic Methods

Compounds of formula (I) can be prepared by methods known to those who are skilled in the art. The following reaction schemes are only meant to represent examples of the invention and are in no way meant to be a limit of the invention.

Compounds of Formula (I) may be prepared generally according to the Scheme 1 and Scheme 2 below:

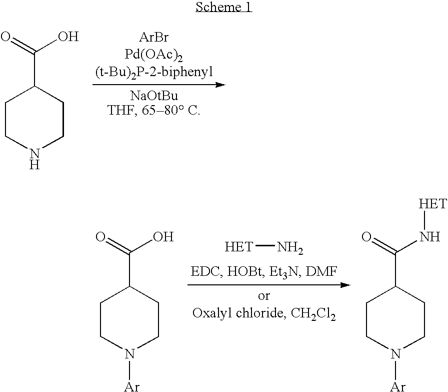

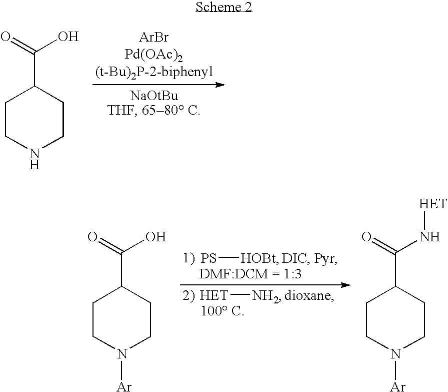

### EXAMPLES

### Example 1

### 1-Biphenyl-4-yl-piperidine-4-carboxylic acid quinolin-3-ylamide

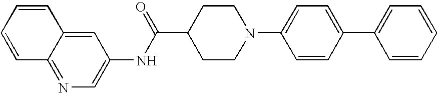 A. Sodium t-butoxide (0.211 g, 2.2 mmol) was added to a solution of 4-bromobiphenyl (0.233 g, 1.0 mmol), Pd(OAc)₂ (0.002 g, 0.008 mmol), and 2-(di-t-butylphosphino)-biphenyl (0.006 g, 0.02 mmol) in THF (5 ml). After 5 minutes of stirring, isonipecotic acid (0.129 g, 1.0 mmol) was added to the solution. The solution was heated to 65° C. for 18 h under an argon atmosphere, then cooled to room temperature and partitioned between EtOAc and water. The aqueous layer was separated and acidified using 3N HCl and then extracted with EtOAc. The organic layer was washed with brine, dried over MgSO₄, filtered and concentrated to give 1-biphenyl-4-yl-piperidine-4-carboxylic acid as a light brown solid (0.10 g, 0.35 mmol). ¹H NMR (400 MHz, DMSO) δ (ppm): 7.58 (d, 1H), 7.53 (d, 2H), 7.40 (t, 1H), 7.00 (d, 2H), 3.70 (d, 2H), 2.80 (m, 2H), 2.38-2.46 (m, 1H), 1.85-1.92 (m, 2H), 1.60-1.70 (m, 2H).
B. A solution of 3-aminoquinoline (0.077 g, 0.54 mmol), 1-biphenyl-4-yl-piperidine-4-carboxylic acid, (0.050 g, 0.18 mmol), Hydroxybenzotriazole (HOBt) (0.072 g, 0.54 mmol) and triethylamine (0.075 ml, 0.54 mmol) in dimethylformamide (DMF) (1 ml) was treated with Ethyl-3-(3 dimethylaminopropyl)-carbodiimide (EDC) (0.102 g, 0.54 mmol). The solution was stirred at room temperature for 18 h. 10% K₂CO₃ (5 ml) was added to the solution, which was then filtered. The solid was sequentially washed with water, MeOH and then dried in vacuo to give the title compound as an off-white solid (0.084 g). ¹H NMR (400 MHz, CDCl₃) δ (ppm): 9.77 (s, 1H), 8.55-8.68 (m, 2H), 7.73 (d, 1H), 7.50-7.55 (m, 1H), 7.20-7.38 (m, 6H), 7.12-7.16 (m, 2H), 6.90-7.03 (m, 1H), 6.79 (d, 2H), 3.55-3.62 (m, 2H), 2.52-2.62 (m, 2H), 2.30-2.40 (m, 1H), 1.70-1.82 (m, 4H). Mass Spectrum (LCMS, ESI pos.) Calcd. for C₂₇H₂₅N₃O: 408.20 (M+H). Found: 408.3.

### Example 2

### 1-(3-Trifluoromethyl-phenyl)-piperidine-4-carboxylic acid quinolin-3-ylamide

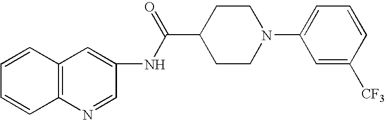 Using the procedure outlined in Example 1A, 1-(3-trifluoromethyl-phenyl)-piperidine-4-carboxylic acid was prepared from 1-bromo-3-trifluoromethylbenzene (1.1 ml, 8 mmol) as an off-white solid (0.16 g). ¹H NMR (400 MHz, CD₃OD) δ (ppm): 7.40 (t, 1H), 7.18-7.24 (m, 2H), 7.09 (d, 1H), 3.75 (d, 2H), 2.85 (t, 2H), 2.48-2.52 (m, 1H), 2.00-2.10 (m, 2H), 1.78-1.88 (m, 2H).
Using the procedure outlined in Example 1B, the title compound was prepared from 3-aminoquinoline (0.087 g, 0.60 mmol) and 1-(3-trifluoromethyl-phenyl)-piperidine-4-carboxylic acid (0.055 g, 0.2 mmol) as an off-white solid (0.024 g). ¹H NMR (400 MHz, DMSO) δ (ppm): 10.50 (s, 1H), 8.93 (d, 1H), 8.75 (d, 1H), 7.90-7.97 (m, 2H), 7.54-7.67 (m, 2H), 7.43 (t, 1H), 7.25-7.30 (m, 1H), 7.20-7.22 (s, 1H), 7.06 (d, 1H), 3.93 (d, 2H), 2.85 (t, 2H), 2.60-2.70 (m, 1H), 1.93-2.00 (m, 2H), 1.70-1.85 (m, 2H). Mass Spectrum (LCMS, ESI pos.) Calcd. for C₂₂H₂₀F₃ N₃O: 400.16 (M+H). Found: 400.3.

### Example 3

### 1-Biphenyl-3-yl-piperidine-4-carboxylic acid quinolin-3-ylamide

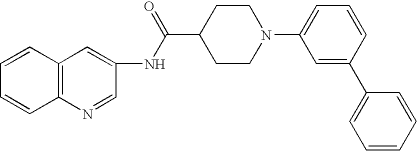 Using the procedure outlined in Example 1A, 1-biphenyl-3-yl-piperidine-4-carboxylic acid was prepared from 3-bromobiphenyl (0.56 g, 2.0 mmol). ¹H NMR (400 MHz, CDCl₃) δ (ppm): 9.10-9.70 (bs, 1H), 7.60 (d, 2H), 7.45 (t, 2H), 7.35 (t, 2H), 7.16-7.20 (m, 1H), 7.13 (d, 1H), 6.97 (m, 1H), 3.75 (d, 2H), 2.85-2.90 (m, 2H), 2.49-2.52 (m, 1H), 1.80-1.90 (m, 2H), 2.08-2.15 (m, 3H), 1.90-2.05 (m, 2H).
Using the procedure outlined in Example 1B, the title compound was prepared from 3-aminoquinoline (0.18, 1.3 mmol) and 1-biphenyl-3-yl-piperidine-4-carboxylic acid, (0.12 g, 0.43 mmol) as an off-white solid (0.069 g). ¹H NMR (400 MHz, DMSO) δ (ppm): 10.50-10.61 (s, 1H), 8.94 (d, 1H), 8.76 (d, 1H), 7.90-7.97 (m, 2H), 7.51-7.54 (m, 4H), 7.42-7.47 (m, 2H), 7.27-7.38 (m, 2H), 7.19-7.21 (bs, 1H), 6.97-7.05 (m, 2H), 3.91 (d, 2H), 2.80 (t, 2H), 2.60-2.70 (m, 1H), 1.79-2.01 (m, 4H).

### Example 4

### 1-(4-Trifluoromethyl-phenyl)-piperidine-4-carboxylic acid quinolin-3-ylamide

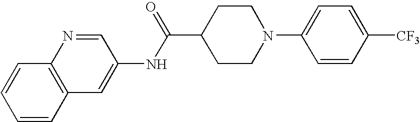 Using the procedure outlined in Example 1A, 1-(4-tifluoromethyl-phenyl)-piperidine-4-carboxylic acid was prepared from 1-bromo-4-trifluoromethyl-benzene (0.560 ml, 4 mmol) as an off-white solid (0.10 g). ¹H NMR (400 MHz, CD₃CN) δ (ppm): 7.49 (d, 2H), 7.02 (d, 2H), 3.80 (d, 2H), 2.90 (t, 2H), 2.49-2.56 (m, 1H), 1.60-1.73 (m, 2H), 1.50-1.59 (m, 2H).
Using the procedure outlined in Example 1B, the title compound was prepared from 3-aminoquinoline (0.15 g, 1.0 mmol) and 1-(4-trifluoromethyl-phenyl)-piperidine-4-carboxylic acid (0.095 g, 0.35 mmol) as an off-white solid (0.075 g). ¹H NMR (400 MHz, DMSO) δ (ppm): 10.60-10.62 (s, 1H), 8.93 (d, 1H), 8.74 (d, 1H), 7.90-7.96 (m, 2H), 7.48-7.65 (m, 4H), 7.10 (d, 2H), 4.00 (d, 2H), 2.90 (t, 2H), 2.67-2.74 (m, 1H), 1.90-1.99 (m, 2H), 1.70-1.80 (m, 2H).

### Example 5

### 1-(2,3-Dimethyl-phenyl)-piperidine-4-carboxylic acid quinolin-3-ylamide

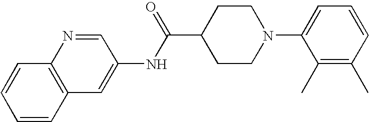 Using the procedure outlined in Example 1A, 1-(2,3-dimethyl-phenyl)-piperidine-4-carboxylic acid was prepared from 1-bromo-2,3-dimethyl-benzene (0.542 ml, 4 mmol) as an off-white solid (0.06 g). ¹H NMR (400 MHz, CD₃CN) δ (ppm): 7.02 (t, 1H), 6.85 (t, 2H), 3.00-3.05 (m, 2H), 2.60-2.68 (m, 2H), 2.24 (s, 3H), 2.19 (s, 3H), 1.78-1.90 (m, 5H).
Using the procedure outlined in Example 1B, the title compound was prepared from 3-aminoquinoline (0.08 g, 0.54 mmol) and 1-(2,3-dimethyl-phenyl)-piperidine-4-carboxylic acid (0.043 g, 0.18 mmol) as an off-white solid (0.006 g). ¹H NMR (400 MHz, CD₃OD) δ (ppm): 8.92 (s, 1H), 8.75 (s, 1H), 7.90 (d, 1H), 7.88 (d, 1H), 7.55-7.73 (m, 2H), 6.83-7.03 (m, 3H), 3.15 (d, 2H), 2.54-2.75 (m, 3H), 2.50 (s, 6H), 1.95-2.10 (m, 4H). Mass Spectrum (LCMS, ESI pos.) Calcd. for C₂₃H₂₅N₃O: 360.20 (M+H). Found: 360.2.

### Example 6

### 1-(3,4-Dimethyl-phenyl)-piperidine-4-carboxylic acid quinolin-3-ylamide

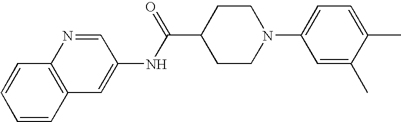 Using the procedure outlined in Example 1A, 1-(3,4-dimethyl-phenyl)-piperidine-4-carboxylic acid was prepared from 1-bromo-3,4-dimethylbenzene (0.540 ml, 4 mmol) as an off-white solid (0.14 g). ¹H NMR (400 MHz, CDCl₃) δ (ppm): 7.93 (dd, 1H), 7.50 (d, 1H), 7.10 (d, 1H), 4.00 (d, 2H), 2.92 (t, 2H), 2.67-2.73 (m, 1H), 1.93-2.00 (m, 2H), 2.68-2.80 (m, 2H).
Using the procedure outlined in Example 1B, the title compound was prepared from 3-aminoquinoline (0.193 g, 1.3 mmol) and 1-(3,4-dimethyl-phenyl)-piperidine-4-carboxylic acid (0.104 g, 0.45 mmol) as an off-white solid (0.006 g). ¹H NMR (400 MHz, solvent) δ (ppm): 8.85 (bs, 1H), 8.73 (bs, 1H), 8.05 (d, 1H), 7.80 (d, 1H), 7.50-7.70 (m, 3H), 7.05 (d, 1H), 6.70-6.80 (m, 2H), 3.74 (d, 2H), 2.75 (t, 2H), 2.40-2.50 (m, 1H), 2.25 (s, 3H), 2.20 (s, 3H), 2.00-2.13 (m, 4H). Mass Spectrum (LCMS, ESI pos.) Calcd. for C₂₃H₂₅N₃O: 360.20 (M+H). Found: 360.2.

### Example 7

### 1-(3-Trifluoromethyl-phenyl)-piperidine-4-carboxylic acid (9-ethyl-9H-purin-6-yl)-amide

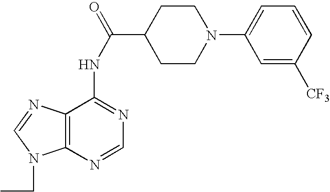 Using the procedure outlined in Example 1B, the title compound was prepared from 7-ethyladenine (0.119 g, 0.73 mmol) and 1-(3-trifluoromethyl-phenyl)-piperidine-4-carboxylic acid (0.10 g, 0.40 mmol) as an off-white solid (0.0023 g). ¹H NMR (400 MHz, CDCl₃) δ (ppm): 8.73 (s, 1H), 8.606 (bs, 1H), 8.00 (s, 1H), 7.30-7.39 (m, 1H), 7.04-7.17 (m, 3H), 4.35 (q, 2H), 3.80 (d, 2H), 3.18-3.34 (m, 1H), 2.85-2.98 (m, 2H), 1.97-2.22 (m, 4H), 1.58 (t, 3H). Mass Spectrum (LCMS, ESI pos.) Calcd. for C₂₀H₂₁F₃N₆O: 419.17 (M+H). Found: 419.2.

### Example 8

### 04/072069

### 1-(3-Trifluoromethyl-phenyl)-piperidine-4-carboxylic acid (1H-indol-4-yl)-amide

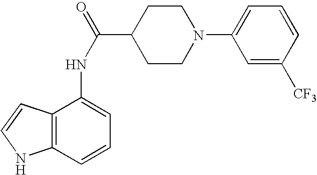 A. Oxalyl chloride (0.262 ml, 3 mmol) was added to a solution of 1-(3-trifluoromethyl-phenyl)-piperidine-4-carboxylic acid, (0.273 g, 1 mmol) in methylene chloride (5 ml). After 1 h the solution was concentrated to yield 1-(3-trifluoromethyl-phenyl)-piperidine-4-carbonyl chloride, which was used as is for the next step.
B. To a solution of 4-aminoindole (0.066 g, 0.5 mmol) and triethylamine (0.070 ml, 0.5 mmol) in methylene chloride (1 ml) was added a solution of 1-(3-trifluoromethyl-phenyl)-piperidine-4-carbonyl chloride, (0.145 g, 0.5 mmol) in methylene chloride (1 ml). After 18 h the solution was washed with K₂CO₃ (10% in water). The methylene chloride layer was separated and concentrated. The residue was purified by preparative TLC (silica gel, 20×20 cm, 2000 microns, EtOAc/hexanes 4:6) to give the title compound (0.035 g). ¹H NMR (400 MHz, CD₃OD) δ (ppm): 7.51 (d, 1H), 7.22-7.33 (m, 1H), 6.96-7.19 (m, 6H), 6.39-6.46 (m, 1H), 3.71 (d, 2H), 2.71-2.83 (m, 2H), 2.43-2.56 (m, 1H), 1.84-2.06 (m, 4H). Mass Spectrum (LCMS, ESI pos.) Calcd. for C₂₁H₂₀F₃N₃O: 388.16 (M+H). Found: 388.1.

### Example 9

### 1-(3-Trifluoromethyl-phenyl)-piperidine-4-carboxylic acid (1-acetyl-2,3-dihydro-1H-indol-7-yl)-amide

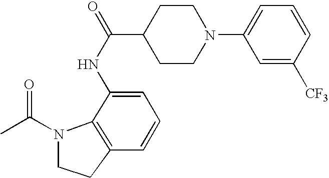 Using the procedure outlined in Example 8, the title compound was prepared from 1-(7-amino-2,3-dihydro-indol-1-yl)-ethanone (0.059 g, 0.33 mmol) as an off-white solid (0.056 g). ¹H NMR (400 MHz, CDCl₃) δ (ppm): 10.43 (s, 1H), 7.89-8.02 (m, 1H), 6.95-7.41 (m, 6H), 4.10 (t, 2H), 3.79 (d, 2H), 3.11 (t, 2H), 2.88 (t, 2H), 2.43-2.54 (m, 1H), 2.38 (s, 3H), 1.90-2.14 (m, 4H). Mass Spectrum (LCMS, ESI pos.) Calcd. for C₂₃H₂₄F₃N₃O₂: 432.18 (M+H). Found: 432.2.

### Example 10

### 1-(3-Trifluoromethyl-phenyl)-piperidine-4-carboxylic acid thieno[2,3-d]pyrimidin-4-ylamide

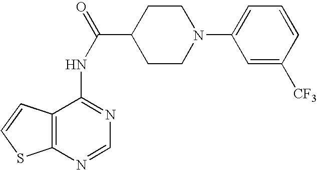 Using the procedure outlined in Example 8, the title compound was prepared from thieno[2,3-d]pyrimidin-4-ylamine (0.050 g, 0.33 mmol) as an off-white solid (0.012 g). ¹H NMR (400 MHz, CDCl₃) δ (ppm): 10.86 (s, 1H), 10.72 (s, 1H), 9.68 (d, 1H), 9.56 (s, 1H), 9.39-9.46 (m, 1H), 9.13-9.24 (m, 3H), 5.89 (d, 2H), 4.87-5.03 (m, 3H), 4.05-4.28 (m, 4H). Mass Spectrum (LCMS, ESI pos.) Calcd. for C₁₉H₁₇F₃N₄OS: 407.11 (M+H). Found: 407.1.

### Example 11

### 04/072069

### 1-(3-Trifluoromethyl-phenyl)-piperidine-4-carboxylic acid (1H-indazol-7-yl)-amide

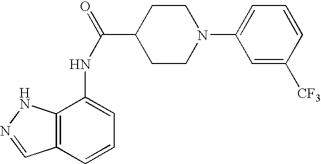 Using the procedure outlined in Example 8, the title compound was prepared from 1H-indazol-7-ylamine (0.044 g, 0.33 mmol) as an off-white solid (0.0068 g). ¹H NMR (400 MHz, DMSO) δ (ppm): 8.01 (s, 1H), 7.62 (d, 1H), 7.50 (d, 1H), 7.40 (s, 1H), 7.34 (t, 1H), 7.01-7.15 (m, 4H), 2.78-2.92 (m, 2H), 2.55-2.68 (m, 1H), 1.97-2.10 (m, 4H). Mass Spectrum (LCMS, ESI pos.) Calcd. for C₂₀H₁₉F₃N₄O: 389.15 (M+H). Found: 389.2.

### Example 12

### 04/072069

### 1-(3-Trifluoromethyl-phenyl)-piperidine-4-carboxylic acid (3-methyl-benzo[d]isothiazol-5-yl)-amide

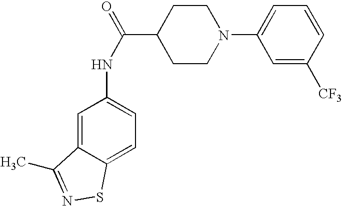 Using the procedure outlined in Example 8, the title compound was prepared from 3-methyl-benzo[d]isothiazol-5-ylamine (0.055 g, 0.33 mmol) as an off-white solid (0.011 g). ¹H NMR (400 MHz, solvent) δ (ppm): 8.47 (s, 1H), 7.87 (d, 1H), 7.34-7.48 (m, 3H), 7.08-7.12 (m, 3H), 3.87 (d, 2H), 2.83-2.96 (m, 2H), 2.76 (s, 3H), 2.43-2.55 (m, 1H), 1.99-2.20 (m, 4H). Mass Spectrum (LCMS, ESI pos.) Calcd. for C₂₁H₂₀F₃N₃OS: 420.13 (M+H). Found: 420.1.

### Example 13

### 1-(3-Trifluoromethyl-phenyl)-piperidine-4-carboxylic acid benzothiazol-6-ylamide

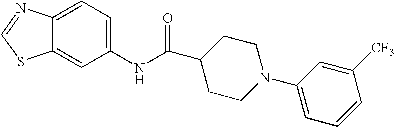 Using the procedure outlined in Example 8, the title compound was prepared from 6-aminobenzothiazole (0.008 g, 0.051 mmol) and 1-(3-trifluoromethyl-phenyl)-piperidine-4-carboxylic acid, (0.013 g, 0.046 mmol) as an off-white solid. ¹H NMR (300 MHz, CDCl₃) δ (ppm): 8.93 (s, 1H), 8.62 (d, 1H), 8.05 (d, 1H), 7.43-7.39 (m, 1H), 7.36-7.31 (m, 2H), 7.15-7.09 (m, 3H), 3.83 (d, 2H), 2.87 (td, 2H), 2.52-2.42 (m, 1H), 2.10-2.01 (m, 4H). Mass Spectrum (LCMS, ESI pos.) Calcd. for C₂₀H₁₈F₃N₃OS: 405.4; Found: 406.3 (M+H).

### Example 14

### 3-{[1-(3-Trifluoromethyl-phenyl)-piperidine-4-carbonyl]-amino}-1H-indole-2-carboxylic acid ethyl ester

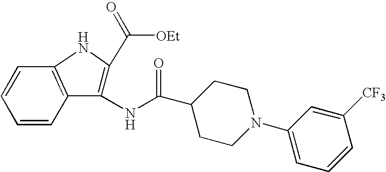 Using the procedure outlined in Example 8, the title compound was prepared from ethyl 3-amino-1H-indole-2-carboxylate (0.010 g, 0.051 mmol) and 1-(3-trifluoromethyl-phenyl)-piperidine-4-carboxylic acid, (0.013 g, 0.046 mmol) as a light brown solid. ¹H NMR (300 MHz, CDCl₃) δ (ppm): 8.27 (s, 1H), 8.44 (s, 1H), 8.26 (d, 1H), 7.33-7.26 (m, 3H), 7.16-7.08 (m, 4H), 4.44 (q, 2H), 3.84 (dt, 2H), 2.92 (t, 2H), 2.68-2.55 (m, 1H), 2.28-2.04 (m, 4H), 1.44 (t, 3H). Mass Spectrum (LCMS, ESI pos.) Calcd. for C₂₄H₂₄F₃N₃O₃: 459.5; Found: 460.4 (M+H).

### Example 15

### 1-(3-Trifluoromethyl-phenyl)-piperidine-4-carboxylic acid benzo[1,3]dioxol-5-ylamide

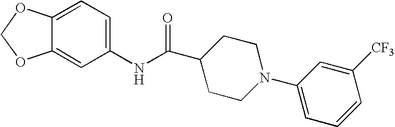 Using the procedure outlined in Example 8, the title compound was prepared from 3,4-methylenedioxyaniline (0.006 g, 0.051 mmol) and 1-(3-trifluoromethyl-phenyl)-piperidine-4-carboxylic acid, (0.013 g, 0.046 mmol) as a light brown solid. ¹H NMR (300 MHz, CDCl₃) δ (ppm): 7.35 (t, 1H), 7.25 (d, 1H), 7.20 (s, 1H), 7.13-7.07 (m, 3H), 6.80 (dd, 1H), 6.74 (d, 1H), 5.95 (s, 2H), 3.80 (dt, 2H), 2.83 (td, 2H), 2.40-2.35 (m, 1H), 2.05-1.95 (m, 4H). Mass Spectrum (LCMS, ESI pos.) Calcd. for C₂₀H₁₉F₃N₂O₃: 392.4; Found: 393.3 (M+H).

### Example 16

### 1-(3-Trifluoromethyl-phenyl)-piperidine-4-carboxylic acid (1-acetyl-2,3-dihydro-1H-indol-6-yl)-amide

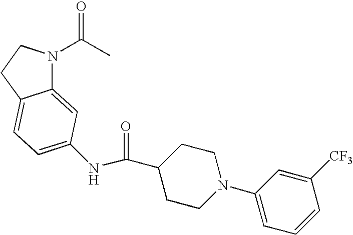 Using the procedure outlined in Example 1B, the title compound was prepared from 1-acetyl-6-amino-2,3-dihydro(1H)-indole (0.009 g, 0.051 mmol) and 1-(3-trifluoromethyl-phenyl)-piperidine-4-carboxylic acid, (0.013 g, 0.046 mmol) as an off-white solid. ¹H NMR (300 MHz, CDCl₃) δ (ppm): 8.12 (d, 1H), 7.75 (s, 1H), 7.37-7.32 (m, 2H), 7.13-7.07 (m, 4H), 6.98 (dd, 1H), 4.05 (t, 2H), 3.80 (d, 2H), 3.18 (t, 2H), 2.83 (td, 2H), 2.41-2.37 (m, 1H), 2.22 (s, 3H), 2.05-1.96 (m, 4H).

### Example 17

### 1-(3-Trifluoromethyl-phenyl)-piperidine-4-carboxylic acid (2-oxo-2,3-dihydro-1H-indol-5-yl)-amide

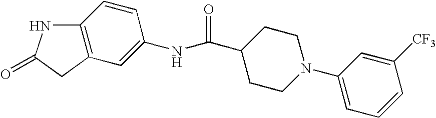 Using the procedure outlined in Example 1B, the title compound was prepared from 5-aminooxindole (0.008 g, 0.051 mmol) and 1-(3-trifluoromethyl-phenyl)-piperidine-4-carboxylic acid, (0.013 g, 0.046 mmol) as an off-white solid. ¹H NMR (300 MHz, CDCl₃) δ (ppm): 7.50 (s, 1H), 7.39-7.33 (m, 2H), 7.22-7.18 (m, 2H), 7.05 (d, 1H), 6.83 (d, 1H), 3.85 (d, 2H), 3.52 (s, 2H), 2.88-2.78 (m, 2H), 2.58-2.48 (m, 1H), 2.04-1.92 (m, 4H). Mass Spectrum (LCMS, ESI pos.) Calcd. for C₂₁H₂₀F₃N₃O₂: 403.4; Found: 388.3 (M−CH₂).

### Example 18

### 1-(3-Trifluoromethyl-phenyl)-piperidine-4-carboxylic acid (2,3-dihydro-benzo[1,4]dioxin-6-yl)-amide

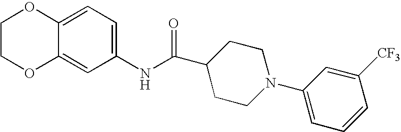 Using the procedure outlined in Example 1B, the title compound was prepared from 3,4-ethylenedioxyaniline (0.008 g, 0.051 mmol) and 1-(3-trifluoromethyl-phenyl)-piperidine-4-carboxylic acid, (0.013 g, 0.046 mmol) as an off-white solid. ¹H NMR (300 MHz, CDCl₃) δ (ppm): 7.35 (t, 1H), 7.16 (d, 1H), 7.13-7.07 (m, 4H), 6.91 (dd, 1H), 6.80 (d, 1H), 4.24 (s, 4H), 3.80 (d, 2H), 2.84 (td, 2H), 2.44-2.36 (m, 1H), 2.05-1.95 (m, 4H). Mass Spectrum (LCMS, ESI pos.) Calcd. for C₂₁H₂₁F₃N₂O₃: 406.4; Found: 407.4 (M+H).

### Example 19

### 1-(3-Trifluoromethyl-phenyl)-piperidine-4-carboxylic acid (2-methyl-benzothiazol-5-yl)-amide

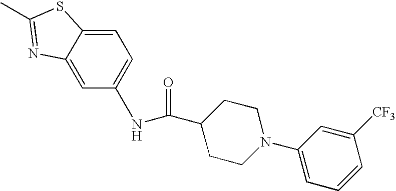 Using the procedure outlined in Example 1B, the title compound was prepared from 5-amino-2-methylbenzothiazole (0.008 g, 0.051 mmol) and 1-(3-trifluoromethyl-phenyl)-piperidine-4-carboxylic acid, (0.013 g, 0.046 mmol) as a light brown solid. ¹H NMR (300 MHz, CDCl₃) δ (ppm): 10.16 (s, 1H), 8.30 (d, 1H), 7.91 (d, 1H), 7.56 (dd, 1H), 7.42 (t, 1H), 7.26 (d, 1H), 7.19 (s, 1H), 7.05 (d, 1H), 3.88 (d, 2H), 2.82 (s, 2H), 2.77 (s, 3H), 2.60-2.46 (m, 1H), 1.96-1.84 (m, 2H), 1.80-1.65(m, 2H). Mass Spectrum (LCMS, ESI pos.) Calcd. for C₂₁H₂₀F₃N₃OS: 419.5; Found: 419.7 (M+).

### Example 20

### 1-(3-Trifluoromethyl-phenyl)-piperidine-4-carboxylic acid (5-nitro-benzo[d]isothiazol-3-yl)-amide

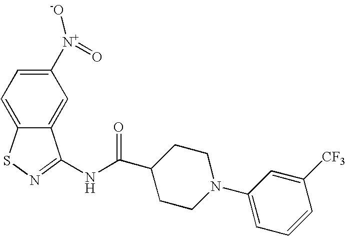 Using the procedure outlined in Example 1B, the title compound was prepared from 3-amino-5-nitrobenzoisothiazole (0.010 g, 0.051 mmol) and 1-(3-trifluoromethyl-phenyl)-piperidine-4-carboxylic acid, (0.013 g, 0.046 mmol) as a light brown solid. ¹H NMR (300 MHz, CDCl₃) δ (ppm): 7.36 (t, 1H), 7.17-7.07 (m, 4H), 6.55 (ddd, 1H), 6.46 (ddd, 1H), 6.41 (t, 1H), 3.73 (dt, 2H), 2.94 (td, 2H), 2.76-2.67 (m, 1H), 2.22-2.16 (m, 2H), 2.07-1.94 (m, 2H). Mass Spectrum (LCMS, ESI pos.) Calcd. for C₂₀H₁₇F₃N₄O₃S: 450.4; Found: 432.3 (M−18).

### Example 21

### 04/072069

### 1-(3-Trifluoromethyl-phenyl)-piperidine-4-carboxylic acid (1H-indol-4-yl)-amide

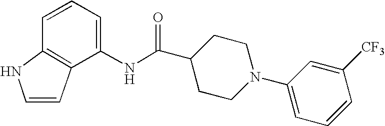 Using the procedure outlined in Example 1B, the title compound was prepared from 4-aminoindole (0.007 g, 0.051 mmol) and 1-(3-trifluoromethyl-phenyl)-piperidine-4-carboxylic acid, (0.013 g, 0.046 mmol) as a brown solid. ¹H NMR (300 MHz, CDCl₃) δ (ppm): 8.35 (s, 1H), 7.80 (d, 1H), 7.47 (s, 1H), 7.36 (t, 1H), 7.23-7.08 (m, 6H), 6.48 (s, 1H), 3.83 (d, 2H), 2.88 (t, 2H), 2.59-2.47 (m, 1H), 2.18-1.96 (m, 4H). Mass Spectrum (LCMS, ESI pos.) Calcd. for C₂₁H₂₀F₃N₃O: 387.4; Found: 388.3 (M+H).

### Example 22

### 04/072069

### 1-(3-Trifluoromethyl-phenyl)-piperidine-4-carboxylic acid (1H-indol-5-yl)-amide

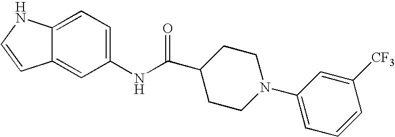 Using the procedure outlined in Example 1B, the title compound was prepared from 5-aminoindole (0.007 g, 0.051 mmol) and 1-(3-trifluoromethyl-phenyl)-piperidine-4-carboxylic acid, (0.013 g, 0.046 mmol) as a light brown solid. ¹H NMR (300 MHz, CDCl₃) δ (ppm): 7.75 (d, 1H), 7.39 (t, 1H), 7.31 (dd, 1H), 7.22-7.16 (m, 4H), 7.05 (d, 1H), 6.39 (dd, 1H), 3.86 (d, 2H), 2.90-2.80 (m, 2H), 2.59-2.52 (m, 1H), 2.00-1.93 (m, 4H). Mass Spectrum (LCMS, ESI pos.) Calcd. for C₂₁H₂₀F₃N₃O: 387.4; Found: 388.3 (M+H).

### Example 23

### 04/072069

### 1-(3-Trifluoromethyl-phenyl)-piperidine-4-carboxylic acid (1H-indol-6-yl)-amide

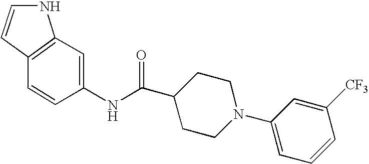 Using the procedure outlined in Example 1, the title compound was prepared from 6-aminoindole (0.007 g, 0.051 mmol) and 1-(3-trifluoromethyl-phenyl)-piperidine-4-carboxylic acid, (0.013 g, 0.046 mmol) as an off-white solid. ¹H NMR (300 MHz, CDCl₃) δ (ppm): 10.99 (s, 1H), 9.84 (s, 1H), 7.98 (s, 1H), 7.41 (d, 2H), 7.27-7.19 (m, 3H), 7.04 (dt, 2H), 6.33 (t, 1H), 3.89 (d, 2H), 2.81 (t, 2H), 2.62-2.48 (m, 1H), 1.89 (d, 2H), 1.76 (td, 2H). Mass Spectrum (LCMS, ESI pos.) Calcd. for C₂₁H₂₀F₃N₃O: 387.4; Found: 388.3 (M+H).

### Example 24

### 04/072069

### 1-(3-Trifluoromethyl-phenyl)-piperidine-4-carboxylic acid (1-methyl-1H-indazol-5-yl)-amide

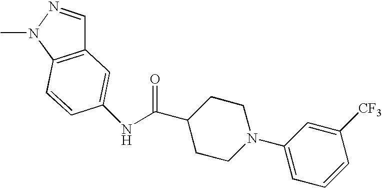 Using the procedure outlined in Example 1B, the title compound was prepared from 1-methyl-1H-indazol-5-amine (0.008 g, 0.051 mmol) and 1-(3-trifluoromethyl-phenyl)-piperidine-4-carboxylic acid, (0.013 g, 0.046 mmol) as a light brown solid. ¹H NMR (300 MHz, CDCl₃) δ (ppm): 8.01 (d, 1H), 7.93 (s, 1H), 7.42-7.32 (m, 4H), 7.14-7.08 (m, 3H), 4.06 (s, 3H), 3.82 (d, 2H), 2.85 (t, 2H), 2.50-2.39 (m, 1H), 2.09-2.00 (m, 4H).

### Example 25

### 1-(3-Trifluoromethyl-phenyl)-piperidine-4-carboxylic acid (6-methoxy-pyridin-3-yl)-amide

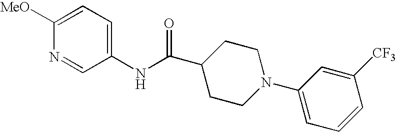 Using the procedure outlined in Example 1B, the title compound was prepared from 5-amino-2-methoxypyridine (0.006 g, 0.051 mmol) and 1-(3-trifluoromethyl-phenyl)-piperidine-4-carboxylic acid, (0.013 g, 0.046 mmol) as an off-white solid. ¹H NMR (300 MHz, CDCl₃) δ (ppm): 8.14 (d, 1H), 7.94 (dd, 1H), 7.35 (t, 1H), 7.13-7.07 (m, 4H), 6.74 (d, 1H), 3.92 (s, 3H), 3.82 (dt, 2H), 2.85 (td, 2H), 2.44-2.39 (m, 1H), 2.09-1.97 (m, 4H). Mass Spectrum (LCMS, ESI pos.) Calcd. for C₁₉H₂₀F₃N₃O₂: 379.4; Found: 380.4 (M+H).

### Example 26

### 1-(3-Trifluoromethyl-phenyl)-piperidine-4-carboxylic acid (2-mercapto-benzothiazol-6-yl)-amide

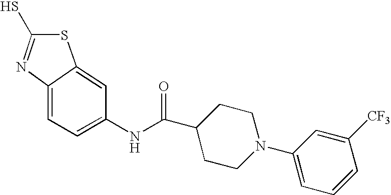 Using the procedure outlined in Example 1B, the title compound was prepared from 6-amino-2-mercaptobenzothiazole (0.009 g, 0.051 mmol) and 1-(3-trifluoromethyl-phenyl)-piperidine-4-carboxylic acid, (0.013 g, 0.046 mmol) as an off-white solid. ¹H NMR (300 MHz, CDCl₃) δ (ppm): 7.91 (d, 1H), 7.48 (dd, 1H), 7.39 (t, 1H), 7.24-7.18 (m, 4H), 7.05 (d, 1H), 3.86 (d, 2H), 2.84 (td, 2H), 2.59-2.52 (m, 1H), 2.01-1.90 (m, 4H).

### Example 27

### 1-(3-Trifluoromethyl-phenyl)-piperidine-4-carboxylic acid (8-hydroxy-quinolin-5-yl)-amide

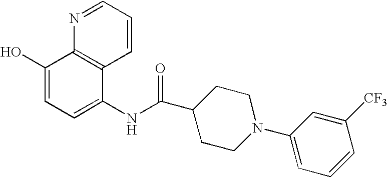 Using the procedure outlined in Example 1B, the title compound was prepared 5-amino-8-hydroxyquinoline (0.008 g, 0.051 mmol) and 1-(3-trifluoromethyl-phenyl)-piperidine-4-carboxylic acid, (0.013 g, 0.046 mmol) as a white solid. ¹H NMR (300 MHz, CDCl₃) δ (ppm): 8.87 (dd, 1H), 8.19 (d, 1H), 7.88 (s, 1H), 7.48-7.33 (m, 2H), 7.25 (d, 1H), 7.17-7.07 (m, 4H), 6.78 (d, 1H), 4.21 (s, 1H), 3.80 (d, 2H), 3.02 (t, 2H), 2.33 (d, 2H), 2.13 (t, 2H). Mass Spectrum (LCMS, ESI pos.) Calcd. for C₂₂H₂₀F₃N₃O₂: 415.4; Found: 416.3 (M+H).

### Example 28

### 1-(3-Trifluoromethyl-phenyl)-piperidine-4-carboxylic acid (3,4-dihydro-2H-benzo[b][1,4]dioxepin-7-yl)-amide

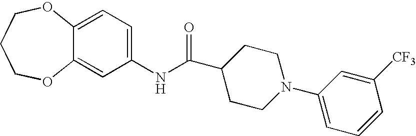 Using the procedure outlined in Example 1B, the title compound was prepared from 3,4-dihydro-2H-1,5-benzodioxepin-7-amine (0.008 g, 0.051 mmol) and 1-(3-trifluoromethyl-phenyl)-piperidine-4-carboxylic acid, (0.013 g, 0.046 mmol) as a pale yellow solid. ¹H NMR (300 MHz, CDCl₃) δ (ppm): 7.35 (t, 1H), 7.21 (d, 1H), 7.13-7.04 (m, 5H), 6.93 (d, 1H), 4.18 (dt, 4H), 3.81 (d, 2H), 2.85 (t, 2H), 2.39 (m, 1H), 2.18 (quin, 2H), 2.03-1.96 (m, 4H). Mass Spectrum (LCMS, ESI pos.) Calcd. for C₂₂H₂₃F₃N₂O₃: 420.4; Found: 421.4 (M+H).

### Example 29

### 1-(3-Trifluoromethyl-phenyl)-piperidine-4-carboxylic acid (1-acetyl-2,3-dihydro-1H-indol-5-yl)-amide

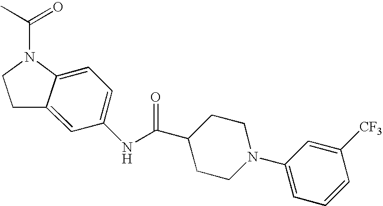 Using the procedure outlined in Example 1B, the title compound was prepared from 1-acetyl-5-amino-2,3-dihydro-(1H)indole (0.009 g, 0.051 mmol) and 1-(3-trifluoromethyl-phenyl)-piperidine-4-carboxylic acid, (0.013 g, 0.046 mmol) as a white solid. ¹H NMR (300 MHz, CDCl₃) δ (ppm): 8.12 (d, 1H), 7.75 (s, 1H), 7.37-7.33 (m, 2H), 7.13-7.09 (m, 2H), 6.98 (d, 1H), 4.05 (t, 2H), 3.81 (d, 2H), 3.18 (t, 2H), 2.84 (t, 2H), 2.41 (m, 1H), 2.22 (s, 3H), 2.04-1.98 (m, 4H). Mass Spectrum (LCMS, ESI pos.) Calcd. for C₂₃H₂₄F₃N₃O₂: 431.5; Found: 432.4 (M+H).

### Example 30

### 04/072069

### 1-(3-Trifluoromethyl-phenyl)-piperidine-4-carboxylic acid (1H-indol-7-yl)-amide

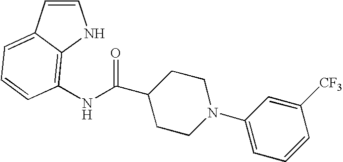 Using the procedure outlined in Example 1B, the title compound was prepared from 7-aminoindole (0.007 g, 0.051 mmol) and 1-(3-trifluoromethyl-phenyl)-piperidine-4-carboxylic acid, (0.013 g, 0.046 mmol) as a white solid. ¹H NMR (300 MHz, CDCl₃) δ (ppm): 10.01 (s, 1H), 7.53 (s, 1H), 7.50 (d, 1H), 7.36 (t, 1H), 7.24 (t, 1H), 7.15 (s, 1H), 7.11 (t, 1H), 7.02 (t, 1H), 6.73 (d, 1H), 6.55 (t, 1H), 3.84 (d, 2H), 2.88 (td, 2H), 2.56-2.52 (m, 1H), 2.14-2.03 (m, 4H). Mass Spectrum (LCMS, ESI pos.) Calcd. for C₂₁H₂₀F₃N₃O: 387.4; Found: 388.3 (M+H).

### Example 31

### 1-(3-Trifluoromethyl-phenyl)-piperidine-4-carboxylic acid (1,4-dioxo-1,2,3,4-tetrahydro-phthalazin-6-yl)-amide

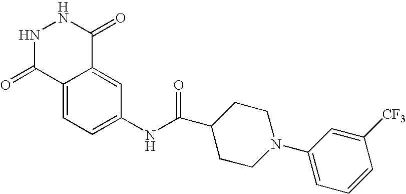 Using the procedure outlined in Example 1B, the title compound was prepared from isoluminol (0.009 g, 0.051 mmol) and 1-(3-trifluoromethyl-phenyl)-piperidine-4-carboxylic acid, (0.013 g, 0.046 mmol) as a white solid. ¹H NMR (300 MHz, CDCl₃) δ (ppm): 8.02 (d, 1H), 7.47-7.38 (m, 3H), 7.25-7.22 (m, 3H), 7.10-7.06 (m, 2H), 6.71-6.70 (m, 1H), 3.81 (m, 2H), 2.97 (t, 3H), 2.63 (m, 2H), 2.03 (m, 2H). Mass Spectrum (LCMS, ESI pos.) Calcd. for C₂₁H₁₉F₃N₄O₃: 432.4; Found: 433.3 (M+H).

### Example 32

### 1-(3-Trifluoromethyl-phenyl)-piperidine-4-carboxylic acid (6-phenoxy-pyridin-3-yl)-amide

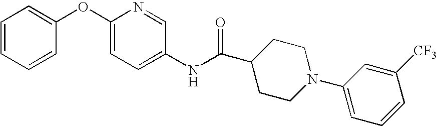 Using the procedure outlined in Example 1B, the title compound was prepared from 6-phenoxy-3-pyridinamine (0.010 g, 0.051 mmol) and 1-(3-trifluoromethyl-phenyl)-piperidine-4-carboxylic acid, (0.013 g, 0.046 mmol) as a brown solid. ¹H NMR (300 MHz, CDCl₃) δ (ppm): 8.33 (s, 1H), 8.07 (dd, 1H), 7.44-7.34 (m, 4H), 7.22-7.14 (m, 3H), 7.09-7.06 (m, 3H), 6.92 (d, 1H), 3.85 (d, 2H), 2.94 (t, 2H), 2.56-2.53 (m, 1H), 1.97-1.89 (m, 4H). Mass Spectrum (LCMS, ESI pos.) Calcd. for C₂₄H₂₂F₃N₃O₂: 441.5; Found: 442.3 (M+H).

### Example 33

### 1-(3-Trifluoromethyl-phenyl)-piperidine-4-carboxylic acid (6-acetyl-benzo[1,3]dioxol-5-yl)-amide

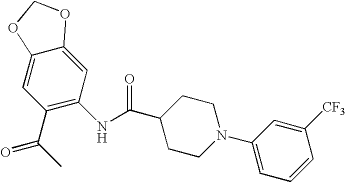 Using the procedure outlined in Example 8, the title compound was prepared from 6′-amino-3′,4′-(methylenedioxy)acetophenone (0.009 g, 0.051 mmol) and 1-(3-trifluoromethyl-phenyl)-piperidine-4-carboxylic acid, (0.013 g, 0.046 mmol) as a yellow brown solid. ¹H NMR (300 MHz, CDCl₃) δ (ppm): 12.29 (s, 1H), 8.43 (s, 1H), 7.34 (t, 1H), 7.28 (s, 1H), 7.14 (s, 1H), 7.08 (t, 2H), 6.04 (s, 2H), 3.80 (dt, 2H), 2.88 (td, 2H), 2.59 (s, 2H), 2.50-2.46 (m, 1H), 2.14 (d, 2H), 2.00 (dt, 2H). Mass Spectrum (LCMS, ESI pos.) Calcd. for C₂₂H₂₁F₃N₂O₄: 434.4; Found: 434.7 (M+).

### Example 34

### 1-(3-Trifluoromethyl-phenyl)-piperidine-4-carboxylic acid (3-oxo-3,4-dihydro-2H-benzo[1,4]oxazin-6-yl)-amide

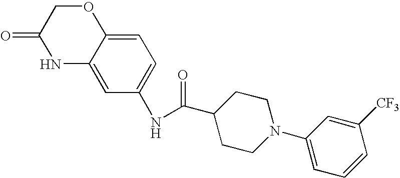 Using the procedure outlined in Example 8, the title compound was prepared from 6-amino-2H-1,4-benzoxazin-3(4H)-one (0.008 g, 0.051 mmol) and 1-(3-trifluoromethyl-phenyl)-piperidine-4-carboxylic acid, (0.013 g, 0.046 mmol) as a white solid. ¹H NMR (300 MHz, CDCl₃) δ (ppm): 7.78 (s, 1H), 7.54 (s, 1H), 7.36 (m, 1H), 7.22-7.06 (m, 4H), 6.92 (d, 1H), 6.77 (d, 1H), 4.59 (s, 2H), 3.81 (d, 2H), 2.87 (m, 2H), 2.42 (m, 1H), 2.05 (m, 4H). Mass Spectrum (LCMS, ESI pos.) Calcd. for C₂₁H₂₀F₃N₃O₃: 419.4; Found: 419.7 (M⁺).

### Example 35

### 1-(3-Trifluoromethyl-phenyl)-piperidine-4-carboxylic acid (4-dimethylamino-5-phenyl-pyrimidin-2-yl)-amide

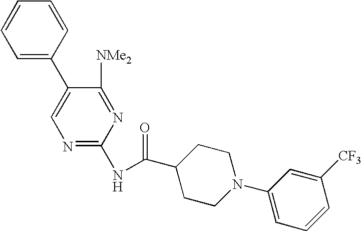 Using the procedure outlined in Example 8, the title compound was prepared from N⁴,N⁴-dimethyl-5-phenyl-2,4-pyrimidinediamine (0.011 g, 0.051 mmol) and 1-(3-trifluoromethyl-phenyl)-piperidine-4-carboxylic acid, (0.013 g, 0.046 mmol) as a pale yellow oil. ¹H NMR (300 MHz, CDCl₃) δ (ppm): 8.13 (s, 1H), 7.44-7.31 (m, 6H), 7.11-7.07 (m, 4H), 3.75 (d, 2H), 2.97-2.94 (m, 1H), 2.84 (s, 6H), 2.79 (t, 2H), 2.11-1.99 (m, 4H). Mass Spectrum (LCMS, ESI pos.) Calcd. for C₂₅H₂₆F₃N₅O: 469.5; Found: 469.9 (M⁺).

### Example 36

### 3,5-Dimethyl-1-(5-{[1-(3-trifluoromethyl-phenyl)-piperidine-4-carbonyl]-amino}-pyridin-2-yl)-1H-pyrazole-4-carboxylic acid ethyl ester

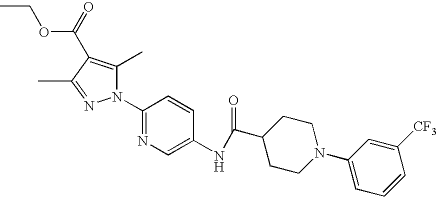 Using the procedure outlined in Example 8, the title compound was prepared from ethyl 1-(5-amino-2-pyridinyl)-3,5-dimethyl-1H-pyrazole-4-carboxylate (0.013 g, 0.051 mmol) and 1-(3-trifluoromethyl-phenyl)-piperidine-4-carboxylic acid, (0.013 g, 0.046 mmol) as a pale brown solid. ¹H NMR (300 MHz, CDCl₃) δ (ppm): 8.41 (d, 1H), 8.24 (dd, 1H), 7.69 (d, 1H), 7.62 (s, 1H), 7.35 (t, 1H), 7.13-7.08 (m, 3H), 4.32 (q, 2H), 4.12 (d, 2H), 2.85 (t, 2H), 2.79 (s, 3H), 2.49 (s, 3H), 2.47-2.44 (m, 1H), 2.06-1.99 (m, 4H), 1.38 (t, 3H). Mass Spectrum (LCMS, ESI pos.) Calcd. for C₂₆H₂₈F₃N₅O₃: 515.5; Found: 515.9 (M⁺).

### Example 37

### 1-(3-Trifluoromethyl-phenyl)-piperidine-4-carboxylic acid (1-acetyl-2-methyl-2,3-dihydro-1H-indol-6-yl)-amide

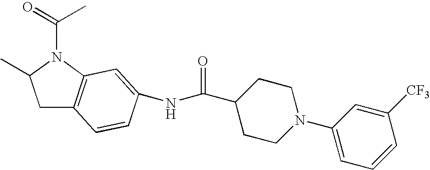 A mixture of 1-(2-Methyl-6-nitro-2,3-dihydro-indol-1-yl)-ethanone (0.010 g, 0.0454 mmol) and 10% Pd/C (0.005 g, 0.00545 mmol) in 1 mL of MeOH was stirred under H_{2(g)} for 1 hour. Catalyst was filtered off and the filtrate was concentrated to provide 1-(6-amino-2-methyl-2,3-dihydro-indol-1-yl)-ethanone as white solid, which is ready for the following amide coupling without further purification.
Using the procedure outlined in Example 8, the title compound was prepared from the above prepared amino product (0.008 g, 0.042 mmol) and 1-(3-trifluoromethyl-phenyl)-piperidine-4-carboxylic acid, (0.01 g, 0.038 mmol) as an off-white solid. ¹H NMR (400 MHz, CDCl₃) δ (ppm): 7.91 (s, 1H), 7.79 (d, 1H), 7.39 (s, 1H), 7.34 (t, 1H), 7.16-7.09 (m, 4H), 4.47 (s, 1H), 3.80 (d, 2H), 3.45 (dd, 1H), 2.85 (t, 2H), 2.64 (d, 1H), 2.04 (s, 1H), 2.28 (s, 3H), 2.04-1.95 (m, 4H), 1.30 (d, 3H). Mass Spectrum (LCMS, ESI pos.) Calcd. for C₂₄H₂₆F₃N₃O₂: 445.5; Found: 446.2 (M+H).

### Example 38

### 1-(3-Trifluoromethyl-phenyl)-piperidine-4-carboxylic acid (2-oxo-2,3-dihydro-1H-indol-6-yl)-amide

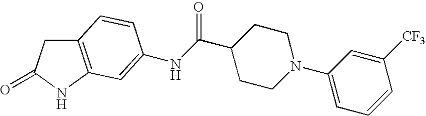 A mixture of 3,3-dichloro-6-nitro-1,3-dihydro-indol-2-one (0.020 g, 0.0809 mmol) and 10% Pd/C (0.009 g, 0.00809 mmol) in 2 mL of MeOH was stirred under H_{2(g)} for 1 hour. The catalyst was filtered off and the filtrate was concentrated to provide 6-amino-1,3-dihydro-indol-2-one as a white solid, which is ready for the following amide coupling without further purification.
Using the procedure outlined in Example 8, the title compound was prepared from the above prepared amino product (0.009 g, 0.061 mmol) and 1-(3-trifluoromethyl-phenyl)-piperidine-4-carboxylic acid, (0.01 g, 0.055 mmol) as an off-white solid. ¹H NMR (400 MHz, CDCl₃) δ (ppm): 7.56 (s, 1H), 7.50 (s, 1H), 7.29 (t, 1H), 7.14-6.98 (m, 4H), 6.74 (d, 1H), 3.75 (d, 2H), 3.42 (s, 2H), 2.80 (t, 2H), 2.36 (m, 1H), 2.04-1.88 (m, 4H). Mass Spectrum (LCMS, ESI pos.) Calcd. for C₂₁H₂₀F₃N₃O₂: 403.4; Found: 404.2 (M+H).

### Example 39

### 1-(3-Trifluoromethyl-phenyl)-piperidine-4-carboxylic acid quinazolin-4-ylamide

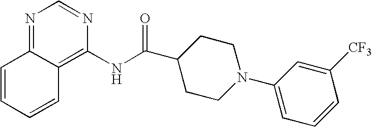 Using the procedure outlined in Example 8, the title compound was prepared from 4-quinazolinamine (0.007 g, 0.051 mmol) and 1-(3-trifluoromethyl-phenyl)-piperidine-4-carboxylic acid, (0.013 g, 0.046 mmol) as an yellow solid. ¹H NMR (400 MHz, CDCl₃) δ (ppm): 8.0-7.88 (m, 3H), 7.66 (s, 1H), 7.36 (t, 2H), 7.24-7.12 (m, 4H), 3.80 (d, 2H), 2.98 (t, 2H), 2.86 (m, 1H), 2.22 (m, 2H), 2.08 (m, 2H). Mass Spectrum (LCMS, ESI pos.) Calcd. for C₂₁H₁₉F₃N₄O: 400.4; Found: 401.2 (M+H).

### Example 40

### 1-(3-Trifluoromethyl-phenyl)-piperidine-4-carboxylic acid (2,3-dimethyl-pyrido[2,3-b]pyrazin-6-yl)-amide

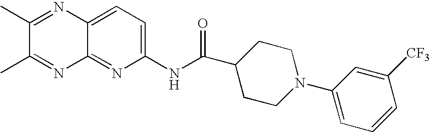 Using the procedure outlined in Example 8, the title compound was prepared from 6-amino-2,3-dimethylpyrido(2,3-b)pyrazine (0.009 g, 0.051 mmol) and 1-(3-trifluoromethyl-phenyl)-piperidine-4-carboxylic acid, (0.013 g, 0.046 mmol) as an yellow oil. ¹H NMR (400 MHz, CDCl₃) δ (ppm): 8.66 (d, 1H), 8.56 (bs, 1H), 8.34 (d, 1H), 7.35 (t, 1H), 7.14 (s, 1H), 7.09 (t, 2H), 3.81 (d, 2H), 2.88 (t, 2H), 2.76 (s, 3H), 2.74 (s, 3H), 2.56 (m, 1H), 2.15-2.12 (m, 2H), 2.06-1.99 (m, 2H). Mass Spectrum (LCMS, ESI pos.) Calcd. for C₂₂H₂₂F₃N₅O: 429.4; Found: 430.2 (M+H).

### Example 41

### 1-(3-Trifluoromethyl-phenyl)-piperidine-4-carboxylic acid (4,6-dimethyl-thieno[2,3-b]pyridin-3-yl)-amide

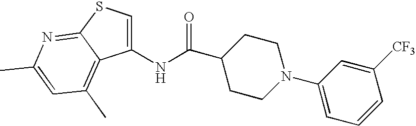 Using the procedure outlined in Example 8, the title compound was prepared from 4,6-dimethyl-thieno[2,3-b]pyridin-3-ylamine (0.009 g, 0.051 mmol) and 1-(3-trifluoromethyl-phenyl)-piperidine-4-carboxylic acid, (0.013 g, 0.046 mmol) as an yellow oil. ¹H NMR (300 MHz, CDCl₃) δ (ppm): 7.80 (s, 1H), 7.71 (bs, 1H), 7.36 (t, 1H), 7.15 9s, 1H), 7.10 (d, 2H), 6.93 (s, 1H), 3.83 (d, 2H), 2.88 (t, 2H), 2.72 (s, 3H), 2.61 (s, 3H), 2.51-2.45 (m, 1H), 2.18-2.10 (m, 2H), 2.04-1.94 (m, 2H). Mass Spectrum (LCMS, ESI pos.) Calcd. for C₂₂H₂₂F₃N₃OS: 433.5; Found: 434.2 (M+H).

### Example 42

### 1-(3-Trifluoromethyl-phenyl)-piperidine-4-carboxylic acid [5-(3,4-dimethoxy-phenyl)-4-methyl-pyrimidin-2-yl]-amide

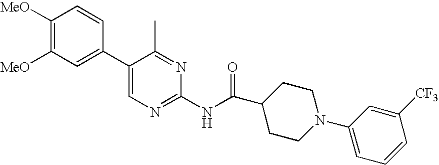 Using the procedure outlined in Example 8, the title compound was prepared from 5-(3,4-dimethoxyphenyl)-4-methyl-2-pyrimidinamine (0.013 g, 0.051 mmol) and 1-(3-trifluoromethyl-phenyl)-piperidine-4-carboxylic acid, (0.013 g, 0.046 mmol) as a white solid. ¹H NMR (400 MHz, CDCl₃) δ (ppm): 8.41 (s, 1H), 8.02 (bs, 1H), 7.28 (m, 1H), 7.02-7.24 (m, 3H), 6.98 (d, 1H), 6.85 (dd, 1H), 6.78 (d, 1H), 3.94 (s, 3H), 3.90 (s, 3H), 3.84 (dd, 2H), 3.12 (m, 1H), 2.93 (m, 2H), 2.47 (s, 3H), 2.24-1.96 (m, 4H). Mass Spectrum (LCMS, ESI pos.) Calcd. for C₂₅H₂₆F₃N₅O: 469.5; Found: 501.3 (M+MeCN+H).

### Example 43

### 1-(3-Trifluoromethyl-phenyl)-piperidine-4-carboxylic acid (4-methyl-6-phenoxy-pyrimidin-2-yl)-amide

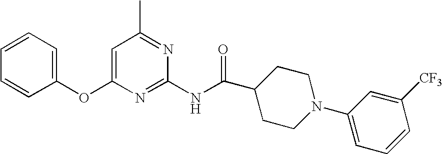 Using the procedure outlined in Example 8, the title compound was prepared from 2-amino-4-phenoxy-6-methylpyrimidine (0.010 g, 0.051 mmol) and 1-(3-trifluoromethyl-phenyl)-piperidine-4-carboxylic acid, (0.013 g, 0.046 mmol) as a white solid. ¹H NMR (400 MHz, CDCl₃) δ (ppm): 7.79 (bs, 1H), 7.40 (t, 2H), 7.33 (t, 1H), 7.19 (t, 1H), 7.13 (dd, 2H), 7.05 (m, 3H), 6.41 (s, 1H), 3.60 (d, 2H), 3.21 (m, 1H), 2.47 (t, 2H), 2.43 (s, 3H), 1.94-1.82 (m, 4H). Mass Spectrum (LCMS, ESI pos.) Calcd. for C₂₄H₂₃F₃N₄O₂: 456.5; Found: 457.3 (M+H).

### Example 44

### 1-(3-Trifluoromethyl-phenyl)-piperidine-4-carboxylic acid (3-oxo-3,4-dihydro-2H-benzo[1,4]oxazin-7-yl)-amide

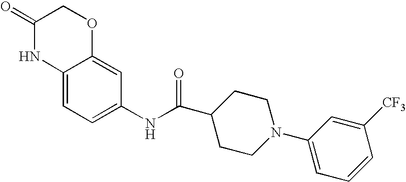 Using the procedure outlined in Example 8, the title compound was prepared from 7-amino-2H-1,4-benzoxazin-3(4H)-one (0.008 g, 0.051 mmol) and 1-(3-trifluoromethyl-phenyl)-piperidine-4-carboxylic acid, (0.013 g, 0.046 mmol) as a white solid. ¹H NMR (400 MHz, CDOD₃) δ (ppm): 7.38 (t, 1H), 7.30 (d, 1H), 7.20 (dd, 1H), 7.17 (bs, 1H), 7.12 (dd, 1H), 7.05 (d, 1H), 6.83 (d, 1H), 4.55 (s, 2H), 3.84 (d, 2H), 2.83 (dt, 2H), 2.53-2.49 (m, 1H), 1.96-1.89 (m, 4H). Mass Spectrum (LCMS, ESI pos.) Calcd. for C₂₁H₂₀F₃N₃O₃: 419.4; Found: 420.2 (M+H).

### Example 45

### 1-(3-Trifluoromethyl-phenyl)-piperidine-4-carboxylic acid (6-methylamino-quinazolin-5-yl)-amide

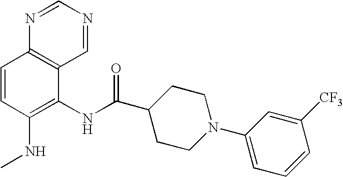 Using the procedure outlined in Example 8, the title compound was prepared from 5-amino-6-methylaminoquinoxaline (0.009 g, 0.051 mmol) and 1-(3-trifluoromethyl-phenyl)-piperidine-4-carboxylic acid, (0.013 g, 0.046 mmol) as a reddish yellow solid. ¹H NMR (400 MHz, CDOD₃) δ (ppm): 8.92 (bs, 1H), 8.63 (d, 1H), 8.60 (d, 1H), 7.92 (d, 1H), 7.46 (d, 1H), 7.37 (t, 1H), 7.25-7.10 (m, 3H), 3.86 (d, 2H), 3.06 (s, 3H), 2.97 (t, 2H), 2.75 (m, 1H), 2.32-2.22 (m, 2H), 2.18-2.06 (m, 2H). Mass Spectrum (LCMS, ESI pos.) Calcd. for C₂₂H₂₂F₃N₅O: 419.4; Found: 420.2 (M+H).

### Example 46

### 1-(3-Trifluoromethyl-phenyl)-piperidine-4-carboxylic acid (1-acetyl-2,3-dihydro-1H-indol-4-yl)-amide

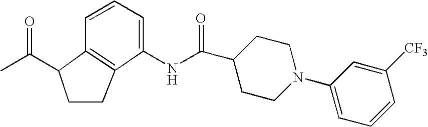 Using the procedure outlined in Example 8, the title compound was prepared from 1-(4-amino-2,3-dihydro-indol-1-yl)-ethanone (0.009 g, 0.051 mmol) and 1-(3-trifluoromethyl-phenyl)-piperidine-4-carboxylic acid, (0.013 g, 0.046 mmol) as a white solid. ¹H NMR (400 MHz, CDCl₃) δ (ppm): 8.06 (d, 1H), 7.36 (t, 1H), 7.32-6.98 (m, 6H), 4.08 (t, 2H), 3.81 (d, 2H), 3.10 (t, 2H), 2.88 (t, 2H), 2.43 (m, 1H), 2.14-1.94 (m, 4H). Mass Spectrum (LCMS, ESI pos.) Calcd. for C₂₃H₂₄F₃N₃O₂: 431.5; Found: 432.2 (M+H).

### Example 47

### 1-(3-Trifluoromethyl-phenyl)-piperidine-4-carboxylic acid (5-furan-2-yl-2H-pyrazol-3-yl)-amide

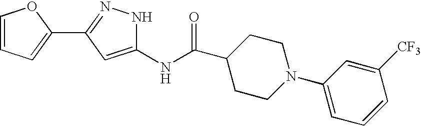 A. To a solution of 1-(3-trifluoromethyl-phenyl)-piperidine-4-carboxylic acid (1.63 g, 5.96 mmol) in a mixed solvent (80 mL, DCM:DMF=3:1) was added PS-HOBT (4.41 g, 3.97 mmol), N,N′-diisopropylcarbodiimide (2.7 mL, 17.5 mmol), and pyridine (0.19 mL, 2.38 mmol) (Scheme 2). After stirring for 8 hours, the resin was filtered off and washed with DCM, DMF, DCM, and THF sequentially. The resin was dried under vacuum, ready for the next coupling step.
B. A mixture of resin bound material (0.040 g) and 5-furan-2-yl-2H-pyrazol-3-ylamine (0.010 g 0.067 mmol) in 1 mL of dioxane was heated to 100° C. for 14 hours. Resin was filtered off and the filtrate was concentrated to provide a crude product which was then purified by silica gel column chromatograph to afford the title compound as light brown solid. ¹H NMR (400 MHz, CD₃CN) δ (ppm): 8.87 (bs, 1H), 7.56 (s, 1H), 7.42 (t, 1H), 7.22-7.20 (m, 2H), 7.09 (d, 1H), 6.73 (d, 1H), 6.64 (bs, 1H), 6.55 (dd, 1H), 3.70 (d, 2H), 2.86 (dt, 2H), 2.58-2.53 (m, 1H), 2.02-1.80 (m, 4H). Mass Spectrum (LCMS, ESI pos.) Calcd. for C₂₀H₁₉F₃N₄O₂: 404.4; Found: 405.2 (M+H).

### Example 48

### 1-(3-Trifluoromethyl-phenyl)-piperidine-4-carboxylic acid (5-thiophen-2-yl-2H-pyrazol-3-yl)-amide

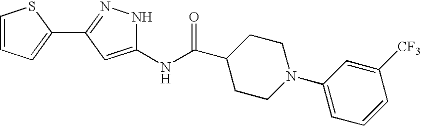 Using the procedure outlined in Example 47, the title compound was prepared from resin bound material (0.050 g) and 5-thiophen-2-yl-2H-pyrazol-3-ylamine (0.011 g, 0.067 mmol) as a white solid (Scheme 2). ¹H NMR (400 MHz, CDOD₃) δ (ppm): 7.44 (m, 4H), 7.21 (d, 1H), 7.18 (s, 1H), 7.08 (t, 1H), 7.05 (d, 1H), 3.84 (d, 2H), 2.85 (dt, 2H), 2.62-2.56 (m, 1H), 2.00-1.89 (m, 4H). Mass Spectrum (LCMS, ESI pos.) Calcd. for C₂₀H₁₉F₃N₄OS: 420.5; Found: 421.1 (M+H).

### Example 49

### 1-(3-Trifluoromethyl-phenyl)-piperidine-4-carboxylic acid thieno[2,3-d]pyrimidin-4-ylamide

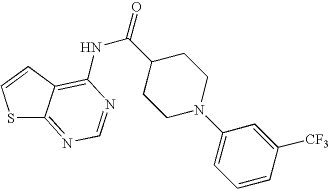 1-(3-Trifluoromethyl-phenyl)-piperidine-4-carbonyl chloride was prepared from 1-(3-trifluoromethyl-phenyl)-piperidine-4-carboxylic acid as described in Example 8A. The title compound was prepared (0.012 g) from thieno[2,3-d]pyrimidin-4-ylamine (0.33 mmol) and 1-(3-trifluoromethyl-phenyl)-piperidine-4-carbonyl chloride (0.33 mmol) using the procedure outlined in Example 8B. ¹H NMR (400 MHz, CDCl₃) δ (ppm): 10.86-10.89 (s, 1H), 10.70-10.75 (s, 1H), 9.67-9.70 (m, 1H), 9.55-9.59 (m, 1H), 9.40-9.45 (m, 1H), 9.10-9.21 (m, 3H), 5.87-5.93 (m, 2H), 4.67-5.02 (m, 3H), 4.05-4.28 (m, 4H). Mass Spectrum (LCMS, ESI pos.) Calcd. for C₁₉H₁₇F₃N₄OS: 406.11 (M+H). Found: 407.20.

### Example 50

### 04/072069

### 1-(3-Trifluoromethyl-phenyl)-piperidine-4-carboxylic acid (3-methyl-benzo[d]isothiazol-5-yl)-amide

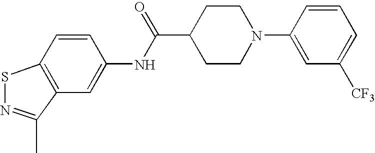 1-(3-Trifluoromethyl-phenyl)-piperidine-4-carbonyl chloride was prepared from 1-(3-trifluoromethyl-phenyl)-piperidine-4-carboxylic acid as described in Example 8A. The title compound was prepared (0.012 g) from 3-methyl-benzo[d]isothiazol-5-ylamine (0.33 mmol) and 1-(3-trifluoromethyl-phenyl)-piperidine-4-carbonyl chloride (0.33 mmol) using the procedure outlined in Example 8B. ¹H NMR (400 MHz, CDCl₃) δ (ppm): 8.45-8.47 (s, 1H), 7.85-7.88 (m, 1H), 7.35-7.47 (m, 3H), 7.10-7.18 (m, 3H), 3.83-3.90(m, 2H), 2.87-2.94 (m, 2H), 2.48-2.53 (m, 1H), 2.04-2.18 (m, 4H). Mass Spectrum (LCMS, ESI pos.) Calcd. for C₂₁H₂₀F₃N₃OS: 419.13 (M+H). Found: 420.20.

### Example 51

### 1-(3-Trifluoromethyl-phenyl)-piperidine-4-carboxylic acid (6-chloro-4-methyl-pyridazin-3-yl)-amide

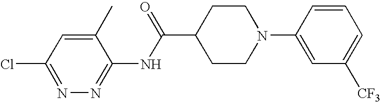 A. A solution of 3,6-Dichloro-4-methyl-pyridazine (1.0 g, 6.1 mmol) in concentrated ammonium hydroxide (40 mL) was heated to 130° C. in a sealed reaction vessel. After 18 h the solution was cooled to room temperature. The solution was filtered. The solid was washed with water and dried in vacuo to give 6-Chloro-4-methyl-pyridazin-3-ylamine. ¹H NMR (400 MHz, CDCl₃) δ (ppm): 2.18-2.20 (d, 3H), 4.80-4.90 (bs, 2H), 7.11-7.12 (s, 1H).
B. 1-(3-Trifluoromethyl-phenyl)-piperidine-4-carbonyl chloride was prepared from 1-(3-trifluoromethyl-phenyl)-piperidine-4-carboxylic acid as described in Example 8A. The title compound was prepared (0.012 g) from 6-chloro-4-methyl-pyridazin-3-ylamine (0.33 mmol) and 1-(3-trifluoromethyl-phenyl)-piperidine-4-carbonyl chloride (0.33 mmol) using the procedure outlined in Example 8B. ¹H NMR (400 MHz, CDCl₃) δ (ppm): 9.77-9.85 (s, 1H), 7.43-7.48 (m, 1H), 7.30-7.38 (m, 1H), 7.00-7.17 (m, 3H), 3.59-3.78(m, 2H), 2.70-2.98 (m, 3H), 2.32-2.40 (s, 3H), 1.93-2.18 (m, 4H). Mass Spectrum (LCMS, ESI pos.) Calcd. For C₁₈H₁₈ClF₃N₄O: 398.11 (M+H). Found: 399.1.

### Example 52

### 04/072069

### 1-(3-Trifluoromethyl-phenyl)-piperidine-4-carboxylic acid (1H-indazol-7-yl)-amide

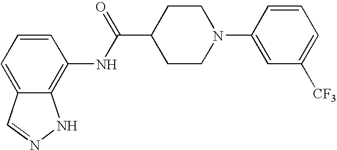 1-(3-Trifluoromethyl-phenyl)-piperidine-4-carbonyl chloride was prepared from 1-(3-trifluoromethyl-phenyl)-piperidine-4-carboxylic acid as described in Example 8A. The title compound was prepared (0.007 g) from 1H-indazol-7-ylamine (0.33 mmol) and 1-(3-trifluoromethyl-phenyl)-piperidine-4-carbonyl chloride (0.33 mmol) using the procedure outlined in Example 8B. ¹H NMR (400 MHz, CDCl₃+ drops CD₃OD and DMSO(d6)) δ (ppm): 8.00 (s, 1H), 7.60-7.66 (m, 1H), 7.47-7.52 (m, 1H), 7.40 (s, 1H), 7.33-7.36 (m, 1H), 7.04-7.13 (m, 4H), 2.80-2.90 (m, 2H), 2.58-2.63 (m, 1H), 2.00-2.10 (m, 4H). Mass Spectrum (LCMS, ESI pos.) Calcd. For C₂₀H₁₉F₃N₄O: 388.15 (M+H). Found: 389.2.

### Example 53

### 1-(2-Bromo-phenyl)-piperidine-4-carboxylic acid quinolin-3-ylamide

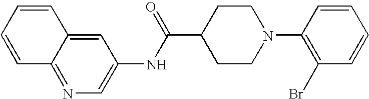 A. 1-(2-Bromo-phenyl)-piperidine-4-carboxylic acid was prepared from 1,2-dibromobenzene (0.483 mL, 4 mmol) and ethylisonipecotate (0.615 ml, 4.0 mmol) as described in Example 1.
B. 1-(2-Bromo-phenyl)-piperidine-4-carbonyl chloride was prepared from 1-(2-bromo-phenyl)-piperidine-4-carboxylic acid (0.08 g, 0.28 mmol) as described in Example 8A.
C. The title compound was prepared (0.025 g) from 3-aminoquinoline (0.14 mmol) and 1-(2-Bromo-phenyl)-piperidine-4-carbonyl chloride (0.14 mmol) using the procedure outlined in Example 8B. ¹H NMR (400 MHz, CD₃OD) δ (ppm): 8.77-8.80 (m, 1H), 8.30-8.33(s, 1H), 7.80-7.85 (m, 1H), 7.55-7.60 (m, 1H), 7.48-7.52 (m, 3H), 7.20-7.26 (m, 1H), 7.00-7.08 (m, 1H), 6.84-6.88 (m, 1H), 3.40-3.46 (m, 2H), 2.65-2.75 (m, 2H), 2.48-2.56 (m, 1H), 1.90-2.20 (4H). Mass Spectrum (LCMS, ESI pos.) Calcd. For C₂₁H₂₀BrN₃O: 409.08 (M+H). Found: 410.1.

### Example 54

### 04/072069

### 1-(9-Methyl-9H-purin-6-yl)-piperidine-4-carboxylic acid (1H-indol-4-yl)-amide

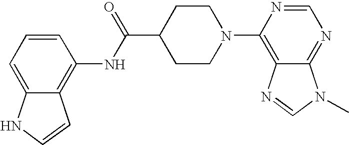 A. A solution of 6-Bromo-9H-purine (2 g, 10 mmol) and potassium carbonate (2.9 g, 22.1 mmol) in acetonitrile (20 mL) was treated with methyl iodide (0.69 mL, 11 mmol). After 18 h solution was filtered. The filtrate was concentrated to give 6-bromo-9-methyl-9H-purine as a 2:1 mixture of regioisomers and was used as is for the next step.
B. 1-(9-Methyl-9H-purin-6-yl)-piperidine-4-carboxylic acid was prepared from 6-bromo-9-methyl-9H-purine (4 mmol) and ethylisonipecotate (0.615 ml, 4.0 mmol) using the procedure in Example 1.
C. The title compound was prepared (0.004 g) from 1-(9-Methyl-9H-purin-6-yl)-piperidine-4-carboxylic acid (0.141 g, 0.5 mmol) and 1H-indol-4-ylamine (0.214 g, 1.5 mmol) as described in Example 8. ¹H NMR (400 MHz, CDCl₃) 6 (ppm): 8.30-8.42 (m, 2H), 7.72-7.81(m, 2H), 7.40-7.47(s, 1H), 7.15-7.20 (m, 2H), 6.50 (s, 1H), 5.50-5.60 (bs, 2H), 3.81-3.84 (s, 3H), 3.23-3.37 (m, 2H), 2.70-2.80 (m, 1H), 1.99-2.25 (m, 4H). Mass Spectrum (LCMS, ESI pos.) Calcd. For C₂₀H₂₁N₇O: 375.18 (M+H). Found: 376.20.

### Example 55

### 1-Phenyl-piperidine-4-carboxylic acid quinolin-3-ylamide

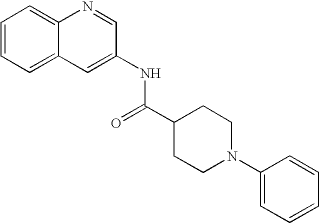 Using the procedure outlined in Example 1A, 1-phenyl-piperidine-4-carboxylic acid is prepared from bromobenzene (4 mmol). Using the procedure outlined in Example 8, the title compound is prepared from 3-aminoquinoline (0.5 mmol) and 1-phenyl-piperidine-4-carboxylic acid (0.18 mmol).

### Example 56

### 1-Benzothiazol-5-yl-piperidine-4-carboxylic acid quinolin-3-ylamide

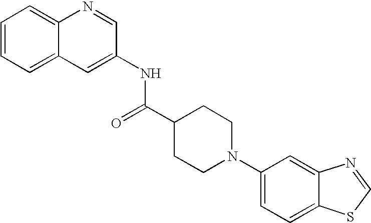 Using the procedure outlined in Example 1, 1-benzothiazol-5-yl-piperidine-4-carboxylic acid is prepared from 5-bromobenzothiazole (4 mmol). Using the procedure outlined in Example 8, the title compound is prepared from 3-aminoquinoline (0.5 mmol) and 1-benzothiazol-5-yl-piperidine-4-carboxylic acid (0.20 mmol).

### Example 57

### 1-Benzothiazol-5-yl-piperidine-4-carboxylic acid (2,3-dihydro-1H-indol-4-yl)-amide

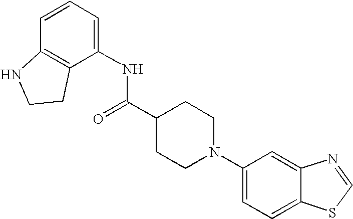 Using the procedure outlined in Example 1A, 1-benzothiazol-5-yl-piperidine-4-carboxylic acid is prepared from 5-bromobenzothiazole (4 mmol). Using the procedure outlined in Example 8, the title compound is prepared from 4-aminoindoline (0.5 mmol) and 1-benzothiazol-5-yl-piperidine-4-carboxylic acid (0.20 mmol).

### Example 58

### 1-Phenyl-piperidine-4-carboxylic acid (2,3-dihydro-1H-indol-4-yl)-amide

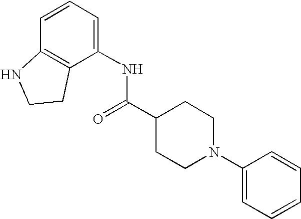 Using the procedure outlined in Example 1A, 1-phenyl-piperidine-4-carboxylic acid is prepared from bromobenzene (4 mmol). Using the procedure outlined in Example 8, the title compound is prepared from 4-aminoindoline (0.5 mmol) and 1-phenyl-piperidine-4-carboxylic acid (0.18 mmol).

### Example 59

### 3,4,5,6-Tetrahydro-2H-[1,3′]bipyridinyl-4-carboxylic acid isoquinolin-1-ylamide

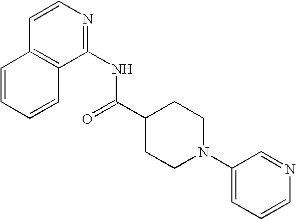 Using the procedure outlined in Example 1A, 3,4,5,6-tetrahydro-2H-[1,3′]bipyridinyl-4-carboxylic acid is prepared from 3-bromopyridine (4 mmol). Using the procedure outlined in Example 8, the title compound is prepared from 1-aminoisoquinoline (0.5 mmol) and 3,4,5,6-tetrahydro-2H-[1,3′]bipyridinyl-4-carboxylic acid (0.18 mmol).

### Example 60

### 1-Phenyl-piperidine-4-carboxylic acid isoquinolin-1-ylamide

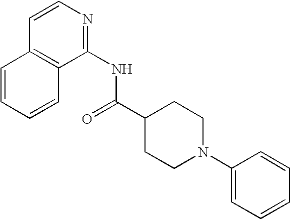 Using the procedure outlined in Example 1, 1-phenyl-piperidine-4-carboxylic acid is prepared from bromobenzene (4 mmol). Using the procedure outlined in Example 8, the title compound is prepared from 1-aminoisoquinoline (0.5 mmol) and 1-phenyl-piperidine-4-carboxylic acid (0.18 mmol).

### Example 61

### 1-Phenyl-piperidine-4-carboxylic acid cinnolin-3-ylamide

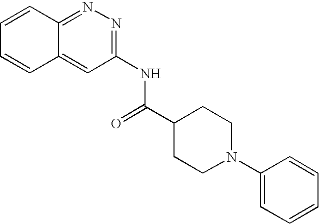 Using the procedure outlined in Example 1A, 1-phenyl-piperidine-4-carboxylic acid is prepared from bromobenzene (4 mmol). Using the procedure outlined in Example 8, the title compound is prepared from 3-aminocinnoline (0.5 mmol) and 1-phenyl-piperidine-4-carboxylic acid (0.18 mmol).

### Example 62

### 1-Benzothiazol-5-yl-piperidine-4-carboxylic acid cinnolin-3-ylamide

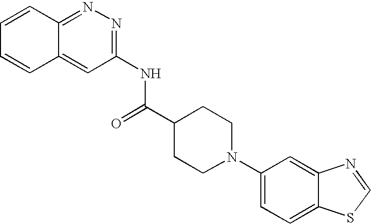 Using the procedure outlined in Example 1A, 1-benzothiazol-5-yl-piperidine-4-carboxylic acid is prepared from 5-bromobenzothiazole (4 mmol). Using the procedure outlined in Example 8, the title compound is prepared from 3-aminocinnoline (0.5 mmol) and 1-benzothiazol-5-yl-piperidine-4-carboxylic acid (0.20 mmol).

### Example 63

### 1-Benzothiazol-5-yl-piperidine-4-carboxylic acid phthalazin-5-ylamide

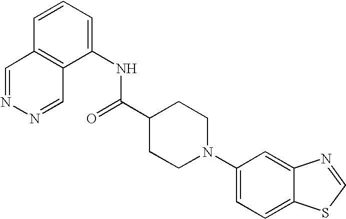 A. A solution of phthalazine (2.5 g, 19.2 mmol) in concentrated sulfuric acid (30 mL) is treated slowly with potassium nitrate (2 g, 19.2 mmol). After 18 h the solution is cooled. Water (30 mL) is added. The solution is basified using 10 M NaOH to pH 13 (litmus). The solution is cooled for 18 h, filtered to give 5-nitro-phthalazine (0.93 g).
B. A solution of 5-nitro-phthalazine (0.175 g, 1 mmol) and SnCl₂ (0.57 g, 3 mmol) in concentrated HCl (10 mL) is stirred at room temperature for 2 h. Ice is added to the solution. A solution of 25% NaOH is added to pH 14 (litmus). The solution is cooled for 18 h. The solution is filtered. Aqueous filtrate is extracted twice with EtOAc. EtOAc layers are combined, washed with water, brine, dried over Na₂SO₄, filtered and concentrated to give phthalazin-5-ylamine (0.050 g).
C. Using the procedure outlined in Example 1A, 1-benzothiazol-5-yl-piperidine-4-carboxylic acid is prepared from 5-bromobenzothiazole (4 mmol). Using the procedure outlined in Example 8, the title compound is prepared from 5-aminophthalazine (0.5 mmol) and 1-benzothiazol-5-yl-piperidine-4-carboxylic acid (0.20 mmol).

### Example 64

### 3,4,5,6-Tetrahydro-2H-[1,4′]bipyridinyl-4-carboxylic acid phthalazin-5-ylamide

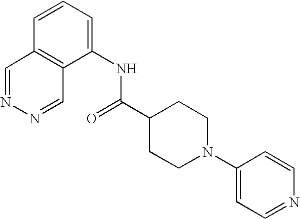 Using the procedure outlined in Example 1A, 3,4,5,6-tetrahydro-2H-[1,4′]bipyridinyl-4-carboxylic acid is prepared from 4-bromopyridine (4 mmol). Using the procedure outlined in Example 8, the title compound is prepared from 3,4,5,6-tetrahydro-2H-[1,4′]bipyridinyl-4-carboxylic acid (0.5 mmol) and 5-aminophthalazine (0.20 mmol).

### Example 65

### 1-Phenyl-piperidine-4-carboxylic acid quinazolin-4-ylamide

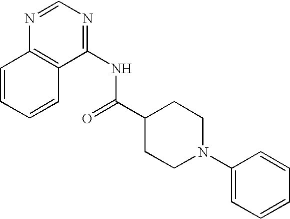 Using the procedure outlined in Example 1A, 1-phenyl-piperidine-4-carboxylic acid is prepared from bromobenzene (4 mmol). Using the procedure outlined in Example 8, the title compound is prepared from 4-aminoquinazoline (0.5 mmol) and 1-phenyl-piperidine-4-carboxylic acid (0.18 mmol).

### Example 66

### 1-Benzothiazol-5-yl-piperidine-4-carboxylic acid quinazolin-4-ylamide

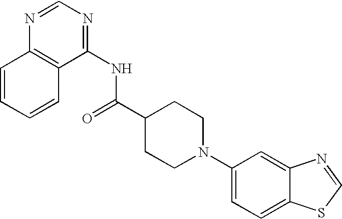 Using the procedure outlined in Example 1A, 1-benzothiazol-5-yl-piperidine-4-carboxylic acid is prepared from 5-bromobenzothiazole (4 mmol). Using the procedure outlined in Example 8, the title compound is prepared from 4-aminoquinazoline (0.5 mmol) and 1-benzothiazol-5-yl-piperidine-4-carboxylic acid (0.20 mmol).

### Example 67

### 1-Benzothiazol-5-yl-piperidine-4-carboxylic acid pyridin-3-ylamide

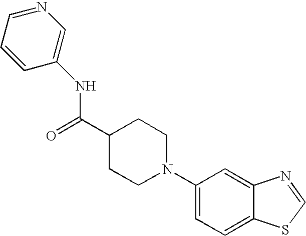 Using the procedure outlined in Example 1A, 1-benzothiazol-5-yl-piperidine-4-carboxylic acid is prepared from 5-bromobenzothiazole (4 mmol). Using the procedure outlined in Example 8, the title compound is prepared from 3-aminopyridine (0.5 mmol) and 1-benzothiazol-5-yl-piperidine-4-carboxylic acid (0.20 mmol).

### Example 68

### 1-Phenyl-piperidine-4-carboxylic acid benzo[d]isothiazol-3-ylamide

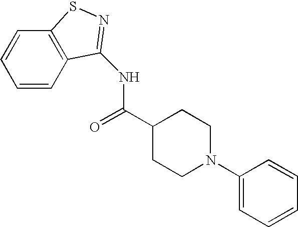 Using the procedure outlined in Example 1A, 1-phenyl-piperidine-4-carboxylic acid is prepared from bromobenzene (4 mmol). Using the procedure outlined in Example 8, the title compound is prepared from benzo[d]isothiazol-3-ylamine (0.5 mmol) and 1-phenyl-piperidine-4-carboxylic acid (0.18 mmol).

### Example 69

### 3,4,5,6-Tetrahydro-2H-[1,2′]bipyridinyl-4-carboxylic acid benzo[d]isothiazol-3-ylamide

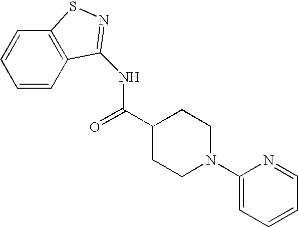 Using the procedure outlined in Example 1A, 3,4,5,6-tetrahydro-2H-[1,2′]bipyridinyl-4-carboxylic acid is prepared from 2-bromopyridine (4 mmol). Using the procedure outlined in Example 8, the title compound is prepared from benzo[d]isothiazol-3-ylamine (0.5 mmol) and 3,4,5,6-tetrahydro-2H-[1,2′]bipyridinyl-4-carboxylic acid (0.18 mmol).

### Example 70

### 1-Phenyl-piperidine-4-carboxylic acid benzothiazol-2-ylamide

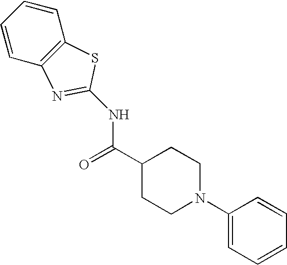 Using the procedure outlined in Example 1A, 1-phenyl-piperidine-4-carboxylic acid is prepared from bromobenzene (4 mmol). Using the procedure outlined in Example 8, the title compound is prepared from benzothiazol-2-ylamine (0.5 mmol) and 1-Phenyl-piperidine-4-carboxylic acid (0.18 mmol).

### Example 71

### 3,4,5,6-Tetrahydro-2H-[1,3′]bipyridinyl-4-carboxylic acid benzothiazol-2-ylamide

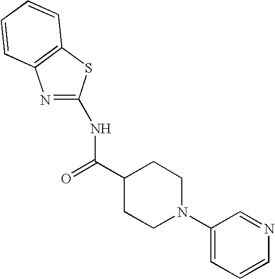 Using the procedure outlined in Example 1A, 3,4,5,6-tetrahydro-2H-[1,3′]bipyridinyl-4-carboxylic acid is prepared from 3-bromopyridine (4 mmol). Using the procedure outlined in Example 8, the title compound is prepared from benzothiazol-2-ylamine (0.5 mmol) and 3,4,5,6-tetrahydro-2H-[1,3′]bipyridinyl-4-carboxylic acid (0.18 mmol).

### Example 72

### 1-Pyridazin-3-yl-piperidine-4-carboxylic acid phthalazin-1-ylamide

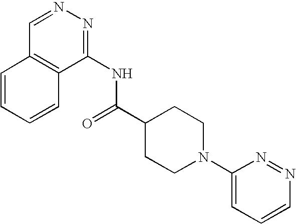 A. A solution of 3-chloro-pyridazine 2-oxide (0.5 mmol) and piperidine-4-carboxylic acid methyl ester (5 mmol) is heated to 80° C. for 18 h. The solution is cooled to room temperature. 2.5N NaOH (15 mmol) is added and the solution is heated to 60° C. for 18 h. The reaction solution is cooled to room temperature and partitioned between EtOAc and water. The aqueous layer is separated and acidified using 3N HCl. The aqueous layer is extracted with EtOAc. EtOAc layer is washed with brine, dried over MgSO₄, filtered and concentrated to give 1-(2-oxy-pyridazin-3-yl)-piperidine-4-carboxylic acid (0.3 mmol). A suspension of 1-(2-oxy-pyridazin-3-yl)-piperidine-4-carboxylic acid (0.1 mmol) and iron powder (0.4 mmol) in 50% aqueous acetic acid (2 mL) is stirred at room temperature for 2 h. The solution is filtered and the filtrate is concentrated to give 1-pyridazin-3-yl-piperidine-4-carboxylic acid (0.07 mmol).
B. Using the procedure outlined in Example 8, the title compound is prepared 1-aminophthalazine (0.5 mmol) and 1-pyridazin-3-yl-piperidine-4-carboxylic acid (0.18 mmol).

### Example 73

### 1-Thiophen-2-yl-piperidine-4-carboxylic acid phthalazin-1-ylamide

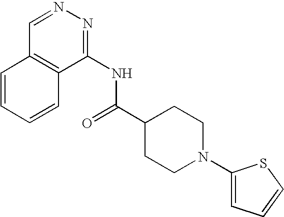 Using the procedure outlined in Example 1A, 1-thiophen-2-yl-piperidine-4-carboxylic acid is prepared from 2-bromothiophene (4 mmol). Using the procedure outlined in Example 8, the title compound is prepared from 1-aminophthalazine (0.5 mmol) and 1-Thiophen-2-yl-piperidine-4-carboxylic acid (0.18 mmol).

### Example 74

### 1-Thiophen-2-yl-piperidine-4-carboxylic acid (7H-purin-6-yl)-amide

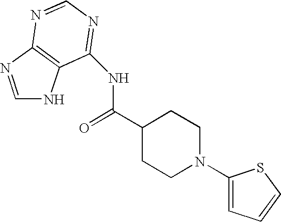 Using the procedure outlined in Example 1A, 1-thiophen-2-yl-piperidine-4-carboxylic acid is prepared from 2-bromothiophene (4 mmol). Using the procedure outlined in Example 8, the title compound is prepared from 7H-purin-6-ylamine (0.5 mmol) and 1-thiophen-2-yl-piperidine-4-carboxylic acid (0.18 mmol).

### Example 75

### 1-Pyrimidin-2-yl-piperidine-4-carboxylic acid (7H-purin-6-yl)-amide

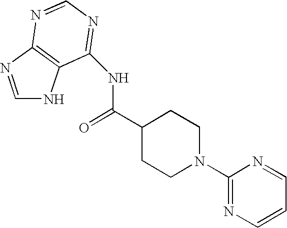 Using the procedure outlined in Example 1A, 1-pyrimidin-2-yl-piperidine-4-carboxylic acid is prepared from 2-bromopyrimidine (4 mmol). Using the procedure outlined in Example 8, the title compound is prepared from 7H-purin-6-ylamine (0.5 mmol) and 1-pyrimidin-2-yl-piperidine-4-carboxylic acid (0.18 mmol).

### Example 76

### 1-Pyrimidin-2-yl-piperidine-4-carboxylic acid cinnolin-4-ylamide

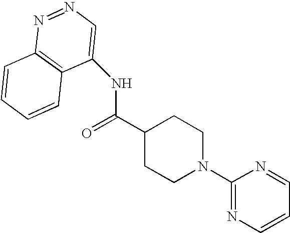 Using the procedure outlined in Example 1A, 1-pyrimidin-2-yl-piperidine-4-carboxylic acid is prepared from 2-bromopyrimidine (4 mmol). Using the procedure outlined in Example 8, the title compound is prepared from cinnolin-4-ylamine (0.5 mmol) and 1-pyrimidin-2-yl-piperidine-4-carboxylic acid (0.18 mmol).

### Example 77

### 1-Thiophen-2-yl-piperidine-4-carboxylic acid cinnolin-4-ylamide

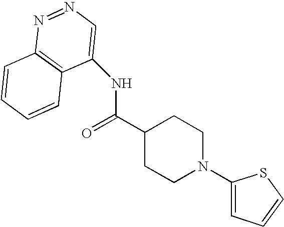 Using the procedure outlined in Example 1A, 1-thiophen-2-yl-piperidine-4-carboxylic acid is prepared from 2-bromothiophene (4 mmol). Using the procedure outlined in Example 8, the title compound is prepared from cinnolin-4-ylamine (0.5 mmol) and 1-thiophen-2-yl-piperidine-4-carboxylic acid (0.18 mmol).

### Example 78

### 1-Thiophen-2-yl-piperidine-4-carboxylic acid quinoxalin-6-ylamide

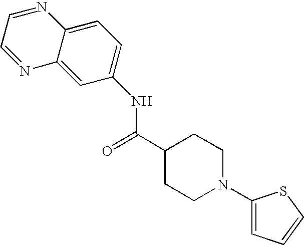 Using the procedure outlined in Example 1A, 1-thiophen-2-yl-piperidine-4-carboxylic acid is prepared from 2-bromothiophene (4 mmol). Using the procedure outlined in Example 8, the title compound is prepared from quinoxalin-6-ylamine (0.5 mmol) and 1-thiophen-2-yl-piperidine-4-carboxylic acid (0.18 mmol).

### Example 79

### 1-Pyridazin-3-yl-piperidine-4-carboxylic acid quinoxalin-6-ylamide

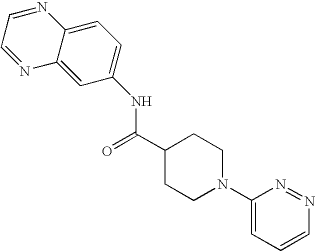 Using the procedure outlined in Example 1A, 1-pyridazin-3-yl-piperidine-4-carboxylic acid (4 mmol) is prepared as in Example 120. Using the procedure outlined in Example 8, the title compound is prepared from quinoxalin-6-ylamine (0.5 mmol) and 1-pyridazin-3-yl-piperidine-4-carboxylic acid (0.18 mmol).

### Example 80

### 1-Pyrimidin-2-yl-piperidine-4-carboxylic acid quinoxalin-6-ylamide

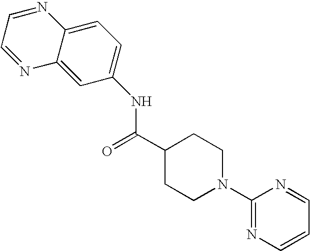 Using the procedure outlined in Example 1A, 1-pyrimidin-2-yl-piperidine-4-carboxylic acid is prepared from 2-bromopyrimidine (4 mmol). Using the procedure outlined in Example 8, the title compound is prepared from quinoxalin-6-ylamine (0.5 mmol) and 1-pyrimidin-2-yl-piperidine-4-carboxylic acid (0.18 mmol).

### Example 81

### 1-Pyrimidin-2-yl-piperidine-4-carboxylic acid pyrazin-2-ylamide

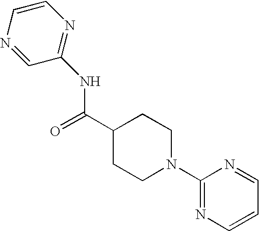 Using the procedure outlined in Example 1A, 1-pyrimidin-2-yl-piperidine-4-carboxylic acid is prepared from 2-bromopyrimidine (4 mmol). Using the procedure outlined in Example 8, the title compound is prepared from pyrazin-2-ylamine (0.5 mmol) and 1-pyrimidin-2-yl-piperidine-4-carboxylic acid (0.18 mmol).

### Example 82

### 1-Thiophen-2-yl-piperidine-4-carboxylic acid pyrazin-2-ylamide

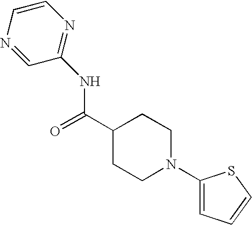 Using the procedure outlined in Example 1A, 1-thiophen-2-yl-piperidine-4-carboxylic acid is prepared from 2-bromothiophene (4 mmol). Using the procedure outlined in Example 8, the title compound is prepared from pyrazin-2-ylamine (0.5 mmol) and 1-thiophen-2-yl-piperidine-4-carboxylic acid (0.18 mmol).

### Example 83

### 1-Thiophen-2-yl-piperidine-4-carboxylic acid quinazolin-7-ylamide

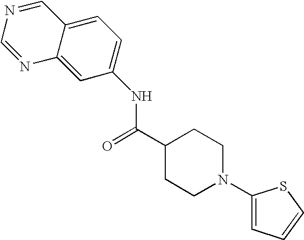 Using the procedure outlined in Example 1A, 1-thiophen-2-yl-piperidine-4-carboxylic acid is prepared from 2-bromothiophene (4 mmol). Using the procedure outlined in Example 8, the title compound is prepared from quinazolin-7-ylamine (0.5 mmol) and 1-thiophen-2-yl-piperidine-4-carboxylic acid (0.18 mmol).

### Example 84

### 1-Pyrimidin-2-yl-piperidine-4-carboxylic acid quinazolin-7-ylamide

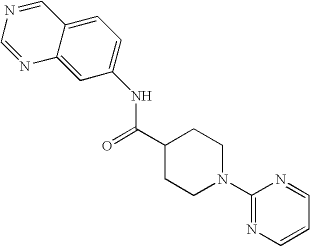 Using the procedure outlined in Example 1, 1-pyrimidin-2-yl-piperidine-4-carboxylic acid is prepared from 2-bromopyrimidine (4 mmol). Using the procedure outlined in Example 8, the title compound is prepared from quinazolin-7-ylamine (0.5 mmol) and 1-pyrimidin-2-yl-piperidine-4-carboxylic acid (0.18 mmol).

### Example 85

### 1-Pyrimidin-2-yl-piperidine-4-carboxylic acid pyridazin-3-ylamide

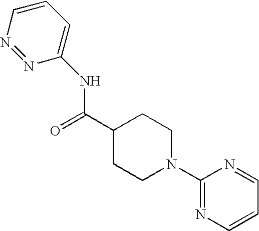 Using the procedure outlined in Example 1A, 1-pyrimidin-2-yl-piperidine-4-carboxylic acid is prepared from 2-bromopyrimidine (4 mmol). Using the procedure outlined in Example 8, the title compound is prepared from pyridazin-3-ylamine (0.5 mmol) and 1-pyrimidin-2-yl-piperidine-4-carboxylic acid (0.18 mmol).

### Example 86

### 1-Thiophen-2-yl-piperidine-4-carboxylic acid pyridazin-3-ylamide

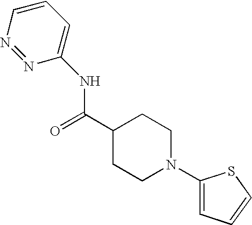 Using the procedure outlined in Example 1A, 1-thiophen-2-yl-piperidine-4-carboxylic acid is prepared from 2-bromothiophene (4 mmol). Using the procedure outlined in Example 8, the title compound is prepared from pyridazin-3-ylamine (0.5 mmol) and 1-thiophen-2-yl-piperidine-4-carboxylic acid (0.18 mmol).

### Example 87

### 1-Benzo[b]thiophen-3-yl-piperidine-4-carboxylic acid (2-oxo-2,3-dihydro-1H-benzoimidazol-5-yl)-amide

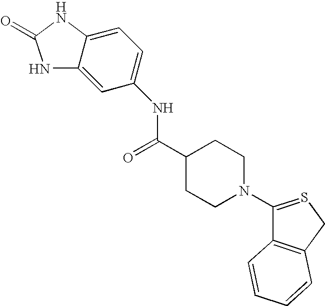 Using the procedure outlined in Example 1A, 1-benzo[b]thiophen-3-yl-piperidine-4-carboxylic acid is prepared from 3-bromo-benzo[b]thiophene (4 mmol). Using the procedure outlined in Example 8, the title compound is prepared from 5-amino-1,3-dihydro-benzoimidazol-2-one (0.5 mmol) and 1-benzo[b]thiophen-3-yl-piperidine-4-carboxylic acid (0.18 mmol).

### Example 88

### 1-Naphthalen-2-yl-piperidine-4-carboxylic acid (2-oxo-2,3-dihydro-1H-benzoimidazol-5-yl)-amide

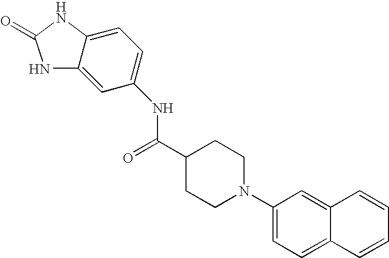 Using the procedure outlined in Example 1A, 1-naphthalen-2-yl-piperidine-4-carboxylic acid is prepared from 2-bromo-naphthalene (4 mmol). Using the procedure outlined in Example 8, the title compound is prepared from 5-amino-1,3-dihydro-benzoimidazol-2-one (0.5 mmol) and 1-naphthalen-2-yl-piperidine-4-carboxylic acid (0.18 mmol).

### Example 89

### 1-Naphthalen-2-yl-piperidine-4-carboxylic acid (3-oxo-3,4-dihydro-2H-benzo[1,4]oxazin-6-yl)-amide

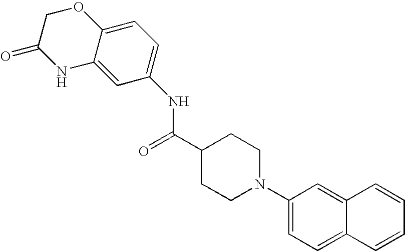 Using the procedure outlined in Example 1A, 1-naphthalen-2-yl-piperidine-4-carboxylic acid is prepared from 2-bromo-naphthalene (4 mmol). Using the procedure outlined in Example 8, the title compound is prepared from 6-amino-4H-benzo[1,4]oxazin-3-one (0.5 mmol) and 1-naphthalen-2-yl-piperidine-4-carboxylic acid (0.18 mmol).

### Example 90

### 1-Biphenyl-3-yl-piperidine-4-carboxylic acid (3-oxo-3,4-dihydro-2H-benzo[1,4]oxazin-6-yl)-amide

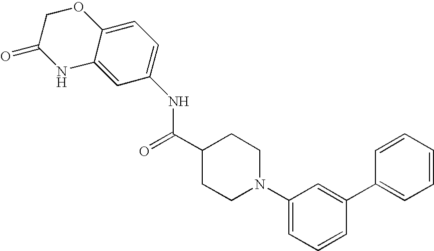 Using the procedure outlined in Example 1A, 1-biphenyl-3-yl-piperidine-4-carboxylic acid is prepared from 3-bromo-biphenyl (4 mmol). Using the procedure outlined in Example 8, the title compound is prepared from 6-amino-4H-benzo[1,4]oxazin-3-one (0.5 mmol) and 1-biphenyl-3-yl-piperidine-4-carboxylic acid (0.18 mmol).

### Example 91

### 1-Biphenyl-3-yl-piperidine-4-carboxylic acid quinazolin-2-ylamide

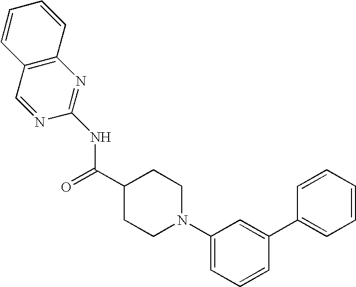 Using the procedure outlined in Example 1A, 1-biphenyl-3-yl-piperidine-4-carboxylic acid is prepared from 3-bromo-biphenyl (4 mmol). Using the procedure outlined in Example 8, the title compound is prepared from quinazolin-2-ylamine (0.5 mmol) and 1-biphenyl-3-yl-piperidine-4-carboxylic acid (0.18 mmol).

### Example 92

### 1-Quinolin-3-yl-piperidine-4-carboxylic acid quinazolin-2-ylamide

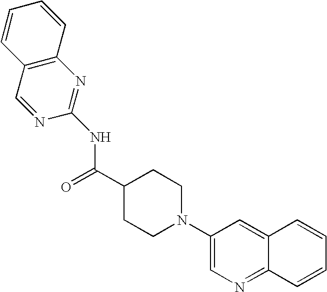 Using the procedure outlined in Example 1A, 1-quinolin-3-yl-piperidine-4-carboxylic acid is prepared from 3-bromo-quinoline (4 mmol). Using the procedure outlined in Example 8, the title compound is prepared from quinazolin-2-ylamine (0.5 mmol) and 1-quinolin-3-yl-piperidine-4-carboxylic acid (0.18 mmol).

### Example 93

### 1-Quinolin-3-yl-piperidine-4-carboxylic acid quinoxalin-2-ylamide

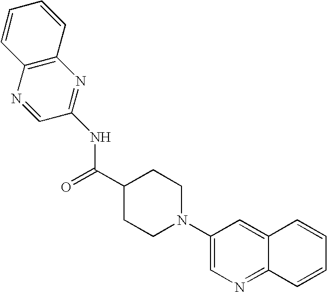 Using the procedure outlined in Example 1A, 1-quinolin-3-yl-piperidine-4-carboxylic acid is prepared from 3-bromo-quinoline (4 mmol). Using the procedure outlined in Example 8, the title compound is prepared from quinoxalin-2-ylamine (0.5 mmol) and 1-quinolin-3-yl-piperidine-4-carboxylic acid (0.18 mmol).

### Example 94

### 1-Isoquinolin-5-yl-piperidine-4-carboxylic acid quinoxalin-2-ylamide

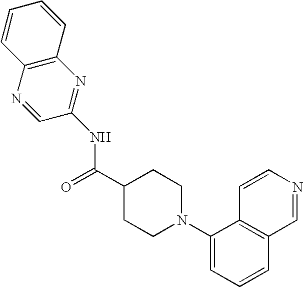 Using the procedure outlined in Example 1A, 1-isoquinolin-5-yl-piperidine-4-carboxylic acid is prepared from 5-Bromo-isoquinoline (4 mmol). Using the procedure outlined in Example 8, the title compound is prepared from quinoxalin-2-ylamine (0.5 mmol) and 1-isoquinolin-5-yl-piperidine-4-carboxylic acid (0.18 mmol).

### Example 95

### 1-Isoquinolin-5-yl-piperidine-4-carboxylic acid (9H-carbazol-3-yl)-amide

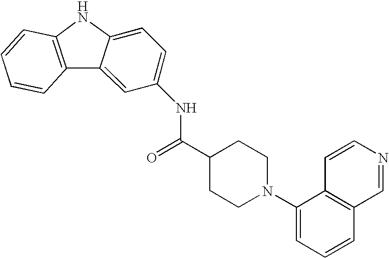 Using the procedure outlined in Example 1A, 1-isoquinolin-5-yl-piperidine-4-carboxylic acid is prepared from 5-bromo-isoquinoline (4 mmol). Using the procedure outlined in Example 8, the title compound is prepared from 9H-carbazol-3-ylamine (0.5 mmol) and 1-isoquinolin-5-yl-piperidine-4-carboxylic acid (0.18 mmol).

### Example 96

### 1-Benzo[b]thiophen-3-yl-piperidine-4-carboxylic acid (9H-carbazol-3-yl)-amide

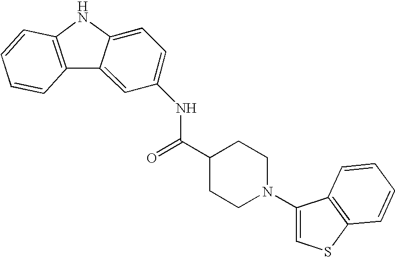 Using the procedure outlined in Example 1A, 1-benzo[b]thiophen-3-yl-piperidine-4-carboxylic acid is prepared from 3-bromo-benzo[b]thiophene (4 mmol). Using the procedure outlined in Example 8, the title compound is prepared from 9H-carbazol-3-ylamine (0.5 mmol) and 1-benzo[b]thiophen-3-yl-piperidine-4-carboxylic acid (0.18 mmol).

### Example 97

### 1-Benzo[b]thiophen-3-yl-piperidine-4-carboxylic acid phenazin-2-ylamide

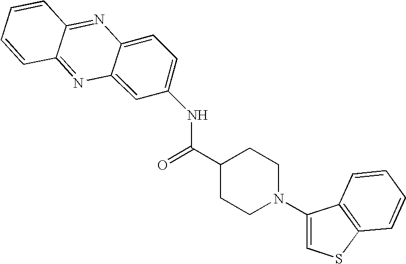 Using the procedure outlined in Example 1A, 1-benzo[b]thiophen-3-yl-piperidine-4-carboxylic acid is prepared from 3-bromo-benzo[b]thiophene (4 mmol). Using the procedure outlined in Example 8, the title compound is prepared from phenazin-2-ylamine (0.5 mmol) and 1-benzo[b]thiophen-3-yl-piperidine-4-carboxylic acid (0.18 mmol).

### Example 98

### 1-Naphthalen-2-yl-piperidine-4-carboxylic acid phenazin-2-ylamide

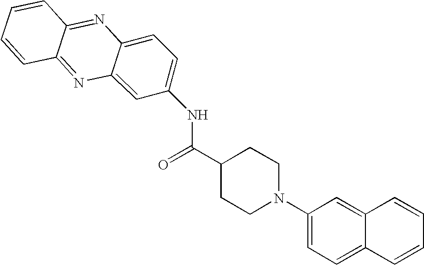 Using the procedure outlined in Example 1A, 1-naphthalen-2-yl-piperidine-4-carboxylic acid is prepared from 2-bromo-naphthalene (4 mmol). Using the procedure outlined in Example 8, the title compound is prepared from phenazin-2-ylamine (0.5 mmol) and 1-naphthalen-2-yl-piperidine-4-carboxylic acid (0.18 mmol).

### Example 99

### 1-Naphthalen-2-yl-piperidine-4-carboxylic acid thiazol-2-ylamide

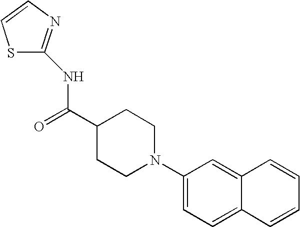 Using the procedure outlined in Example 1A, 1-naphthalen-2-yl-piperidine-4-carboxylic acid is prepared from 2-bromo-naphthalene (4 mmol). Using the procedure outlined in Example 8, the title compound is prepared from thiazol-2-ylamine (0.5 mmol) and 1-naphthalen-2-yl-piperidine-4-carboxylic acid (0.18 mmol).

### Example 100

### 1-Biphenyl-3-yl-piperidine-4-carboxylic acid thiazol-2-ylamide

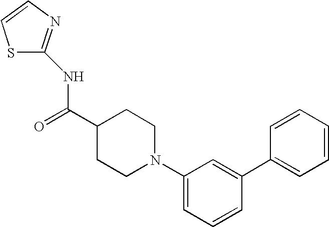 Using the procedure outlined in Example 1, 1-biphenyl-3-yl-piperidine-4-carboxylic acid is prepared from 3-bromobiphenyl (4 mmol). Using the procedure outlined in Example 8, the title compound is prepared from thiazol-2-ylamine (0.5 mmol) and 1-biphenyl-3-yl-piperidine-4-carboxylic acid (0.18 mmol).

### Example 101

### 1-Biphenyl-3-yl-piperidine-4-carboxylic acid (1H-imidazol-2-yl)-amide

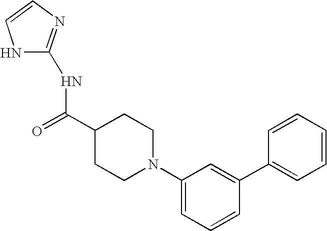 Using the procedure outlined in Example 1A, 1-biphenyl-3-yl-piperidine-4-carboxylic acid is prepared from 3-bromobiphenyl (4 mmol). Using the procedure outlined in Example 8, the title compound is prepared from 1H-imidazol-2-ylamine (0.5 mmol) and 1-biphenyl-3-yl-piperidine-4-carboxylic acid (0.18 mmol).

### Example 102

### 1-Quinolin-3-yl-piperidine-4-carboxylic acid (1H-imidazol-2-yl)-amide

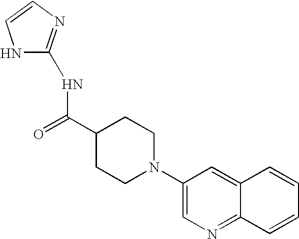 Using the procedure outlined in Example 1A, 1-quinolin-3-yl-piperidine-4-carboxylic acid is prepared from 3-bromo-quinoline (4 mmol). Using the procedure outlined in Example 8, the title compound is prepared from 1H-imidazol-2-ylamine (0.5 mmol) and 1-quinolin-3-yl-piperidine-4-carboxylic acid (0.18 mmol).

### Example 103

### 1-Quinolin-3-yl-piperidine-4-carboxylic acid (2H-pyrazol-3-yl)-amide

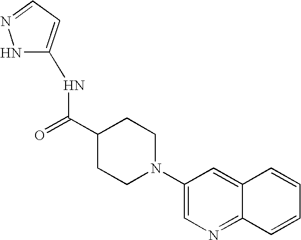 Using the procedure outlined in Example 1A, 1-quinolin-3-yl-piperidine-4-carboxylic acid is prepared from 3-bromo-quinoline (4 mmol). Using the procedure outlined in Example 8, the title compound is prepared from 2H-pyrazol-3-ylamine (0.5 mmol) and 1-quinolin-3-yl-piperidine-4-carboxylic acid (0.18 mmol).

### Example 104

### 1-Isoquinolin-5-yl-piperidine-4-carboxylic acid (2H-pyrazol-3-yl)-amide

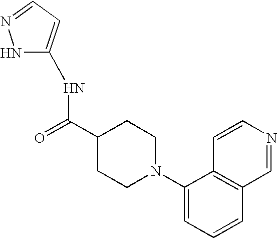 Using the procedure outlined in Example 1A, 1-isoquinolin-5-yl-piperidine-4-carboxylic acid is prepared from 5-bromo-isoquinoline (4 mmol). Using the procedure outlined in Example 8, the title compound is prepared from 2H-pyrazol-3-ylamine (0.5 mmol) and 1-isoquinolin-5-yl-piperidine-4-carboxylic acid (0.18 mmol).

### Example 105

### 1-Isoquinolin-5-yl-piperidine-4-carboxylic acid isoxazol-3-ylamide

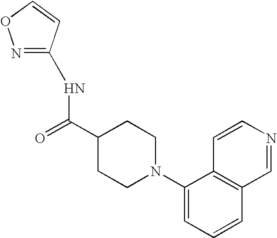 Using the procedure outlined in Example 1A, 1-isoquinolin-5-yl-piperidine-4-carboxylic acid is prepared from 5-bromo-isoquinoline (4 mmol). Using the procedure outlined in Example 8, the title compound is prepared from isoxazol-3-ylamine (0.5 mmol) and 1-isoquinolin-5-yl-piperidine-4-carboxylic acid (0.18 mmol).

### Example 106

### 1-Benzo[b]thiophen-3-yl-piperidine-4-carboxylic acid isoxazol-3-ylamide

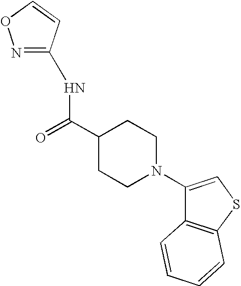 Using the procedure outlined in Example 1A, 1-benzo[b]thiophen-3-yl-piperidine-4-carboxylic acid is prepared from 3-bromo-benzo[b]thiophene (4 mmol). Using the procedure outlined in Example 8, the title compound is prepared from isoxazol-3-ylamine (0.5 mmol) and 1-benzo[b]thiophen-3-yl-piperidine-4-carboxylic acid (0.18 mmol).

### Example 107

### 1-Benzo[b]thiophen-3-yl-piperidine-4-carboxylic acid (3-methyl-isothiazol-5-yl)-amide

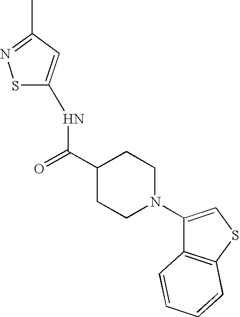 Using the procedure outlined in Example 1A, 1-benzo[b]thiophen-3-yl-piperidine-4-carboxylic acid is prepared from 3-bromo-benzo[b]thiophene (4 mmol). Using the procedure outlined in Example 8, the title compound is prepared from 3-methyl-isothiazol-5-ylamine (0.5 mmol) and 1-benzo[b]thiophen-3-yl-piperidine-4-carboxylic acid (0.18 mmol).

### Example 108

### 1-Naphthalen-2-yl-piperidine-4-carboxylic acid (3-methyl-isothiazol-5-yl)-amide

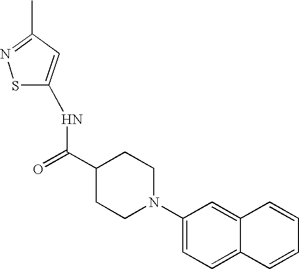 Using the procedure outlined in Example 1A, 1-naphthalen-2-yl-piperidine-4-carboxylic acid is prepared from 2-bromo-naphthalene (4 mmol). Using the procedure outlined in Example 8, the title compound is prepared from 3-methyl-isothiazol-5-ylamine (0.5 mmol) and 1-naphthalen-2-yl-piperidine-4-carboxylic acid (0.18 mmol).

### Example 109

### 1-Isoquinolin-5-yl-piperidine-4-carboxylic acid [1,6]naphthyridin-4-ylamide

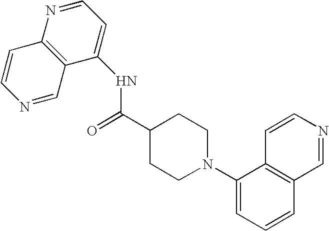 Using the procedure outlined in Example 1A, 1-isoquinolin-5-yl-piperidine-4-carboxylic acid is prepared from 5-bromo-isoquinoline (4 mmol). Using the procedure outlined in Example 8, the title compound is prepared from [1,6]naphthyridin-4-ylamine (0.5 mmol) and 1-isoquinolin-5-yl-piperidine-4-carboxylic acid (0.18 mmol).

### Example 110

### 1-Benzo[b]thiophen-3-yl-piperidine-4-carboxylic acid [1,6]naphthyridin-4-ylamide

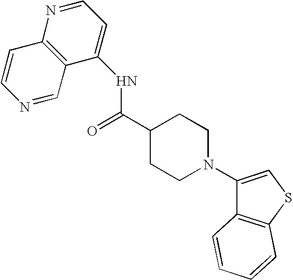 Using the procedure outlined in Example 1A, 1-benzo[b]thiophen-3-yl-piperidine-4-carboxylic acid is prepared from 3-bromo-benzo[b]thiophene (4 mmol). Using the procedure outlined in Example 8, the title compound is prepared from [1,6]naphthyridin-4-ylamine (0.5 mmol) and 1-benzo[b]thiophen-3-yl-piperidine-4-carboxylic acid (0.18 mmol).

### Example 111

### 1-Benzo[b]thiophen-3-yl-piperidine-4-carboxylic acid [1,7]naphthyridin-8-ylamide

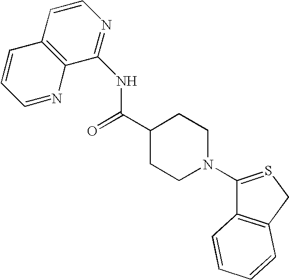 Using the procedure outlined in Example 1A, 1-benzo[b]thiophen-3-yl-piperidine-4-carboxylic acid is prepared from 3-bromo-benzo[b]thiophene (4 mmol). Using the procedure outlined in Example 8, the title compound is prepared from [1,7]naphthyridin-8-ylamine (0.5 mmol) and 1-benzo[b]thiophen-3-yl-piperidine-4-carboxylic acid (0.18 mmol).

### Example 112

### 1-Naphthalen-2-yl-piperidine-4-carboxylic acid [1,7]naphthyridin-8-ylamide

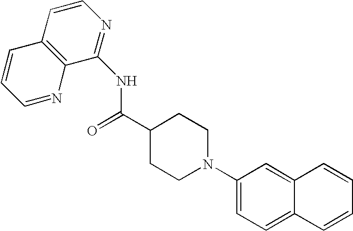 Using the procedure outlined in Example 1A, 1-naphthalen-2-yl-piperidine-4-carboxylic acid is prepared from 2-bromo-naphthalene (4 mmol). Using the procedure outlined in Example 8, the title compound is prepared from [1,7]naphthyridin-8-ylamine (0.5 mmol) and 1-naphthalen-2-yl-piperidine-4-carboxylic acid (0.18 mmol).

### Example 113

### 1-Naphthalen-2-yl-piperidine-4-carboxylic acid acridin-9-ylamide

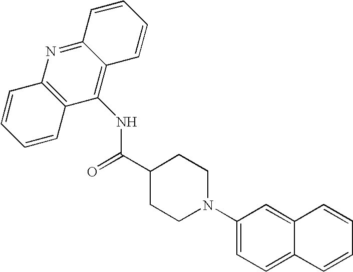 Using the procedure outlined in Example 1A, 1-naphthalen-2-yl-piperidine-4-carboxylic acid is prepared from 2-bromo-naphthalene (4 mmol). Using the procedure outlined in Example 8, the title compound is prepared from acridin-9-ylamine (0.5 mmol) and 1-naphthalen-2-yl-piperidine-4-carboxylic acid (0.18 mmol).

### Example 114

### 1-Biphenyl-3-yl-piperidine-4-carboxylic acid acridin-9-ylamide

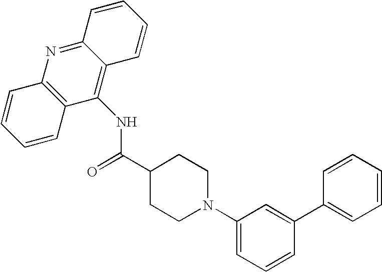 Using the procedure outlined in Example 1A, 1-biphenyl-3-yl-piperidine-4-carboxylic acid is prepared from 3-biphenyl (4 mmol). Using the procedure outlined in Example 8, the title compound is prepared from acridin-9-ylamine (0.5 mmol) and 1-biphenyl-3-yl-piperidine-4-carboxylic acid (0.18 mmol).

### Example 115

### 1-Biphenyl-3-yl-piperidine-4-carboxylic acid (1-methyl-4,5-dihydro-1H-imidazol-2-yl)-amide

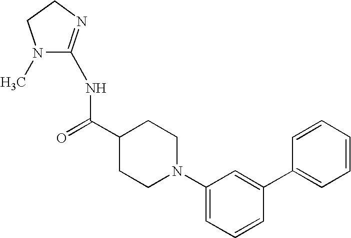 Using the procedure outlined in Example 1A, 1-biphenyl-3-yl-piperidine-4-carboxylic acid is prepared from 3-biphenyl (4 mmol). Using the procedure outlined in Example 8, the title compound is prepared from 1-methyl-4,5-dihydro-1H-imidazol-2-ylamine (0.5 mmol) and 1-biphenyl-3-yl-piperidine-4-carboxylic acid (0.18 mmol).

### Example 116

### 1-Quinolin-3-yl-piperidine-4-carboxylic acid (1-methyl-4,5-dihydro-1H-imidazol-2-yl)-amide

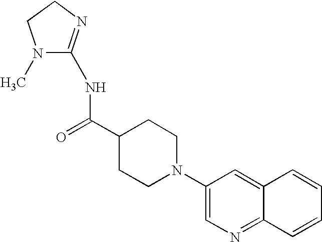 Using the procedure outlined in Example 1A, 1-quinolin-3-yl-piperidine-4-carboxylic acid is prepared from 3-bromo-quinoline (4 mmol). Using the procedure outlined in Example 8, the title compound is prepared from 1-methyl-4,5-dihydro-1H-imidazol-2-ylamine (0.5 mmol) and 1-quinolin-3-yl-piperidine-4-carboxylic acid (0.18 mmol).

### Example 117

### 1-Quinolin-3-yl-piperidine-4-carboxylic acid (4,5-dihydro-1H-pyrazol-3-yl)-amide

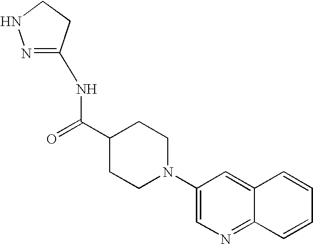 A. Using the procedure outlined in Example 1A, 1-quinolin-3-yl-piperidine-4-carboxylic acid is prepared from 3-bromo-quinoline (4 mmol). Using the procedure outlined in Example 8, 1-quinolin-3-yl-piperidine-4-carboxylic acid [1-(toluene-4-sulfonyl)-4,5-dihydro-1H-pyrazol-3-yl]-amide is prepared from 1-(toluene-4-sulfonyl)-4,5-dihydro-1H-pyrazol-3-ylamine (0.5 mmol) and 1-quinolin-3-yl-piperidine-4-carboxylic acid (0.18 mmol).
B. A solution of 1-quinolin-3-yl-piperidine-4-carboxylic acid [1-(toluene-4-sulfonyl)-4,5-dihydro-1H-pyrazol-3-yl]-amide (0.2 mmol) in 30% HBr in acetic acid (1 mL) is stirred at 70° C. for 8 h. The solution is concentrated and the residue is chromatographed on a prep TLC plate (2000 microns, silica as stationary phase, methylene chloride/MeOH 9:1) to give the title compound.

### Example 118

### 1-Isoquinolin-5-yl-piperidine-4-carboxylic acid (4,5-dihydro-1H-pyrazol-3-yl)-amide

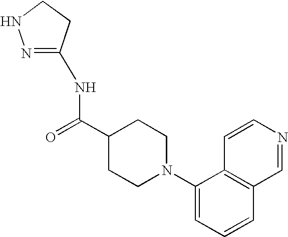 A. Using the procedure outlined in Example 1A, 1-isoquinolin-5-yl-piperidine-4-carboxylic acid is prepared from 5-bromo-isoquinoline (4 mmol). Using the procedure outlined in Example 8, 1-isoquinolin-5-yl-piperidine-4-carboxylic acid [1-(toluene-4-sulfonyl)-4,5-dihydro-1H-pyrazol-3-yl]-amide is prepared from 1-(toluene-4-sulfonyl)-4,5-dihydro-1H-pyrazol-3-ylamine (0.5 mmol) and 1-isoquinolin-5-yl-piperidine-4-carboxylic acid (0.18 mmol).
B. A solution of 1-isoquinolin-5-yl-piperidine-4-carboxylic acid [1-(toluene-4-sulfonyl)-4,5-dihydro-1H-pyrazol-3-yl]-amide (0.2 mmol) in 30% HBr in acetic acid (1 mL) is stirred at 70° C. for 8 h. The solution is concentrated and the residue is chromatographed on a prep TLC plate (2000 microns, silica as stationary phase, methylene chloride/MeOH 9:1) to give the title compound.

### Example 119

### 1-Isoquinolin-5-yl-piperidine-4-carboxylic acid [1,8]naphthyridin-2-ylamide

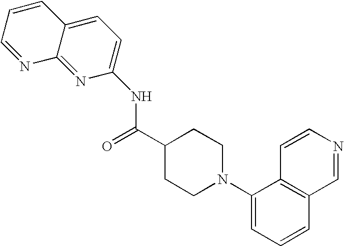 A. A solution of [1,8]naphthyridine (0.2 mmol) and potassium amide (0.8 mmol) in liquid ammonia (20 mL) is stirred at −40° C. for 3 h. The solution is concentrated. Residue is partitioned between EtOAc and water. EtOAc layer is separated and washed successively with water and brine. EtOAc layer is dried over sodium sulfate, filtered and concentrated to give [1,8]naphthyridin-2-ylamine which is used as is for the next step.
B. Using the procedure outlined in Example 1A, 1-isoquinolin-5-yl-piperidine-4-carboxylic acid is prepared from 5-bromo-isoquinoline (4 mmol). Using the procedure outlined in Example 8, the title compound is prepared from [1,8]naphthyridin-2-ylamine (0.5 mmol) and 1-isoquinolin-5-yl-piperidine-4-carboxylic acid (0.18 mmol).

### Example 120

### 1-Benzo[b]thiophen-3-yl-piperidine-4-carboxylic acid [1,8]naphthyridin-2-ylamide

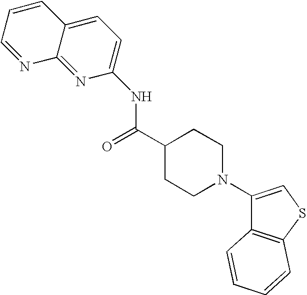 Using the procedure outlined in Example 1, 1-benzo[b]thiophen-3-yl-piperidine-4-carboxylic acid is prepared from 3-bromo-benzo[b]thiophene (4 mmol). Using the procedure outlined in Example 8, the title compound is prepared from [1,8]naphthyridin-2-ylamine (0.5 mmol) (prepared as in Example 165a) and 1-benzo[b]thiophen-3-yl-piperidine-4-carboxylic acid (0.18 mmol).

### Example 121

### 1-Benzo[b]thiophen-3-yl-piperidine-4-carboxylic acid [2,6]naphthyridin-1-ylamide

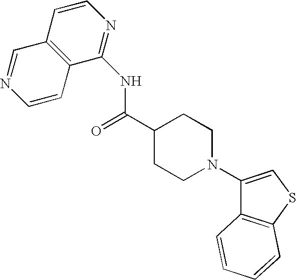 A. 1-Bromo-[2,6]naphthyridine is prepared from 1-bromo-[2,6]naphthyridin-3-ylamine using the procedure from Vogel's Textbook of Practical Organic Chemistry p. 945, fifth ed., 1989.
B. [2,6]Naphthyridin-1-ylamine is prepared from 1-bromo-[2,6]naphthyridine using the procedure given in Organic Letters, 3(17), 2729-2732 (2001).
C. Using the procedure outlined in Example 1C, 1-benzo[b]thiophen-3-yl-piperidine-4-carboxylic acid is prepared from 3-bromo-benzo[b]thiophene (4 mmol). Using the procedure outlined in Example 8, the title compound is prepared from [2,6]naphthyridin-1-ylamine (0.5 mmol) (prepared as in Example 167b) and 1-benzo[b]thiophen-3-yl-piperidine-4-carboxylic acid (0.18 mmol).

### Example 122

### 1-Naphthalen-2-yl-piperidine-4-carboxylic acid [2,6]naphthyridin-1-ylamide

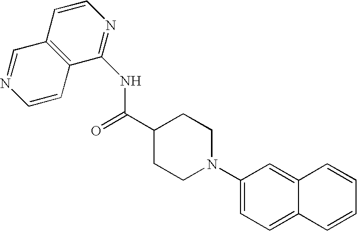 Using the procedure outlined in Example 1A, 1-naphthalen-2-yl-piperidine-4-carboxylic acid is prepared from 2-bromo-naphthalene (4 mmol). Using the procedure outlined in Example 8, the title compound is prepared from [2,6]naphthyridin-1-ylamine (0.5 mmol) (prepared as in Example 167b) and 1-naphthalen-2-yl-piperidine-4-carboxylic acid (0.18 mmol).

### Example 123

### 1-Naphthalen-2-yl-piperidine-4-carboxylic acid benzo[d]isoxazol-5-ylamide

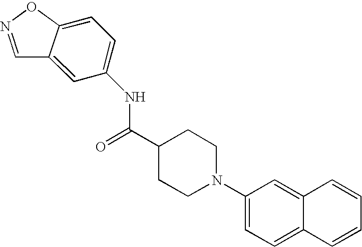 A. 5-Nitro-benzo[d]isoxazole is prepared from benzo[d]isoxazole as described in Example 63A.
B. Benzo[d]isoxazol-5-ylamine is prepared from 5-nitro-benzo[d]isoxazole as described in Example 63B.
C. Using the procedure outlined in Example 1A, 1-naphthalen-2-yl-piperidine-4-carboxylic acid is prepared from 2-Bromo-naphthalene (4 mmol). Using the procedure outlined in Example 8, the title compound is prepared from benzo[d]isoxazol-5-ylamine (0.5 mmol) and 1-naphthalen-2-yl-piperidine-4-carboxylic acid (0.18 mmol).

### Example 124

### 1-Biphenyl-3-yl-piperidine-4-carboxylic acid benzo[d]isoxazol-5-ylamide

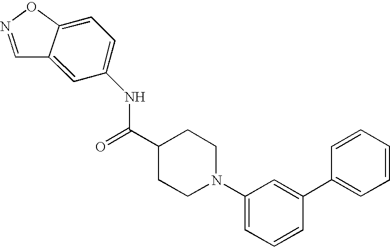 Using the procedure outlined in Example 1A, 1-biphenyl-3-yl-piperidine-4-carboxylic acid is prepared from 3-bromo-biphenyl (4 mmol). Using the procedure outlined in Example 8, the title compound is prepared from benzo[d]isoxazol-5-ylamine (0.5 mmol) (prepared as in Example 169b) and 1-biphenyl-3-yl-piperidine-4-carboxylic acid (0.18 mmol).

### Example 125

### 1-Biphenyl-3-yl-piperidine-4-carboxylic acid (1H-pyrrol-3-yl)-amide

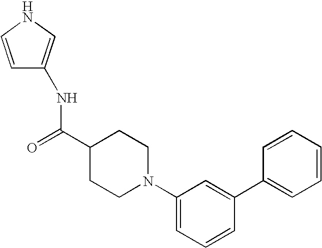 A. 1H-Pyrrol-3-ylamine is prepared from 3-nitro-1H-pyrrole following the procedure from Example 63B.
B. Using the procedure outlined in Example 1A, 1-biphenyl-3-yl-piperidine-4-carboxylic acid is prepared from 3-bromo-biphenyl (4 mmol). Using the procedure outlined in Example 8, the title compound is prepared from 1H-pyrrol-3-ylamine (0.5 mmol) (prepared as in Example 172a) and 1-biphenyl-3-yl-piperidine-4-carboxylic acid (0.18 mmol).

### Example 126

### 1-Quinolin-3-yl-piperidine-4-carboxylic acid (1H-pyrrol-3-yl)-amide

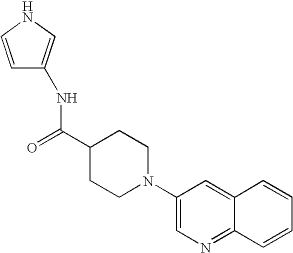 Using the procedure outlined in Example 1A, 1-quinolin-3-yl-piperidine-4-carboxylic acid is prepared from 3-bromo-quinoline (4 mmol). Using the procedure outlined in Example 8, the title compound is prepared from 1H-pyrrol-3-ylamine (0.5 mmol) and 1-quinolin-3-yl-piperidine-4-carboxylic acid (0.18 mmol).

### Example 127

### 1-Quinolin-3-yl-piperidine-4-carboxylic acid (1H-pyrrolo[2,3-b]pyridin-3-yl)-amide

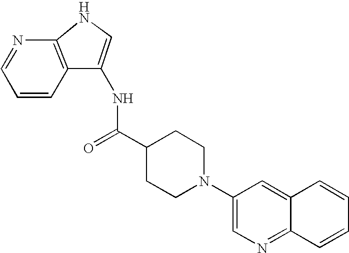 A. 3-Bromo-1H-pyrrolo[2,3-b]pyridine and ammonia (0.5M in 1,4-dioxane) is heated to reflux. After 5 h the solution is concentrated. The residue is chromatographed on a silica gel plug to give 1H-pyrrolo[2,3-b]pyridin-3-ylamine.
B. Using the procedure outlined in Example 1A, 1-quinolin-3-yl-piperidine-4-carboxylic acid is prepared from 3-bromo-quinoline (4 mmol). Using the procedure outlined in Example 8, the title compound is prepared from 1H-pyrrolo[2,3-b]pyridin-3-ylamine (0.5 mmol) and 1-quinolin-3-yl-piperidine-4-carboxylic acid (0.18 mmol).

### Example 128

### 1-Isoquinolin-5-yl-piperidine-4-carboxylic acid (1H-pyrrolo[2,3-b]pyridin-3-yl)-amide

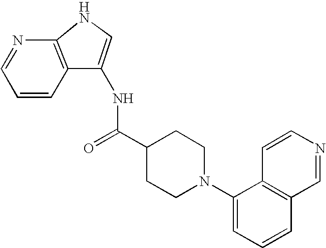 Using the procedure outlined in Example 1A, 1-isoquinolin-5-yl-piperidine-4-carboxylic acid is prepared from 3-bromo-quinoline (4 mmol). Using the procedure outlined in Example 8, the title compound is prepared from 1H-pyrrolo[2,3-b]pyridin-3-ylamine (0.5 mmol) (prepared as in Example 174a) and 1-isoquinolin-5-yl-piperidine-4-carboxylic acid (0.18 mmol).

### Example 129

### 1-Isoquinolin-5-yl-piperidine-4-carboxylic acid pteridin-4-ylamide

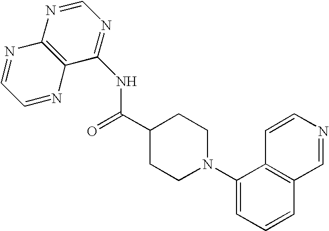 A. A solution of pteridin-4-ol (1 mmol) in 10 mL of POCl₃ is stirred at 0° C. for 10 min then at 100° C. for 6 h. The solution is concentrated. The residue is partitioned between ethyl acetate and water. Ethyl acetate layer is separated, dried over sodium sulfate, filtered and concentrated to give 4-chloro-pteridine.
B. A solution of 4-chloro-pteridine (0.5 mmol) and ammonia (0.5M in 1,4-dioxane, 2 mmol) in DMF is heated to 100° C. After 5 h the solution is concentrated. Residue is chromatographed on a silica gel plug to give pteridin-4-ylamine.
C. Using the procedure outlined in Example 1A, 1-isoquinolin-5-yl-piperidine-4-carboxylic acid is prepared from 3-bromo-quinoline (4 mmol). Using the procedure outlined in Example 8, the title compound is prepared from pteridin-4-ylamine (0.5 mmol) (prepared as in Example 176b) and 1-isoquinolin-5-yl-piperidine-4-carboxylic acid (0.18 mmol).

### Example 130

### 1-Benzo[b]thiophen-3-yl-piperidine-4-carboxylic acid pteridin-4-ylamide

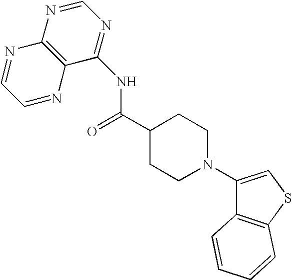 Using the procedure outlined in Example 1A, 1-benzo[b]thiophen-3-yl-piperidine-4-carboxylic acid is prepared from 3-bromo-benzo[b]thiophene (4 mmol). Using the procedure outlined in Example 8, the title compound is prepared from pteridin-4-ylamine (0.5 mmol) (prepared as in Example 176b) and 1-benzo[b]thiophen-3-yl-piperidine-4-carboxylic acid (0.18 mmol).

### Example 131

### 1-Benzo[b]thiophen-3-yl-piperidine-4-carboxylic acid (1,4-dihydro-quinazolin-6-yl)-amide

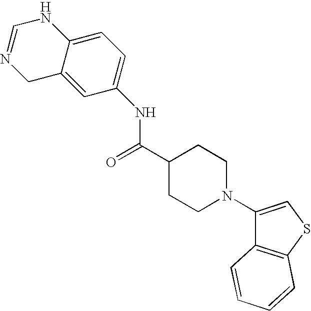 A. 1,4-Dihydro-quinazoline is prepared from 2-aminomethyl-phenylamine (1 mmol) following the procedure given in Journal of Organic Chemistry, 5, 133-141 (1940).
B. 6-Nitro-1,4-dihydro-quinazoline is prepared from 1,4-dihydro-quinazoline (1 mmol) following the procedure given in Example 69A.
C. 1,4-Dihydro-quinazolin-6-ylamine is prepared from 6-nitro-1,4-dihydro-quinazoline (1 mmol) following the procedure given in Example 69B.
D. Using the procedure outlined in Example 1A, 1-benzo[b]thiophen-3-yl-piperidine-4-carboxylic acid is prepared from 3-bromo-benzo[b]thiophene (4 mmol). Using the procedure outlined in Example 8, the title compound is prepared from 1,4-dihydro-quinazolin-6-ylamine (0.5 mmol) (prepared as in Example 178c) and 1-benzo[b]thiophen-3-yl-piperidine-4-carboxylic acid (0.18 mmol).

### Example 132

### 1-Naphthalen-2-yl-piperidine-4-carboxylic acid (1,4-dihydro-quinazolin-6-yl)-amide

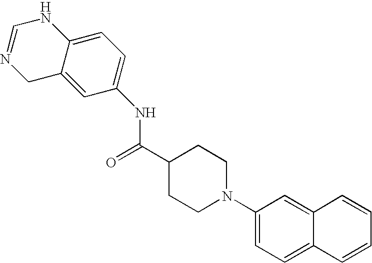 Using the procedure outlined in Example 1A, 1-naphthalen-2-yl-piperidine-4-carboxylic acid is prepared from 2-bromo-naphthalene (4 mmol). Using the procedure outlined in Example 8, the title compound is prepared from 1,4-dihydro-quinazolin-6-ylamine (0.5 mmol) and 1-naphthalen-2-yl-piperidine-4-carboxylic acid (0.18 mmol).

### Example 133

### 1-Naphthalen-2-yl-piperidine-4-carboxylic acid [1,7]naphthyridin-4-ylamide

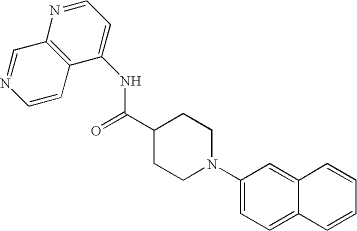 A. To a solution of 2-bromo-3-hydroxypyridine (5.00 g, 0.029 mmol) in 40 mL of anhydrous Et₂O is added Et₃N (4.4 mL, 0.032 mmol) and trifluoromethanesulfonic anhydride (5.3 mL, 0.032 mmol) at 0° C. The mixture is warmed to 25° C. and stirred 3 h. Brine is added and the aqueous layer is extracted with Et₂O. The combined organic solvent is washed with brine, dried under MgSO₄, and concentrated to dryness. The residue is purified by silica gel column chromatography with hexanes:EtOAc=10:1 as eluent to provide trifluoro-methanesulfonic acid 2-bromo-pyridin-3-yl ester as colorless oil.
B. To a solution of trifluoro-methanesulfonic acid 2-bromo-pyridin-3-yl ester (10.00 g, 0.033 mol) in 160 mL of anhydrous DMF is added (trimethylsilyl)acetylene (5.1 mL, 0.036 mol), Pd(PPh₃)₂Cl₂ (1.15 g, 0.0016 mol), LiCl (3.33 g, 0.0785 mol), and Et₃N (6.8 mL, 0.049 mol). A mixture is warmed to 55° C. and stirred 14 h. Water is added to quench the reaction mixture. The aqueous layer is extracted Et₂O. The combined solvent is dried and concentrated to dryness. The residue is purified by silica gel column chromatography with hexanes:EtOAc=10:1 as eluent to provide 2-bromo-3-trimethylsilanylethynyl-pyridine as brown oil.
C. To a solution of 2-bromo-3-trimethylsilanylethynyl-pyridine (1.66 g, 6.53 mmol) in 35 mL of Et₂O is added n-BuLi (4.5 mL, 7.18 mmol) at −78° C. After stirring for an hour, DMF (0.7 mL, 7.49 mmol) is added at the same temperature. The reaction mixture is warmed to room temperature after 30 min. Stirring 20 min, the reaction is quenched by brine. The aqueous layer is extracted with Et₂O. The combined solvent is dried and concentrated to dryness. The residue is purified by silica gel column chromatography with hexanes:EtOAc=10:1 as eluent to provide 3-trimethylsilanylethynyl-pyridine-2-carbaldehyde as brown oil.
D. To a solution of 3-trimethylsilanylethynyl-pyridine-2-carbaldehyde (1.25 g, 6.15 mmol) in 40 ml of 2.0 M NH₃ in EtOH. Heated to 80° C. for 9 h. Solvent is evaporated to give residue which is purified by silica gel column chromatography with hexanes:EtOAc=2:1 as eluent to provide [1,7]naphthyridine as brown solid.
E. To a solution of [1,7]naphthyridine (2.36 g, 0.018 mol) in 5 mL of acetic acid is added 2.5 mL of 32% percacetic acid. A mixture is heated to 75° C. for 3 h. The mono- and dioxides are separated by fractional crystallization using methylcyclohexane to yield [1,7]naphthyridine 1-oxide.
F. A mixture of [1,7]naphthyridine 1-oxide (1.27 g, 8.69 mmol) in 5 mL of fuming nitric acid is heated to 55° C. for 5 h. Nitric acid is removed and the resulting residue is neutralized by aqueous NaHCO₃. The aqueous layer is extracted by Et₂O. The combined solvent is dried and concentrated to dryness. The residue is purified by silica gel column chromatography with hexanes:EtOAc=1:1 as eluent to provide 4-nitro-[1,7]naphthyridine 1-oxide.
G. A mixture of 4-nitro-[1,7]naphthyridine 1-oxide (0.82 g, 4.29 mmol) and Raney nickel (2.32 g) in 12 mL of MeOH is hydrogenated at 25° C. The catalyst is filtered off and the filtrated is concentrated to dryness which is purified by silica gel column chromatography with EtOAc as eluent to provide [1,7]naphthyridin-4-ylamine.
H. Using the procedure outlined in Example 1A, 1-naphthalen-2-yl-piperidine-4-carboxylic acid is prepared from 2-bromo-naphthalene (4 mmol). Using the procedure outlined in Example 8, the title compound is prepared from [1,7]naphthyridin-4-ylamine (0.5 mmol) (prepared as in Example 180g) and 1-naphthalen-2-yl-piperidine-4-carboxylic acid (0.18 mmol).

### Example 134

### 1-Biphenyl-3-yl-piperidine-4-carboxylic acid [1,7]naphthyridin-4-ylamide

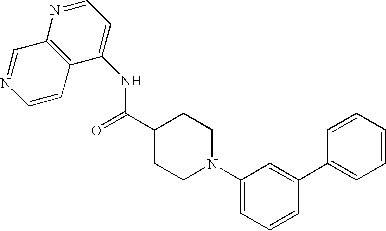 Using the procedure outlined in Example 1A, 1-biphenyl-3-yl-piperidine-4-carboxylic acid is prepared from 3-bromo-biphenyl (4 mmol). Using the procedure outlined in Example 8, the title compound is prepared from [1,7]naphthyridin-4-ylamine (0.5 mmol) (prepared as in Example 180g) and 1-biphenyl-3-yl-piperidine-4-carboxylic acid (0.18 mmol).

### Example 135

### 1-Biphenyl-3-yl-piperidine-4-carboxylic acid [2,7]naphthyridin-4-ylamide

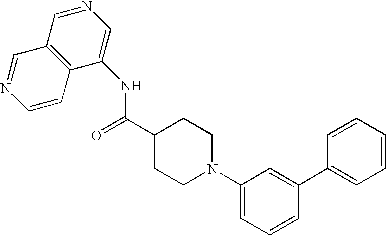 A. To a solution of diisopropylamine (5.3 mL, 0.0380 mol) in 120 mL of anhydrous THF is added n-BuLi (21.8 mL, 0.0348 mol) at 0° C. After stirring 20 minutes, 4-bromopyridine (5.00 g, 0.0316 mol) is added and the mixture is stirred for an hour. DMF (9.8 mL, 0.126 mol) is added. Stirring for 20 minutes, the mixture is warmed up to 25° C. and stirred for 30 minutes. Saturated aqueous NH₄Cl solution is added to quench the reaction and the aqueous layer is extracted with Et₂O. The combined solvent is dried and concentrated to dryness. The residue is purified by silica gel column chromatography with hexanes:EtOAc=10:1 as eluent to provide 4-bromo-pyridine-3-carbaldehyde.
B. To a solution of 4-bromo-pyridine-3-carbaldehyde (9.46 g, 0.0509 mol) in 150 mL of anhydrous DMF is added (trimethylsilyl)acetylene (7.9 mL, 0.0559 mol), Pd(PPh₃)₂Cl₂ (1.79 g, 0.00255 mol), LiCl (5.18 g, 0.122 mol), and Et₃N (10.6 mL, 0.0764 mol). A mixture is warmed to 55° C. and stirred 12 h. Water is added to quenched the reaction mixture. The aqueous layer is extracted Et₂O. The combined solvent is dried and concentrated to dryness. The residue is purified by silica gel column chromatography with hexanes:EtOAc=10:1 as eluent to provide 4-trimethylsilanylethynyl-pyridine-3-carbaldehyde.
C. To a solution of 4-trimethylsilanylethynyl-pyridine-3-carbaldehyde (1.07 g, 5.26 mmol) in 40 ml of 2.0 M NH₃ in EtOH. Heated to 80° C. for 9 h. Solvent is evaporated to give residue which is purified by silica gel column chromatography with hexanes:EtOAc=2:1 as eluent to provide [2,7]naphthyridine.
D. To a solution of dinitrogen pentoxide (1.16 g, 10.7 mmol) in 8 ml of liquid sulfur dioxide is added [2,7]naphthyridine (0.874 g, 6.72 mmol) at −38° C. The mixture is stirred for 5 minutes and then poured onto ice (9 g). After overnight stirring, the mixture is extracted with CH₂Cl₂. The combined solvent is dried and concentrated to dryness. The residue is purified by silica gel column chromatography with hexanes:EtOAc=2:1 as eluent to provide 4-nitro-[2,7]naphthyridine.
E. A mixture of 4-nitro-[2,7]naphthyridine (0.52 g, 2.97 mmol) and Raney nickel (1.6 g) in 10 mL of MeOH is hydrogenated at 25° C. The catalyst is filtered off and the filtrated is concentrated to dryness which is purified by silica gel column chromatography with EtOAc as eluent to provide [2,7]naphthyridin-4-ylamine.
F. Using the procedure outlined in Example 1A, 1-biphenyl-3-yl-piperidine-4-carboxylic acid is prepared from 3-Bromo-biphenyl (4 mmol). Using the procedure outlined in Example 8, the title compound is prepared from [2,7]naphthyridin-4-ylamine (0.5 mmol) (prepared as in Example 182e) and 1-biphenyl-3-yl-piperidine-4-carboxylic acid (0.18 mmol).

### Example 136

### 1-Quinolin-3-yl-piperidine-4-carboxylic acid [2,7]naphthyridin-4-ylamide

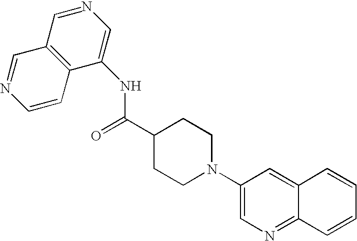 Using the procedure outlined in Example 1A, 1-quinolin-3-yl-piperidine-4-carboxylic acid is prepared from 3-Bromo-quinoline (4 mmol). Using the procedure outlined in Example 8, the title compound is prepared from [2,7]naphthyridin-4-ylamine (0.5 mmol) and 1-quinolin-3-yl-piperidine-4-carboxylic acid (0.18 mmol).

### Example 137

### 1-Quinolin-3-yl-piperidine-4-carboxylic acid [2,6]naphthyridin-1-ylamide

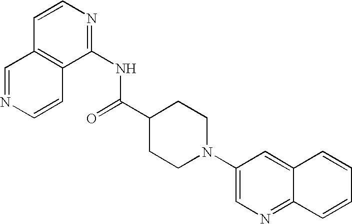 A. A mixture of 3-methyl-pyridine 1-oxide (20 g, 0.183 mol) and iodoethane (32 mL, 0.403 mmol) is stirred 16 h. 200 mL water is added, the aqueous layer is separated and washed with Et₂O. The collected aqueous layer is warmed to 50° C. and a solution of KCN (24 g, 0.366 mol) in 60 mL of water is added slowly in one hour. After an additional one hour stirring, the mixture is extracted with Et₂O. The combined solvent is dried and concentrated to dryness. The residue is purified by silica gel column chromatography with hexanes:EtOAc=4:1 as eluent to provide 3-methyl-isonicotinonitrile.
B. To a solution of 3-methyl-isonicotinonitrile (12.25 g, 0.104 mmol) in 100 mL of DMF is added dimethylformamide dimethyl acetal (22 mL, 0.166 mol) portionwise in 5 days. The solvent is evaporated to dryness. The residue is purified by silica gel column chromatography with hexanes:EtOAc=4:1 as eluent to provide 3-(2-dimethylamino-vinyl)-isonicotinonitrile.
C. To a solution of 3-(2-dimethylamino-vinyl)-isonicotinonitrile (5.26 g, 0.0304 mol) in 6 mL of glacial acetic acid is added 100 mL of 30% hydrogen bromide in acetic acid at 40° C. The mixture is then warmed up to 55° C. for an additional 2 hours. Solvent is evaporated to generate the residue which is poured onto ice, neutralized with NaHCO₃, and extracted with CH₂Cl₂. The combined solvent is dried and concentrated to dryness. The residue is purified by silica gel column chromatography with hexanes:EtOAc=2:1 as eluent to provide 2H-isoquinolin-1-one.
D. A mixture of 2H-isoquinolin-1-one (3.46 g, 0.0238 mol) and 20 mL of phosphorous oxybromide is refluxed for 2 hours. The excess phosphorous oxybromide is evaporated off, the residue is treated with ice and neutralized by sodium bicarbonate. The combined solvent is dried and concentrated to dryness. The residue is purified by silica gel column chromatography with hexanes:EtOAc=2:1 as eluent to provide 1-bromo-[2,6]naphthyridine.
E. A mixture of 1-bromo-[2,6]naphthyridine (1.26 g, 6.03 mol) and 10 mL of 2.0 M ammonia in 2-propanol is heated to 120° C. for 8 h. Solvent is evaporated off to dryness. The residue is purified by silica gel column chromatography with EtOAc as eluent to provide [2,6]naphthyridin-1-ylamine.
F. Using the procedure outlined in Example 1, 1-quinolin-3-yl-piperidine-4-carboxylic acid is prepared from 3-bromo-quinoline (4 mmol). Using the procedure outlined in Example 8, the title compound is prepared from [2,6]naphthyridin-1-ylamine (0.5 mmol) (prepared as in Example 184e) and 1-quinolin-3-yl-piperidine-4-carboxylic acid (0.18 mmol).

### Example 138

### 1-Isoquinolin-5-yl-piperidine-4-carboxylic acid [2,6]naphthyridin-1-ylamide

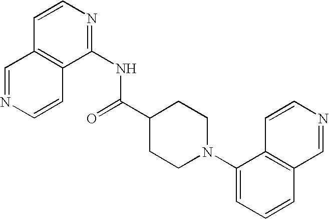 Using the procedure outlined in Example 1A, 1-isoquinolin-5-yl-piperidine-4-carboxylic acid is prepared from 5-bromo-isoquinoline (4 mmol). Using the procedure outlined in Example 8, the title compound is prepared from [2,6]naphthyridin-1-ylamine (0.5 mmol) (prepared as in Example 184e) and 1-Isoquinolin-5-yl-piperidine-4-carboxylic acid (0.18 mmol).
Compounds of Formulas (II), (IIa), (III) and (IIIa) may be prepared generally according to the Schemes 1 and 2 (vide supra) and also by Schemes 3, 4 or 5 below:

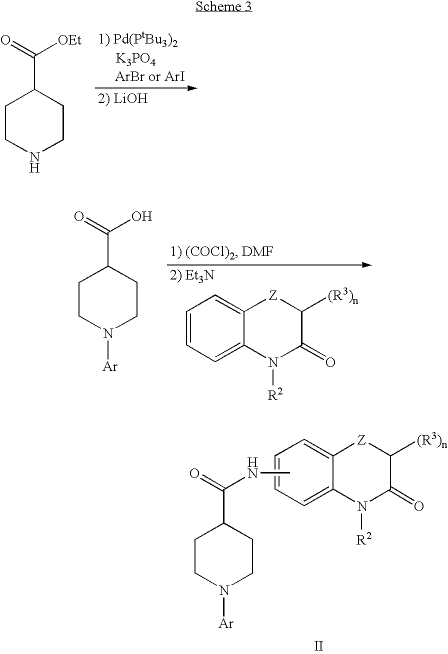

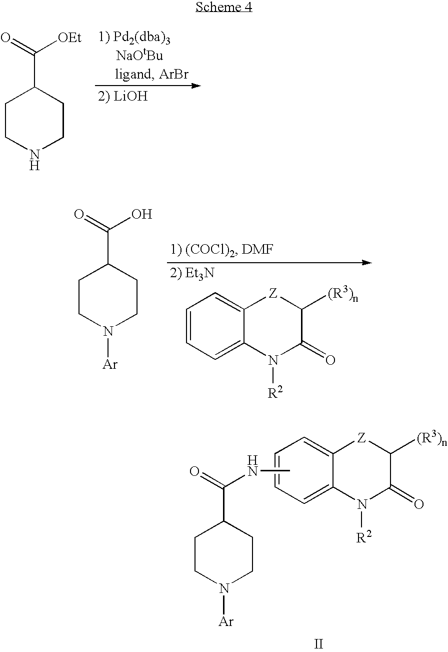

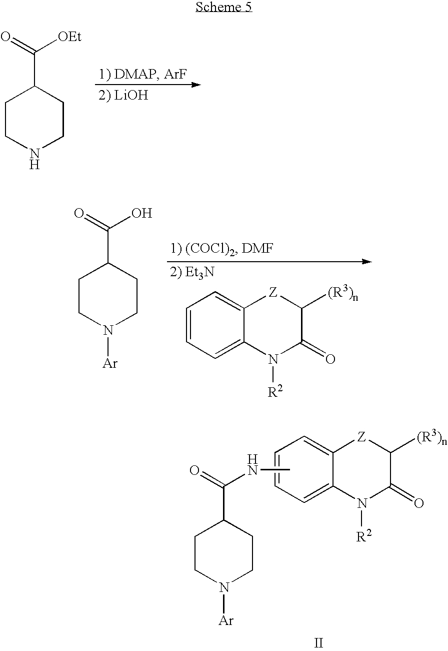

### Example 139

### 1-Phenyl-piperidine-4-carboxylic acid (3-oxo-3,4-dihydro-2H-benzo[1,4]oxazin-6-yl)-amide

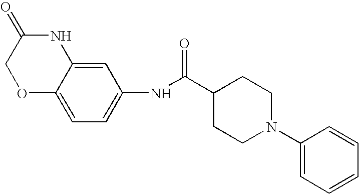 [0505] A. To a solution of ethyl isonipecotate (1.00 g, 6.36 mmol) in 10 mL of anhydrous DME was added iodobenzene (1.95 g, 9.54 mmol), bis(tri-t-butylphosphine)palladium(0) (0.33 g, 0.636 mmol), and potassium phosphate (2.70 g, 12.7 mmol). The reaction mixture was heated to 100° C. and stirred for 14 hours. Solids were filtered off from the resulting mixture and the collected filtrate was concentrated under reduced pressure to provide the crude product, which was then redissolved into 10 mL of methanol. 2 mL of 1N lithium hydroxide was added to hydrolyze the ester at 80° C. for 3 hours. Solvent was evaporated off and the basic residue was acidified by 2N HCl. The aqueous mixture was extracted with EtOAc. The combined organic layers was washed with brine, dried over MgSO₄, filtered, and concentrated under reduced pressure to afford the crude product which was then purified by flash column chromatography with CH₂Cl₂ and MeOH to yield 1-phenyl-piperidine-4-carboxylic acid as yellow solid.
B. To a solution of 1-phenyl-piperidine-4-carboxylic acid (0.03 g, 0.122 mmol) in 5 mL of CH₂Cl₂ was added oxalyl chloride (0.02 mL, 0.244 mmol) and a catalytic amount of DMF. The reaction mixture was stirred for 2 hours. Solvent was evaporated to dryness under reduced pressure. The residue was redissolved in 5 mL of CH₂Cl₂. 6-amino-2H-1,4-benzoxazine-3(4H)-one (0.02 g, 0.122 mmol) and triethylamine (0.03 mL, 0.183 mmol) were added sequentially into the acyl chloride solution at 0° C. The reaction mixture was warmed to 25° C. and stirred for 20 min. Concentration of the solvent provided the residue which was then purified by flash column chromatography with hexanes and ethyl acetate to afford 1-phenyl-piperidine-4-carboxylic acid (3-oxo-3,4-dihydro-2H-benzo[1,4]oxazin-6-yl)-amide as white solid. ¹H NMR (400 MHz, CD₃OD) δ (ppm): 7.61-7.58 (m, 4H), 7.54-7.51 (m, 1H), 7.39 (t, 1H), 7.04-7.01 (m, 1H), 6.91 (d, 1H), 4.54 (s, 2H), 3.84 (d, 2H), 3.66-3.59 (m, 2H), 2.82-2.79 (m, 1H), 2.28-2.23 (m, 4H). Mass Spectrum (LCMS, ESI pos.) Calcd. for C₂₀H₂₁N₃O₃: 351.4. Found: 352.1 (M⁺+1).

### Example 140

### 1-m-Tolyl-piperidine-4-carboxylic acid (3-oxo-3,4-dihydro-2H-benzo[1,4]oxazin-6-yl)-amide

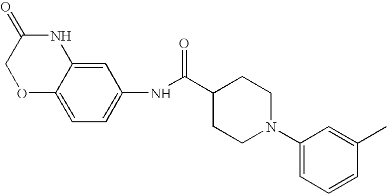 Using the procedure outlined in Example 139A, 1-m-tolyl-piperidine-4-carboxylic acid was prepared from 3-iodotoulene (2.08 g, 9.54 mmol) as a yellow solid.
Using the procedure outlined in Example 139B, the title compound was prepared from 1-m-tolyl-piperidine-4-carboxylic acid (0.03 g, 0.122 mmol) as an off-white solid. ¹H NMR (400 MHz, CD₃OD) δ (ppm): 7.38 (d, 1H), 7.01 (t, 1H), 6.98 (dd, 1H), 6.88 (dd, 1H), 6.82-6.78 (m, 2H), 6.67 (d, 1H), 4.53 (s, 2H), 3.73 (d, 2H), 2.76-2.69 (m, 2H), 2.49-2.45 (m, 1H), 2.29 (s, 3H), 2.01-1.90 (m, 4H). Mass Spectrum (LCMS, ESI pos.) Calcd. for C₂₁H₂₃N₃O₃: 365.4. Found: 366.1 (M⁺+1).

### Example 141

### 1-o-Tolyl-piperidine-4-carboxylic acid (3-oxo-3,4-dihydro-2H-benzo[1,4]oxazin-6-yl)-amide

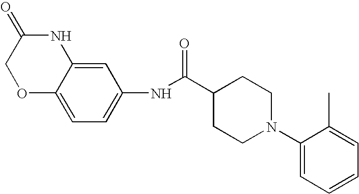 Using the procedure outlined in Example 139A, 1-o-tolyl-piperidine-4-carboxylic acid was prepared from 3-iodotoulene (2.08 g, 9.54 mmol) as a yellow solid.
Using the procedure outlined in Example 139B, the title compound was prepared from 1-o-tolyl-piperidine-4-carboxylic acid (0.03 g, 0.122 mmol) as light brown solid. ¹H NMR (400 MHz, CD₃OD) δ (ppm): 7.40 (d, 1H), 7.16-7.10 (m, 2H), 7.04 (d, 1H), 7.00 (dd, 1H), 6.95-6.91 (m, 1H), 6.89 (d, 1H), 4.55 (s, 2H), 3.16-3.12 (m, 2H), 2.70 (td, 2H), 2.47 (m, 1H), 2.30 (s, 3H), 2.01-1.93 (m, 4H). Mass Spectrum (LCMS, ESI pos.) Calcd. for C₂₁H₂₃N₃O₃: 365.4. Found: 366.0 (M⁺+1).

### Example 142

### 1-(2-Ethyl-phenyl)-piperidine-4-carboxylic acid (3-oxo-3,4-dihydro-2H-benzo[1,4]oxazin-6-yl)-amide

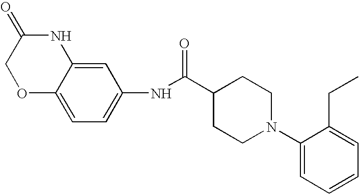 Using the procedure outlined in Example 139A, 1-(2-ethyl-phenyl)-piperidine-4-carboxylic acid was prepared from 1-ethyl-2-iodobenzene (2.21 g, 9.54 mmol) as a yellow solid.
Using the procedure outlined in Example 139B, the title compound was prepared from 1-(2-ethyl-phenyl)-piperidine-4-carboxylic acid (0.03 g, 0.122 mmol) as white solid. ¹H NMR (400 MHz, CD₃OD) δ (ppm): 7.40 (d, 1H), 7.20 (dd, 1H), 7.13-7.08 (m, 2H), 7.03-6.99 (m, 2H), 6.89 (d, 1H), 4.54 (s, 2H), 3.10 (d, 2H), 2.77-2.69 (m, 4H), 2.49-2.43 (m, 1H), 2.01-1.90 (m, 4H), 1.23 (t, 3H). Mass Spectrum (LCMS, ESI pos.) Calcd. for C₂₂H₂₅N₃O₃: 379.5. Found: 380.1 (M⁺+1).

### Example 143

### 1-(2-Isopropyl-phenyl)-piperidine-4-carboxylic acid (3-oxo-3,4-dihydro-2H-benzo[1,4]oxazin-6-yl)-amide

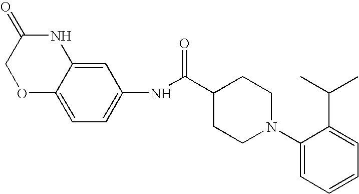 Using the procedure outlined in Example 139A, 1-(2-isopropyl-phenyl)-piperidine-4-carboxylic acid was prepared from 2-iodoisopropylbenzene (2.35 g, 9.54 mmol) as a yellow solid.
Using the procedure outlined in Example 139B, the title compound was prepared from 1-(2-isopropyl-phenyl)-piperidine-4-carboxylic acid (0.03 g, 0.122 mmol) as off-white solid. ¹H NMR (400 MHz, CD₃OD) δ (ppm): 7.40 (d, 1H), 7.24 (d, 1H), 7.12-7.10 (m, 2H), 7.07-7.04 (m, 1H), 7.00 (dd, 1H), 6.89 (d, 1H), 4.53 (s, 2H), 3.54 (h, 1H), 3.06 (d, 2H), 2.76 (td, 2H), 2.46 (m, 1H), 2.01-1.89 (m, 4H), 1.21 (s, 3H), 1.19 (s, 3H). Mass Spectrum (LCMS, ESI pos.) Calcd. for C₂₃H₂₇N₃O₃: 393.5. Found: 394.1 (M⁺+1).

### Example 144

### 1-(3,4-Dimethyl-phenyl)-piperidine-4-carboxylic acid (3-oxo-3,4-dihydro-2H-benzo[1,4]oxazin-6-yl)-amide

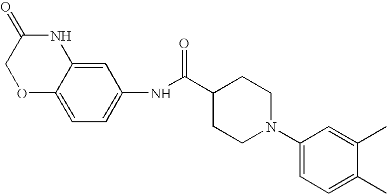 Using the procedure outlined in Example 139A, 1-(3,4-dimethyl-phenyl)-piperidine-4-carboxylic acid was prepared from 4-iodo-o-xylene (2.21 g, 9.54 mmol) as a yellow solid.
Using the procedure outlined in Example 139B, the title compound was prepared from 1-(3,4-dimethyl-phenyl)-piperidine-4-carboxylic acid (0.03 g, 0.122 mmol) as white solid. ¹H NMR (400 MHz, CDCl₃) δ (ppm): 7.61 (s, 1H), 7.52 (d, 1H), 7.14 (s, 1H), 7.03 (d, 1H), 6.91 (d, 1H), 6.78-6.71 (m, 3H), 4.59 (s, 2H), 3.70 (d, 2H), 2.72 (t, 2H), 2.35 (m, 1H), 2.23 (s, 3H), 2.19 (s, 3H), 2.04-1.98 (m, 4H). Mass Spectrum (LCMS, ESI pos.) Calcd. for C₂₂H₂₅N₃O₃: 379.5. Found: 379.9 (M⁺).

### Example 145

### 1-(2,3-Dimethyl-phenyl)-piperidine-4-carboxylic acid (3-oxo-3,4-dihydro-2H-benzo[1,4]oxazin-6-yl)-amide

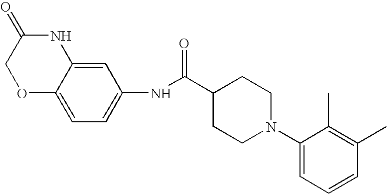 Using the procedure outlined in Example 139A, 1-(2,3-dimethyl-phenyl)-piperidine-4-carboxylic acid was prepared from 3-bromo-o-xylene (1.77 g, 9.54 mmol) as a yellow solid.
Using the procedure outlined in Example 139B, the title compound was prepared from 1-(2,3-dimethyl-phenyl)-piperidine-4-carboxylic acid (0.03 g, 0.122 mmol) as white solid. ¹H NMR (400 MHz, CD₃OD) δ (ppm): 7.39 (d, 1H), 7.03-6.99 (m, 2H), 6.92-6.84 (m, 3H), 4.53 (s, 2H), 3.49 (d, 2H), 2.67 (t, 2H), 2.46 (m, 1H), 2.25 (s, 3H), 2.23 (s, 3H), 2.03-1.90 (m, 4H). Mass Spectrum (LCMS, ESI pos.) Calcd. for C₂₂H₂₅N₃O₃: 379.5. Found: 380.1 (M⁺).

### Example 146

### 1-(4-tert-Butyl-phenyl)-piperidine-4-carboxylic acid (3-oxo-3,4-dihydro-2H-benzo[1,4]oxazin-6-yl)-amide

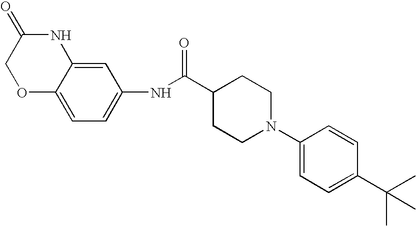 Using the procedure outlined in Example 139A, 1-(4-tert-butyl-phenyl)-piperidine-4-carboxylic acid was prepared from 1-t-butyl-4-iodobenzene (2.48 g, 9.54 mmol) as a yellow solid.
Using the procedure outlined in Example 139B, the title compound was prepared from 1-(4-tert-butyl-phenyl)-piperidine-4-carboxylic acid (0.03 g, 0.122 mmol) as white solid. ¹H NMR (400 MHz, CD₃OD) δ (ppm): 7.38 (s, 1H), 7.28 (d, 2H), 6.99-6.87 (m, 4H), 4.53 (s, 2H), 3.70 (d, 2H), 2.75-2.68 (m, 2H), 2.47 (m, 1H), 1.94 (m, 4H), 1.28 (s, 9H). Mass Spectrum (LCMS, ESI pos.) Calcd. for C₂₄H₂₉N₃O₃: 407.5. Found: 408.1 (M⁺).

### Example 147

### 1-(3-Fluoro-phenyl)-piperidine-4-carboxylic acid (3-oxo-3,4-dihydro-2H-benzo[1,4]oxazin-6-yl)-amide

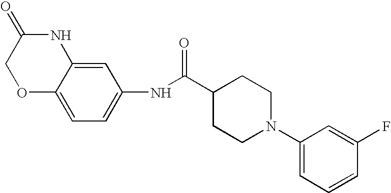 Using the procedure outlined in Example 139A, 1-(3-fluoro-phenyl)-piperidine-4-carboxylic acid was prepared from 3-bromochlorobenzene (2.12 g, 9.54 mmol) as a brown solid.
Using the procedure outlined in Example 139B, the title compound was prepared from 1-(3-fluoro-phenyl)-piperidine-4-carboxylic acid (0.03 g, 0.122 mmol) as white solid. ¹H NMR (400 MHz, CD₃OD) δ (ppm): 7.37 (d, 1H), 7.19 (q, 1H), 6.98 (dd, 1H), 6.88 (d, 1H), 6.76 (dd, 1H), 6.68 (dt, 1H), 6.49 (dt, 1H), 4.53 (s, 2H), 3.80 (d, 2H), 2.82-2.75 (m, 2H), 2.50 (m, 1H), 1.93-1.90 (m, 4H). Mass Spectrum (LCMS, ESI pos.) Calcd. for C₂₀H₂₀FN₃O₃: 369.4. Found: 370.1 (M⁺+1).

### Example 148

### 1-(3-Chloro-phenyl)-piperidine-4-carboxylic acid (3-oxo-3,4-dihydro-2H-benzo[1,4]oxazin-6-yl)-amide

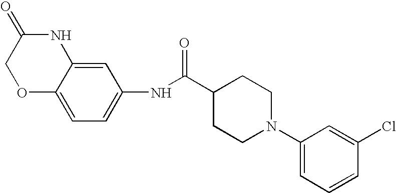 Using the procedure outlined in Example 139A, 1-(3-chloro-phenyl)-piperidine-4-carboxylic acid was prepared from 3-bromochlorobenzene (1.83 g, 9.54 mmol) as a brown solid.
Using the procedure outlined in Example 139B, the title compound was prepared from 1-(3-chloro-phenyl)-piperidine-4-carboxylic acid (0.03 g, 0.122 mmol) as white solid. ¹H NMR (400 MHz, CD₃OD) δ (ppm): 7.38 (d, 1H), 7.14 (t, 1H), 6.98 (dd, 1H), 6.95 (t, 1H), 6.91-6.87 (m, 2H), 6.78 (dd, 1H), 4.53 (s, 2H), 3.79 (d, 2H), 2.78 (td, 2H), 2.52-2.48 (m, 1H), 1.93-1.87 (m, 4H). Mass Spectrum (LCMS, ESI pos.) Calcd. for C₂₀H₂₀ClN₃O₃: 385.8. Found: 386.0 (M⁺).

### Example 149

### 1-(3-Bromo-phenyl)-piperidine-4-carboxylic acid (3-oxo-3,4-dihydro-2H-benzo[1,4]oxazin-6-yl)-amide

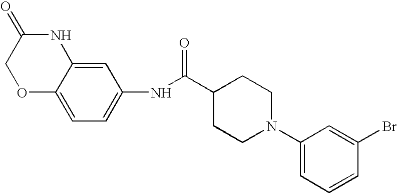 Using the procedure outlined in Example 139A, 1-(3-bromo-phenyl)-piperidine-4-carboxylic acid was prepared from 1,3-dibromobenzene (2.25 g, 9.54 mmol) as a brown solid.
Using the procedure outlined in Example 139B, the title compound was prepared from 1-(3-bromo-phenyl)-piperidine-4-carboxylic acid (0.03 g, 0.122 mmol) as white solid. ¹H NMR (400 MHz, CD₃OD) δ (ppm): 9.80 (s, 1H), 7.37 (d, 1H), 7.25 (s, 1H), 7.22-7.18 (m, 1H), 7.08 (dd, 2H), 6.99 (dd, 1H), 6.89 (d, 1H), 4.53 (s, 2H), 3.79 (d, 2H), 2.95 (m, 2H), 2.56 (m, 1H), 2.01-1.93 (m, 4H). Mass Spectrum (LCMS, ESI pos.) Calcd. for C₂₀H₂₀BrN₃O₃: 430.3. Found: 430.0 (M⁺).

### Example 150

### 1-(3-Methoxy-phenyl)-piperidine-4-carboxylic acid (3-oxo-3,4-dihydro-2H-benzo[1,4]oxazin-6-yl)-amide

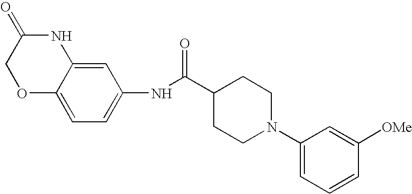 Using the procedure outlined in Example 139A, 1-(3-methoxy-phenyl)-piperidine-4-carboxylic acid was prepared from 3-iodoanisole (2.23 g, 9.54 mmol) as a yellow solid.
Using the procedure outlined in Example 139B, the title compound was prepared from 1-(3-methoxy-phenyl)-piperidine-4-carboxylic acid (0.03 g, 0.122 mmol) as off-white solid. ¹H NMR (400 MHz, CD₃OD) δ (ppm): 7.37 (d, 1H), 7.12 (t, 1H), 6.98 (dd, 1H), 6.88 (d, 1H), 6.58 (dd, 1H), 6.51 (t, 1H), 6.23 (dd, 1H), 4.53 (s, 2H), 3.76 (s, 3H), 3.74 (m, 2H), 2.78-2.71 (m, 2H), 2.48-2.46 (m, 1H), 1.93-1.89 (m, 4H). Mass Spectrum (LCMS, ESI pos.) Calcd. for C₂₁H₂₃N₃O₄: 381.4. Found: 382.0 (M⁺+1).

### Example 151

### 1-(2-Fluoro-phenyl)-piperidine-4-carboxylic acid (3-oxo-3,4-dihydro-2H-benzo[1,4]oxazin-6-yl)-amide

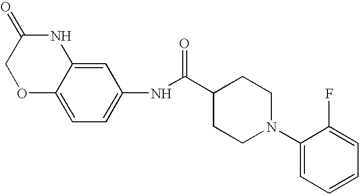 Using the procedure outlined in Example 139A, 1-(2-fluoro-phenyl)-piperidine-4-carboxylic acid was prepared from 1-fluoro-2-iodobenzene (2.12 g, 9.54 mmol) as a yellow solid.
Using the procedure outlined in Example 139B, the title compound was prepared from 1-(2-fluoro-phenyl)-piperidine-4-carboxylic acid (0.03 g, 0.122 mmol) as off-white solid. ¹H NMR (400 MHz, CD₃OD) δ (ppm): 7.39 (d, 1H), 7.08-6.95 (m, 5H), 6.89 (d, 1H), 4.53 (s, 2H), 3.51 (d, 2H), 2.74 (td, 2H), 2.48 (m, 1H), 2.01-1.95 (m, 4H). Mass Spectrum (LCMS, ESI pos.) Calcd. for C₂₀H₂₀FN₃O₃: 369.4. Found: 370.1 (M⁺+1).

### Example 152

### 1-(2-Chloro-phenyl)-piperidine-4-carboxylic acid (3-oxo-3,4-dihydro-2H-benzo[1,4]oxazin-6-yl)-amide

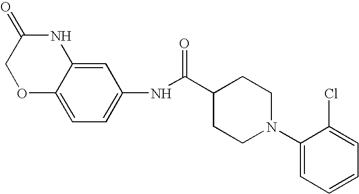 Using the procedure outlined in Example 139A, 1-(2-chloro-phenyl)-piperidine-4-carboxylic acid was prepared from 1-chloro-2-iodobenzene (2.27 g, 9.54 mmol) as a yellow solid.
Using the procedure outlined in Example 139B, the title compound was prepared from 1-(2-chloro-phenyl)-piperidine-4-carboxylic acid (0.03 g, 0.122 mmol) as off-white solid. ¹H NMR (400 MHz, CD₃OD) δ (ppm): 7.40 (d, 1H), 7.35 (dd, 1H), 7.25 (dd, 1H), 7.14 (dd, 1H), 7.01-6.97 (m, 2H), 6.89 (d, 1H), 4.54 (s, 2H), 3.43 (d, 2H), 2.73 (td, 2H), 2.49 (m, 1H), 2.05-1.94 (m, 4H). Mass Spectrum (LCMS, ESI pos.) Calcd. for C₂₀H₂₀FN₃O₃: 385.8. Found: 386.0 (M⁺).

### Example 153

### 1-(2-Bromo-phenyl)-piperidine-4-carboxylic acid (3-oxo-3,4-dihydro-2H-benzo[1,4]oxazin-6-yl)-amide

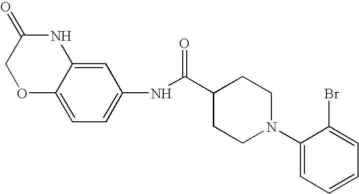 Using the procedure outlined in Example 139A, 1-(2-bromo-phenyl)-piperidine-4-carboxylic acid was prepared from 1-bromo-2-iodobenzene (2.70 g, 9.54 mmol) as a yellow solid.
Using the procedure outlined in Example 139B, the title compound was prepared from 1-(2-bromo-phenyl)-piperidine-4-carboxylic acid (0.03 g, 0.122 mmol) as white solid. ¹H NMR (400 MHz, CD₃OD) δ (ppm): 7.55 (dd, 1H), 7.40 (d, 1H), 7.30 (td, 1H), 7.14 (dd, 1H), 7.00 (dd, 1H), 6.93 (td, 1H), 6.89 (d, 1H), 4.51 (s, 2H), 3.40 (d, 2H), 2.73 (td, 2H), 2.49 (m, 1H), 2.07-1.87 (m, 4H). Mass Spectrum (LCMS, ESI pos.) Calcd. for C₂₀H₂₀BrN₃O₃: 430.3. Found: 430.0 (M⁺).

### Example 154

### 1-(3,5-Difluoro-phenyl)-piperidine-4-carboxylic acid (3-oxo-3,4-dihydro-2H-benzo[1,4]oxazin-6-yl)-amide

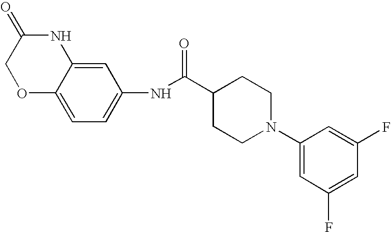 Using the procedure outlined in Example 139A, 1-(3,5-difluoro-phenyl)-piperidine-4-carboxylic acid was prepared from 1-bromo-3,5-difluorobenzene (1.84 g, 9.54 mmol) as a yellow solid.
Using the procedure outlined in Example 139B, the title compound was prepared from 1-(3,5-difluoro-phenyl)-piperidine-4-carboxylic acid (0.03 g, 0.122 mmol) as off-white solid. ¹H NMR (400 MHz, CD₃OD) δ (ppm): 7.37 (d, 1H), 6.98 (dd, 1H), 6.88 (d, 1H), 6.51 (dd, 2H), 6.28-6.23 (m, 1H), 4.53 (s, 2H), 3.82 (d, 2H), 2.83 (td, 2H), 2.56-2.50 (m, 1H), 1.93-1.84 (m, 4H). Mass Spectrum (LCMS, ESI pos.) Calcd. for C₂₀H₁₉F₂N₃O₃: 387.4. Found: 388.0 (M⁺+1).

### Example 155

### 1-(3,4-Difluoro-phenyl)-piperidine-4-carboxylic acid (3-oxo-3,4-dihydro-2H-benzo[1,4]oxazin-6-yl)-amide

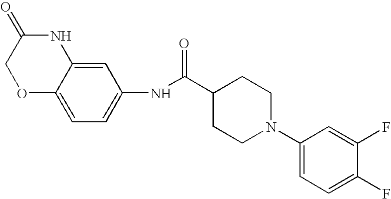 Using the procedure outlined in Example 139A, 1-(3,5-difluoro-phenyl)-piperidine-4-carboxylic acid was prepared from 1-bromo-3,5-difluorobenzene (1.84 g, 9.54 mmol) as a yellow solid.
Using the procedure outlined in Example 139B, the title compound was prepared from 1-(3,5-difluoro-phenyl)-piperidine-4-carboxylic acid (0.03 g, 0.122 mmol) as white solid. ¹H NMR (400 MHz, CD₃OD) δ (ppm): 7.37 (d, 1H), 7.08 (dt, 1H), 6.98 (dd, 1H), 6.88 (d, 1H), 6.85 (ddd, 1H), 6.75-6.71 (m, 1H), 4.53 (s, 2H), 3.69 (d, 2H), 2.77-2.70 (m, 2H), 2.49-2.45 (m, 1H), 1.94-1.88 (m, 4H). Mass Spectrum (LCMS, ESI pos.) Calcd. for C₂₀H₁₉F₂N₃O₃: 387.4. Found: 388.0 (M⁺+1).

### Example 156

### 1-(3,4,5-Trifluoro-phenyl)-piperidine-4-carboxylic acid (3-oxo-3,4-dihydro-2H-benzo[1,4]oxazin-6-yl)-amide

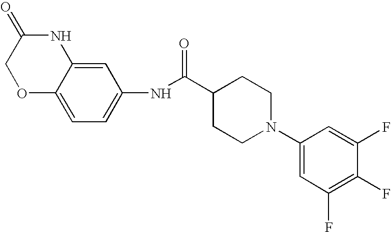 Using the procedure outlined in Example 139A, 1-(3,4,5-trifluoro-phenyl)-piperidine-4-carboxylic acid was prepared from 5-bromo-1,2,3-trifluorobenzene (2.01 g, 9.54 mmol) as a yellow solid.
Using the procedure outlined in Example 139B, the title compound was prepared from 1-(3,4,5-trifluoro-phenyl)-piperidine-4-carboxylic acid (0.03 g, 0.122 mmol) as white solid. ¹H NMR (400 MHz, CD₃OD) δ (ppm): 7.38 (d, 1H), 6.98 (dd, 1H), 6.88 (d, 1H), 6.67 (dd, 2H), 4.53 (s, 2H), 3.74 (d, 2H), 2.77 (td, 2H), 2.52-2.46 (m, 1H), 1.94-1.84 (m, 4H). Mass Spectrum (LCMS, ESI pos.) Calcd. for C₂₀H₂₀BrN₃O₃: 405.4. Found: 406.0 (M⁺+1).

### Example 157

### 1-(3-Chloro-2-methyl-phenyl)-piperidine-4-carboxylic acid (3-oxo-3,4-dihydro-2H-benzo[1,4]oxazin-6-yl)-amide

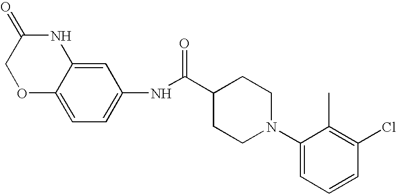 Using the procedure outlined in Example 139A, 1-(3-chloro-2-methyl-phenyl)-piperidine-4-carboxylic acid was prepared from 2-chloro-6-iodotoluene (2.41 g, 9.54 mmol) as a yellow solid.
Using the procedure outlined in Example 139B, the title compound was prepared from 1-(3-chloro-2-methyl-phenyl)-piperidine-4-carboxylic acid (0.03 g, 0.122 mmol) as off-white solid. ¹H NMR (400 MHz, CD₃OD) δ (ppm): 9.79 (s, 1H), 7.39 (d, 1H), 7.11-7.08 (m, 2H), 7.00-6.98 (m, 2H), 7.07-7.04 (d, 1H), 4.53 (s, 2H), 2.74 (t, 2H), 2.34 (m, 1H), 2.35 (s, 3H), 1.98 (m, 4H). Mass Spectrum (LCMS, ESI pos.) Calcd. for C₂₁H₂₂ClN₃O₃: 399.9. Found: 399.9 (M⁺).

### Example 158

### 3′-Methyl-3,4,5,6-tetrahydro-2H-[1,2′]bipyridinyl-4-carboxylic acid (3-oxo-3,4-dihydro-2H-benzo[1,4]oxazin-6-yl)-amide

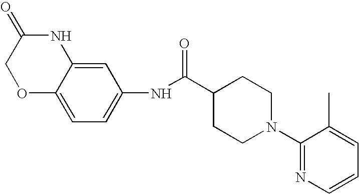 Using the procedure outlined in Example, 139A, 3′-methyl-3,4,5,6-tetrahydro-2H-[1,2′]bipyridinyl-4-carboxylic acid was prepared from 2-bromo-3-methyl-pyridine (0.98 g, 5.73 mmol) as a yellow solid.
Using the procedure outlined in Example 139B, the title compound was prepared from 3′-methyl-3,4,5,6-tetrahydro-2H-[1,2′]bipyridinyl-4-carboxylic acid (0.03 g, 0.122 mmol) as light brown solid. ¹H NMR (400 MHz, CD₃OD) δ (ppm): 9.82 (s, 1H), 8.03 (d, 1H), 7.91 (d, 1H), 7.40 (t, 1H), 7.17 (t, 2H), 7.00 (dd, 1H), 6.74 (d, 1H), 4.53 (s, 2H), 3.74 (d, 2H), 3.16 (m, 2H), 2.65 (m, 1H), 2.04-2.00 (m, 4H). Mass Spectrum (LCMS, ESI pos.) Calcd. for C₂₀H₂₂N₄O₃: 366.4. Found: 367.0 (M⁺+1).

### Example 159

### 1-Benzothiazol-2-yl-piperidine-4-carboxylic acid (3-oxo-3,4-dihydro-2H-benzo[1,4]oxazin-6-yl)-amide

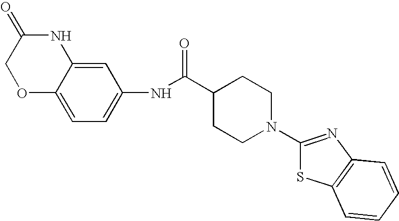 Using the procedure outlined in Example 139A, 1-benzothiazol-2-yl-piperidine-4-carboxylic acid was prepared from 2-bromo-benzothiazole (2.04 g, 9.54 mmol) as a yellow solid.
Using the procedure outlined in Example 139B, the title compound was prepared from 1-benzothiazol-2-yl-piperidine-4-carboxylic acid (0.03 g, 0.122 mmol) as white solid. ¹H NMR (400 MHz, CD₃OD) δ (ppm): 7.73 (d, 1H), 7.50 (dd, 1H), 7.41-7.37 (m, 2H), 7.21 (t, 1H), 6.99 (dd, 1H), 6.89 (d, 1H), 4.53 (s, 2H), 4.19 (d, 2H), 3.45-3.32 (m, 2H), 2.73-2.65 (m, 1H), 2.06-1.93 (m, 4H). Mass Spectrum (LCMS, ESI pos.) Calcd. for C₂₁H₂₀N₄O₃S: 408.5. Found: 409.1 (M⁺+1).

### Example 160

### 1-(2-Bromo-3-fluoro-phenyl)-piperidine-4-carboxylic acid (3-oxo-3,4-dihydro-2H-benzo[1,4]oxazin-6-yl)-amide

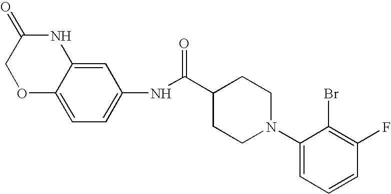 [0528] A. A mixture of ethyl isonipecotate (1.00 g, 6.36 mmol), 2-bromo-1,3-difluorobenzene (0.82 g, 4.24 mmol), and 4-(dimethylamino)pyridine (0.26 g, 2.12 mmol) was heated to 100° C. and stirred for 2 hours. The resulting reaction mixture was subjected to flash chromatography with hexanes and ethyl acetate to provide the ester, which was then dissolved into 10 mL of methanol. 2 mL of 1N lithium hydroxide was added to hydrolyze the ester at 80° C. for 3 hours. Solvent was evaporated off and the basic residue was acidified by 2N HCl. The aqueous mixture was extracted with EtOAc. The combined organic layers was washed with brine, dried over MgSO₄, filtered, and concentrated under reduced pressure to afford the crude product which was then purified by flash column chromatography with CH₂Cl₂ and MeOH to yield 1-(2-bromo-3-fluoro-phenyl)-piperidine-4-carboxylic acid as yellow solid.
B. Using the procedure outlined in Example 139B, the title compound was prepared from 1-(2-bromo-3-fluoro-phenyl)-piperidine-4-carboxylic acid (0.04 g, 0.122 mmol) as white solid. ¹H NMR (400 MHz, CD₃OD) δ (ppm): 7.40 (d, 1H), 7.29 (td, 1H), 7.00 (dd, 1H), 6.95 (d, 1H), 6.90-6.86 (m, 2H), 4.54 (s, 2H), 3.43 (d, 2H), 2.75 (td, 2H), 2.53-2.46 (m, 1H), 2.10-1.92 (m, 4H). Mass Spectrum (LCMS, ESI pos.) Calcd. for C₂₀H₁₉BrFN₃O₃: 448.3. Found: 449.9 (M⁺+1).

### Example 161

### 3′-Nitro-3,4,5,6-tetrahydro-2H-[1,2′]bipyridinyl-4-carboxylic acid (3-oxo-3,4-dihydro-2H-benzo[1,4]oxazin-6-yl)-amide

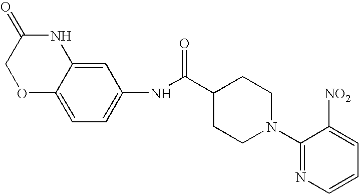 Using the procedure outlined in Example 160A, 3′-nitro-3,4,5,6-tetrahydro-2H-[1,2′]bipyridinyl-4-carboxylic acid was prepared from 2-bromo-3-nitropyridine (1.06 g, 4.24 mmol) as a yellow solid.
Using the procedure outlined in Example 139B, the title compound was prepared from 3′-nitro-3,4,5,6-tetrahydro-2H-[1,2′]bipyridinyl-4-carboxylic acid (0.03 g, 0.122 mmol) as yellow solid. ¹H NMR (400 MHz, CD₃OD) δ (ppm): 8.35 (dd, 1H), 8.18 (dd, 1H), 7.38 (d, 1H), 6.98 (dd, 2H), 6.88 (d, 1H), 6.84 (dd, 1H), 4.53 (s, 2H), 3.89 (d, 2H), 3.13-3.05 (m, 2H), 2.63 (m, 1H), 1.91-1.85 (m, 4H). Mass Spectrum (LCMS, ESI pos.) Calcd. for C₁₉H₁₉N₅O₅: 397.4. Found: 398.0 (M⁺+1).

### Example 162

### 1-(2-Cyano-3-trifluoromethyl-phenyl)-piperidine-4-carboxylic acid (3-oxo-3,4-dihydro-2H-benzo[1,4]oxazin-6-yl)-amide

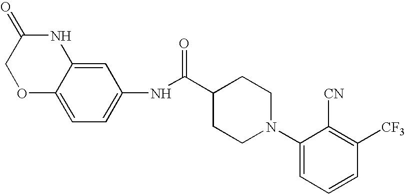 Using the procedure outlined in Example 160A, 1-(2-cyano-3-trifluoromethyl-phenyl)-piperidine-4-carboxylic acid was prepared from 2-fluoro-6-(trifluoromethyl)benzonitrile (1.26 g, 4.24 mmol) as a yellow solid.
Using the procedure outlined in Example 139B, the title compound was prepared from 1-(2-cyano-3-trifluoromethyl-phenyl)-piperidine-4-carboxylic acid (0.04 g, 0.122 mmol) as off-white solid. ¹H NMR (400 MHz, CD₃OD) δ (ppm): 7.71 (t, 1H), 7.47 (d, 1H), 7.43 (d, 1H), 7.40 (d, 1H), 6.99 (dd, 1H), 6.89 (d, 1H), 4.54 (s, 2H), 3.65 (d, 2H), 2.98 (td, 2H), 2.55 (m, 1H), 2.08-1.98 (m, 4H). Mass Spectrum (LCMS, ESI pos.) Calcd. for C₂₂H₁₉F₃N₄O₃: 444.4. Found: 445.1 (M⁺+1).

### Example 163

### 1-(2-Cyano-5-trifluoromethyl-phenyl)-piperidine-4-carboxylic acid (3-oxo-3,4-dihydro-2H-benzo[1,4]oxazin-6-yl)-amide

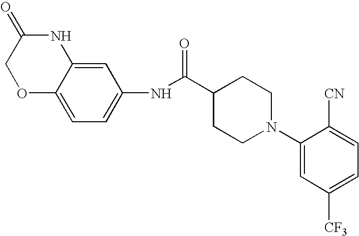 Using the procedure outlined in Example 160A, 1-(2-cyano-5-trifluoromethyl-phenyl)-piperidine-4-carboxylic acid was prepared from 2-fluoro-4-(trifluoromethyl)benzonitrile (1.26 g, 2.42 mmol) as a yellow solid.
Using the procedure outlined in Example 139B, the title compound was prepared from 1-(2-cyano-5-trifluoromethyl-phenyl)-piperidine-4-carboxylic acid (0.04 g, 0.122 mmol) as white solid. ¹H NMR (400 MHz, CD₃OD) δ (ppm): 7.80 (d, 1H), 7.40-7.32 (m, 3H), 6.99 (dd, 1H), 6.89 (d, 1H), 4.54 (s, 2H), 3.74 (d, 2H), 2.99 (td, 2H), 2.56 (m, 1H), 2.06-2.00 (m, 4H). Mass Spectrum (LCMS, ESI pos.) Calcd. for C₂₂H₁₉F₃N₄O₃: 444.4. Found: 445.0 (M⁺+1).

### Example 164

### 1-(4-Bromo-2,3,5,6-tetrafluoro-phenyl)-piperidine-4-carboxylic acid (3-oxo-3,4-dihydro-2H-benzo[1,4]oxazin-6-yl)-amide

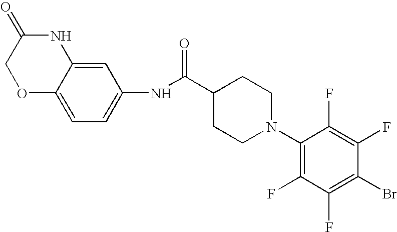 Using the procedure outlined in Example 160A, 1-(4-bromo-2,3,5,6-tetrafluoro-phenyl)-piperidine-4-carboxylic acid was prepared from bromo-pentafluorobenzene (1.51 g, 4.24 mmol) as a yellow solid.
Using the procedure outlined in Example 139B, the title compound was prepared from 1-(4-bromo-2,3,5,6-tetrafluoro-phenyl)-piperidine-4-carboxylic acid (0.04 g, 0.122 mmol) as white solid. ¹H NMR (400 MHz, DMSO-d₆) δ (ppm): 10.71 (s, 1H), 9.88 (s, 1H), 7.56 (d, 1H), 7.04 (dd, 1H), 6.85 (d, 1H), 4.89 (s, 2H), 3.10 (td, 2H), 1.84-1.71 (m, 4H). Mass Spectrum (LCMS, ESI pos.) Calcd. for C₂₀H₁₆F₄N₃O₃: 502.3. Found: 503.9 (M⁺+1).

### Example 165

### 1-(2-Trifluoromethyl-phenyl)-piperidine-4-carboxylic acid (3-oxo-3,4-dihydro-2H-benzo[1,4]oxazin-6-yl)-amide

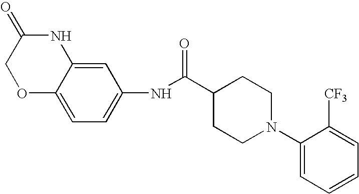 [0535] A. To a solution of ethyl isonipecotate (0.42 g, 2.67 mmol) in 7 mL of anhydrous toluene was added bromobenzotrifluoride (0.50 g, 2.22 mmol), tris(dibenzylideneacetone)dipalladium(0) (0.10 g, 0.109 mmol), 2-(di-t-butylphosphino)biphenyl (0.13 g, 0.437 mmol), and sodium t-butoxide (0.32 g, 3.33 mmol). The reaction mixture was heated to 80° C. and stirred for 12 hours. Solids were filtered off from the resulting mixture and the collected filtrate was concentrated under reduced pressure to dryness, which was then purified by flash column chromatography with hexanes and ethyl acetate to provide the ester. Then, it was dissolved into 6 mL of methanol. 1 mL of 1N lithium hydroxide was added to hydrolyze the ester at 80° C. for 2 hours. Solvent was evaporated off and the basic residue was acidified by 2N HCl. The aqueous mixture was extracted with EtOAc. The combined organic layers was washed with brine, dried over MgSO₄, filtered, and concentrated under reduced pressure to afford the crude product which was then purified by flash column chromatography with CH₂Cl₂ and MeOH to yield 1-(2-trifluoromethyl-phenyl)-piperidine-4-carboxylic acid as yellow solid.
B. Using the procedure outlined in Example 139B, the title compound was prepared from 1-(2-trifluoromethyl-phenyl)-piperidine-4-carboxylic acid (0.03 g, 0.122 mmol) as yellow solid. ¹H NMR (400 MHz, CD₃OD) δ (ppm): 7.64-7.57 (m, 2H), 7.48 (d, 1H), 7.40 (d, 1H), 7.28 (t, 1H), 6.99 (dd, 1H), 6.88 (d, 1H), 4.53 (s, 2H), 3.10 (d, 2H), 2.84 (t, 2H), 2.49-2.45 (m, 1H), 2.01-1.88 (m, 4H). Mass Spectrum (LCMS, ESI pos.) Calcd. for C₂₁H₂₀F₃N₃O₃: 419.4. Found: 420.0 (M⁺+1).

### Example 166

### 1-(2,5-Bis-trifluoromethyl-phenyl)-piperidine-4-carboxylic acid (3-oxo-3,4-dihydro-2H-benzo[1,4]oxazin-6-yl)-amide

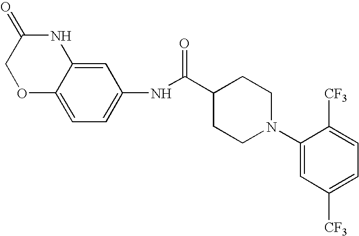 Using the procedure outlined in Example 165A, 1-(2,5-bis-trifluoromethyl-phenyl)-piperidine-4-carboxylic acid was prepared from 2,5-bis(trifluoromethyl)bromobenzene (0.51 g, 2.23 mmol) as a yellow solid.
Using the procedure outlined in Example 139B, the title compound was prepared from 1-(2,5-bis-trifluoromethyl-phenyl)-piperidine-4-carboxylic acid (0.04 g, 0.122 mmol) as off-white solid. ¹H NMR (400 MHz, CD₃OD) δ (ppm): 7.86 (d, 1H), 7.75 (s, 1H), 7.60 (d, 1H), 7.40 (d, 1H), 6.99 (dd, 1H), 6.89 (d, 1H), 4.53 (s, 2H), 3.16 (d, 2H), 2.89 (td, 2H), 2.51 (m, 1H), 2.02-1.90 (m, 4H). Mass Spectrum (LCMS, ESI pos.) Calcd. for C₂₂H₁₉F₆N₃O₃: 487.4. Found: 488.0 (M⁺+1).

### Example 167

### 1-(3-Trifluoromethyl-phenyl)-piperidine-4-carboxylic acid (2-oxo-2,3-dihydro-1H-pyrido[2,3-b][1,4]oxazin-7-yl)-amide

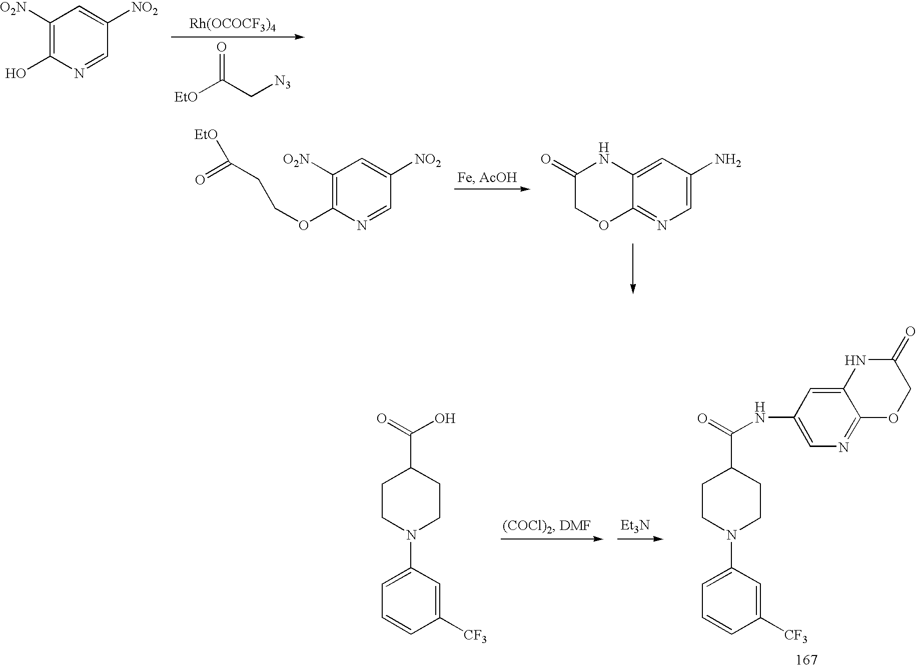 [0539] A. To a solution mixture of 2-hydroxy-3,5-dinitropyridine (2.00 g, 10.8 mmol) and rhodium(II) trifluoroacetate dimmer (0.14 g, 2.16 mmol) in 40 mL of 1,2-dichloroethane was added ethyl diazoacetate (1.11 g, 9.72 mmol) in 8 mL of 1,2-dichloroethane slowly by spyring pump over 6 hours at 40° C. The reaction was stirred for another 3 hours at 25° C. after addition. Solvent was evaporated off to give the residue, which was then purified by flash column chromatography with hexanes and ethyl acetate to afford the ester.
B. To a solution of the ester (0.34 g, 1.25 mmol) in 7 mL of solvent mixture (AcOH:EtOH=1:2) was added iron powder (0.35 g, 6.27 mmol). The reaction mixture was refluxed for 10 hours. After simple filtration, the filtrate was concentrated under reduced pressure to provide the residue, which was then purified by flash column chromatography with hexanes and ethyl acetate to afford 7-amino-1H-pyrido[2,3-b][1,4]oxazin-2-one.
C. Using the procedure outlined in Example 1A, 1-(3-trifluoromethyl-phenyl)-piperidine-4-carboxylic acid was prepared from 3-bromobenzotrifluoride (0.21 g, 0.95 mmol) as a yellow solid.
D. To a solution of 1-(3-trifluoromethyl-phenyl)-piperidine-4-carboxylic acid (0.025 g, 0.0914 mmol) in 4 mL of CH₂Cl₂ was added oxalyl chloride (0.02 mL, 0.183 mmol) and a catalytic amount of DMF. The reaction mixture was stirred for 2 hours. Solvent was evaporated to dryness under reduced pressure. The residue was redissolved in 4 mL of CH₂Cl₂. 7-amino-1H-pyrido[2,3-b][1,4]oxazin-2-one obtained in step B (0.017 g, 0.101 mmol) and triethylamine (0.02 mL, 0.137 mmol) were added sequentially into the acyl chloride solution at 0° C. The reaction mixture was warmed to 25° C. and stirred for 20 min. Concentration of the solvent provided the residue which was then purified by flash column chromatography with hexanes and ethyl acetate to afford 1-(3-trifluoromethyl-phenyl)-piperidine-4-carboxylic acid (2-oxo-2,3-dihydro-1H-pyrido[2,3-b][1,4]oxazin-7-yl)-amide. ¹H NMR (400 MHz, CD₃OD) δ (ppm): 7.87 (d, 1H), 7.83 (d, 1H), 7.39 (t, 1H), 7.20 (d, 1H), 7.17 (s, 1H), 7.05 (d, 1H), 4.78 (s, 2H), 3.85 (d, 2H), 2.84 (td, 2H), 2.58-2.52 (m, 1H), 2.01-1.90 (m, 4H). Mass Spectrum (LCMS, ESI pos.) Calcd. for C₂₀H₁₉F₃N₄O₃: 420.4. Found: 421.0 (M⁺+1).

### Example 168

### 1-(3-Trifluoromethyl-phenyl)-piperidine-4-carboxylic acid [4-(2-hydroxy-ethyl)-3-oxo-3,4-dihydro-2H-benzo[1,4]oxazin-6-yl]-amide

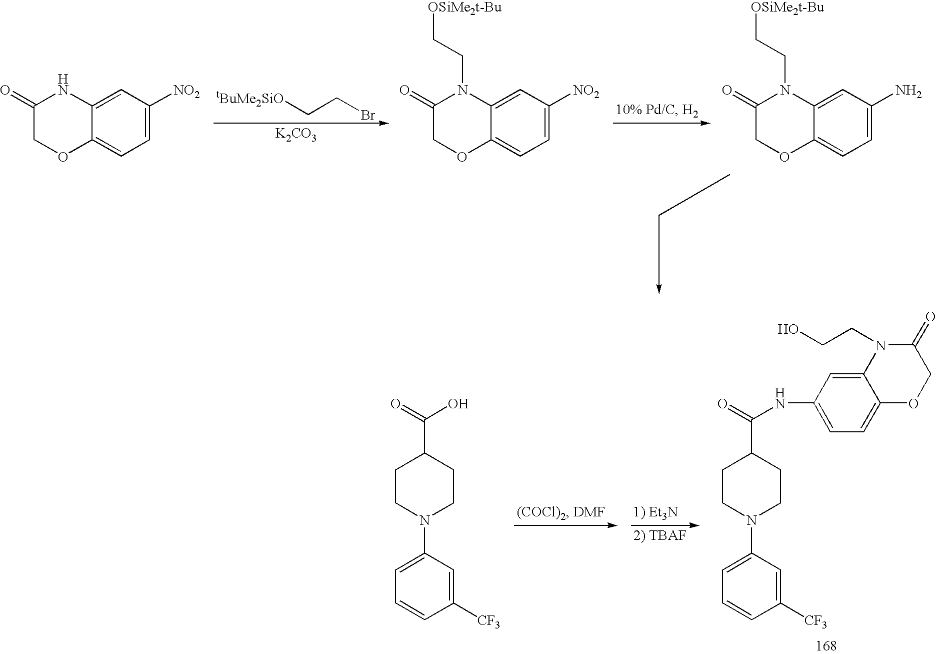 [0544] A. To a solution of 6-nitro-2H-1,4-benzoxazin-3(4H)-one (0.50 g, 2.58 mmol) in 10 mL of MeCN was added potassium carbonate (0.53 g, 3.86 mmol) and (2-bromoethoxy)-t-butyldimethylsilane (0.55 mL, 2.58 mmol). The mixture was heated to 80° C. and stirred for 12 hours. The filtrate was collected from removing the solid and was concentrated under reduced pressure to residue, which was then purified by flash column chromatography with hexanes and ethyl acetate to afford the nitro compound.
B. To a solution of nitro compound (0.32, 0.908 mmol) in 10 mL of EtOH was added 10% palladium on charcoal (0.10 g, 0.0908 mmol). The mixture was hydrogenated under balloon pressure of hydrogen gas for one hour. The catalyst was removed and the filtrate was concentrated under reduced pressure to provide the amino compound.
C. Using the procedure outlined in Example 1A, 1-(3-trifluoromethyl-phenyl)-piperidine-4-carboxylic acid was prepared from 3-bromobenzotrifluoride (0.21 g, 0.95 mmol) as a yellow solid.
D. To a solution of 1-(3-trifluoromethyl-phenyl)-piperidine-4-carboxylic acid (0.026 g, 0.0951 mmol) in 4 mL of CH₂Cl₂ was added oxalyl chloride (0.02 mL, 0.190 mmol) and a catalytic amount of DMF. The reaction mixture was stirred for 2 hours. Solvent was evaporated to dryness under reduced pressure. The residue was redissolved in 4 mL of CH₂Cl₂. The amino compound obtained in step B (0.031 g, 0.0951 mmol) and triethylamine (0.02 mL, 0.143 mmol) were added sequentially into the acyl chloride solution at 0° C. The reaction mixture was warmed to 25° C. and stirred for 20 min. Concentration of the solvent provided the residue which was then purified by flash column chromatography with hexanes and ethyl acetate to afford the silyl protected compound.
E. To a solution of the silyl protected compound (0.023 g, 0.0398 mmol) in 4 mL of THF was added 1M tetrabutylammonium fluoride in THF (0.05 mL, 0.0477 mmol) at 0° C. The reaction mixture was warmed to 25° C. and stirred for 3 hours. Solvent was evaporated to dryness, which was then purified by flash column chromatography with hexanes and ethyl acetate to afford 1-(3-rifluoromethyl-phenyl)-piperidine-4-carboxylic acid [4-(2-hydroxy-ethyl)-3-oxo-3,4-dihydro-2H-benzo[1,4]oxazin-6-yl]-amide as white solid. ¹H NMR (400 MHz, CD₃OD) δ (ppm): 7.67 (d, 1H), 7.39 (t, 1H), 7.32 (dd, 1H), 7.12 (s, 1H), 7.09 (dd, 1H), 7.05 (d, 1H), 6.93 (d, 1H), 4.57 (s, 2H), 4.03 (t, 2H), 3.60 (d, 2H), 3.79 (t, 2H), 2.84 (td, 2H), 2.55-2.50 (m, 1H), 2.01-1.91 (m, 4H). Mass Spectrum (LCMS, ESI pos.) Calcd. for C₂₃H₂₄F₃N₃O₄: 463.5. Found: 464.0 (M⁺).

### Example 169

### 1-(3,4,5-Trifluoro-phenyl)-piperidine-4-carboxylic acid (3-oxo-3,4-dihydro-2H-benzo[1,4]oxazin-5-yl)-amide

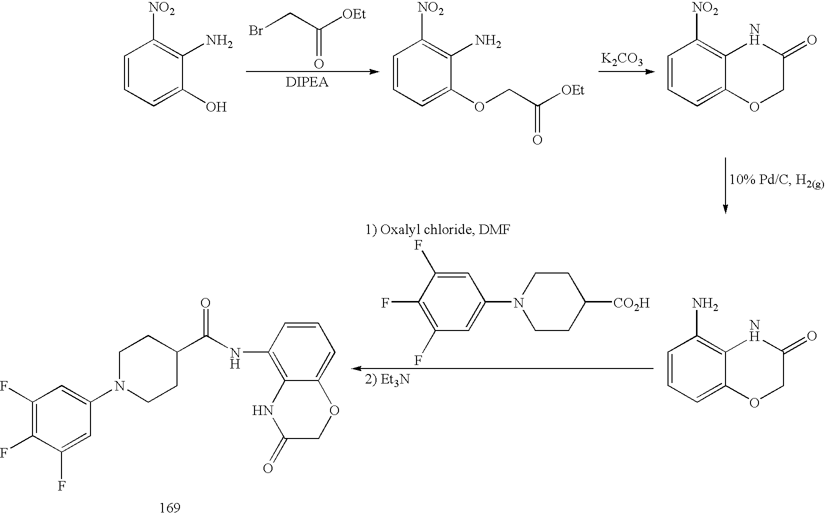 [0550] A. A mixture of 2-amino-3-nitrophenol (3.00 g, 0.0195 mol), ethyl bromoacetate (6.0 mL, 0.0545 mol), and diisopropylethylamine (4.8 mL, 0.0273 mol) was heated to 140° C. for 5 hours. The reaction mixture was cooled down, diluted with EtOAc, and washed with brine. The organic layer was dried over MgSO₄, filtered and the filtrate was concentrated under reduced pressure. The residue was purified by chromatography (silica, hexanes and ethyl acetate) to provide (2-amino-3-nitro-phenoxy)-acetic acid ethyl ester as a yellow oil.
B. To a solution of (2-amino-3-nitro-phenoxy)-acetic acid ethyl ester (2.57 g, 0.107 mol) in 120 mL of EtOH at rt was added potassium carbonate (2.96 g, 2.17 mol). The reaction mixture was refluxed for 72 hours. The reaction mixture was concentrated in vacuo, and the residue was purified chromatography (silica, hexanes and ethyl acetate) to provide 5-nitro-4H-benzo[1,4]oxazin-3-one as yellow solid.
C. To a solution of 5-nitro-4H-benzo[1,4]oxazin-3-one (0.314 g, 1.62 mmol) in 7 mL of MeOH was added 10% palladium on charcoal (0.172 g, 0.162 mmol). The reaction mixture was stirred under balloon pressure of H₂ gas for 2 hours. The catalyst was filtered and the filtrate was concentrated under reduced pressure to provide 5-amino-4H-benzo[1,4]oxazin-3-one as light brown solid.
D. Using the procedure outlined in Example 139A, 1-(3,4,5-trifluoro-phenyl)-piperidine-4-carboxylic acid was prepared from 5-bromo-1,2,3-trifluorobenzene (2.01 g, 9.54 mmol) as a yellow solid.
E. To a solution of 1-(3,4,5-trifluoro-phenyl)-piperidine-4-carboxylic acid (0.052 g, 0.201 mmol) in 4 mL of CH₂Cl₂ was added oxalyl chloride (0.04 mL, 0.402 mmol) and a catalytic amount of DMF. The reaction mixture was stirred for 2 hours. The reaction mixture was concentrated under reduced pressure. The residue was redissolved in 4 mL of CH₂Cl₂, and 5-Amino-4H-benzo[1,4]oxazin-3-one (obtained in step C) (0.033 g, 0.201 mmol) and triethylamine (0.06 mL, 0.402 mmol) were added sequentially to the acyl chloride solution at 0° C. The reaction mixture was warmed to 25° C. and stirred for 20 min. The reaction mixture was concentrated, and the residue which was purified by chromatography (silica, hexanes and ethyl acetate) to afford 1-(3,4,5-trifluoro-phenyl)-piperidine-4-carboxylic acid (3-oxo-3,4-dihydro-2H-benzo[1,4]oxazin-5-yl)-amide as white solid. ¹H NMR (400 MHz, CD₃OD) δ (ppm): 7.6.99-6.94 (m, 2H), 6.89 (dd, 1H), 6.69 (dd, 2H), 4.55 (s, 2H), 3.75 (d, 2H), 2.80 (td, 2H), 2.62-2.58 (m, 1H), 2.05-2.01 (m, 2H), 1.90 (td, 2H). Mass Spectrum (LCMS, ESI pos.) Calcd. for C₂₀H₁₈F₃N₃O₃: 405.4. Found: 406.1 (M⁺+1).

### Example 170

### 1-(3,4,5-Trifluoro-phenyl)-piperidine-4-carboxylic acid (3-oxo-3,4-dihydro-2H-benzo[1,4]oxazin-7-yl)-amide

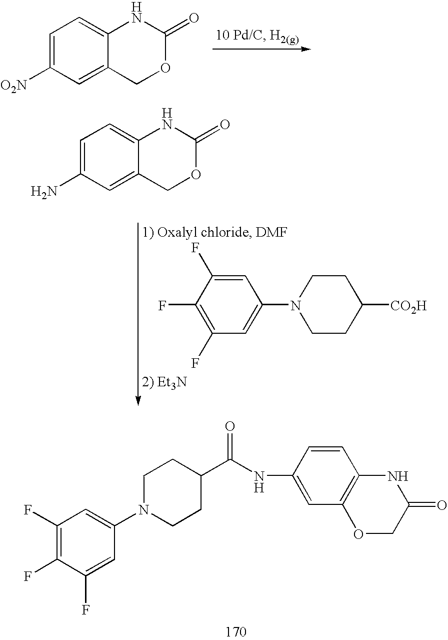 [0556] E. To a solution of 5-nitro-4H benzo[1,4]oxazin-3-one (0.314 g, 1.62 mmol) in 7 mL of MeOH was added 10% palladium on charcoal (0.172 g, 0.162 mmol). The reaction mixture was hydrogenated under balloon pressure of H₂ gas for 2 hours. The catalyst was removed by filtration, and the filtrate was concentrated under reduced pressure to provide 7-amino-4H-benzo[1,4]oxazin-3-one as light brown solid.
F. To a solution of 1-(3,4,5-trifluoro-phenyl)-piperidine-4-carboxylic acid, prepared in Example 168D, (0.048 g, 0.185 mmol) in 4 mL of CH₂Cl₂ was added oxalyl chloride (0.03 mL, 0.370 mmol) and a catalytic amount of DMF. The reaction mixture was stirred for 2 hours. The reaction mixture was concentrated under reduced pressure, and the residue was redissolved in 4 mL of CH₂Cl₂. 7-Amino-4H-benzo[1,4]oxazin-3-one obtained in step A (0.030 g, 0.185 mmol) and triethylamine (0.05 mL, 0.370 mmol) were added sequentially to the acyl chloride solution at 0° C. The reaction mixture was warmed to 25° C. and stirred for 20 min. The reaction mixture was concentrated, and the residue the residue was purified by chromatography (silica, with hexanes and ethyl acetate) to afford 1-(3,4,5-Trifluoro-phenyl)-piperidine-4-carboxylic acid (3-oxo-3,4-dihydro-2H-benzo[1,4]oxazin-7-yl)-amide as white solid. ¹H NMR (400 MHz, CD₃OD) δ (ppm): 7.30 (d, 1H), 7.11 (dd, 1H), 6.82 (d, 1H), 6.67 (dd, 2H), 4.56 (s, 2H), 3.74 (d, 2H), 2.76 (td, 2H), 2.52-2.46 (m, 1H), 1.94-1.84 (m, 4H). Mass Spectrum (LCMS, ESI pos.) Calcd. for C₂₀H₁₈F₃N₃O₃: 405.4. Found: 406.2 (M⁺+1)

### Example 171

### 1-(3,4,5-Trifluoro-phenyl)-piperidine-4-carboxylic acid (3-oxo-3,4-dihydro-2H-benzo[1,4]oxazin-8-yl)-amide

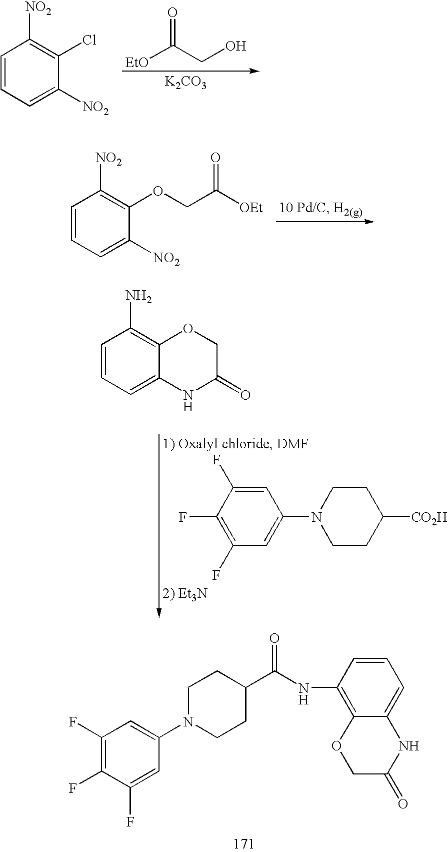 [0559] A. To a solution of 2-chloro-1,3-dinitrobenzene (1.00 g, 4.94 mmol) in 14 mL of DMF was added ethyl glycolate (0.5 mL, 5.43 mmol) and potassium carbonate (0.75 g, 5.43 mmol). The reaction mixture was stirred for 8 hours at 25° C. It was then diluted with EtOAc and washed with water. The organic layer was dried over MgSO₄, filtered and the filtrate concentrated under reduced pressure. The residue was purified by chromatography (silica with hexanes and ethyl acetate) to provide (2,6-dinitro-phenoxy)-acetic acid ethyl ester as yellow solid.
B. To a solution of (2,6-dinitro-phenoxy)-acetic acid ethyl ester (0.262 g, 0.970 mmol) in 12 mL of MeOH was added 10% palladium on charcoal (0.103 g, 0.0970 mmol). The reaction mixture was hydrogenated under balloon pressure of H₂(g) at 60° C. for 6 hours. The catalyst was removed by filtration and the filtrate was concentrated under reduced pressure to provide 8-amino-4H-benzo[1,4]oxazin-3-one as light brown solid.
C. To a solution of 1-(3,4,5-trifluoro-phenyl)-piperidine-4-carboxylic acid, prepared in Example 168D, (0.047 g, 0.181 mmol) in 4 mL of CH₂Cl₂ was added oxalyl chloride (0.03 mL, 0.362 mmol) and a catalytic amount of DMF. The reaction mixture was stirred for 2 hours. The reaction mixture was concentrated under reduced pressure. The residue was redissolved in 4 mL of CH₂Cl₂. 8-Amino-4H-benzo[1,4]oxazin-3-one obtained in step B (0.030 g, 0.181 mmol) and triethylamine (0.05 mL, 0.362 mmol) were added sequentially to the acyl chloride solution at 0° C. The reaction mixture was warmed to 25° C. and stirred for 20 min. The reaction mixture was concentrated, and the residue which was purified by chromatography (silica, with hexanes and ethyl acetate) to afford 1-(3,4,5-trifluoro-phenyl)-piperidine-4-carboxylic acid (3-oxo-3,4-dihydro-2H-benzo[1,4]oxazin-8-yl)-amide as white solid. ¹H NMR (400 MHz, CD₃OD) δ (ppm): 7.38 (d, 1H), 6.98 (dd, 1H), 6.85 (d, 1H), 6.68 (dd, 2H), 4.53 (s, 2H), 3.74 (d, 2H), 2.77 (td, 2H), 2.53-2.46 (m, 1H), 1.91-1.81 (m, 4H). Mass Spectrum (LCMS, ESI pos.) Calcd. for C₂₀H₁₈F₃N₃O₃: 405.4. Found: 406.1 (M⁺+1).

### Example 172

### 1-(3,4,5-Trifluoro-phenyl)-piperidine-4-carboxylic acid (4-methyl-3-oxo-3,4-dihydro-2H-benzo[1,4]oxazin-6-yl)-amide

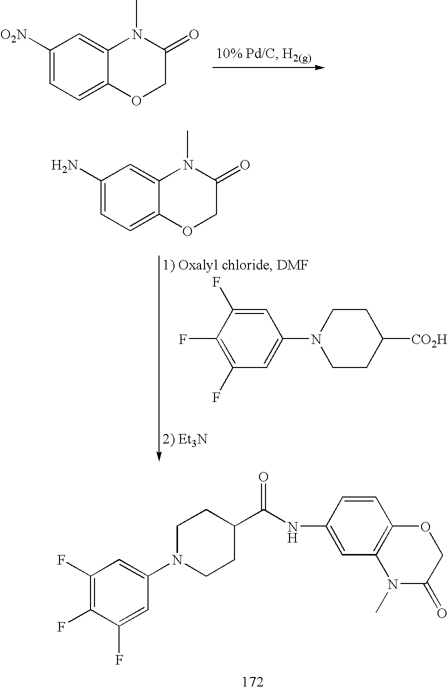 [0563] A. To a solution of 4-methyl-6-nitro-4H-benzo[1,4]oxazin-3-one (0.100 g, 0.480 mmol) in 7 mL of MeOH was added palladium on charcoal (0.051 g, 0.048 mmol). The reaction mixture was hydrogenated under balloon pressure of H₂(g) 1 hours. The catalyst was removed by filtration, and the filtrate was concentrated under reduced pressure to provide 6-amino-4-methyl-4H-benzo[1,4]oxazin-3-one as brown solid.
B. To a solution of 1-(3,4,5-trifluoro-phenyl)-piperidine-4-carboxylic acid, prepared in Example 168D, (0.048 g, 0.185 mmol) in 4 mL of CH₂Cl₂ was added oxalyl chloride (0.03 mL, 0.370 mmol) and a catalytic amount of DMF. The reaction mixture was stirred for 2 hours. The reaction mixture was concentrated under reduced pressure. The residue was redissolved in 4 mL of CH₂Cl₂. 8-Amino-4H-benzo[1,4]oxazin-3-one obtained in step A (0.030 g, 0.181 mmol) and triethylamine (0.05 mL, 0.362 mmol) were added sequentially to the acyl chloride solution at 0° C. The reaction mixture was warmed to 25° C. and stirred for 20 min. The reaction mixture was concentrated under reduced pressure, and the residue was purified chromatography (silica, with hexanes and ethyl acetate) to afford 1-(3,4,5-trifluoro-phenyl)-piperidine-4-carboxylic acid (4-methyl-3-oxo-3,4-dihydro-2H-benzo[1,4]oxazin-6-yl)-amide as white solid. ¹H NMR (400 MHz, CD₃OD) δ (ppm): 7.56 (d, 1H), 7.10 (dd, 1H), 6.93 (d, 1H), 6.68 (dd, 2H), 4.58 (s, 2H), 3.75 (d, 2H), 2.78 (td, 2H), 2.54-2.48 (m, 1H), 2.01-1.82 (m, 4H). Mass Spectrum (LCMS, ESI pos.) Calcd. for C₂₁H₂₀F₃N₃O₃: 419.4. Found: 420.2 (M⁺+1).

### Example 173

### 1-(3,4,5-Trifluoro-phenyl)-piperidine-4-carboxylic acid (3-oxo-3,4-dihydro-2H-benzo[1,4]thiazin-6-yl)-amide

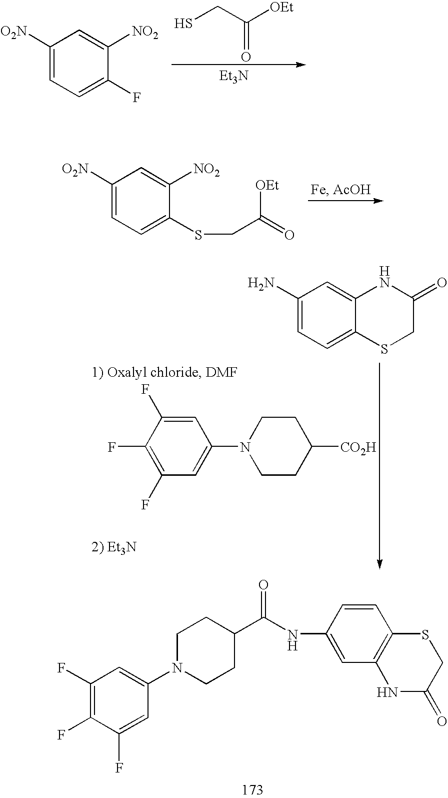 [0566] A. A solution of 2,4-dinitrofluorobenzene (5.5 mL, 0.0455 mol), ethyl mercaptoacetate (5.0 g, 0.0455 mol) and triethylamine (5.3 mL, 0.0455 mol) in 14 mL of THF was stirred at 0° C. for 12 hours. Ice was added to quench the reaction and the reaction mixture was extracted with EtOAc. The combined organic fractions were dried over MgSO₄, filtered and the filtrate was concentrated. The residue purified by chromatography (silica, with hexanes and ethyl acetate) to afford (2,4-dinitro-phenylsulfanyl)-acetic acid ethyl ester as brown oil.
B. A solution of (2,4-dinitro-phenylsulfanyl)-acetic acid ethyl ester (9.90 g, 0.0346 mol) in 40 mL EtOAc and 40 mL AcOH was added dropwise to a suspension at 60° C. prepared by mixing iron (25 g, 0.450 mol) with 40 mL H₂O and 2 mL AcOH. After the addition was complete, the reaction mixture was heated at 80° C. for 9 hours. The solid was removed by filtration, and the filtrate was concentrated under reduced pressure. The residue was purified chromatography (silica with hexanes and ethyl acetate) to afford 6-amino-4H-benzo[1,4]thiazin-3-one as dark brown solid.
C. To a solution of 1-(3,4,5-Trifluoro-phenyl)-piperidine-4-carboxylic acid, prepared in Example 168D, (0.060 g, 0.231 mmol) in 5 mL of CH₂Cl₂ was added oxalyl chloride (0.04 mL, 0.462 mmol) and a catalytic amount of DMF. The reaction mixture was stirred for 2 hours. The reaction mixture was concentrated under reduced pressure. The residue was redissolved in 5 mL of CH₂Cl₂. 6-Amino-4H-benzo[1,4]thiazin-3-one obtained in step B (0.042 g, 0.231 mmol) and triethylamine (0.06 mL, 0.462 mmol) were added sequentially to the acyl chloride solution at 0° C. The reaction mixture was warmed to 25° C. and stirred for 20 min. The reaction mixture was concentrated under reduced pressure, and the residue was purified by chromatography (silica, with hexanes and ethyl acetate) to afford 1-(3,4,5-trifluoro-phenyl)-piperidine-4-carboxylic acid (3-oxo-3,4-dihydro-2H-benzo[1,4]thiazin-6-yl)-amide as white solid. ¹H NMR (400 MHz, CD₃OD) δ (ppm): 4.74 (d, 1H), 7.23 (d, 1H), 7.08 (dd, 1H), 6.68 (dd, 2H), 3.74 (d, 2H), 3.40 (s, 2H), 2.77 (td, 2H), 2.52-2.49 (m, 1H), 1.91-1.81 (m, 4H). Mass Spectrum (LCMS, ESI pos.) Calcd. for C₂₀H₁₈F₃N₃O₂S: 421.4. Found: 422.1 (M⁺+1).

### Example 174

### 1-(3,4,5-Trifluoro-phenyl)-piperidine-4-carboxylic acid [2-(2-hydroxy-ethyl)-3-oxo-3,4-dihydro-2H-benzo[1,4]oxazin-6-yl]-amide

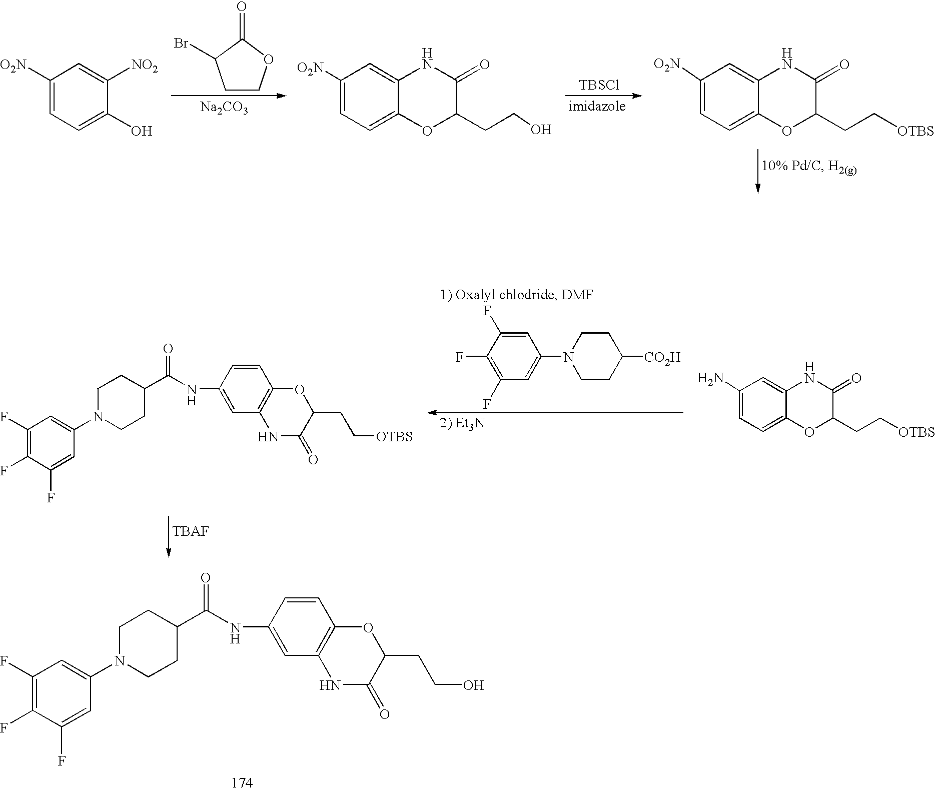 [0570] A. To a solution of 2-amino-5-nitrophenol (20.0 g, 0.109 mol) in 120 mL of DMF was added □-bromo-□-butyrolactone (11.1 mL, 0.119 mol) and sodium carbonate (11.6 g, 0.109 mol). The reaction mixture was heated at reflux for 5 hours. Water was added to quench the reaction and the reaction mixture was extracted with EtOAc. The combined organic fractions were dried over MgSO₄, filtered and the filtrate was concentrated under educed pressure. The residue was purified by chromatography (silica, with hexanes and ethyl acetate) to afford 2-(2-hydroxy-ethyl)-6-nitro-4H-benzo[1,4]oxazin-3-one as brown oil.
B. To a solution of 2-(2-hydroxy-ethyl)-6-nitro-4H-benzo[1,4]oxazin-3-one (1.09 g, 4.58 mmol) in 18 mL of DMF was added tert-butyldimethylsilyl chloride (0.83 g, 5.49 mmol) and imidazole (0.78 g, 2.5 mmol). The reaction mixture was stirred for 3 hours. Water was added to quench the reaction and the reaction mixture was extracted with EtOAc. The combined organic fractions were dried over MgSO₄, filtered and the filtrate and concentrated to dryness. The residue was purified by chromatography (silica, with hexanes and ethyl acetate) to afford 2-[2-(tert-butyl-dimethyl-silanyloxy)-ethyl]-6-nitro-4H-benzo[1,4]oxazin-3-one.
C. To a solution of 2-[2-(tert-butyl-dimethyl-silanyloxy)-ethyl]-6-nitro-4H-benzo[1,4]oxazin-3-one (0.137 g, 0.389 mmol) in 4 mL of MeOH was added palladium on charcoal (0.039 g, 0.048 mmol). The reaction mixture was hydrogenated under balloon pressure of H₂(g) for 1 hour. The catalyst was removed by filtration, and the filtrate was concentrated under reduced pressure to provide 6-amino-2-[2-(tert-butyl-dimethyl-silanyloxy)-ethyl]-4H-benzo[1,4]oxazin-3-one as brown solid.
D. To a solution of 1-(3,4,5-trifluoro-phenyl)-piperidine-4-carboxylic acid, prepared in Example 168D, (0.056 g, 0.216 mmol) in 4 mL of CH₂Cl₂ was added oxalyl chloride (0.04 mL, 0.432 mmol) and a catalytic amount of DMF. The reaction mixture was stirred for 2 hours. The reaction mixture was evaporated to dryness under reduced pressure. The residue was redissolved in 4 mL of CH₂Cl₂. 6-Amino-2-[2-(tert-butyl-dimethyl-silanyloxy)-ethyl]-4H-benzo[1,4]oxazin-3-one obtained in step C (0.070 g, 0.216 mmol) and triethylamine (0.06 mL, 0.432 mmol) were added sequentially to the acyl chloride solution at 0° C. The reaction mixture was warmed to 25° C. and stirred for 20 min. The reaction mixture was concentrated, and the residue was purified by chromatography (silica with hexanes and ethyl acetate) to afford 1-(3,4,5-trifluoro-phenyl)-piperidine-4-carboxylic acid {2-[2-(tert-butyl-dimethyl-silanyloxy)-ethyl]-3-oxo-3,4-dihydro-2H-benzo[1,4]oxazin-6-yl}-amide as white solid.
E. To a solution of 1-(3,4,5-trifluoro-phenyl)-piperidine-4-carboxylic acid {2-[2-(tert-butyl-dimethyl-silanyloxy)-ethyl]-3-oxo-3,4-dihydro-2H-benzo[1,4]oxazin-6-yl}-amide (0.046 g, 0.0816 mmol) in 3 mL of THF was added a solution of 1M tetrabutylammonium fluoride in THF (0.05 mL, 0.0898 mmol) at 0° C. The reaction mixture was warmed to 25° C. and stirred for 1 hour. The reaction mixture was evaporated to dryness, and the residue was purified by chromatography (silica with hexanes and ethyl acetate) to afford 1-(3,4,5-rifluoro-phenyl)-piperidine-4-carboxylic acid [2-(2-hydroxy-ethyl)-3-oxo-3,4-dihydro-2H-benzo[1,4]oxazin-6-yl]-amide as white solid. ¹H NMR (400 MHz, CD₃OD) δ (ppm): 7.37 (d, 1H), 6.99 (dd, 1H), 6.90 (d, 1H), 6.82 (dd, 2H), 4.65 (dd, 1H), 3.77 (m, 4H), 2.77 (td, 2H), 2.52-2.46 (m, 1H), 2.14 (m, 1H), 2.01-1.81 (m, 5H). Mass Spectrum (LCMS, ESI pos.) Calcd. for C₂₂H₂₂F₃N₃O₄: 499.4. Found: 450.2 (M⁺+1).

### Examples 175 and 176

### 2-Methyl-3-oxo-6-{[1-(3,4,5-trifluoro-phenyl)-piperidine-4-carbonyl]-amino}-3,4-dihydro-2H-benzo[1,4]oxazine-2-carboxylic acid ethyl ester (175) and 2-Methyl-3-oxo-6-{[1-(3,4,5-trifluoro-phenyl)-piperidine-4-carbonyl]-amino}-3,4-dihydro-2H-benzo[1,4]oxazine-2-carboxylic acid (176)

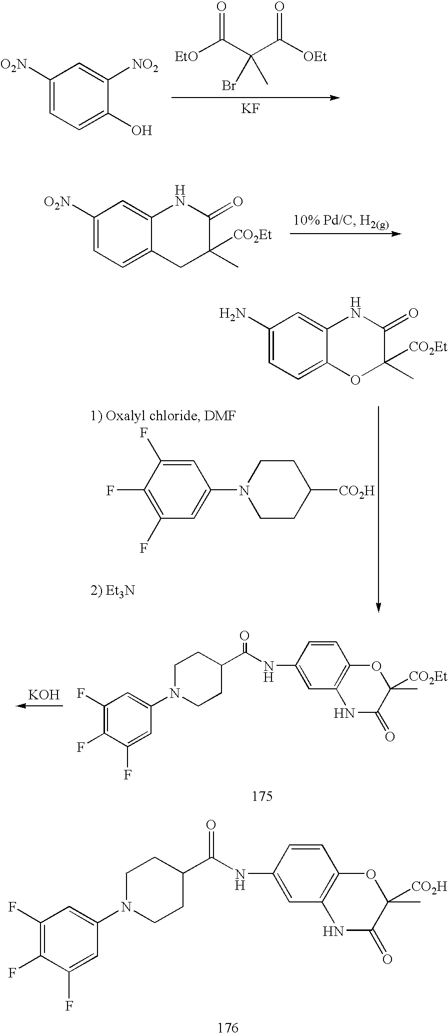 [0576] A. To a solution of 2-amino-5-nitrophenol (6.00 g, 0.0326 mol) in 80 mL of DMF was added diethyl 2-bromo-2-methyl malonate (6.2 mL, 0.0326 mol) and potassium fluoride (4.74 g, 0.0815 mol). The reaction mixture was heated at 60° C. for 6 hours. Water was added to quench the reaction and the reaction mixture was extracted with EtOAc. The combined organic fractions were dried over MgSO₄, filtered and the filtrate was concentrated to dryness. The residue was purified by chromatography (silica with hexanes and ethyl acetate) to afford 2-methyl-6-nitro-3-oxo-3,4-dihydro-2H-benzo[1,4]oxazine-2-carboxylic acid ethyl ester.
B. To a solution of 2-methyl-6-nitro-3-oxo-3,4-dihydro-2H-benzo[1,4]oxazine-2-carboxylic acid ethyl ester (0.182 g, 0.649 mmol) in 4 mL of MeOH was added palladium on charcoal (0.069 g, 0.0649 mmol). The reaction mixture was hydrogenated under balloon pressure of H₂(g) for 1 hour. The catalyst was removed by filtration, and the filtrate was concentrated under reduced pressure to provide 6-amino-2-methyl-3-oxo-3,4-dihydro-2H-benzo[1,4]oxazine-2-carboxylic acid ethyl ester as brown solid.
C. To a solution of 1-(3,4,5-trifluoro-phenyl)-piperidine-4-carboxylic acid, prepared in Example 168D, (0.052 g, 0.201 mmol) in 4 mL of CH₂Cl₂ was added oxalyl chloride (0.04 mL, 0.402 mmol) and a catalytic amount of DMF. The reaction mixture was stirred for 2 hours, and the reaction mixture was concentrated under reduced pressure. The residue was redissolved in 4 mL of CH₂Cl₂. 6-Amino-2-methyl-3-oxo-3,4-dihydro-2H-benzo[1,4]oxazine-2-carboxylic acid ethyl ester obtained in step B (0.050 g, 0.201 mmol) and triethylamine (0.06 mL, 0.402 mmol) were added sequentially to the acyl chloride solution at 0° C. The reaction mixture was warmed to 25° C. and stirred for 20 min. The reaction mixture was concentrated, and the residue was purified by chromatography (silica with hexanes and ethyl acetate) to afford 2-methyl-3-oxo-6-{[1-(3,4,5-trifluoro-phenyl)-piperidine-4-carbonyl]-amino}-3,4-dihydro-2H-benzo[1,4]oxazine-2-carboxylic acid ethyl ester as white solid. ¹H NMR (400 MHz, CD₃OD) δ (ppm): 7.40 (d, 1H), 7.00 (dd, 1H), 6.94 (d, 1H), 6.55 (dd, 2H), 4.12 (qd, 2H), 3.30 (d, 2H), 2.76 (td, 2H), 2.52-2.45 (m, 1H), 1.94-1.80 (m, 4H), 1.73 (s, 3H), 1.13 (t, 3H). Mass Spectrum (LCMS, ESI pos.) Calcd. for C₂₄H₂₄F₃N₃O₅: 491.5. Found: 492.2 (M⁺+1).
D. A mixture of 2-methyl-3-oxo-6-{[1-(3,4,5-trifluoro-phenyl)-piperidine-4-carbonyl]-amino}-3,4-dihydro-2H-benzo[1,4]oxazine-2-carboxylic acid ethyl ester (0.011 g, 0.0223 mmol) and 1 mL of potassium hydroxide solution, prepared by mixing KOH (1.98 g), MeOH (46 mL), and H₂O (6 mL), was stirred for 2 hours. The reaction mixture was acidified to pH 1-2 and then extracted with EtOAc. The combined organic fractions were washed with brine, dried over MgSO₄, filtered and the filtrate was concentrated to provide 2-methyl-3-oxo-6-{[1-(3,4,5-trifluoro-phenyl)-piperidine-4-carbonyl]-amino}-3,4-dihydro-2H-benzo[1,4]oxazine-2-carboxylic acid as white solid. ¹H NMR (400 MHz, CD₃OD) δ (ppm): 7.40 (dd, 2H), 7.38 (d, 1H), 7.05 (dd, 1H), 6.96 (d, 1H), 3.82 (d, 2H), 3.44 (td, 2H), 2.81-2.73 (m, 1H), 2.19-2.13 (m, 4H), 1.75 (s, 3H). Mass Spectrum (LCMS, ESI pos.) Calcd. for C₂₂H₂₀F₃N₃O₅: 463.4. Found: 464.2 (M⁺+1).

### Examples 177 and 178

### 3-Oxo-6-{[1-(3,4,5-trifluoro-phenyl)-piperidine-4-carbonyl]-amino}-3,4-dihydro-2H-benzo[1,4]oxazine-2-carboxylic acid ethyl ester (177) and 3-Oxo-6-{[1-(3,4,5-trifluoro-phenyl)-piperidine-4-carbonyl]-amino}-3,4-dihydro-2H-benzo[1,4]oxazine-2-carboxylic acid (178)

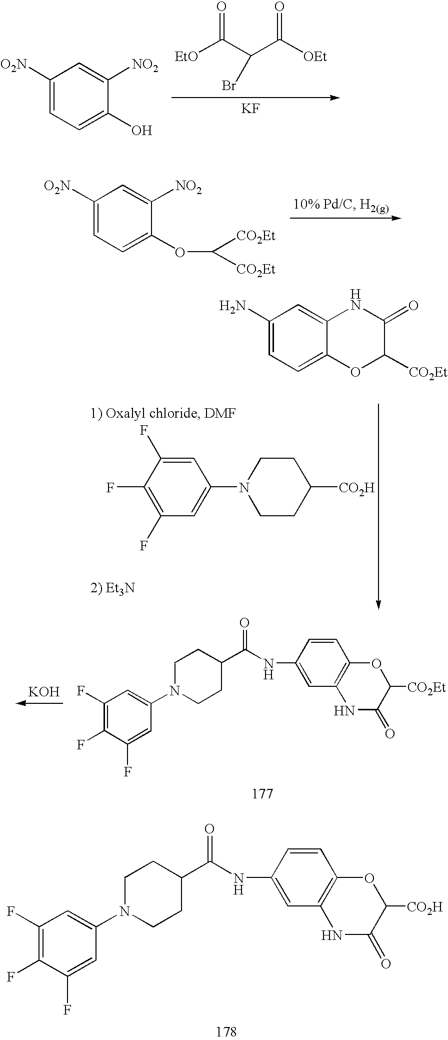 [0581] A. To a solution of diethyl 2-bromomalonate (9.3 mL, 0.0543 mol) in 40 mL of DMF was added potassium fluoride (7.89 g, 0.136 mol). After stirring for an hour, 2,4-dinitrophenol (10.00 g, 0.0543 mol) was added. The reaction mixture was heated at 70° C. for 30 hours. Water was added to quench the reaction and the reaction mixture was extracted with EtOAc. The combined organic fractions were dried over MgSO₄, filtered and the filtrate was concentrated to dryness. The residue was purified by chromatography (silica with hexanes and ethyl acetate) to afford 2-(2,4-dinitro-phenoxy)-malonic acid diethyl ester.
B. To a solution of 2-(2,4-dinitro-phenoxy)-malonic acid diethyl ester (0.346 g, 1.01 mmol) in 8 mL of MeOH was added palladium on charcoal (0.107 g, 0.101 mmol). The reaction mixture was hydrogenated under balloon pressure of H₂(g) at 60° C. for 48 hours. The catalyst was filtered off and the filtrate was concentrated under reduced pressure to provide 6-amino-3-oxo-3,4-dihydro-2H-benzo[1,4]oxazine-2-carboxylic acid ethyl ester as brown solid.
C. To a solution of 1-(3,4,5-trifluoro-phenyl)-piperidine-4-carboxylic acid, prepared in Example 168D, (0.057 g, 0.220 mmol) in 4 mL of CH₂Cl₂ was added oxalyl chloride (0.04 mL, 0.442 mmol) and a catalytic amount of DMF. The reaction mixture was stirred for 2 hours. The reaction mixture was evaporated to dryness under reduced pressure. The residue was redissolved in 4 mL of CH₂Cl₂. 6-Amino-3-oxo-3,4-dihydro-2H-benzo[1,4]oxazine-2-carboxylic acid ethyl ester obtained in step B (0.052 g, 0.220 mmol) and triethylamine (0.06 mL, 0.440 mmol) were added sequentially to the acyl chloride solution at 0° C. The reaction mixture was warmed to 25° C. and stirred for 20 min. The reaction mixture was concentrated, and the residue was purified by chromatography (silica with hexanes and ethyl acetate) to afford 3-oxo-6-{[1-(3,4,5-trifluoro-phenyl)-piperidine-4-carbonyl]-amino}-3,4-dihydro-2H-benzo[1,4]oxazine-2-carboxylic acid ethyl ester as white solid. ¹H NMR (400 MHz, CD₃OD) δ (ppm): 7.40 (d, 1H), 7.01 (dd, 1H), 6.82 (d, 1H), 6.68 (dd, 2H), 5.22 (s, 1H), 4.20 (q, 2H), 3.74 (d, 2H), 2.77 (td, 2H), 2.52-2.45 (m, 1H), 1.91-1.82 (m, 4H), 1.23 (s, 3H). Mass Spectrum (LCMS, ESI pos.) Calcd. for C₂₃H₂₂F₃N₃O₅: 477.4. Found: 478.2 (M⁺+1).
D. A mixture of 3-oxo-6-{[1-(3,4,5-trifluoro-phenyl)-piperidine-4-carbonyl]-amino}-3,4-dihydro-2H-benzo[1,4]oxazine-2-carboxylic acid ethyl ester (0.013 g, 0.0272 mmol) and 1 mL of potassium hydroxide solution, prepared by mixing KOH (1.98 g), MeOH (46 mL), and H₂O (6 mL), was stirred for 2 hours. The reaction mixture was acidified to pH 1-2 and extracted with EtOAc. The combined organic fractions were washed with brine, dried over MgSO₄, filtered and the filtrate was concentrated to provide 3-oxo-6-{[1-(3,4,5-trifluoro-phenyl)-piperidine-4-carbonyl]-amino}-3,4-dihydro-2H-benzo[1,4]oxazine-2-carboxylic acid as white solid. ¹H NMR (400 MHz, CD₃OD) δ (ppm): 7.43 (dd, 2H), 7.39 (d, 1H), 7.06 (dd, 1H), 6.98 (d, 1H), 5.18 (s, 1H), 3.83 (d, 2H), 3.49-3.43 (m, 2H), 2.78-2.73 (m, 1H), 2.22-2.02 (m, 4H). Mass Spectrum (LCMS, ESI pos.) Calcd. for C₂₁H₁₈F₃N₃O₅: 449.4. Found: 450.2 (M⁺+1).

### Examples 179 and 180

### (3-Oxo-6-{[1-(3,4,5-trifluoro-phenyl)-piperidine-4-carbonyl]-amino}-3,4-dihydro-2H-benzo[1,4]oxazin-2-yl)-acetic acid methyl ester (179) and (3-Oxo-6-{[1-(3,4,5-trifluoro-phenyl)-piperidine-4-carbonyl]-amino}-3,4-dihydro-2H-benzo[1,4]oxazin-2-yl)-acetic acid (180)

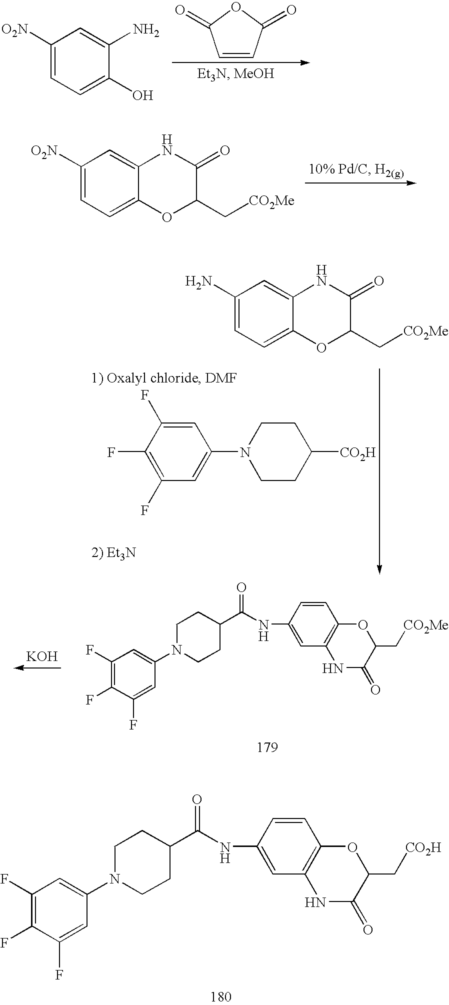 [0586] A. To a solution of 2-amino-5-nitrophenol (4.0 g, 0760 mol) in 180 mL of MeOH was added maleic anhydride (7.5 g, 0.0760 mol) and triethylamine (10.6 mL, 0.760 mol). The reaction mixture was heated at reflux for 5 hours. The reaction mixture was concentrated, and the residue was partitioned between EtOAc and water. The aqueous layer was extracted by EtOAc. The combined organic fractions were dried over MgSO₄, filtered and the filtrate was concentrated to dryness. The residue was purified by chromatography (silica with hexanes and ethyl acetate) to afford (6-nitro-3-oxo-3,4-dihydro-2H-benzo[1,4]oxazin-2-yl)-acetic acid methyl ester.
B. To a solution of (6-nitro-3-oxo-3,4-dihydro-2H-benzo[1,4]oxazin-2-yl)-acetic acid methyl ester (0.294 g, 1.10 mmol) in 8 mL of MeOH was added palladium on charcoal (0.117 g, 0.110 mmol). The reaction mixture was hydrogenated under balloon pressure of H₂(g) for 1 hour. The catalyst was removed by filtration, and the filtrate was concentrated under reduced pressure to provide (6-amino-3-oxo-3,4-dihydro-2H-benzo[1,4]oxazin-2-yl)-acetic acid methyl ester as brown solid.
C. To a solution of 1-(3,4,5-trifluoro-phenyl)-piperidine-4-carboxylic acid, prepared in Example 168D, (0.055 g, 0.212 mmol) in 4 mL of CH₂Cl₂ was added oxalyl chloride (0.04 mL, 0.424 mmol) and a catalytic amount of DMF. The reaction mixture was stirred for 2 hours. The reaction mixture was then evaporated to dryness under reduced pressure. The residue was redissolved in 4 mL of CH₂Cl₂. (6-Amino-3-oxo-3,4-dihydro-2H-benzo[1,4]oxazin-2-yl)-acetic acid methyl ester obtained in step B (0.050 g, 0.212 mmol) and triethylamine (0.06 mL, 0.424 mmol) were added sequentially to the acyl chloride solution at 0° C. The reaction mixture was warmed to 25° C. and stirred for 20 min. The reaction mixture was concentrated, and the residue was purified by chromatography (silica with hexanes and ethyl acetate) to afford (3-oxo-6-{[1-(3,4,5-trifluoro-phenyl)-piperidine-4-carbonyl]-amino}-3,4-dihydro-2H-benzo[1,4]oxazin-2-yl)-acetic acid methyl ester as white solid. ¹H NMR (400 MHz, CD₃OD) δ (ppm): 7.39 (d, 1H), 6.98 (dd, 1H), 6.87 (d, 1H), 6.68 (dd, 2H), 3.74 (d, 2H), 3.71 (s, 3H), 3.06-2.93 (m, 2H), 2.77 (td, 2H), 2.53-2.46 (m, 1H), 1.93-1.78 (m, 4H). Mass Spectrum (LCMS, ESI pos.) Calcd. for C₂₃H₂₂F₃N₃O₅: 477.4. Found: 478.2 (M⁺+1).
D. A mixture of 3-oxo-6-{[1-(3,4,5-trifluoro-phenyl)-piperidine-4-carbonyl]-amino}-3,4-dihydro-2H-benzo[1,4]oxazine-2-carboxylic acid ethyl ester (0.014 g, 0.0293 mmol) and 1 mL of potassium hydroxide solution, prepared by mixing KOH (1.98 g), MeOH (46 mL), and H₂O (6 mL), was stirred for 2 hours. The reaction mixture was acidified to pH 1-2 and then extracted with EtOAc. The combined organic fractions were washed with brine, dried over MgSO₄, filtered and the filtrate wass concentrated to provide (3-oxo-6-{[1-(3,4,5-trifluoro-phenyl)-piperidine-4-carbonyl]-amino}-3,4-dihydro-2H-benzo[1,4]oxazin-2-yl)-acetic acid as white solid. ¹H NMR (400 MHz, CD₃OD) δ (ppm): 7.73 (d, 1H), 7.21 (dd, 1H), 7.05 (d, 1H), 6.74 (dd, 2H), 3.52 (d, 2H), 2.65 (td, 2H), 2.36-2.25 (m, 1H), 1.89-1.76 (m, 4H). Mass Spectrum (LCMS, ESI pos.) Calcd. for C₂₂H₂₀F₃N₃O₅: 463.4. Found: 464.2 (M⁺+1).

### BIOLOGICAL EXAMPLES

### Example 1

### Human VR1 Binding Assay

Compounds of the present invention were tested for their ability to inhibit the binding of [³H] RTX to hVR1 receptors in a [³H] RTX binding assay as previously described (Zhang, Sui-Po. Improved ligand binding assays for vanilloid receptors. PCT Int. Appl. (2002), WO 0233411 A1 20020425 AN 2002:315209, and Grant, Elfrida R., Dubin, Adrienne E., Zhang, Sui-Po, Zivin, Robert A., Zhong, Zhong Simultaneous intracellular calcium and sodium flux imaging in human vanilloid receptor 1 (VR1)-transfected human embryonic kidney cells: a method to resolve ionic dependence of VR1-mediated cell death. J. Pharmacol. Exp. Ther., 2002, 300(1), 9-17.)

HEK293 cells were transfected with human VR1 vanilloid receptors and washed with Hank's balanced Salt Solution, dissociated with cell dissociation buffer (Sigma), and then centrifuged at 1000×g for 5 min. Cell pellets were homogenized in cold 20 mM HEPES buffer, pH 7.4, containing 5.8 mM NaCl, 320 mM sucrose, 2 mM MgCl₂, 0.75 CaCl₂ and 5 mM KCl and centrifuged at 1000×g for 15 min. The resultant supernate was then centrifuged at 40000×g for 15 min. The pelleted membranes were stored in a freezer at −80° C.

Approximately 120 μg protein/ml from membranes were incubated with indicated concentrations of [³H]RTX in 0.5 ml of the HEPES buffer (pH 7.4) containing 0.25 mg/mL fatty acid-free bovine serum albumin at 37° C. for 60 min. The reaction mixture was then cooled to 4° C., added 0.1 mg α₁-acid glycoprotein to each sample and incubated at 4° C. for 15 min. The samples were centrifuged at 18500×g for 15 min. The tip of the microcentrifuge tube containing the pellet was cut off. Bound radioactivity was quantified by scintillation counting. Non-specific binding was tested in the presence of 200 nM unlabeled RTX.

Alternatively, a binding assay using rat tissue was used. Rat spinal cord was homogenized twice with a Polytron and centrifuged at 3000 rpm for 10 min in HEPES buffer containing 20 mM HEPES, pH 7.4, NaCl 5.8 mM, sucrose 320 mM, MgCl₂ 2 mM, CaCl₂ 0.75 mM and KCl 5 mM. The supernatant was then centrifuged at 40000×g for 15 min. The pellet was saved in a tube and 10 ml assay buffer was added into the tube. The pellet and buffer were mixed with a Polytron. The assay contained 120 μg/ml membrane protein and 0.3-0.6 nM [³H]-RTX (PerkinElmer, Boston) in a total volume of 0.5 ml HEPES buffer. Following incubation for 60 min at 37° C., the samples were cooled on ice, and 100 mg of α-acid glycoprotein were added into the samples. After centrifugation at 18500×g for 15 min, the supernatant was aspirated and the tips of tubes were cut off and placed into 6 ml vials.

Data were calculated according to the equation:
% inhibition=(total binding−binding)*100/(total binding−non specific binding).
Kᵢ values were calculated using a Prism program.

### Example 2

### Protocol 1

### Human VR1 Functional Assay Using FLIPR™

The functional activity of the test compounds was determined by measuring changes in intracellular calcium concentration using FLIPR™ Calcium-3 Assay Kit and FLIPR™ technology.

HEK 293 cells transfected with Human Vanilloid Receptor 1 were maintained at 37° C. and 5% CO₂ in: Dulbecco's Modification of Eagle's Medium (high glucose, L-glutamine, pyridoxine HCl, No sodium pyruvate), 10% FBS, 1× Penicillin-Streptomycin-Amphotericin B, 200 ug/mL zeocin. Approximately 24 hours prior to assay, 30,000 cells/well were plated onto a 384-well black clear bottom poly-D-lysine-coated assay plate with a well volume of 25 □L using Titertek Multidrop.

On the day of the assay, the medium was dumped and 50 μL 1× dye were loaded using Multidrop. Plates were incubated with dye at 37° C. for 30 minutes followed by 30 minutes incubation at room temperature. Then the plates were assayed by FLIPR™ with 2 additions: 12.5 μL test compound with 5 minutes data acquisition followed by 12.5 μL capsaicin at the EC₈₀ concentration.

The EC₅₀ values of test compounds were calculated based on a test compound-induced calcium response at the 5-minute time point. The IC₅₀ values of compounds were calculated based on a decreased capsaicin-induced calcium response in the presence of test compound. These values are tabulated in Table 1, below.

TABLE 1

| | | | % maximal | | | |
|---|---|---|---|---|---|---|
| | | | reponse to | | h Binding | h |
| Ex- | h % I | h IC50 | capsaicin | h EC50 | % I | Binding |
| ample | (9 uM) | (uM) | (12.2 uM) | (uM) | (1 uM) | Ki (uM) |
| 1 | 7.0 | | | | | |
| 2 | 109.0 | 0.60 | | | | |
| 3 | 44.0 | 15.52 | | | | |
| 4 | 7.5 | | | | | |
| 5 | 102.5 | 0.23 | | | | |
| 6 | 92.2 | 0.35 | | | 66.5 | 0.215 |
| 7 | 5.0 | | | | | |
| 8 | 101.0 | 0.23 | | | 87.0 | 0.134 |
| 9 | 48.5 | 5.61 | | | 4.8 | |
| 10 | | | 81.0 | 0.86 | | |
| 11 | 72.0 | 2.84 | | | | |
| 12 | | | 74.0 | 0.86 | | |
| 13 | 88.0 | 0.48 | | | 47.7 | |
| 14 | 4.0 | | | | | |
| 15 | 61.0 | 4.57 | | | | |
| 16 | 83.0 | 0.80 | | | 15.0 | |
| 17 | | | 49.0 | 0.47 | | |
| 18 | 12.5 | | | | | |
| 19 | 7.5 | | | | | |
| 20 | 2.5 | | | | | |
| 21 | 102.0 | 0.16 | | | 86.9 | 0.134 |
| 22 | 78.0 | 1.07 | | | | |
| 23 | 19.0 | | | | | |
| 24 | | | 68.5 | 1.50 | | |
| 25 | | | 74.0 | 0.85 | | |
| 26 | | | 35.5 | 2.55 | | |
| 27 | 13.0 | | | | | |
| 28 | 77.0 | 2.18 | | | | |
| 29 | 13.0 | | | | | |
| 30 | | | 40.0 | 5.31 | | |
| 31 | 16.0 | | | | | |
| 32 | 13.0 | | | | | |
| 33 | 12.6 | | | | 12.8 | |
| 34 | 94.5 | 0.21 | | | | 0.065 |
| 35 | 14.0 | | | | | |
| 36 | 14.0 | | | | | |
| 37 | 46.5 | 2.64 | | | | |
| 38 | | | 62.8 | 0.32 | | |
| 39 | 3.5 | | | | | |
| 40 | 11.5 | | | | | |
| 41 | 12.0 | | | | | |
| 42 | 11.0 | | | | | |
| 43 | 10.0 | | | | | |
| 44 | 33.0 | | | | | |
| 45 | 5.0 | | | | | |
| 46 | 61.4 | 1.20 | | | | |
| 47 | 12.0 | | | | | |
| 48 | 9.5 | | | | | |
| 49 | | | 81.0 | 0.86 | | |
| 50 | | | 74.0 | 0.86 | | |
| 51 | 8.0 | | | | | |
| 52 | 72.0 | 2.84 | | | | |
| 53 | 104.0 | 0.16 | | | 47.3 | |
| 54 | 16.0 | | | | | |
| 139 | 85.0 | 1.81 | | | | |
| 140 | 84.0 | 0.49 | | | | 0.225 |
| 141 | 100.0 | 0.15 | | | | 0.061 |
| 142 | 96.3 | 0.15 | | | | 0.125 |
| 143 | 100.7 | 0.18 | | | | 0.078 |
| 144 | 77.0 | 0.38 | | | | 0.314 |
| 145 | 99.5 | 0.06 | | | | 0.027 |
| 146 | 111.5 | 0.09 | | | | 0.132 |
| 147 | 90.0 | 0.52 | | | | 0.084 |
| 148 | 88.7 | 0.19 | | | | 0.034 |
| 149 | 91.3 | 0.33 | | | | |
| 150 | 79.3 | 1.42 | | | | |
| 151 | 62.3 | 0.67 | | | | 1.47 |
| 152 | 90.7 | 0.22 | | | | 0.093 |
| 153 | 91.7 | 0.16 | | | | 0.071 |
| 154 | 96.0 | 0.37 | | | | 0.137 |
| 155 | 89.0 | 0.85 | | | | 0.056 |
| 156 | 93.3 | 0.06 | | | | |
| 157 | 90.3 | 0.10 | | | | |
| 158 | 39.0 | | | | | |
| 159 | 20.7 | | | | | |
| 160 | 93.0 | 0.13 | | | | |
| 161 | 28.7 | | | | | |
| 162 | 37.0 | | | | | |
| 163 | 19.7 | | | | | |
| 164 | 87.0 | 0.11 | | | | |
| 165 | 94.8 | 0.38 | | | | |
| 166 | 100.2 | 0.29 | | | | |
| 167 | 45.7 | | | | | |
| 168 | 59.6 | | | | | |

### Example 2

### Protocol 2

### Human VR1 Functional Assay Using FLIPR™

The functional activity of the test compounds was determined by measuring changes in intracellular calcium concentration using FLIPR™ Calcium-3 Assay Kit and FLIPR™ technology.

HEK 293 cells stably transfected with Human Vanilloid Receptor 1 were maintained at 37° C. and 5% CO₂ in: Dulbecco's Modification of Eagle's Medium (4.5 g/L glucose, L-glutamine, sodium pyruvate), 10% FBS, 1× Penicillin-Streptomycin, 400 ug/mL G418 Sulfate. Approximately 24 hours prior to assay, 5,000 cells/well were plated onto a 384-well black clear bottom poly-D-lysine-coated assay plate with a well volume of 25 μL using Titertek Multidrop.

On the day of the assay, the medium was dumped and 50 μL of dye (bulk kit vials diluted with 200 mL buffer) was loaded using Multidrop. Plates were incubated with dye at 37° C. for 30 minutes followed by 30 minutes incubation at room temperature. Then the plates were assayed by FLIPR™ with 2 additions: 12.5 μL test compound with 5 minutes data acquisition followed by 12.5 μL capsaicin at the EC₈₀ concentration.

The EC₅₀ values of test compounds were calculated based on a test compound-induced calcium response at the 5-minute time point. The IC₅₀ values of compounds were calculated based on a decreased capsaicin-induced calcium response in the presence of test compound. These values are tabulated in Table 2, below.

TABLE 2

| | | | | | | |
|---|---|---|---|---|---|---|
| | | | % E80 | | h Binding | h Binding |
| Ex- | h % I | h IC50 | Capsaicin | h EC50 | % I | Ki |
| ample | (1 uM) | (uM) | (1.2 uM) | (uM) | (1 uM) | (uM) |

| | | | |
|---|---|---|---|
| 169 | 9 | | |
| 170 | 55 | 0.735 | |
| 171 | | | 17 |
| 172 | | | 39 |
| 173 | 37 | 1.959 | |
| 174 | 99 | 0.0048 | |
| 175 | 54 | 0.873 | |
| 176 | | | 116 |
| 177 | | | 13 |
| 178 | | | 80 |
| 179 | 54 | 0.89 | |
| 180 | 5 | | |

1. A compound of formula (II) 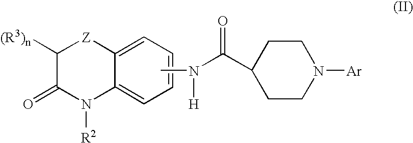
wherein
Ar is an aryl selected from the group consisting of benzo[b]thiophenyl, naphthyl, biphenyl, isoquinolinyl, thiophenyl, pyridazinyl, and benzothiazolyl;
Z is O or S;
R² is hydrogen or C₁₋₆alkyl optionally substituted with —OR⁴;
n is 1 or 2;
R³ is independently hydrogen, C₁₋₆alkyl, —COOR⁴, or —CH₂COOR⁴;
R⁴ is hydrogen or C₁₋₃alkyl; and
enantiomers, diastereomers, tautomers, solvates, and pharmaceutically acceptable salts thereof.

2. A compound according to claim 1 wherein Z is O and n is 1.

3. A compound according to claim 1 wherein Z is O and R² is hydrogen.

4. A compound of formula (III) 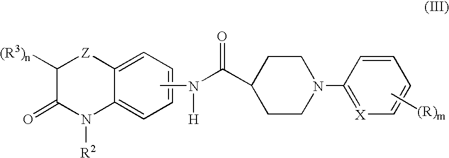
wherein
X is CH or N;
m is an integer from 0 to 4;
R is independently selected from the group consisting of halogen; R¹; fluorinated C₁₋₁₀alkyl; phenyl; amino; cyano; CF₃O—; a 3 membered cyclic heteroalkyl containing 1 heteroatom that is N, O or S wherein said 3 membered cyclic heteroalkyl is optionally substituted with a substituent that is halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a 4 to 5 membered cyclic heteroalkyl containing 1-3 heteroatoms that independently are N, O or S wherein said 4 to 5 membered cyclic heteroalkyl is optionally substituted with 1 to 2 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a 6 to 7 membered cyclic heteroalkyl containing 1-3 heteroatoms that independently are N, O or S wherein said 6 to 7 membered cyclic heteroalkyl is optionally substituted with 1 to 3 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a heteroaryl wherein said heteroaryl is cinnoline, furan, imidazole, indazole, indole, indoline, indolizine, isobenzofuran, isoindole, isoindoline, isoquinoline, isothiazole, isoxazole, naphthyridine, oxadiazole, oxazole, pthalazine, pteridine, pyran, pyrazine, pyrazole, pyridazine, pyridine, pyrimidine, pyrrole, pyrrolizine, quinoline, quinolizine, quinazoline, quinoxaline, tetrazole, thiadiazole, triazine, or triazole wherein said heteroaryl is optionally substituted with 1 to 3 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; hydroxyl; R¹O—; R¹S—; R¹SO₂—; R¹S(O)—; R¹SO₂NH—; -LCOY; and C₆₋₁₀aryl;
R¹ is C₁₋₁₀alkyl;
L is —NH—, a direct bond, —O—, or —CH₂—;
Y is H, R¹, HO, R¹O—, R¹S—, —NH₂, R¹NH—, or (R¹)₂N—;
Z is O or S;
R² is hydrogen or C₁₋₆alkyl optionally substituted with —OR⁴;
n is 1 or 2;
R³ is independently hydrogen, C₁₋₆alkyl, —COOR⁴, or —CH₂COOR⁴;
R⁴ is hydrogen or C₁₋₃alkyl; and
enantiomers, diastereomers, tautomers, solvates, and pharmaceutically acceptable salts thereof.

5. A compound according to claim 4 wherein Z is O and n is 1.

6. A compound according to claim 4 wherein Z is O and R² is hydrogen.

7. A a compound selected from the group consisting of
1-(3-Trifluoromethyl-phenyl)-piperidine-4-carboxylic acid quinolin-3-ylamide;
1-Biphenyl-3-yl-piperidine-4-carboxylic acid quinolin-3-ylamide;
1-(4-Trifluoromethyl-phenyl)-piperidine-4-carboxylic acid quinolin-3-ylamide;
1-(2,3-Dimethyl-phenyl)-piperidine-4-carboxylic acid quinolin-3-ylamide;
1-(3,4-Dimethyl-phenyl)-piperidine-4-carboxylic acid quinolin-3-ylamide;
1-(3-Trifluoromethyl-phenyl)-piperidine-4-carboxylic acid (9-ethyl-9H-purin-6-yl)-amide;
1-(3-Trifluoromethyl-phenyl)-piperidine-4-carboxylic acid (1H-indol-4-yl)-amide;
1-(3-Trifluoromethyl-phenyl)-piperidine-4-carboxylic acid (1-acetyl-2,3-dihydro-1H-indol-7-yl)-amide;
1-(3-Trifluoromethyl-phenyl)-piperidine-4-carboxylic acid thieno[2,3-d]pyrimidin-4-ylamide;
1-(3-Trifluoromethyl-phenyl)-piperidine-4-carboxylic acid (1H-indazol-7-yl)-amide;
1-(3-Trifluoromethyl-phenyl)-piperidine-4-carboxylic acid (3-methyl-benzo[d]isothiazol-5-yl)-amide;
1-(3-Trifluoromethyl-phenyl)-piperidine-4-carboxylic acid benzothiazol-6-ylamide;
3-{[1-(3-Trifluoromethyl-phenyl)-piperidine-4-carbonyl]-amino}-1H-indole-2-carboxylic acid ethyl ester;
1-(3-Trifluoromethyl-phenyl)-piperidine-4-carboxylic acid benzo[1,3]dioxol-5-ylamide;
1-(3-Trifluoromethyl-phenyl)-piperidine-4-carboxylic acid (1-acetyl-2,3-dihydro-1H-indol-6-yl)-amide;
1-(3-Trifluoromethyl-phenyl)-piperidine-4-carboxylic acid (1-acetyl-2,3-dihydro-1H-indol-7-yl)-amide;
1-(3-Trifluoromethyl-phenyl)-piperidine-4-carboxylic acid (2-oxo-2,3-dihydro-1H-indol-4-yl)-amide;
1-(3-Trifluoromethyl-phenyl)-piperidine-4-carboxylic acid (2,3-dihydro-benzo[1,4]dioxin-6-yl)-amide;
1-(3-Trifluoromethyl-phenyl)-piperidine-4-carboxylic acid (2-methyl-benzothiazol-5-yl)-amide;
1-(3-Trifluoromethyl-phenyl)-piperidine-4-carboxylic acid (5-nitro-benzo[d]isothiazol-3-yl)-amide;
1-(3-Trifluoromethyl-phenyl)-piperidine-4-carboxylic acid (1H-indol-4-yl)-amide;
1-(3-Trifluoromethyl-phenyl)-piperidine-4-carboxylic acid (1H-indol-5-yl)-amide;
1-(3-Trifluoromethyl-phenyl)-piperidine-4-carboxylic acid (1H-indol-6-yl)-amide;
1-(3-Trifluoromethyl-phenyl)-piperidine-4-carboxylic acid (1-methyl-1H-indazol-5-yl)-amide;
1-(3-Trifluoromethyl-phenyl)-piperidine-4-carboxylic acid (2-mercapto-benzothiazol-6-yl)-amide;
1-(3-Trifluoromethyl-phenyl)-piperidine-4-carboxylic acid (8-hydroxy-quinolin-5-yl)-amide;
1-(3-Trifluoromethyl-phenyl)-piperidine-4-carboxylic acid (3,4-dihydro-2H-benzo[b][1,4]dioxepin-7-yl)-amide;
1-(3-Trifluoromethyl-phenyl)-piperidine-4-carboxylic acid (1-acetyl-2,3-dihydro-1H-indol-5-yl)-amide;
1-(3-Trifluoromethyl-phenyl)-piperidine-4-carboxylic acid (1H-indol-7-yl)-amide;
1-(3-Trifluoromethyl-phenyl)-piperidine-4-carboxylic acid (1,4-dioxo-1,2,3,4-tetrahydro-phthalazin-6-yl)-amide;
1-(3-Trifluoromethyl-phenyl)-piperidine-4-carboxylic acid (6-phenoxy-pyridin-3-yl)-amide;
1-(3-Trifluoromethyl-phenyl)-piperidine-4-carboxylic acid (6-acetyl-benzo[1,3]dioxol-5-yl)-amide;
1-(3-Trifluoromethyl-phenyl)-piperidine-4-carboxylic acid [4-(4,6-dimethoxy-pyrimidin-2-yl)-phenyl]-amide;
1-(3-Trifluoromethyl-phenyl)-piperidine-4-carboxylic acid (3-oxo-3,4-dihydro-2H-benzo[1,4]oxazin-6-yl)-amide;
1-(3-Trifluoromethyl-phenyl)-piperidine-4-carboxylic acid (4-dimethylamino-5-phenyl-pyrimidin-2-yl)-amide; and
3,5-Dimethyl-1-(5-{[1-(3-trifluoromethyl-phenyl)-piperidine-4-carbonyl]-amino}-pyridin-2-yl)-1H-pyrazole-4-carboxylic acid ethyl ester.

8. A compound selected from the group consisting of
1-Phenyl-piperidine-4-carboxylic acid quinolin-3-ylamide;
1-Benzothiazol-5-yl-piperidine-4-carboxylic acid quinolin-3-ylamide;
1-Benzothiazol-5-yl-piperidine-4-carboxylic acid (2,3-dihydro-1H-indol-4-yl)-amide;
1-Phenyl-piperidine-4-carboxylic acid (2,3-dihydro-1H-indol-4-yl)-amide;
3,4,5,6-Tetrahydro-2H-[1,3′]bipyridinyl-4-carboxylic acid isoquinolin-1-ylamide;
1-Phenyl-piperidine-4-carboxylic acid isoquinolin-1-ylamide;
1-Phenyl-piperidine-4-carboxylic acid cinnolin-3-ylamide;
1-Benzothiazol-5-yl-piperidine-4-carboxylic acid cinnolin-3-ylamide;
1-Benzothiazol-5-yl-piperidine-4-carboxylic acid phthalazin-5-ylamide;
3,4,5,6-Tetrahydro-2H-[1,4′]bipyridinyl-4-carboxylic acid phthalazin-5-ylamide;
1-Phenyl-piperidine-4-carboxylic acid quinazolin-4-ylamide;
1-Benzothiazol-5-yl-piperidine-4-carboxylic acid quinazolin-4-ylamide;
1-Benzothiazol-5-yl-piperidine-4-carboxylic acid pyridin-3-ylamide;
1-Phenyl-piperidine-4-carboxylic acid benzo[d]isothiazol-3-ylamide;
3,4,5,6-Tetrahydro-2H-[1,2′]bipyridinyl-4-carboxylic acid benzo[d]isothiazol-3-ylamide;
1-Phenyl-piperidine-4-carboxylic acid benzothiazol-2-ylamide;
3,4,5,6-Tetrahydro-2H-[1,3′]bipyridinyl-4-carboxylic acid benzothiazol-2-ylamide;
1-Pyridazin-3-yl-piperidine-4-carboxylic acid phthalazin-1-ylamide;
1-Thiophen-2-yl-piperidine-4-carboxylic acid phthalazin-1-ylamide;
1-Thiophen-2-yl-piperidine-4-carboxylic acid (7H-purin-6-yl)-amide;
1-Pyrimidin-2-yl-piperidine-4-carboxylic acid (7H-purin-6-yl)-amide;
1-Pyrimidin-2-yl-piperidine-4-carboxylic acid cinnolin-4-ylamide;
1-Thiophen-2-yl-piperidine-4-carboxylic acid cinnolin-4-ylamide;
1-Thiophen-2-yl-piperidine-4-carboxylic acid quinoxalin-6-ylamide;
1-Pyridazin-3-yl-piperidine-4-carboxylic acid quinoxalin-6-ylamide;
1-Pyrimidin-2-yl-piperidine-4-carboxylic acid quinoxalin-6-ylamide;
1-Pyrimidin-2-yl-piperidine-4-carboxylic acid pyrazin-2-ylamide;
1-Thiophen-2-yl-piperidine-4-carboxylic acid pyrazin-2-ylamide;
1-Thiophen-2-yl-piperidine-4-carboxylic acid quinazolin-7-ylamide;
1-Pyrimidin-2-yl-piperidine-4-carboxylic acid quinazolin-7-ylamide;
1-Pyrimidin-2-yl-piperidine-4-carboxylic acid pyridazin-3-ylamide;
1-Thiophen-2-yl-piperidine-4-carboxylic acid pyridazin-3-ylamide;
1-Benzo[b]thiophen-3-yl-piperidine-4-carboxylic acid (2-oxo-2,3-dihydro-1H-benzoimidazol-5-yl)-amide;
1-Naphthalen-2-yl-piperidine-4-carboxylic acid (2-oxo-2,3-dihydro-1H-benzoimidazol-5-yl)-amide;
1-Naphthalen-2-yl-piperidine-4-carboxylic acid (3-oxo-3,4-dihydro-2H-benzo[1,4]oxazin-6-yl)-amide;
1-Biphenyl-3-yl-piperidine-4-carboxylic acid (3-oxo-3,4-dihydro-2H-benzo[1,4]oxazin-6-yl)-amide;
1-Biphenyl-3-yl-piperidine-4-carboxylic acid quinazolin-2-ylamide;
1-Quinolin-3-yl-piperidine-4-carboxylic acid quinazolin-2-ylamide;
1-Quinolin-3-yl-piperidine-4-carboxylic acid quinoxalin-2-ylamide;
1-Isoquinolin-5-yl-piperidine-4-carboxylic acid quinoxalin-2-ylamide;
1-Isoquinolin-5-yl-piperidine-4-carboxylic acid (9H-carbazol-3-yl)-amide;
1-Benzo[b]thiophen-3-yl-piperidine-4-carboxylic acid (9H-carbazol-3-yl)-amide;
1-Benzo[b]thiophen-3-yl-piperidine-4-carboxylic acid phenazin-2-ylamide;
1-Naphthalen-2-yl-piperidine-4-carboxylic acid phenazin-2-ylamide;
1-Naphthalen-2-yl-piperidine-4-carboxylic acid thiazol-2-ylamide;
1-Biphenyl-3-yl-piperidine-4-carboxylic acid thiazol-2-ylamide;
1-Biphenyl-3-yl-piperidine-4-carboxylic acid (1H-imidazol-2-yl)-amide;
1-Quinolin-3-yl-piperidine-4-carboxylic acid (1H-imidazol-2-yl)-amide;
1-Quinolin-3-yl-piperidine-4-carboxylic acid (2H-pyrazol-3-yl)-amide;
1-Isoquinolin-5-yl-piperidine-4-carboxylic acid (2H-pyrazol-3-yl)-amide;
1-Isoquinolin-5-yl-piperidine-4-carboxylic acid isoxazol-3-ylamide;
1-Benzo[b]thiophen-3-yl-piperidine-4-carboxylic acid isoxazol-3-ylamide;
1-Benzo[b]thiophen-3-yl-piperidine-4-carboxylic acid (3-methyl-isothiazol-5-yl)-amide;
1-Naphthalen-2-yl-piperidine-4-carboxylic acid (3-methyl-isothiazol-5-yl)-amide;
1-Isoquinolin-5-yl-piperidine-4-carboxylic acid [1,6]naphthyridin-4-ylamide;
1-Benzo[b]thiophen-3-yl-piperidine-4-carboxylic acid [1,6]naphthyridin-4-ylamide;
1-Benzo[b]thiophen-3-yl-piperidine-4-carboxylic acid [1,7]naphthyridin-8-ylamide;
1-Naphthalen-2-yl-piperidine-4-carboxylic acid [1,7]naphthyridin-8-ylamide;
1-Naphthalen-2-yl-piperidine-4-carboxylic acid acridin-9-ylamide;
1-Biphenyl-3-yl-piperidine-4-carboxylic acid acridin-9-ylamide;
1-Biphenyl-3-yl-piperidine-4-carboxylic acid (1-methyl-4,5-dihydro-1H-imidazol-2-yl)-amide;
1-Quinolin-3-yl-piperidine-4-carboxylic acid (1-methyl-4,5-dihydro-1H-imidazol-2-yl)-amide;
1-Quinolin-3-yl-piperidine-4-carboxylic acid (4,5-dihydro-1H-pyrazol-3-yl)-amide;
1-Isoquinolin-5-yl-piperidine-4-carboxylic acid (4,5-dihydro-1H-pyrazol-3-yl)-amide;
1-Isoquinolin-5-yl-piperidine-4-carboxylic acid [1,8]naphthyridin-2-ylamide;
1-Benzo[b]thiophen-3-yl-piperidine-4-carboxylic acid [1,8]naphthyridin-2-ylamide;
1-Benzo[b]thiophen-3-yl-piperidine-4-carboxylic acid [2,6]naphthyridin-1-ylamide;
1-Naphthalen-2-yl-piperidine-4-carboxylic acid [2,6]naphthyridin-1-ylamide;
1-Naphthalen-2-yl-piperidine-4-carboxylic acid benzo[d]isoxazol-5-ylamide;
1-Biphenyl-3-yl-piperidine-4-carboxylic acid benzo[d]isoxazol-5-ylamide;
1-Biphenyl-3-yl-piperidine-4-carboxylic acid (1H-pyrrol-3-yl)-amide;
1-Quinolin-3-yl-piperidine-4-carboxylic acid (1H-pyrrol-3-yl)-amide;
1-Quinolin-3-yl-piperidine-4-carboxylic acid (1H-pyrrolo[2,3-b]pyridin-3-yl)-amide;
1-Isoquinolin-5-yl-piperidine-4-carboxylic acid (1H-pyrrolo[2,3-b]pyridin-3-yl)-amide;
1-Isoquinolin-5-yl-piperidine-4-carboxylic acid pteridin-4-ylamide;
1-Benzo[b]thiophen-3-yl-piperidine-4-carboxylic acid pteridin-4-ylamide;
1-Benzo[b]thiophen-3-yl-piperidine-4-carboxylic acid (1,4-dihydro-quinazolin-6-yl)-amide;
1-Naphthalen-2-yl-piperidine-4-carboxylic acid (1,4-dihydro-quinazolin-6-yl)-amide;
1-Naphthalen-2-yl-piperidine-4-carboxylic acid [1,7]naphthyridin-4-ylamide;
1-Biphenyl-3-yl-piperidine-4-carboxylic acid [1,7]naphthyridin-4-ylamide;
1-Biphenyl-3-yl-piperidine-4-carboxylic acid [2,7]naphthyridin-4-ylamide;
1-Quinolin-3-yl-piperidine-4-carboxylic acid [2,7]naphthyridin-4-ylamide;
1-Quinolin-3-yl-piperidine-4-carboxylic acid [2,6]naphthyridin-1-ylamide; and
1-Isoquinolin-5-yl-piperidine-4-carboxylic acid [2,6]naphthyridin-1-ylamide.

9. A compound of formula (IIa) 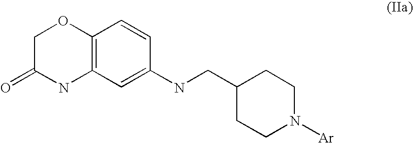
wherein Ar is an aryl selected from the group consisting of benzo[b]thiophenyl, naphthyl, biphenyl, isoquinolinyl, thiophenyl, pyridazinyl, and benzothiazolyl; and
enantiomers, diastereomers, tautomers, solvates, and pharmaceutically acceptable salts thereof.

10. A compound of formula (IIIa): 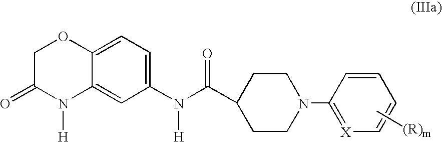
wherein
X is CH or N;
m is an integer from 0 to 4;
R is independently selected from the group consisting of halogen; R¹; fluorinated C₁₋₁₀alkyl; phenyl; amino; cyano; CF₃O—; a 3 membered cyclic heteroalkyl containing 1 heteroatom that is N, O or S wherein said 3 membered cyclic heteroalkyl is optionally substituted with a substituent that is halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a 4 to 5 membered cyclic heteroalkyl containing 1-3 heteroatoms that independently are N, O or S wherein said 4 to 5 membered cyclic heteroalkyl is optionally substituted with 1 to 2 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a 6 to 7 membered cyclic heteroalkyl containing 1-3 heteroatoms that independently are N, O or S wherein said 6 to 7 membered cyclic heteroalkyl is optionally substituted with 1 to 3 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; a heteroaryl wherein said heteroaryl is cinnoline, furan, imidazole, indazole, indole, indoline, indolizine, isobenzofuran, isoindole, isoindoline, isoquinoline, isothiazole, isoxazole, naphthyridine, oxadiazole, oxazole, pthalazine, pteridine, pyran, pyrazine, pyrazole, pyridazine, pyridine, pyrimidine, pyrrole, pyrrolizine, quinoline, quinolizine, quinazoline, quinoxaline, tetrazole, thiadiazole, triazine, or triazole wherein said heteroaryl is optionally substituted with 1 to 3 substituents that independently are halogen, R¹, fluorinated C₁₋₁₀alkyl, amino, cyano, CF₃O—, R¹O—, R¹S—, R¹SO₂—, R¹S(O)—, R¹SO₂NH—, or -LCOY; hydroxyl; R¹O—; R¹S—; R¹SO₂—; R¹S(O)—; R¹SO₂NH—; -LCOY; and C₆₋₁₀aryl;
R¹ is C₁₋₁₀alkyl;
L is —NH—, a direct bond, —O—, or —CH₂—;
Y is H, R¹, HO, R¹O—, R¹S—, —NH₂, R¹NH—, or (R¹)₂N—; and
enantiomers, diastereomers, tautomers, solvates, and pharmaceutically acceptable salts thereof.

11. A compound according to claim 10 wherein X is CH.

12. A compound according to claim 10 wherein m is 1 or 2.

13. A compound according to claim 10 wherein R is independently C₁₋₄alkanyl, CF₃, or halo.

14. A compound according to claim 10 wherein X is CH, m is 1 or 2 and R is independently C₁₋₄alkanyl, CF₃, or halo.

15. A compound selected from the group consisting of
1-(2,3-Dimethyl-phenyl)-piperidine-4-carboxylic acid (3-oxo-3,4-dihydro-2H-benzo[1,4]oxazin-6-yl)-amide;
1-(2,5-Bis-trifluoromethyl-phenyl)-piperidine-4-carboxylic acid (3-oxo-3,4-dihydro-2H-benzo[1,4]oxazin-6-yl)-amide;
1-(2-Bromo-3-fluoro-phenyl)-piperidine-4-carboxylic acid (3-oxo-3,4-dihydro-2H-benzo[1,4]oxazin-6-yl)-amide;
1-(2-Bromo-phenyl)-piperidine-4-carboxylic acid (3-oxo-3,4-dihydro-2H-benzo[1,4]oxazin-6-yl)-amide;
1-(2-Chloro-phenyl)-piperidine-4-carboxylic acid (3-oxo-3,4-dihydro-2H-benzo[1,4]oxazin-6-yl)-amide;
1-(2-Cyano-3-trifluoromethyl-phenyl)-piperidine-4-carboxylic acid (3-oxo-3,4-dihydro-2H-benzo[1,4]oxazin-6-yl)-amide;
1-(2-Cyano-5-trifluoromethyl-phenyl)-piperidine-4-carboxylic acid (3-oxo-3,4-dihydro-2H-benzo[1,4]oxazin-6-yl)-amide;
1-(2-Ethyl-phenyl)-piperidine-4-carboxylic acid (3-oxo-3,4-dihydro-2H-benzo[1,4]oxazin-6-yl)-amide;
1-(2-Fluoro-phenyl)-piperidine-4-carboxylic acid (3-oxo-3,4-dihydro-2H-benzo[1,4]oxazin-6-yl)-amide;
1-(2-Isopropyl-phenyl)-piperidine-4-carboxylic acid (3-oxo-3,4-dihydro-2H-benzo[1,4]oxazin-6-yl)-amide;
1-(2-Trifluoromethyl-phenyl)-piperidine-4-carboxylic acid (3-oxo-3,4-dihydro-2H-benzo[1,4]oxazin-6-yl)-amide;
1-(3,4,5-Trifluoro-phenyl)-piperidine-4-carboxylic acid (3-oxo-3,4-dihydro-2H-benzo[1,4]oxazin-6-yl)-amide;
1-(3,4-Difluoro-phenyl)-piperidine-4-carboxylic acid (3-oxo-3,4-dihydro-2H-benzo[1,4]oxazin-6-yl)-amide;
1-(3,4-Dimethyl-phenyl)-piperidine-4-carboxylic acid (3-oxo-3,4-dihydro-2H-benzo[1,4]oxazin-6-yl)-amide;
1-(3,5-Difluoro-phenyl)-piperidine-4-carboxylic acid (3-oxo-3,4-dihydro-2H-benzo[1,4]oxazin-6-yl)-amide;
1-(3-Bromo-phenyl)-piperidine-4-carboxylic acid (3-oxo-3,4-dihydro-2H-benzo[1,4]oxazin-6-yl)-amide;
1-(3-Chloro-2-methyl-phenyl)-piperidine-4-carboxylic acid (3-oxo-3,4-dihydro-2H-benzo[1,4]oxazin-6-yl)-amide;
1-(3-Chloro-phenyl)-piperidine-4-carboxylic acid (3-oxo-3,4-dihydro-2H-benzo[1,4]oxazin-6-yl)-amide;
1-(3-Fluoro-phenyl)-piperidine-4-carboxylic acid (3-oxo-3,4-dihydro-2H-benzo[1,4]oxazin-6-yl)-amide;
1-(3-Methoxy-phenyl)-piperidine-4-carboxylic acid (3-oxo-3,4-dihydro-2H-benzo[1,4]oxazin-6-yl)-amide;
1-(3-Trifluoromethyl-phenyl)-piperidine-4-carboxylic acid (2-oxo-2,3-dihydro-1H-pyrido[2,3-b][1,4]oxazin-7-yl)-amide;
1-(3-Trifluoromethyl-phenyl)-piperidine-4-carboxylic acid [4-(2-hydroxy-ethyl)-3-oxo-3,4-dihydro-2H-benzo[1,4]oxazin-6-yl]-amide;
1-(4-Bromo-2,3,5,6-tetrafluoro-phenyl)-piperidine-4-carboxylic acid (3-oxo-3,4-dihydro-2H-benzo[1,4]oxazin-6-yl)-amide;
1-(4-tert-Butyl-phenyl)-piperidine-4-carboxylic acid (3-oxo-3,4-dihydro-2H-benzo[1,4]oxazin-6-yl)-amide;
1-Benzothiazol-2-yl-piperidine-4-carboxylic acid (3-oxo-3,4-dihydro-2H-benzo[1,4]oxazin-6-yl)-amide;
1-m-Tolyl-piperidine-4-carboxylic acid (3-oxo-3,4-dihydro-2H-benzo[1,4]oxazin-6-yl)-amide;
1-o-Tolyl-piperidine-4-carboxylic acid (3-oxo-3,4-dihydro-2H-benzo[1,4]oxazin-6-yl)-amide;
1-Phenyl-piperidine-4-carboxylic acid (3-oxo-3,4-dihydro-2H-benzo[1,4]oxazin-6-yl)-amide;
3′-Methyl-3,4,5,6-tetrahydro-2H-[1,2′]bipyridinyl-4-carboxylic acid (3-oxo-3,4-dihydro-2H-benzo[1,4]oxazin-6-yl)-amide; and
3′-Nitro-3,4,5,6-tetrahydro-2H-[1,2′]bipyridinyl-4-carboxylic acid (3-oxo-3,4-dihydro-2H-benzo[1,4]oxazin-6-yl)-amide.

16. A compound selected from the group consisting of
1-(3,4,5-Trifluoro-phenyl)-piperidine-4-carboxylic acid (3-oxo-3,4-dihydro-2H-benzo[1,4]oxazin-5-yl)-amide;
1-(3,4,5-Trifluoro-phenyl)-piperidine-4-carboxylic acid (3-oxo-3,4-dihydro-2H-benzo[1,4]oxazin-7-yl)-amide;
1-(3,4,5-Trifluoro-phenyl)-piperidine-4-carboxylic acid (3-oxo-3,4-dihydro-2H-benzo[1,4]oxazin-8-yl)-amide;
1-(3,4,5-Trifluoro-phenyl)-piperidine-4-carboxylic acid (4-methyl-3-oxo-3,4-dihydro-2H-benzo[1,4]oxazin-6-yl)-amide;
1-(3,4,5-Trifluoro-phenyl)-piperidine-4-carboxylic acid (3-oxo-3,4-dihydro-2H-benzo[1,4]thiazin-6-yl)-amide;
1-(3,4,5-Trifluoro-phenyl)-piperidine-4-carboxylic acid [2-(2-hydroxy-ethyl)-3-oxo-3,4-dihydro-2H-benzo[1,4]oxazin-6-yl]-amide;
2-Methyl-3-oxo-6-{[1-(3,4,5-trifluoro-phenyl)-piperidine-4-carbonyl]-amino}-3,4-dihydro-2H-benzo[1,4]oxazine-2-carboxylic acid ethyl ester;
2-Methyl-3-oxo-6-{[1-(3,4,5-trifluoro-phenyl)-piperidine-4-carbonyl]-amino}-3,4-dihydro-2H-benzo[1,4]oxazine-2-carboxylic acid;
3-Oxo-6-{[1-(3,4,5-trifluoro-phenyl)-piperidine-4-carbonyl]-amino}-3,4-dihydro-2H-benzo[1,4]oxazine-2-carboxylic acid ethyl ester;
3-Oxo-6-{[1-(3,4,5-trifluoro-phenyl)-piperidine-4-carbonyl]-amino}-3,4-dihydro-2H-benzo[1,4]oxazine-2-carboxylic acid;
(3-Oxo-6-{[1-(3,4,5-trifluoro-phenyl)-piperidine-4-carbonyl]-amino}-3,4-dihydro-2H-benzo[1,4]oxazin-2-yl)-acetic acid methyl ester; and
(3-Oxo-6-{[1-(3,4,5-trifluoro-phenyl)-piperidine-4-carbonyl]-amino}-3,4-dihydro-2H-benzo[1,4]oxazin-2-yl)-acetic acid.

17. A pharmaceutical composition comprising a compound, salt or solvate according to claim 1 admixed with a pharmaceutically acceptable carrier, excipient or diluent.

18. A veterinary composition comprising a compound, salt or solvate according to claim 1 admixed with a veterinarily acceptable carrier, excipient or dilluent.

19. A kit comprising in one or more containers an amount of the composition of claim 1 effective to treat or prevent a disease or condition selected from the group consisting of osteoarthritis, rheumatoid arthritis, fibromyalgia, migraine, headache, toothache, burn, sunburn, snake bite (in particular, venomous snake bite), spider bite, insect sting, neurogenic bladder, benign prostatic hypertrophy, interstitial cystitis, urinary tract infection, cough, asthma, chronic obstructive pulmonary disease, rhinitis, contact dermatitis/hypersensitivity, itch, eczema, anxiety, panic disorders, pharyngitis, mucositis, enteritis, cellulites, peripheral neuropathy, bilateral peripheral neuropathy, diabetic neuropathy, postherpetic neuralgia, trigeminal neuralgia, causalgia, sciatic neuritis, mandibular joint neuralgia, peripheral neuritis, polyneuritis, stump pain, phantom limb pain, bony fractures, post-operative ileus, irritable bowel syndrome, inflammatory bowel diseases such as Crohn's Disease and ulcerative colitis, cholecystitis, pancreatitis, postmastectomy pain syndrome, oral neuropathic pain, Charcot's pain, reflex sympathetic dystrophy, Guillain-Barre syndrome, meralgia paresthetica, burning-mouth syndrome, optic neuritis, postfebrile neuritis, migrating neuritis, segmental neuritis, Gombault's neuritis, neuronitis, cervicobrachial neuralgia, cranial neuralgia, geniculate neuralgia, glossopharyngial neuralgia, migrainous neuralgia, idiopathic neuralgia, intercostals neuralgia, mammary neuralgia, Morton's neuralgia, nasociliary neuralgia, occipital neuralgia, red neuralgia, Sluder's neuralgia, splenopalatine neuralgia, supraorbital neuralgia, vidian neuralgia, sinus headache, tension headache, labor, childbirth, intestinal gas, menstruation, hot flash, cancer, and trauma.
